(19) United States
(12) Patent Application Publication
(10) Pub. No.: US 2021/0030735 A1
(43) Pub. Date: Feb. 4, 2021

(54) PEDIATRIC NIRAPARIB FORMULATIONS AND PEDIATRIC TREATMENT METHODS

(71) Applicant: Tesaro, Inc., Waltham, MA (US)

(72) Inventors: Simon McGurk, Waltham, MA (US); David Lust, Waltham, MA (US); Kevin Johnston, Waltham, MA (US); Duantel Verwijs, Waltham, MA (US); Aaron Nelson, Waltham, MA (US); Clare Medendorp, Waltham, MA (US); Melanie Ronsheim, Waltham, MA (US); John Chaber, Waltham, MA (US); Steve Ruddy, Waltham, MA (US); Katie Poutsiaka, Waltham, MA (US); Danny van Hoorn, Waltham, MA (US); Aileen Dowling, Waltham, MA (US)

(21) Appl. No.: 16/967,351

(22) PCT Filed: Feb. 5, 2019
(86) PCT No.: PCT/US2019/016648

§ 371 Date: Aug. 4, 2020

Related U.S. Application Data
(60) Provisional application No. 62/626,644, filed on Feb. 5, 2018.
(60) Provisional application No. 62/626,646, filed on Feb. 5, 2018.

(51) Int. Cl. A61K 31/454; A61K 39/395; A61K 9/00

(52) U.S. Cl. CPC A61K 31/454; A61K 9/0056; A61K 39/3955

(57) ABSTRACT

The present invention relates to methods of treating cancer in pediatric subjects comprising administration of compound niraparib in a suitable oral dosage form and optionally in combination with a second therapeutic agent such as a PD-1 inhibitor.

The present application claims benefit of U.S. Patent Application No. 62/626,644, filed Feb. 5, 2018, and U.S. Patent Application No. 62/626,646, filed Feb. 5, 2018, each of which is incorporated by reference in its entirety.

### SUMMARY OF THE INVENTION

Niraparib is an orally active and potent poly (ADP-ribose) polymerase, or PARP, inhibitor. Niraparib and pharmaceutically acceptable salts thereof, are disclosed in International Publication No. WO2007/113596 and European Patent No. EP2007733B1; International Publication No. WO2008/084261 and U.S. Pat. No. 8,071,623; and International Publication No. WO2009/087381 and U.S. Pat. No. 8,436,185. Methods of making niraparib and pharmaceutically acceptable salts thereof are disclosed in International Publication Nos. WO2014/088983 and WO2014/088984. Methods to treat cancer with niraparib and pharmaceutically acceptable salts thereof are disclosed in U.S. Provisional Patent Application Nos. 62/356,461 and 62/402,427. The contents of each of the foregoing references are incorporated herein by reference in their entirety.

PARP is a family of proteins involved in many functions in a cell, including DNA repair, gene expression, cell cycle control, intracellular trafficking and energy metabolism. PARP proteins play key roles in single strand break repair through the base excision repair pathway. PARP inhibitors have shown activity as a monotherapy against tumors with existing DNA repair defects, such as BRCA1 and BRCA2, and as a combination therapy when administered together with anti-cancer agents that induce DNA damage.

Despite several advances in treatment of ovarian cancer, most patients eventually relapse, and subsequent responses to additional treatment are often limited in duration. Women with germline BRCA1 or BRCA2 mutations have an increased risk for developing high grade serous ovarian cancer (HGSOC), and their tumors appear to be particularly sensitive to treatment with a PARP inhibitor. In addition, published scientific literature indicates that patients with platinum sensitive HGSOC who do not have germline BRCA1 or BRCA2 mutations may also experience clinical benefit from treatment with a PARP inhibitor.

It is estimated that 5% to 10% of women who are diagnosed with breast cancer, or more than 15,000 women each year, carry a germline mutation in either their BRCA1 or BRCA2 genes. The development of cancer in these women involves the dysfunction of a key DNA repair pathway known as homologous recombination. While cancer cells can maintain viability despite disruption of the homologous recombination pathway, they become particularly vulnerable to chemotherapy if an alternative DNA repair pathway is disrupted. This is known as synthetic lethality a situation where the individual loss of either repair pathway is compatible with cell viability; but the simultaneous loss of both pathways results in cancer cell deaths. Since PARP inhibitors block DNA repair, in the context of cancer cells with the BRCA mutation, PARP inhibition results in synthetic lethality. For this reason, patients with germline mutations in a BRCA gene show marked clinical benefit that follows treatment with a PARP inhibitor.

These principles can apply to treatments of other cancers (e.g., as described herein). In particular, methods described herein can be particularly suitable for the treatment of pediatric patients (e.g., ≥6 months to <18 years of age) who have been diagnosed with cancer (e.g., recurrent solid tumors that exhibit a breast cancer susceptibility gene (BRCA)ness mutational signature). Exemplary cancers include osteosarcoma and certain types of brain tumors.

It has surprisingly been found that the dosage forms (including solid dosage forms) according to the present invention have desirable properties, including for methods of treatment of pediatric subjects as described herein.

In one aspect, the disclosure provides a method of treating cancer, comprising administering to a pediatric subject in need thereof an effective amount of a niraparib (e.g., as described herein).

The exemplary methods described herein can be used to treat a pediatric subject having any type of cancer which is responsive to niraparib, either alone or in combination with one or more further therapeutic agents or treatments (e.g., as described herein).

In embodiments, a pediatric subject is a subject that is a newborn to about 21 years of age (e.g., a subject from the day of their birth to about 21 years of age or to about 18 years of age). In embodiments, a pediatric subject is a subject that is about six months of age to about 21 years of age. In embodiments, a pediatric subject is a subject that is about six months of age to about 21 years of age. In embodiments, a pediatric subject is about six months of age to about 18 years of age, about one year of age to about 18 years of age, about 1 year of age to about 6 years of age, or about 6 years of age to about 18 years of age.

In embodiments, niraparib is administered to a pediatric subject of about six months of age to about 18 years of age.

In embodiments, niraparib is administered to a pediatric subject of about six years of age to about 18 years of age.

In embodiments, niraparib also can be administered in combination with another therapeutic agent or treatment. In embodiments, a pediatric subject is administered niraparib in combination with one or more of surgery, a radiotherapy, a chemotherapy, an immunotherapy, an anti-angiogenic agent, or an anti-inflammatory.

In embodiments, a pediatric subject has been further administered or will be further administered an immune checkpoint inhibitor.

In embodiments, an immune checkpoint inhibitor is an inhibitor of PD-1, LAG-3, CTLA-4, TIM-3, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R. In embodiments, an immune checkpoint inhibitor is an agent that inhibits PD-1, LAG-3, TIM-3, CTLA-4, TIGIT, IDO, or CSF1R.

In embodiments, an immune checkpoint inhibitor is an agent that inhibits PD-1 (e.g., a small molecule, a nucleic acid, a polypeptide, a carbohydrate, a lipid, a metal, a toxin, a PD-1 binding agent, or a PD-L1 binding agent).

In embodiments, a PD-1 inhibitor is a PD-L1/L2 binding agent (e.g., an antibody, an antibody conjugate, or an antigen-binding fragment thereof such as durvalumab, atezolizumab, avelumab, BGB-A333, SHR-1316, FAZ-053, CK-301, or, PD-L1 millamolecule, or derivatives thereof).

In embodiments, a PD-1 inhibitor is a PD-1 binding agent (e.g., an antibody, an antibody conjugate, or an antigen-binding fragment thereof such as nivolumab, pembrolizumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, AMP-514/MEDI-0680, AGEN-2034, CS1001, TSR-042, Sym-021, PF-06801591, LZMO09, KN-035, AB122, genolimzumab (CBT-501), AK 104, or GLS-010, or derivatives thereof). In embodiments, a PD-1 inhibitor is TSR-042.

In embodiments, a PD-1 inhibitor is administered to the subject periodically at a dose of about 0.5 mg/kg to about 10 mg/kg.

In embodiments, a PD-1 inhibitor is administered at a dose of about 1.0 mg/kg to about 8.0 mg/kg or about 1.0 mg/kg to about 5.0 mg/kg.

In embodiments, a PD-1 inhibitor is administered to the subject periodically at a dose of about 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, or 9.5 mg/kg.

In embodiments, a PD-1 inhibitor is administered to the subject periodically at a dose of about 50 mg to about 2000 mg, about 50 mg to about 1000 mg, or about 100 mg to about 500 mg.

In embodiments, a PD-1 inhibitor is administered to the subject periodically at a dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, or about 1700 mg.

In embodiments, a PD-1 inhibitor is administered to the subject once every week, once every two weeks, once every three weeks, once every four weeks, once every five weeks, once every six weeks, once every seven weeks, once every eight weeks, once every nine weeks, or once every ten weeks. In embodiments, a PD-1 inhibitor is administered to the subject periodically at an administration interval that is once every three weeks.

In embodiments, a PD-1 inhibitor is administered as a first dose once every 3 weeks for 3, 4, or 5 cycles followed by a second dose administered once every six weeks. In embodiments, a first dose is about 500 mg of the PD-1 inhibitor. In embodiments, a second dose is about 1000 mg of the PD-1 inhibitor.

In embodiments, a cancer is cancer is characterized by a homologous recombination repair (HRR) gene deletion, a mutation in the DNA damage repair (DDR) pathway, homologous recombination deficiency (HRD), BRCA deficiency (e.g., as evidenced by a breast cancer susceptibility gene (BRCA)ness mutational signature), isocitrate dehydrogenase (IDH) mutation, high tumor mutation burden (TMB), and/or a chromosomal translocation. In embodiments, a cancer is a hypermutant cancer, a MSI-H cancer, a MSI-L cancer, or a MSS cancer. In embodiments, a cancer is characterized by one or more of these characteristics.

In embodiments, a cancer is a solid tumor.

In embodiments, a cancer is a non-CNS cancer (e.g., a non-CNS solid tumor). In embodiments, a cancer is neuroblastoma, hepatoblastoma, hepatocellular carcinoma, Wilms tumor, renal cell carcinoma, melanoma, adrenocortical carcinoma, adenocarcinoma of the colon, myoepithelial carcinoma, thymic cell carcinoma, nasopharyngeal carcinoma, squamous cell carcinoma, mesothelioma, or clivus chordoma. In embodiments, a cancer is extracranial embryonal neuroblastoma.

In embodiments, a cancer is a CNS cancer (e.g., a primary CNS malignancy). In embodiments, a cancer is ependymoma. In embodiments, a cancer is a brain cancer (e.g., glioblastoma multiforme, gliosarcoma, astrocytoma, glioblastoma, medulloblastoma, glioma, supratentorial primitive neuroectodermal tumor, atypical teratoid rhabdoid tumor, choroid plexus carcinoma, malignant ganglioma, gliomatosis cerebri, meningioma, or paraganglioma). In embodiments, a cancer is high-grade astrocytoma, low-grade astrocytoma, anaplastic astrocytoma, fibrillary astrocytoma, pilocytic astrocytoma, a high-grade glioma, low-grade glioma, diffuse intrinsic pontine glioma (DIPG), or anaplastic mixed glioma.

In embodiments, a cancer is a carcinoma.

In embodiments a cancer is a gonadal tumor.

In embodiments, a cancer is a hematological cancer. In embodiments, a cancer is a lymphoma (e.g., Hodgkin's lymphoma (e.g., relapsed or refractory classic Hodgkin's Lymphoma (cHL)), non-Hodgkin's lymphoma, diffuse large B-cell lymphoma, precursor T-lymphoblastic lymphoma, lymphoepithelial carcinoma, or malignant histiocytosis).

In embodiments, a cancer is a sarcoma (e.g., Ewings sarcoma, osteosarcoma, rhabdomyosarcoma, embryonal rhabdomyosarcoma, synovial sarcoma, alveolar rhabdomyosarcoma, alveolar soft part sarcoma, spindle cell sarcoma, angiosarcoma, epithelialoid sarcoma, inflammatory myofibroblastic tumor, or malignant rhadoid tumor).

In embodiments, a cancer is Ewing's sarcoma, osteosarcoma, ERS, a CNS tumor, or neuroblastoma.

In embodiments, a cancer is Ewing's sarcoma, osteosarcoma, rhabdomyosarcoma, neuroblastoma, medulloblastoma, high-grade glioma, or adrenocortical carcinoma.

In embodiments, a cancer is characterized by BRCA deficiency, high tumor mutation burden (TMB), and/or increased PD-L1 expression.

In embodiments, a cancer is Ewing's sarcoma, osteosarcoma, ERS, a CNS tumor, or neuroblastoma.

In embodiments, a cancer is recurrent.

In embodiments, a pediatric subject has not received at least one other line of treatment (LOT).

In embodiments, a pediatric subject has previously received at least one other line of treatment (LOT). In embodiments, a previous line of treatment is immunotherapy. In embodiments, a previous line of treatment is not immunotherapy. In embodiments, a pediatric subject is refractory to a previously-received line of treatment (e.g., a previously-administered chemotherapy). In embodiments, a pediatric subject is resistant to a previously-received line of treatment (e.g., a previously-administered chemotherapy).

In embodiments, niraparib is administered according to a dosage regimen that is determined by a subject body weight, by a subject's body surface area (B S A), or according to a flat dose.

In embodiments, niraparib can be administered in an amount that is about about 25 mg/m² to about 300 mg/m², about 25 mg/m² to about 275 mg/m², about 25 mg/m² to about 250 mg/m², about 25 mg/m² to about 200 mg/m², about 50 mg/m² to about 300 mg/m², about 50 mg/m² to about 275 mg/m², about 50 mg/m² to about 250 mg/m², about 50 mg/m² to about 200 mg/m², about 75 mg/m² to about 300 mg/m², about 75 mg/m² to about 275 mg/m², about 75 mg/m² to about 250 mg/m², about 75 mg/m² to about 200 mg/m², about 100 mg/m² to about 300 mg/m², about 100 mg/m² to about 275 mg/m², about 100 mg/m² to about 250 mg/m², about 100 mg/m² to about 200 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², or about 200 mg/m².

In embodiments, niraparib is orally administered in an amount that is about 25 mg to about 300 mg or about 25 mg to about 500 mg.

In embodiments, niraparib is administered in an amount that is about 25 mg, about 50 mg, about 75 mg, about 100 mg, about 125 mg, about 150 mg, about 175 mg, or about 200 mg.

In embodiments, niraparib is orally administered in an amount that is about 100 mg or about 200 mg of niraparib based on free base.

In embodiments, niraparib is administered in an amount that is about 75 mg, about 100 mg, about 130 mg, or about 160 mg.

In embodiments, niraparib is administered in an amount that is about 150 mg, about 200 mg, about 260 mg, or about 320 mg.

In embodiments, niraparib is administered in an amount that is about 225 mg, about 300 mg, about 390 mg, or about 480 mg.

In embodiments, niraparib is administered as a unit dose form that is a capsule comprising about 50 mg of niraparib.

In embodiments, niraparib is administered periodically to a pediatric subject. In embodiments, niraparib is administered once daily. In embodiments, niraparib is once every two days, once every three days, once every four days, once every five days, once every six days, or once every seven days.

In embodiments, two different amounts of niraparib are administered to the subject on alternating days on which dosages are administered to said subject.

In embodiments, said niraparib is administered as a unit dose form that is a solid.

In embodiments, niraparib is administered as a unit dose form that is a capsule. In embodiments, a capsule is a powder-, sprinkle, semisolid or liquid-filled capsule. In embodiments, a capsule is a seamless capsule (e.g., one or more seamless capsules filled into a hard capsule, a soft capsule, or a sachet).

In embodiments, contents of a capsule (e.g., contents of a seamless capsule) are sprinkled onto food or administered via a feeding tube.

In embodiments, said niraparib is administered as a unit dose form that is a capsule comprising about 50 mg niraparib based on free base.

In embodiments, niraparib is administered as a unit dose form that is a capsule comprising about 100 mg niraparib based on free base.

In embodiments, said niraparib is administered as a unit dose form that is a tablet.

In embodiments, a tablet is an orally dispersible or dissolvable tablet.

In embodiments, niraparib is administered as a unit dose form that is a tablet comprising about 50 mg, about 100 mg, 200 mg, or 300 mg niraparib based on free base.

In embodiments, niraparib is administered as a minitablet. In embodiments, a minitablet is filled into a capsule or a sachet.

In embodiments, niraparib is administered as a multiparticulate system. In embodiments, a multiparticulate system is filled into a capsule or a sachet.

In embodiments, niraparib is administered as a lozenge.

In embodiments, niraparib is administered as a sublingual tablet.

In embodiments, niraparib is administered as a gummy.

In embodiments, niraparib is administered as a film.

In embodiments, niraparib is administered as an oral liquid formulation. In embodiments, an oral liquid formulation is prepared from a tablet (e.g., a crushed tablet) or capsule (e.g., contents of a capsule) form.

In embodiments, an oral liquid formulation is a solution

In embodiments, oral liquid formulation is a suspension.

In embodiments, niraparib is administered as niraparib tosylate monohydrate.

In embodiments, a dosage of niraparib as described herein (e.g., a unit dose that is a tablet comprising about 50 mg niraparib) is administered with food (e.g., a dose is mixed with food). In embodiments, the contents of a capsule comprising niraparib are administered with food.

In embodiments, niraparib is administered via a feeding tube.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an exemplary Kaplan-Meier plot for progression-free survival in the gBRCAmut cohort based on IRC assessment (ITT Population, N=203).

FIG. 2 is an exemplary Kaplan-Meier for progression-free survival in the Non-gBRCAmut cohort overall based on IRC assessment (ITT Population, N=350).

FIG. 3 is schematic of an exemplary wet granulation manufacturing process of the niraparib tablet.

FIG. 4 is schematic of an exemplary moisture-activated dry granulation (MADG) manufacturing process of the niraparib tablet.

FIG. 5 is schematic of an exemplary dry granulation manufacturing process of the niraparib tablet.

FIG. 6A is a schematic of an exemplary manufacturing process of the niraparib capsule.

FIG. 6B is a schematic of an exemplary manufacturing process of the niraparib capsule.

FIG. 7 is an exemplary graph of results of stratified uniformity testing during encapsulation of batch D. It shows the average, minimum, and maximum percent label claim values across the encapsulation process.

FIG. 8 is an exemplary graph of particle size of powder blends of batches E, F, G, J, K, and L.

FIG. 9A is an exemplary diagram of a level of a blend in blender showing an exemplary point where capsule fill may be cutoff in some embodiments.

FIG. 9B is a diagram of an exemplary blender attached to a transfer chute.

FIG. 9C is a diagram of an exemplary transfer chute. The transfer chute can be attached to a blender and a powder blend can be transferred from the blender to an encapsulator through the transfer chute.

FIG. 9D is a diagram of an exemplary transfer chute.

FIG. 10 is an exemplary graph of individual stratified content uniformity data from different batches tested. One capsule (from batch K) tested at 170 minutes resulted in an assay value of 88.3%, but this capsule would have been rejected during weight sorting because it was outside of the in-process range. Stratified content uniformity (SCU) samples are not weight sorted.

FIG. 11 is an exemplary graph of sampling location of the encapsulator dosing bowl for batches E, F, G, J, K, and L.

FIG. 12 is an exemplary illustration of an apparatus used in an USP dissolution evaluation.

FIG. 13 is an exemplary illustration of an apparatus used in an USP dissolution evaluation.

FIG. 14 is an exemplary illustration of an apparatus used in an USP dissolution evaluation.

FIG. 15A depicts an exemplary scanning electron microscope (SEM) image of niraparib particles used in a batch.

FIG. 15B depicts an exemplary scanning electron microscope (SEM) image of niraparib particles used in a batch.

FIG. 15C depicts an exemplary scanning electron microscope (SEM) image of niraparib particles used in a batch.

FIG. 15D depicts an exemplary scanning electron microscope (SEM) image of niraparib particles used in a batch.

FIG. 15E depicts an exemplary scanning electron microscope (SEM) image of niraparib particles used in a batch.

FIG. 15F depicts an exemplary scanning electron microscope (SEM) image of niraparib particles used in a batch

FIG. 15G depicts an exemplary scanning electron microscope (SEM) image of niraparib particles used in a batch.

FIG. 1511 depicts an exemplary scanning electron microscope (SEM) image of niraparib particles used in a batch.

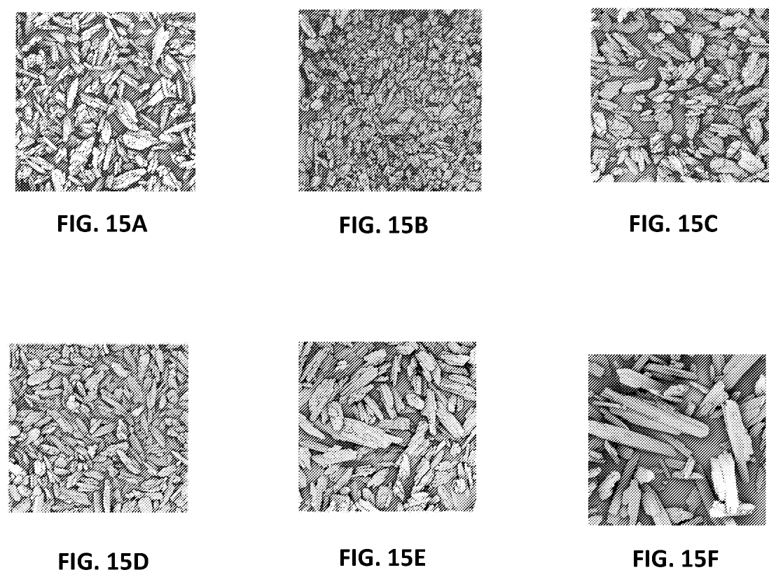

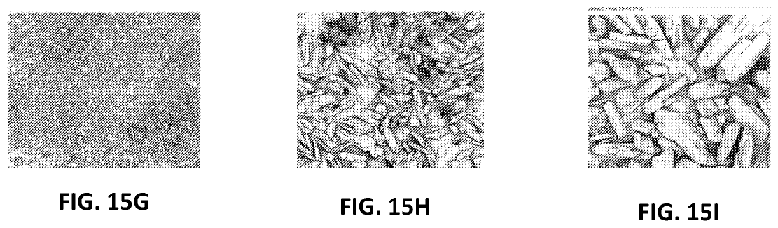

FIG. 15I depicts an exemplary scanning electron microscope (SEM) image of niraparib particles used in a batch.

### DETAILED DESCRIPTION OF THE INVENTION

Provided herein are exemplary methods for the treatment of cancers in a pediatric subject comprising administration of niraparib.

The treatment of cancer in pediatric population remains a significant unmet need. While relatively rare, cancer is the leading cause of death in children over 1 year of age in Europe and children past infancy in the US. For example, it was estimated that in 2018 that 15,590 children and adolescents of age 0-19 in the US would be diagnosed with cancer and 1,780 would die of the disease (https://www.cancer.gov/types/childhood-cancers/child-adolescent-cancers-fact-sheet). Advancements in cancer therapies have improved survival over the last few decades, but survival rates have plateaued over the last 5 years for difficult-to-treat diseases such as acute myeloid leukemia (AML), several CNS tumors, NB, and bone and soft tissue sarcomas. Approximately 20% to 30% of pediatric solid tumors will recur, and the recurrence rate can be as high as 70% to 80% in specific tumor types, such as high grade glioma. Thus, there is a significant unmet medical need for the treatment of recurrent solid tumours in the pediatric population.

In certain embodiments, niraparib is administered in combination with another line of therapy (e.g., another therapeutic agent as described herein). In embodiments, niraparib is orally administered in combination with a checkpoint inhibitor (e.g., intravenous administration of a PD-1 inhibitor such as TSR-042).

Methods described herein (e.g., oral administration of niraparib alone or in combination with another therapeutic agent such as TSR-042). Methods described herein can produce an antitumor immune response that can lead to long-term tumor regression. Such methods also can be particularly advantageous for the treatment of solid tumors such as medulloblastoma, high-grade glioma, neuroblastoma, osteosarcoma, Ewing's sarcoma, rhabdomysarcoma, or adrenocortical carcinoma. In particular, the methods can be beneficial for the treatment of cancers (e.g., solid tumors) characterized by one or more biomarkers such as BRCA deficiency (e.g., as determined by a mutational signature), high tumor mutational burden (TMB), and/or PD-L1 expression (e.g., positive PD-L1 expression such as high PD-L1 expression). The methods can also be useful for the treatment of recurrent cancers.

Various pharmaceutical products can be used for oral dosage and release of a pharmaceutically active composition comprising niraparib within an individual's body, including forms described herein. Exemplary suitable oral dosage forms comprising niraparib include solid oral dosage forms (e.g., tablets or capsules) and liquid dosage forms (e.g., suspensions or solutions).

Oral dosage pharmaceutical tablets typically contain a select amount of one or more pharmaceutically active compositions along with one or more inert excipient materials. In some embodiments, the oral dosage pharmaceutical tablets disclosed herein improve the manufacturability of the tablet by reducing the stickiness/adherence of the active pharmaceutical ingredient during the table manufacturing process. In some embodiments, the oral dosage pharmaceutical tablets disclosed herein have improved desirable properties, those related to flow, tensile strength, hardness, disintegration and bonding of intragranular and extragranular materials. In some embodiments, the oral dosage pharmaceutical tablets disclosed herein impart desirable properties to the final blend used to compress to tablets improve tablet formation. In some embodiments, the oral dosage pharmaceutical tablets are prepared from granules with the desirable granulation size that provides good flow, tablet bonding, and desirable disintegration profiles of the tablet. In some embodiments, the oral dosage pharmaceutical tablets have a distribution of the intragranular phase vs. extragranular phase components that provides desirable disintegration profiles.

Oral dosage pharmaceutical capsules are typically filled with microparticulate material or granules on the order of several microns in diameter or length. The encapsulated particles typically contain a select amount of one or more pharmaceutically active compositions along with one or more inert excipient materials. In a typical encapsulation process, a source of particulate material or particles to be encapsulated is transferred from a blender to a encapsulator, where the encapsulator determines the amount of particles to be added to each capsule. The encapsulator transfers the requisite amount of particles into an open capsule (e.g., an open shell portion of the capsule), and the open capsule is then sealed (e.g., by placing a shell cap over the open shell portion filled with particles).

### Definitions

The term “AUC” refers to the area under the time/plasma concentration curve after administration of the pharmaceutical composition. AUC_{0-Infinity} denotes the area under the plasma concentration versus time curve from time 0 to infinity; AUC₀₋ₜ denotes the area under the plasma concentration versus time curve from time 0 to time t.

“Binders” are used to hold the components in a composition, such as a tablet composition, together. In some embodiments, binders are used to form granules. Examples of suitable binders include but are not limited to disaccharides, such as sucrose and lactose; polysaccharides and derivatives thereof, such as starches, microcrystalline cellulose, methyl cellulose, ethyl cellulose, hydroxy propyl methyl cellulose, hydroxypropyl cellulose; sugar alcohols, such as xylitol, sorbitol, or maltitol, gelatin, polyvinylpyrrolidone (polyvidone or povidone), polyethylene glycol, polyvinyl alcohol, and polymethacrylates. In some embodiments, the binder is liquid binder or a solution binder. Examples of liquid binders include but are not limited to water, gelatin, cellulose, cellulose derivatives, povidone, starch, sucrose and polyethylene glycol. In some embodiments, the gelatin, cellulose, cellulose derivatives, povidone, starch, sucrose or polyethylene glycol may be dissolved. For example, they may be dissolved in water. In some embodiments, the liquid binder is povidone (PVP). In some embodiments, the binder is a dry binder. Examples of suitable dry binder include but are not limited to cellulose, methyl cellulose, hydroxyl propyl cellulose, povidone, polyethylene glycol. In some embodiments, the dry binder is hydroxypropyl cellulose (HPC). In some embodiments, the liquid binder is a melted binder utilizing a molten liquid as a binder. With melted binders, there may be no need for aqueous or organic solvents. Accordingly, no drying step may be required which shortens the total processing time and lowers the cost of operation. Furthermore, water-sensitive materials can be processed using this nonaqueous method of granulation. Melted binders may include hydrophilic polyethylene glycols (PEGs) and poloxamers, and hydrophobic fatty acids, fatty alcohols, waxes, hydrogenated vegetable oils and glycerides.

“Blood plasma concentration” refers to the concentration of compounds provided herein in the plasma component of blood of a subject

The term “bioequivalent” means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. In practice, two products are considered bioequivalent if the 90% confidence interval of the Cₘₐₓ, AUC, or, optionally, Tₘₐₓ is within the range of 80.00% to 125.00%.

“Bulk density”, as used herein, refers to the ratio of the mass of an untapped powder sample and its volume including the contribution of the interparticulate void volume. Bulk density indicates mass of a powder material that can be filled in per unit volume. For example, granules present in the pharmaceutical composition can have a bulk density more than or equal to 0.5 g/cm³.

The term “Cₘₐₓ” refers to the maximum concentration of isotretinoin in the blood following administration of the pharmaceutical composition.

The term “cancer” includes both solid tumors and hematological malignancies. Cancers include, but are not limited to, ovarian cancer, breast cancer, cervical cancer, endometrial cancer, prostate cancer, testicular cancer, pancreatic cancer, esophageal cancer, head and neck cancer, gastric cancer, bladder cancer, lung cancer (e.g., adenocarcinoma, NSCLC and SCLC), bone cancer (e.g., osteosarcoma), colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma (e.g., liposarcoma), bladder cancer, liver cancer (e.g., hepatocellular carcinoma), kidney cancer (e.g., renal cell carcinoma), myeloid disorders (e.g., AML, CML, myelodysplastic syndrome and promyelocytic leukemia), and lymphoid disorders (e.g., leukemia, multiple myeloma, mantle cell lymphoma, ALL, CLL, B-cell lymphoma, T-cell lymphoma, Hodgkin's lymphoma, non-Hodgkin's lymphoma, hairy cell lymphoma).

The term “capsule” is intended to encompass any encapsulated shell filled with medicines in powder form. Generally, capsules are made of liquid solutions of gelling agents like as gelatin (animal protein) and plant polysaccharides. These include modified forms of starch and cellulose and other derivatives like carrageenans. Capsule ingredients may be broadly classified as: (1) Gelatin Capsules: Gelatin capsules are made of gelatin manufactured from the collagen of animal skin or bone. Also known as gel caps or gelcaps. In gelatin capsules, other ingredients can also be added for their color and hardness like as plasticizers, water, glycerin, sorbitol, propylene glycol to modulatethe capsule's hardness, preservatives, coloring agents, opacifying agents, flavoring agents, sweeteners, lubricants and disintegrants; (2) Vegetable capsules: They are made of starch or a polymer formulated from cellulose, or alternatively can be made from hypromellose or polyvinyl alcohol (PVA).

The term “composition”, as in pharmaceutical composition, is intended to encompass a drug product comprising niraparib or its pharmaceutically acceptable salts, esters, solvates, polymorphs, stereoisomers or mixtures thereof, and the other inert ingredient(s) (pharmaceutically acceptable excipients). Such pharmaceutical compositions may be, in certain embodiments, synonymous with “formulation” and “dosage form”. Pharmaceutical composition of the invention include, but is not limited to, granules, tablets (single layered tablets, multilayered tablets, mini tablets, bioadhesive tablets, caplets, matrix tablets, tablet within a tablet, mucoadhesive tablets, modified release tablets, orally disintegrating tablets, pulsatile release tablets, timed release tablets, delayed release, controlled release, extended release and sustained release tablets), capsules (hard and soft, powder-, pellet- or liquid filled capsules), pills, troches, sachets, powders, microcapsules tablets in capsules and microspheres, matrix composition and the like. In some embodiments, the pharmaceutical composition refers to tablets. In some embodiments, pharmaceutical composition encompasses the bulk blend of the compositions provided herein prior to processing into final dosage form. In some embodiments, pharmaceutical composition encompasses an intermediate blend or composition comprising niraparib in formulation with one or more excipients of the compositions provided herein.

By “D₅₀”, it is meant that 50% of the particles are below and 50% of the particles are above a defined measurement. D₅₀ can be used to describe different parameters (volume, length, number, area, etc.). D₅₀ as used herein indicates the volume-weighted median diameter, for example, as measured by a laser/light scattering method or equivalent, wherein 50% of the particles, by volume, have a smaller diameter, while 50% by volume have a larger diameter. The volume weighted D₅₀ also relates to the percentage of weight of the particle under a certain size. For example, a D₅₀ of 500 nm means that 50% of the particulate mass is less than 500 nm in diameter and 50% of the particulate mass is greater than 500 nm in diameter. The particle size can be measured by conventional particle size measuring techniques well known to those skilled in the art. Such techniques include, for example, sedimentation field flow fractionation, photon correlation spectroscopy, light scattering (e.g., with a Microtrac UPA 150), laser diffraction and disc centrifugation. For the purposes of the compositions, formulations and methods described herein, effective particle size is the volume median diameter as determined using laser/light scattering instruments and methods, e.g. a Horiba LA-910, or Horiba LA-950. Similarly, “D₉₀” is the volume-weighted diameter, wherein 90% of the particles, by volume, have a smaller diameter, while 10% by volume have a larger diameter and “D₁₀” is the volume-weighted diameter, wherein 10% of the particles, by volume, have a smaller diameter, while 90% by volume have a larger diameter. It is sometimes useful to express the D₅₀ value after sonication for 1 minute or less using about 40 watts of sonicating power at room temperature (15° C. to 30° C.). This low power and short period can break up very loose aggregates which will not typically have a negative impact on the in vivo performance of the composition in a subject.

“Diluents” increase bulk of the composition to facilitate compression or create sufficient bulk for homogenous blend for tablet formulations. As used herein, diluents are synonyms with “filler”. Such compounds include e.g., lactose such as lactose monohydrate, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like. Combinations of one or more diluents can also be used. In some embodiments, the diluent is lactose monohydrate. In some embodiments, the diluent is lactose anhydrous. In some embodiments, the diluent is mannitol. In some embodiments, the diluent is calcium phosphate dibasic. In some embodiments, the diluent is microcrystalline cellulose. In some embodiments, one or more diluents affect the brittleness of the composition. In some embodiments, one or more diluents contribute to the plasticity of the composition. In some embodiments, the first diluent is used to adjust the brittleness of the composition and the second diluent is used to adjust the plasticity of the composition. In some embodiments, the first diluent is lactose monohydrate, lactose anhydrous, mannitol, or calcium phosphate dibasic. In some embodiments, the second diluent is microcrystalline cellulose, starch, polyethylene oxide, hydroxypropyl methylcellulose (HPMC).

“Disintegrant” expands and dissolves when wet causing a solid dosage form or tablet to break apart, for example, in the digestive tract, releasing the active ingredients for absorption. Disintegrants ensure that when the tablet is in contact with water, it rapidly breaks down into smaller fragments, facilitating dissolution. In some embodiments, the disintegrant is crospovidone or croscarmellose.

The terms “effective amount” or “therapeutically effective amount,” as used herein, refer to a sufficient amount of the niraparib being administered that would be expected to relieve to some extent one or more of the symptoms of the disease or condition being treated. For example, the result of administration of niraparib disclosed herein is reduction and/or alleviation of the signs, symptoms, or causes of cancer. For example, an “effective amount” for therapeutic uses is the amount of niraparib, including a formulation as disclosed herein required to provide a decrease or amelioration in disease symptoms without undue adverse side effects. The term “therapeutically effective amount” includes, for example, a prophylactically effective amount. It is understood that an “an effective amount” or a “therapeutically effective amount” varies, in some embodiments, from subject to subject, due to variation in metabolism of the compound administered, age, weight, general condition of the subject, the condition being treated, the severity of the condition being treated, and the judgment of the prescribing physician.

The terms “enhance” or “enhancing” refers to an increase or prolongation of either the potency or duration of a desired effect of niraparib, or a diminution of any adverse symptomatology that is consequent upon the administration of the therapeutic agent. Thus, in regard to enhancing the effect of niraparib disclosed herein, the term “enhancing” refers to the ability to increase or prolong, either in potency or duration, the effect of other therapeutic agents that are used in combination with niraparib disclosed herein. An “enhancing-effective amount,” as used herein, refers to an amount of niraparib or other therapeutic agent which is adequate to enhance the effect of another therapeutic agent or niraparib in a desired system. When used in a patient, amounts effective for this use will depend on the severity and course of the disease, disorder or condition, previous therapy, the patient's health status and response to the drugs, and the judgment of the treating physician.

The term “excipient” means a pharmacologically inactive component such as a diluent, lubricant, surfactant, carrier, or the like. Excipients that are useful in preparing a pharmaceutical composition are generally safe, non-toxic and are acceptable for human pharmaceutical use. Reference to an excipient includes both one and more than one such excipient. Co-processed excipients are also covered under the scope of present invention.

“Filling agents” or “fillers” include compounds such as lactose, lactose monohydrate, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Friability” means the condition of being friable, which is the ability of a solid substance to be reduced to smaller pieces. Friability as related to certain solid dosage forms may be evaluated according to: 1) European Pharmacopoeia (Ph. Eur.): Supplement 6.6 (published June 2009, official January 2010), Friability of Uncoated Tablets (reference 01/2010:20907); 2) Japanese Pharmacopoeia (JP): The JP General Information 26. Tablet Friability Test as it appears in the JP Fifteenth Edition (Mar. 31, 2006, The Ministry of Health, Labour and Welfare Ministerial Notification No. 285), officially updated by errata published by MHLW at http://www.std.pmda.go.jp/jpPUB/Data/ENG/jpdata/H201105_p15_errata.pdf on Nov. 5, 2008; or 3) 5.2.3 United States Pharmacopeia (USP): <1216> Tablet Friability, official in USP 32, May 1, 2009. Each of the afore-mentioned references are incorporated by reference herein. Friability may also be determined by updated versions of these references cited above, as applicable.

“Granulation” as used herein refers to process of binding particles of a dry powder composition through agglomeration to provide larger particles, known as granules that allow for production of pharmaceutical dosage form, such as tablets. Granulation is most often divided into two types: wet granulation, which requires a liquid in the process, and dry granulation, which does not require any liquid. Wet granulation uses a granulation liquid (binder/solvent) to facilitate the agglomeration by formation of a wet mass by adhesion while dry granulation uses mechanical compression, such as slugging, or compaction, such as roller compaction, to facilitate agglomeration. In roller compaction, ribbons are produced by passing the blend between the roller compactor rolls. The roll pressure and gap distance (set between the two rolls) are key parameters that influence the ribbon thickness. The ribbon thickness is important in tailoring the final particle size of the granulation, as it will affect the milling efficiency of the ribbons. Ribbon thickness may be measured with a caliper throughout the process. One method of measuring thickness is to obtain a rectangular sample of ribbon, at least 1 in (2.54 cm) from the compaction process. The dimensions (length, width, and thickness) are measured using a caliper or other device for measuring accurately to between one tenth or hundredth of an inch. Another parameter that may be measured is ribbon density, which is calculated by dividing the mass of the ribbon sample divided by the approximate volume (length×width×thickness).

“Intragranular phase” refers to the intragranular phase of the tablet, which comprises the granules that are prepared for tableting and comprises the components or excipients in the composition prior to granule formation. “Extragranular phase” refers to the extragranular phase of the tablet and comprises the excipients or components that are added to the composition after granule formation and before compression to provide a tablet.

“Lubricants” and “glidants” are compounds that prevent, reduce or inhibit adhesion or friction of materials. Without being limited as to theory, glidants prevent, reduce or inhibit adhesion of powders in a blend. For example, they may prevent, reduce or inhibit intra-particulate friction or may prevent, reduce or inhibit electrostatic charging of a powder. Lubricants may prevent, reduce or inhibit the adhesion of a powder to the surfaces into which it comes in contact. While glidants and lubricants may be any compound that provided the desired function, exemplary lubricants and glidants include, e.g., stearic acid, magnesium stearate, calcium hydroxide, talc, sodium stearyl fumarate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, glyceryl stearate, glyceryl palmitostearate, glyceryl distearate, polyethylene glycol, magnesium or sodium lauryl sulfate, colloidal silica such as Syloid™ Cab-O-Sil®, a starch such as corn starch, silicone oil, a surfactant, and the like. In some embodiments, a glidant is silicon dioxide. In some embodiments, a glidant is an intermediate meso-porous silica excipient.

“Particle size” refers to a measured distribution of particles and is usually expressed as the “volume weighted median” size unless specified otherwise.

“Pharmacodynamics” refers to the factors which determine the biologic response observed relative to the concentration of drug.

“Pharmacokinetics” refers to the factors which determine the attainment and maintenance of the appropriate concentration of drug.

“Moisture-activated dry granulation” (MADG) or “moist granulation” refers process for granulation that uses liquid, such as water, to activate the binder and initiate agglomeration. This process involves wet agglomeration of the powder particles, which is facilitated by the addition of an amount of a liquid, such as water, and moisture adsorption or distribution. Moisture adsorption or distribution comprises the addition of a moisture-absorbing material or adsorbant or absorbant after agglomeration to facilitate the absorption of excess moisture. Examples of suitable moisture-absorbing materials or adsorbant or absorbant include but are not limited to microcrystalline cellulose or silicon dioxide. In some embodiments, the adsorbant or absorbant is a large meso-porous silica excipient, bentonite, talc, microcrystalline cellulose, charcoal, fumed silica, magnesium carbonate, or similar excipients.

“Ready-to-use” refers to pharmaceutical compositions or medical products that can be used without the needs of further changing, modifying, or optimizing the composition or the product prior to administration, for example through dilution, reconstitution, sterilization, etc.

“Ribbon” and “ribbon thickness” are referred to with respect to a type of dry granulation that utilizes roll or roller compaction. In some embodiments of roll or roller compaction, powder is fed by gravity or by means of a screw through two counter-rotating rollers, rearranging the particles by the compaction pressure applied by the rollers, thus inducing a densification of the resulting material. The resulting material of roll or roller compaction is known as a “ribbon”, wherein a uniform and continuous flow of material is provided by the feeding system to form a “ribbon” of desired “ribbon thickness”. Ribbon thickness may be measured by any of the typical methods utilized in the art.

“Stable” or “stability” with respect to particle size distribution means the particle size distribution, e.g. D50 or D90 does not substantially change (greater than 50%) after an initial time is defined (e.g., after milling or a curing period (1 to 3 weeks)). For example, the stable niraparib particles described herein in a solid oral dosage form will not show an increase in effective particle size of greater than 50% up to 3, 6, 9, 12, 24 or 36 months storage at room temperature (15° C. to 25° C.). “Stable” or “stability” with respect to degradation of niraparib means that the number of impurities or degradation products does not substantially change (greater than 50%) after an initial time is defined. In some embodiments, the formulations described herein will not produce niraparib degradation impurities up to 3, 6, 9, 12, 24 or 36 months storage at room temperature (15° C. to 25° C.) at individual levels of about greater than 0.1% by weight as compared to the impurity levels at the initial time designation.

“Storage” with respect to the composition, including in solid dosage form, means storage in any container system or type for pharmaceutical use is an article which holds or is intended to contain a drug and is or may be in direct contact with it. In certain storage conditions, the container should provide the dosage form with adequate protection from factors (e.g., temperature, light) that can cause a degradation in the quality of that dosage form over its shelf life. Storage may occur in a blister (e.g. a multi-dose container consisting of two layers, of which one is shaped to contain the individual doses), a bottle (e.g. a container with a more or less pronounced neck and usually a flat bottom), a single-dose container (e.g. a container for single doses of solid, semi-solid or liquid preparations, a strip (e.g. a multi-dose container consisting of two layers, usually provided with perforations, suitable for containing single doses of solid or semi-solid preparations, a bag (e.g. a container consisting of surfaces, whether or not with a flat bottom, made of flexible material, closed at the bottom and at the sides by sealing; the top may be closed by fusion of the material, depending on the intended use), or an open dish.

The term “subject” is used to mean an animal, preferably a mammal, including a human or non-human. The terms patient and subject may be used interchangeably.

“Tablet” as used herein refers to a dosage form in which particles of a drug substance or pharmaceutical agent, such as niraparib, and certain excipients, such as any one of the excipients described herein, are pressed, compacted, or extruded together. In some embodiments, the tablet is prepared from direct compression using suitable punches or dies. In some embodiments, the tablet is prepared from injection or compression molding using suitable molds fitted to a compression unit. In some embodiments, the tablet is prepared from granulation, such as but not limited to fluid bed or high shear granulation or roller compaction, followed by compression. In some embodiments, the tablet is prepared from extrusion of a paste into a mold or to an extrudate to be cut into lengths. In some embodiments, the tablet is a solid tablet.

A “therapeutically effective amount” or “effective amount” is that amount of a pharmaceutical agent to achieve a pharmacological effect. The term “therapeutically effective amount” includes, for example, a prophylactically effective amount. An “effective amount” of niraparib is an amount needed to achieve a desired pharmacologic effect or therapeutic improvement without undue adverse side effects. The effective amount of a niraparib will be selected by those skilled in the art depending on the particular patient and the disease. It is understood that “an effective amount” or a “therapeutically effective amount” can vary from subject to subject, due to variation in metabolism of niraparib, age, weight, general condition of the subject, the condition being treated, the severity of the condition being treated, and the judgment of the prescribing physician. As used herein, amelioration or lessening of the symptoms of a particular disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

The term “tₘₐₓ” refers to the time in hours when Cₘₐₓ is achieved following administration of the pharmaceutical composition.

The terms “treat,” “treating” or “treatment,” as used herein, include alleviating, abating or ameliorating a disease or condition, for example cancer, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, “weight percent,” “wt %,” “percent by weight,” “% by weight,” and variations thereof refer to the concentration of a substance as the weight of that substance divided by the total weight of the composition and multiplied by 100.

Other objects, features, and advantages of the methods and compositions described herein will become apparent from the following detailed description. It should be understood, however, that the detailed description and the specific examples, while indicating specific embodiments, are given by way of illustration only.

Pediatric Subjects

The exemplary methods described herein can be used to treat a pediatric subject having any type of cancer.

In embodiments, a pediatric subject is a subject from the day of its birth (e.g., 0 days of age) to about 21 years of age. In embodiments, a pediatric subject is a subject from the day of its birth (e.g., 0 days of age) to about 18 years of age. In embodiments, a pediatric subject is a subject from about 1 day of age to about 21 years of age. In embodiments, a pediatric subject is a subject from about 1 day of age to about 18 years of age.

In embodiments, a pediatric subject is a subject that is about six months of age to about 21 years of age. In embodiments, a pediatric subject is about six months of age to about 18 years of age, about one year of age to about 18 years of age, about 1 year of age to about 6 years of age, or about 6 years of age to about 18 years of age.

In embodiments, a pediatric subject is about 4 years of age to about 18 years of age. In embodiments, a pediatric subject is about 4 years of age to about 10 years of age. In embodiments, a pediatric subject is about 10 years of age to about 15 years of age. In embodiments, a pediatric subject is about 10 years of age to about 18 years of age.

In embodiments, a pediatric subject is about six months of age to about 18 years of age.

In embodiments, a pediatric subject is about one year of age to about 18 years of age.

In embodiments, a pediatric subject is about 1 year of age to about 6 years of age.

In embodiments, a pediatric subject is about 6 years of age to about 18 years of age.

In embodiments, a pediatric subject is no less than about 6 months of age.

In embodiments, a pediatric subject is no less than about 4 years of age.

In embodiments, a pediatric subject is no less than about 6 years of age.

In embodiments, a pediatric subject is no more than about 18 years of age.

Indications Suitable for Treatment

Any subject having cancer, including breast cancer, ovarian cancer, cervical cancer, epithelial ovarian cancer, fallopian tube cancer, primary peritoneal cancer, endometrial cancer, prostate cancer, testicular cancer, pancreatic cancer, esophageal cancer, head and neck cancer, gastric cancer, bladder cancer, lung cancer (e.g., adenocarcinoma, NSCLC and SCLC), bone cancer (e.g., osteosarcoma), colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma (e.g., liposarcoma), bladder cancer, liver cancer (e.g., hepatocellular carcinoma), kidney cancer (e.g., renal cell carcinoma), myeloid disorders (e.g., AML, CML, myelodysplastic syndrome and promyelocytic leukemia), and lymphoid disorders (e.g., leukemia, multiple myeloma, mantle cell lymphoma, ALL, CLL, B-cell lymphoma, T-cell lymphoma, Hodgkin's lymphoma, non-Hodgkin's lymphoma, hairy cell lymphoma) may be treated with compounds and methods described herein.

In some embodiments, the methods of the invention treat subjects with ovarian cancer. In some embodiments, the methods of the invention treat subjects with epithelial ovarian cancer. In some embodiments, the methods of the invention treat subjects with fallopian tube cancer. In some embodiments, the methods of the invention treat subjects with primary peritoneal cancer.

In some embodiments, the methods of the invention treat subjects with recurrent ovarian cancer. In some embodiments, the methods of the invention treat subjects with recurrent epithelial ovarian cancer. In some embodiments, the methods of the invention treat subjects with recurrent fallopian tube cancer. In some embodiments, the methods of the invention treat subjects with recurrent primary peritoneal cancer.

In some embodiments, the methods of the invention treat subjects with recurrent ovarian cancer following a complete or partial response to a chemotherapy, such as a platinum-based chemotherapy. In some embodiments, the methods of the invention treat subjects with recurrent epithelial ovarian cancer following a complete or partial response to a chemotherapy, such as a platinum-based chemotherapy. In some embodiments, the methods of the invention treat subjects with recurrent fallopian tube cancer following a complete or partial response to a chemotherapy, such as a platinum-based chemotherapy. In some embodiments, the methods of the invention treat subjects with recurrent primary peritoneal cancer following a complete or partial response to a chemotherapy, such as a platinum-based chemotherapy.

In some embodiments, the methods of the invention treat subjects with recurrent ovarian cancer, recurrent epithelial ovarian cancer, recurrent fallopian tube cancer and/or recurrent primary peritoneal cancer following a complete or partial response to a platinum-based chemotherapy, wherein the subjects begin the treatment no later than 8 weeks after their most recent platinum-containing regimen. For example, subjects can begin treatment with niraparib about 7 weeks after their most recent platinum-containing regimen. For example, subjects can begin treatment with niraparib about 6 weeks after their most recent platinum-containing regimen. For example, subjects can begin treatment with niraparib about 6 weeks after their most recent platinum-containing regimen. For example, subjects can begin treatment with niraparib about 5 weeks after their most recent platinum-containing regimen. For example, subjects can begin treatment with niraparib about 4 weeks after their most recent platinum-containing regimen. For example, subjects can begin treatment with niraparib about 3 weeks after their most recent platinum-containing regimen. For example, subjects can begin treatment with niraparib about 2 weeks after their most recent platinum-containing regimen. For example, subjects can begin treatment with niraparib about 1 week after their most recent platinum-containing regimen.

In some embodiments, the methods of the invention treat subjects with prostate cancer.

In some embodiments, the methods of the invention treat subjects with a pediatric cancer. Exemplary pediatric cancers include, but are not limited to adrenocortical carcinoma, astrocytoma, atypical teratoid rhabdoid tumor, brain tumors, chondroblastoma, choroid plexus tumor, craniopharyngioma, desmoid tumor, dysembryplastic neuroepithelial tumor (DNT), ependymoma, fibrosarcoma, germ cell tumor of the brain, glioblastoma multiforme, diffuse pontine glioma, low grade glioma, gliomatosis cerebri, hepatoblastoma, histiocytosis, kidney tumor, acute lymphoblastic leukemia (ALL), acute myeloid leukemia (AML), chronic myelogenous leukemia (CML), liposarcoma, liver cancer, Burkitt lymphoma, Hodgkin lymphoma, non-Hodgkin lymphoma, malignant fibrous histiocytoma, melanoma, myelodysplastic syndrome, nephroblastoma, neuroblastoma, neurofibrosarcoma, osteosarcoma, pilocytic astrocytoma, retinoblastoma, rhabdoid tumor of the kidney, rhabdomyosarcoma, Ewing sarcoma, soft tissue sarcoma, synovial sarcoma, spinal cord tumor and Wilm's tumor.

In embodiments, a cancer is Ewing's sarcoma, osteosarcoma, rhabdomyosarcoma (RMS) such as embryonal rhabdomyosarcoma (ERS), a CNS tumor, or neuroblastoma. In embodiments, a cancer is a CNS tumor.

In embodiments, a cancer is Ewing's sarcoma (ES), osteosarcoma (OS), rhabdomyosarcoma (RMS), neuroblastoma (NB), medulloblastoma (MB), high-grade glioma (HGG), or adrenocortical carcinoma (ACC).

Biomarkers

Biomarker levels may also be used as a factor for determining administration to a subject, including route and/or intervals.

In some embodiments, biomarker levels may be used in combination with other factors such as the nature, severity of the disease and extent of the subject's condition, and/or to identify an appropriate treatment regimen.

In embodiments, a subject receives treatment independent of biomarker status. In embodiments, a subject receives treatment without determination of biomarker status. In embodiments, a subject receives treatment prior to determination of biomarker status.

As used herein, a “biomarker” or “marker” is a gene, mRNA, or protein which can be altered, wherein said alteration is associated with cancer. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, a marker associated with cancer, or predictive of responsiveness to anti-cancer therapeutics, can have an altered nucleotide sequence, amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell as compared to a normal, healthy tissue or cell. Furthermore, a “marker” includes a molecule whose structure is altered, e.g., mutated (contains a mutation), e.g., differs from the wild-type sequence at the nucleotide or amino acid level, e.g., by substitution, deletion, or insertion, when present in a tissue or cell associated with a cancer.

The target gene or gene product can include a single nucleotide polymorphism (SNP). In another embodiment, the gene or gene product has a small deletion, e.g., a small intragenic deletion (e.g., an in-frame or frame-shift deletion). In yet another embodiment, the target sequence results from the deletion of an entire gene. In still another embodiment, the target sequence has a small insertion, e.g., a small intragenic insertion. In one embodiment, the target sequence results from an inversion, e.g., an intrachromosal inversion.

In embodiments, a cancer is cancer is characterized by a homologous recombination repair (HRR) gene deletion, a mutation in the DNA damage repair (DDR) pathway, homologous recombination deficiency (HRD), BRCA deficiency, isocitrate dehydrogenase (IDH) mutation, high tumor mutation burden (TMB), and/or a chromosomal translocation. In embodiments, a cancer is a hypermutant cancer, a MSI-H cancer, a MSI-L cancer, or a MSS cancer. In embodiments, a cancer is characterized by BRCA deficiency, high TMB, or PD-L1 expression. In embodiments, a cancer is characterized by one or more of these characteristics.

In some embodiments, an expression level of one biomarker may be used in combination with expression levels of other biomarkers. In some embodiments, expression of a biomarker may be used independently of expression levels of other biomarkers. In embodiments, a cancer is characterized by BRCA deficiency, high tumor mutation burden (TMB), and/or increased PD-L1 expression.

In embodiments, a cancer is characterized by a mutational signature (e.g., any one of the thirty mutational signatures identified in the Catalogue of Somatic Mutations in Cancer (COSMIC)). In embodiments, a cancer is characterized by COSMIC Signature 3 (e.g., a cancer is associated with failure of DNA double-strand break repair by homologous recombination).

BRCA

BRCA deficiency can result from a BRCA mutation. As used herein, “BRCA mutation” or “mutation of BRCA” refers to a change or difference in the sequence of at least one copy of either or both of the BRCA1 or BRCA2 genes relative to an appropriate reference sequence (e.g., a wild type reference and/or a sequence that is present in non-cancerous cells in the subject). A mutation in the BRCA1/2 gene may result in a BRCA1/2 deficiency, which may include, for example a loss or reduction in the expression or function of the BRCA gene and/or encoded protein. Such mutations may also be referred to as “deleterious mutations” or may be suspected to be deleterious mutations. A BRCA mutation can be a “germline BRCA mutation,” which indicates it was inherited from one or both parents. Germline mutations affect every cell in an organism and are passed on to offspring. A BRCA mutation can also be acquired during one's lifetime, i.e. spontaneously arising in any cell in the body (“soma”) at any time during the patient's life, (i.e., non-inherited), which is referred to herein as a “sporadic BRCA mutation” or a “somatic BRCA mutation” interchangeably. Genetic tests are available, and known by those of skill in the art. For example, the BRACAnalysis CDx® kit is an in vitro diagnostic for detection and classification of BRCA1/2 variants. Using isolated genomic DNA, the BRACAnalysis CDx identifies mutations in the protein coding regions and intron/exon boundaries of the BRCA1 and BRCA2 genes. Single nucleotide variants and small insertions and deletions (indels) may be identified by polymerase chain reaction (PCR) and nucleotide sequencing. Large deletions and duplications in BRCA1 and BRCA2 may be detected using multiplex PCR.

Indication of a “BRCA status” refers to, in at least some cases, whether a mutation is present in at least one copy of either BRCA1 or BRCA2. In some embodiments, indication of a BRCA status may refer to the mRNA expression level, methylation level or other epigenetic modification of either or both of BRCA1 and BRCA2. In some embodiments, a patient with a “positive BRCA status” refers to a patient from whom a sample has been determined to contain a mutation in BRCA1 and/or BRCA2. In some embodiments, a positive BRCA status refers to the presence of either a germline BRCA mutation (gBRCA^{mut}) or a somatic BRCA mutation (sBRCA^{mut}). In some embodiments, a patient with a “positive BRCA status” refers to a patient from whom a sample has been determined to have a reduced expression of BRCA1 and/or BRCA2. In some embodiments, BRCA status is determined for germline BRCA mutations (e.g., gBRCA^{mut}) and is performed on a blood sample of a subject. In some embodiments, BRCA status is determined for somatic BRCA mutations (sBRCA^{mut}) or total BRCA mutations (tBRCA^{mut}, which includes both somatic and BRCA germline mutations).

In embodiments, a BRCA deficiency corresponds to or is identified by a particular mutational signature, which can also be referred to as a “breast cancer susceptibility gene (BRCA)ness mutational signature.”

Tumor Mutational Burden (TMB)

Tumor mutational burden measures the number of genomic mutations present in a tumor. Without wishing to be bound by theory, the higher the mutational burden, the more neo-antigens (or “non-self” proteins) a tumor may generate. The more neo-antigens, the greater the likelihood that the immune system will see the tumor as “non-self” and attack it.

As described herein, TMB is a biomarker that can be used as an indicator of a disease state of cancer, the severity of the cancer, or responses to a therapeutic intervention. TMB levels can be used alone or in combination as an indicator to evaluate and select cancer patients for treatment as described herein. In some embodiments, TMB levels can be used in conjunction with one or more additional markers, in particular, those markers known to be associated with certain cancers and/or treatment response to a particular line of therapy (LOT) such as immunotherapy.

In some embodiments, the TMB level for cancer is compared between cancer patients and normal healthy individuals. In some embodiments, the TMB level is compared between patients with different subtypes of cancer.

In some embodiments, the TMB level is compared to a reference level. In some embodiments, the reference level is determined based on TMB data from a population of samples. In some embodiments, the sample obtained from the subject in need of treatment is characterized by a lower level of TMB than the reference level. In some embodiments, the sample obtained from the subject in need of treatment is characterized by a lower level of TMB than the reference level.

Next generation sequencing (NGS) of whole exome, WES, or targeted panels, circulating tumor DNA based tests circulating tumor DNA based assay (ctDNA) can be used to measure TMB.

PD-L1 Expression

Programmed death ligand 1 (PD-L1) is a protein that interacts with programmed cell death protein 1 (PD-1) and is expressed on, e.g., immune cells and tumor cells (see, e.g., Kim et al., Sci. Rep. 6, 36956; doi:10.1038/srep36956 (2016). In particular, expression of PD-L1 on tumors provides a mechanism of cancer-induced immune suppression, and targeting this pathway can be effective for treating certain cancers (Shukuya et al., Journal of Thoracic Oncology, 11(7):976-988, 2016.

In embodiments, a subject has a cancer characterized by PD-L1 expression.

In embodiments, a subject is selected for treatment based on the measured PD-L1 expression of a sample as compared to a reference level.

The Tumor Proportion Score (TPS) of a sample can be determined by the percentage of viable tumor cells showing partial or complete membrane staining at any intensity. In embodiments, the TPS of a sample is determined using IHC. In embodiments, a positive PD-L1 expression is characterized by a TPS of at least about 1% (i.e., a TPS≥1%). In embodiments, a positive PD-L1 expression is characterized by a TPS of about 1% to 49%. In embodiments, high expression of PD-L1 is characterized by a TPS that is at least about 50% (i.e., a TPS≥50%).

In embodiments, PD-L1 expression is expressed as Combined Positive Score (CPS). The Combined Positive Score (CPS) of a sample can be determined by the number of PD-L1 staining cells (tumor cells, lymphocytes, and macrophages) divided by the total number of viable tumor cells and then multiplied by 100. In embodiments, the TPS of a sample is determined using IHC. In embodiments, a sample that expresses PD-L1 has a CPS of at least about 1 (i.e., a CPS≥1). In embodiments, a sample that expresses PD-L1 has a CPS of at least about 10 (i.e., a CPS≥10).

In embodiments, PD-L1 expression is expressed as the proportion of tumor area occupied by PD-L1 expressing tumor-infiltrating immune cells (% IC) of any intensity. In embodiments, a positive PD-L1 expression is characterized by a % IC of at least about 1% (i.e., a % IC≥1%). In embodiments, a positive PD-L1 expression is characterized by a % IC of about 1% to 49%. In embodiments, a sample that expresses PD-L1 has a % IC of at least about 50% (i.e., a % IC≥50%).

In embodiments, PD-L1 expression is expressed as the percentage of PD-L1 expressing tumor cells (% TC) of any intensity. In embodiments, a positive PD-L1 expression is characterized by a % TC of at least about 1% (i.e., a % TC≥1%). In embodiments, a positive PD-L1 expression is characterized by a % TC of about 1% to 49%. In embodiments, a sample that expresses PD-L1 has a % TC of at least about 50% (i.e., a % TC≥50%).

In embodiments, PD-L1 expression is determined using immunohistochemistry (IHC), flow cytometry, PET imaging, immunofluorescence, and/or western blotting. See, e.g., Rom-Jurek et al., Int. J. Mol. Sci., 19:563, 2018. In embodiments, PD-L1 expression is determined using immunohistochemistry (IHC). In embodiments, PD-L1 expression is determined using flow cytometry. In embodiments, PD-L1 expression is determined using PET imaging. In embodiments, PD-L1 expression is determined using immunofluorescence. In embodiments, PD-L1 expression is determined using western blotting. In embodiments, determination of PD-L1 expression comprises the use of a PD-L1 binding agent (e.g., a diagnostic antibody or antibody fragment).

Ewing's Sarcoma

In embodiments, a cancer is Ewing's sarcoma (ES).

ES is a rare tumor that affects primarily bones and, less commonly, soft tissue. It is estimated that 1 to 3 cases per 1 million people per year are diagnosed with ES. The ES cell of origin has been thought of to be either a mesenchymal stem cell or a neural crest-derived stem cell, with a pathognomonic chimeric transcription factor oncogene as a result of a somatic reciprocal chromosomal translocation between EWSR1 and an ETS family member gene (in this case, ERG). ES typically develops from bone and occasionally with a pathologic fracture. However, in approximately 20% of patients, the primary tumor evolves from soft tissue.

In embodiments, Ewing's sarcoma is an advanced Ewing's sarcoma. In embodiments, Ewing's sarcoma is a metastatic Ewing's sarcoma. In embodiments, Ewing's sarcoma is recurrent Ewing's sarcoma.

In embodiments, Ewing's sarcoma is a MSI-H Ewing's sarcoma. In embodiments, Ewing's sarcoma is a MSS Ewing's sarcoma. In embodiments, Ewing's sarcoma is a POLE-mutant Ewing's sarcoma. In embodiments, Ewing's sarcoma is a POLD-mutant Ewing's sarcoma. In embodiments, Ewing's sarcoma is a high TMB Ewing's sarcoma. In embodiments, Ewing's sarcoma is associated with homologous recombination repair deficiency/homologous repair deficiency (“HRD”) or is characterized by a homologous recombination repair (HRR) gene mutation or deletion. In embodiments, Ewing's sarcoma is BRCA-deficient Ewing's sarcoma. In embodiments, Ewing's sarcoma is characterized by PD-L1 expression (e.g., high PD-L1 expression).

In embodiments, a subject having Ewing's sarcoma is a pediatric subject (e.g., as described herein). In embodiments, a subject is no more than about 15 years of age. In embodiments, a subject is about 8 years of age to about 18 years of age. In embodiments, a subject is about 8 years of age to about 16 years of age, about 8 years of age to about 14 years of age, about 10 years of age to about 18 years of age, or about 10 years of age to about 15 years of age.

In embodiments, a subject is male. In embodiments, a subject is female.

In embodiments, a subject with Ewing's sarcoma has ES lesions in the extremeties (e.g., distal extremeities). In embodiments, a subject with Ewing's sarcoma has lesions in the pelvis. In embodiments, a subject with Ewing's sarcoma has extraskeletal primary tumors.

In embodiments, a subject with Ewing's sarcoma has a tumor less than about 200 mL in volume. In embodiments, a subject with Ewing's sarcoma has a tumor less than or equal to about 200 mL in volume. In embodiments, a subject with Ewing's sarcoma has a tumor greater than about 200 mL in volume. In embodiments, a subject with Ewing's sarcoma has a tumor greater than or equal to about 200 mL in volume.

In embodiments, a subject with Ewing's sarcoma has a tumor with a single dimension of less than about 8 cm. In embodiments, a subject with Ewing's sarcoma has a tumor with a single dimension of less than or equal to about 8 cm. In embodiments, a subject with Ewing's sarcoma has a tumor with a single dimension of greater than about 8 cm. In embodiments, a subject with Ewing's sarcoma has a tumor with a single dimension of greater than or equal to about 8 cm.

In embodiments, a subject with Ewing's sarcoma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof.

Osteosarcoma

In embodiments, a cancer is osteosarcoma (OS).

Osteosarcomas are characterised by the production of osteoid or immature bone. Unbalanced karyotypes are frequently observed in osteosarcomas, with loss of heterozygosity of tumor suppressor genes RBI (retinoblastoma 1) and TP53 making up the majority of the observed germline mutations.

In embodiments, an osteosarcoma is an advanced osteosarcoma. In embodiments, an osteosarcoma is a metastatic osteosarcoma. In embodiments, an osteosarcoma is recurrent osteosarcoma.

In embodiments, an osteosarcoma is a MSI-H osteosarcoma. In embodiments, an osteosarcoma is a MSS osteosarcoma. In embodiments, an osteosarcoma is a POLE-mutant osteosarcoma. In embodiments, an osteosarcoma is a POLD-mutant osteosarcoma. In embodiments, an osteosarcoma is a high TMB osteosarcoma. In embodiments, an osteosarcoma is associated with homologous recombination repair deficiency/homologous repair deficiency (“HRD”) or is characterized by a homologous recombination repair (HRR) gene mutation or deletion. In embodiments, an osteosarcoma is BRCA-deficient osteosarcoma. In embodiments, an osteosarcoma is characterized by PD-L1 expression (e.g., high PD-L1 expression).

In embodiments, a subject having osteosarcoma is a pediatric subject (e.g., as described herein). In embodiments, a subject is no more than about 19 years of age. In embodiments, a subject is about 8 years of age to about 19 years of age, about 10 years of age to about 19 years of age, about 13 years of age to about 19 years of age, or about 15 years of age to about 19 years of age. In embodiments, a subject is about 10 years of age to about 16 years of age, about 8 years of age to about 14 years of age, about 10 years of age to about 18 years of age, about 10 years of age to about 15 years of age, about 12 years of age to about 18 years of age, about 12 years of age to about 17 years of age, about 12 years of age to about 16 years of age, or about 13 years of age to about 16 years of age.

In embodiments, a subject is male. In embodiments, a subject is female.

In embodiments, a subject with osteosarcoma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof. In embodiments, a LOT is surgery and/or chemotherapy.

Rhabdomyosarcoma

In embodiments, a cancer is rhabdomyosarcoma (RMS).

In embodiments, a rhabdomyosarcoma is an advanced rhabdomyosarcoma. In embodiments, a rhabdomyosarcoma is a metastatic rhabdomyosarcoma. In embodiments, a rhabdomyosarcoma is recurrent rhabdomyosarcoma.

In embodiments, a rhabdomyosarcoma is a MSI-H rhabdomyosarcoma. In embodiments, a rhabdomyosarcoma is a MSS rhabdomyosarcoma. In embodiments, a rhabdomyosarcoma is a POLE-mutant rhabdomyosarcoma. In embodiments, a rhabdomyosarcoma is a POLD-mutant rhabdomyosarcoma. In embodiments, a rhabdomyosarcoma is a high TMB rhabdomyosarcoma. In embodiments, a rhabdomyosarcoma is associated with homologous recombination repair deficiency/homologous repair deficiency (“HRD”) or is characterized by a homologous recombination repair (HRR) gene mutation or deletion. In embodiments, a rhabdomyosarcoma is BRCA-deficient rhabdomyosarcoma. In embodiments, a rhabdomyosarcoma is characterized by PD-L1 expression (e.g., high PD-L1 expression).

In embodiments, a subject having rhabdomyosarcoma is a pediatric subject (e.g., as described herein). In embodiments, a subject is no more than about eighteen years of age. In embodiments, a subject is no more than about fifteen years of age. In embodiments, a subject is no more than about six years of age. In embodiments, a subject is about six years of age to about 18 years of age. In embodiments, a subject is about 4 years of age to about 14 years of age, about 2 years of age to about 12 years of age, or about 1 year of age to about 10 years of age. In embodiments, a subject is about 2 years of age to about 10 years of age, about 2 years of age to about 8 years of age, about 4 years of age to about 10 years of age, or about 4 years of age to about 8 years of age.

In embodiments, a subject is male. In embodiments, a subject is female.

In embodiments, a subject with osteosarcoma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof. In embodiments, a LOT is chemotherapy.

Neuroblastoma

In embodiments, a cancer is neuroblastoma (NB).

NBs are neuroblastic tumors that arise from primitive sympathetic ganglion cells. NB is a heterogeneous tumor type, and tumors vary in location, histopathologic appearance, and biologic characteristics. Cytogenetic and molecular genetic factors influencing the clinical tumor behaviour and treatment outcome include MYCN amplification, DNA content (ploidy), and gain or loss of whole or partial chromosomes.

In embodiments, a neuroblastoma is an advanced neuroblastoma. In embodiments, a neuroblastoma is a metastatic neuroblastoma. In embodiments, a neuroblastoma is recurrent neuroblastoma.

In embodiments, a neuroblastoma is a MSI-H neuroblastoma. In embodiments, a neuroblastoma is a MSS neuroblastoma. In embodiments, a neuroblastoma is a POLE-mutant neuroblastoma. In embodiments, a neuroblastoma is a POLD-mutant neuroblastoma. In embodiments, a neuroblastoma is a high TMB neuroblastoma. In embodiments, a neuroblastoma is associated with homologous recombination repair deficiency/homologous repair deficiency (“HRD”) or is characterized by a homologous recombination repair (HRR) gene mutation or deletion. In embodiments, a neuroblastoma is BRCA-deficient neuroblastoma. In embodiments, a neuroblastoma is characterized by PD-L1 expression (e.g., high PD-L1 expression).

In embodiments, a subject having neuroblastoma is a pediatric subject (e.g., as described herein). In embodiments, a subject is no more than about eighteen years of age. In embodiments, a subject is no more than about 10 years of age. In embodiments, a subject is no more than about 4 years of age. In embodiments, a subject is no more than about 3 years of age. In embodiments, a subject is about 6 months of age to about 18 years of age. In embodiments, a subject is about 6 months of age to about 10 years of age. In embodiments, a subject is about 6 months of age to about 5 years of age. In embodiments, a subject is about 5 years of age to about 10 years of age.

In embodiments, a subject is male. In embodiments, a subject is female.

In embodiments, a subject with neuroblastoma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof.

Medulloblastoma

In embodiments, a cancer is medulloblastoma (MB).

MB is the most common pediatric brain tumor. MB and other neuroectodermal tumors account for 16% to 25% of all childhood cancers. MB can be subdivided into histologically or genetically defined categories. Histologically, there are 4 categories of MB: classic MB, desmoplastic/nodular MB, MB with extensive nodularity, and large cell/anaplastic MB. Genetically, there are roughly 4 categories of MB: tumors with activated WNT (wingless), tumors with activated sonic hedgehog (SHH) and mutated TP53, tumors with activated SHH with unmutated TP53, and tumors that do not have WNT or SHH activated. In embodiments, a medulloblastoma is any of these histological and/or genetic categories.

In embodiments, a medulloblastoma is an advanced medulloblastoma. In embodiments, a medulloblastoma is a metastatic medulloblastoma. In embodiments, a medulloblastoma is recurrent medulloblastoma.

In embodiments, a medulloblastoma is a MSI-H medulloblastoma. In embodiments, a medulloblastoma is a MSS medulloblastoma. In embodiments, a medulloblastoma is a POLE-mutant medulloblastoma. In embodiments, a medulloblastoma is a POLD-mutant medulloblastoma. In embodiments, a medulloblastoma is a high TMB medulloblastoma. In embodiments, a medulloblastoma is associated with homologous recombination repair deficiency/homologous repair deficiency (“HRD”) or is characterized by a homologous recombination repair (HRR) gene mutation or deletion. In embodiments, a medulloblastoma is BRCA-deficient medulloblastoma. In embodiments, a medulloblastoma is characterized by PD-L1 expression (e.g., high PD-L1 expression).

In embodiments, a subject having medulloblastoma is a pediatric subject (e.g., as described herein). In embodiments, a subject is no more than about eighteen years of age. In embodiments, a subject is no more than about 10 years of age. In embodiments, a subject is no more than about 8 years of age. In embodiments, a subject is no more than about 4 years of age. In embodiments, a subject is about 6 months of age to about 10 years of age.

In embodiments, a subject is male. In embodiments, a subject is female.

In embodiments, a subject with medulloblastoma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof. In embodiments, a LOT is hematopoietic cell transplantation (e.g., bone marrow transplantation or stem cell transplantation).

High-Grade Glioma

In embodiments, a cancer is high-grade glioma (HGG).

HGG is an umbrella term for all high-grade malignancies of glial origin, including glioblastomas, anaplastic astrocytomas, and diffuse intrinsic pontine gliomas. HGG comprises approximately 14% of all pediatric brain tumors. Among children aged ≤18 years, HGG most commonly occurs in teenagers and young adults. The 5-year overall survival (OS) rates are less than 20%.

HGG can be divided into 4 subgroups: proneural, neural, classical, and mesenchymal. The subgroup categorization is based on the cell type of origin. Specific mutations have been identified for the subgroups of proneural (PDGFR/IDH 1), classical (EGFR), and mesenchymal (NF1). In older adolescents or young adults with glioblastoma multiforme, a subtype of HGG, mutations in the histone gene (H3F3A) were observed in approximately 31% of tumors. Additional mutations in these tumors occur in TP53, ATRX, and DAXX. The presence of H3F3A1 ATRXIDAXXI TP53 mutations was associated with the ability of tumor cells to use an alternative pathway to lengthen their telomeres.

In embodiments, a high grade glioma is a glioblastoma.

In embodiments, a high grade glioma is an anaplastic astrocytoma.

In embodiments, a high grade glioma is a diffuse intrinsic pontine glioma (DIPG).

In embodiments, a high grade glioma is an advanced high grade glioma. In embodiments, a high grade glioma is a metastatic high grade glioma. In embodiments, a high grade glioma is recurrent high grade glioma.

In embodiments, a high grade glioma is a MSI-H high grade glioma. In embodiments, a high grade glioma is a MSS high grade glioma. In embodiments, a high grade glioma is a POLE-mutant high grade glioma. In embodiments, a high grade glioma is a POLD-mutant high grade glioma. In embodiments, a high grade glioma is a high TMB high grade glioma. In embodiments, a high grade glioma is associated with homologous recombination repair deficiency/homologous repair deficiency (“HRD”) or is characterized by a homologous recombination repair (HRR) gene mutation or deletion. In embodiments, a high grade glioma is BRCA-deficient high grade glioma. In embodiments, a high grade glioma is characterized by PD-L1 expression (e.g., high PD-L1 expression).

In embodiments, a subject having high grade glioma is a pediatric subject (e.g., as described herein). In embodiments, a subject is no more than about eighteen years of age. In embodiments, a subject is about 6 months of age to about 18 years of age. In embodiments, a subject is about 6 months of age to about 16 years of age. In embodiments, a subject is about 6 months of age to about 14 years of age.

In embodiments, a subject is male. In embodiments, a subject is female.

In embodiments, a subject with high grade glioma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof.). In embodiments, a LOT is chemotherapy.

Adrenocortical Carcinoma

In embodiments, a cancer is adrenocortical carcinoma (ACC).

ACC is a very rare type of tumor. Results from a 2013 National Cancer Institute SEER analysis estimated that the annual incidence of ACC in patients under 20 years of age was 0.2 patients per million. Five-year survival rates were strongly correlated with age: 91% for patients ≤4 years and 30% in patients between 5 and 19 years of age. A retrospective study of ACC in patients <20 years of age in The Netherlands also demonstrated a strong correlation with age. Of 12 patients with ACC, all 7 patients aged ≤4 years survived, while all 5 patients aged >4 years died.

The etiology of pediatric ACC is different from that of adult ACC. Most children with adrenocortical tumors (carcinoma or adenoma) have associated familial cancer syndromes such as Li-Fraumeni syndrome (Else et al. 2014), which is caused by mutations in the TP53 gene. While not all pediatric ACC is associated with Li-Fraumeni syndrome, 50% to 80% of all pediatric tumors are associated with germline mutations in the TP53 gene.

In embodiments, a adrenocortical carcinoma is an advanced adrenocortical carcinoma. In embodiments, a adrenocortical carcinoma is a metastatic adrenocortical carcinoma. In embodiments, a adrenocortical carcinoma is recurrent adrenocortical carcinoma.

In embodiments, a adrenocortical carcinoma is a MSI-H adrenocortical carcinoma. In embodiments, a adrenocortical carcinoma is a MSS adrenocortical carcinoma. In embodiments, a adrenocortical carcinoma is a POLE-mutant adrenocortical carcinoma. In embodiments, a adrenocortical carcinoma is a POLD-mutant adrenocortical carcinoma. In embodiments, a adrenocortical carcinoma is a high TMB adrenocortical carcinoma. In embodiments, a adrenocortical carcinoma is associated with homologous recombination repair deficiency/homologous repair deficiency (“HRD”) or is characterized by a homologous recombination repair (HRR) gene mutation or deletion. In embodiments, a adrenocortical carcinoma is BRCA-deficient adrenocortical carcinoma. In embodiments, a adrenocortical carcinoma is characterized by PD-L1 expression (e.g., high PD-L1 expression).

In embodiments, a subject having adrenocortical carcinoma is a pediatric subject (e.g., as described herein). In embodiments, a subject is no more than about eighteen years of age. In embodiments, a subject is no more than about 10 years of age. In embodiments, a subject is no more than about 4 years of age. In embodiments, a subject is at least about 4 years of age. In embodiments, In embodiments, a subject is at least about 5 years of age. In embodiments, a subject is about 6 months of age to about 4 years of age. In embodiments, a subject is about 6 months of age to about 18 years of age.

In embodiments, a subject is male. In embodiments, a subject is female.

In embodiments, a subject with adrenocortical carcinoma has received a previous line of treatment (LOT). In embodiments, a therapeutic regimen described herein (e.g., treatment with niraparib and/or a PD-1 inhibitor such as TSR-042) is administered in combination with a further line of treatment (LOT). In embodiments, a LOT is surgery, a radiotherapy, a chemotherapy, an immunotherapy, an anti-angiogenic agent, or an anti-inflammatory, or any combination thereof. In embodiments, a LOT is surgery and/or chemotherapy.

In some embodiments, the methods of the invention treat subjects with a cancer with a dosage of 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg of niraparib or pharmaceutically acceptable salt thereof once-daily, twice-daily, or thrice-daily. In some embodiments, the methods of the invention treat subjects with a cancer with a dosage of 150 mg to 175 mg, 170 mg to 195 mg, 190 mg to 215 mg, 210 mg to 235 mg, 230 mg to 255 mg, 250 mg to 275 mg, 270 to 295 mg, 290 mg to 315 mg, 310 mg to 335 mg, 330 mg to 355 mg, 350 mg to 375 mg, or 370 mg to 400 mg of niraparib or pharmaceutically acceptable salt thereof once-daily, twice-daily, or thrice-daily. In some embodiments, the methods of the invention treat subjects with a cancer with a dosage of 5 mg, 7.5 mg, 10 mg, 12.5 mg, 15 mg. 17.5 mg, 20 mg, 22.5 mg, 25 mg, 27.5 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of niraparib or pharmaceutically acceptable salt thereof once-daily, twice-daily, or thrice-daily.

In some embodiments, the methods of the invention treat subjects with a cancer with a dosage of from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to about 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg of niraparib or pharmaceutically acceptable salt thereof once-daily, twice-daily, or thrice-daily. In some embodiments, the methods of the invention treat subjects with a cancer with a dosage of from about 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23/5 mg, 23 mg to 25.5 mg, 25 mg to 27.5 mg, 27 mg to 30 mg, 30 mg to 35 mg, 35 mg to 40 mg, 40 mg to 45 mg, 45 mg to 50 mg, 50 mg to 55 mg, 55 mg to 60 mg, 60 to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg of niraparib or pharmaceutically acceptable salt thereof once-daily, twice-daily, or thrice-daily.

Administration of the Compositions

In embodiments, niraparib is orally administered to a subject.

In embodiments, niraparib is orally administered to a subject in a solid oral dosage form. In embodiments, a solid oral dosage form is a tablet (e.g., any of the tablet formulations described herein). In embodiments, a solid oral dosage form is a tablet (e.g., any of the capsule dosage forms described herein).

In embodiments, niraparib is orally administered to a subject in a liquid oral dosage form. In embodiments, a liquid oral dosage form is a solution comprising niraparib. In embodiments, a liquid oral dosage form is a suspension comprising niraparib.

One of the recommended dosages the niraparib described herein as monotherapy is three 100 mg doses taken orally once daily, equivalent to a total daily dose of 300 mg. Patients may be encouraged to take their dose at approximately the same time each day. Bedtime administration may be a potential method for managing nausea.

As described herein, doses of 1 to 2000 mg of niraparib or a pharmaceutically acceptable salt thereof may be administered for treatment of subjects, and methods and compositions described herein may comprise once-daily, twice-daily, or thrice-daily administration of a dose of up to 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg once-daily, twice-daily, or thrice-daily. In some embodiments, the dose of niraparib or pharmaceutically acceptable salt thereof is from 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg, once-daily, twice-daily, or thrice-daily. In some embodiments, the methods of the invention treat subjects with a cancer with a dosage of 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg of niraparib or pharmaceutically acceptable salt thereof once-daily, twice-daily, or thrice-daily.

In some embodiments, a total daily dose of niraparib or a pharmaceutically acceptable salt thereof of 1 mg to 2000 mg. In some embodiments, a total daily dose of niraparib or a pharmaceutically acceptable salt thereof of 1 mg to 1000 mg, for example, or 50 to 300 mg, is administered. In some embodiments, the total daily dose of niraparib or a pharmaceutically acceptable salt thereof administered exceeds 100 mg per day. In some embodiments, the total daily dose of niraparib or a pharmaceutically acceptable salt thereof administered exceeds 200 mg per day. In some embodiments, the total daily dose of niraparib or a pharmaceutically acceptable salt thereof administered exceeds 300 mg per day. In some embodiments, the total daily dose of niraparib or a pharmaceutically acceptable salt thereof administered exceeds 400 mg per day. In some embodiments, the total daily dose of niraparib or a pharmaceutically acceptable salt thereof administered exceeds 500 mg per day.

In some embodiments, the total daily dose of niraparib or a pharmaceutically acceptable salt thereof administered does not exceed 500 mg per day. In some embodiments, the total daily dose of niraparib or a pharmaceutically acceptable salt thereof administered does not exceed 300 mg per day. In some embodiments, the total daily dose of niraparib or a pharmaceutically acceptable salt thereof administered does not exceed 100 mg per day. In some embodiments, the total daily dose of niraparib or a pharmaceutically acceptable salt thereof administered does not exceed 50 mg per day. In some embodiments, the total daily dose of niraparib or pharmaceutically acceptable salt thereof is from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg the total daily dose of niraparib or a pharmaceutically acceptable salt thereof is about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

A therapeutically effective dose of niraparib or a pharmaceutically acceptable salt thereof may be about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg per day. In some embodiments, the amount of niraparib or a pharmaceutically acceptable salt thereof administered daily is from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg per day.

In some embodiments, the amount of niraparib or a pharmaceutically acceptable salt thereof administered one time daily is 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some embodiments, the amount of niraparib or a pharmaceutically acceptable salt thereof administered one time daily is 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, the amount of niraparib or a pharmaceutically acceptable salt thereof administered two times daily is 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some embodiments, the amount of niraparib or a pharmaceutically acceptable salt thereof administered two times daily is 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, the amount of niraparib or a pharmaceutically acceptable salt thereof administered three times daily is 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some embodiments, the amount of niraparib or a pharmaceutically acceptable salt thereof administered three times daily is 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, the niraparib or a pharmaceutically acceptable salt thereof is present at a dose from about 1 mg to about 2000 mg, including, but not limited to, about 1 mg, 5 mg, 10.0 mg, 10.5 mg, 11.0 mg, 11.5 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100, 105 mg, 110 mg, 115 mg, 120 mg, 120.5 mg, 121 mg, 121.5 mg, 122 mg, 122.5 mg, 123 mg, 123.5 mg, 124 mg, 124.5 mg, 125 mg, 125.5 mg, 126 mg, 126.5 mg, 127 mg, 127.5 mg, 128 mg, 128.5 mg, 129 mg, 129.5 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, the niraparib or a pharmaceutically acceptable salt thereof is present at a dose from about 1 mg to 5 mg, 5 mg to 10 mg, 10 mg to 20 mg, 20 mg to 25 mg, 25 mg to 100 mg, 35 mg to 140 mg, 70 mg to 140 mg, 80 mg to 135 mg, 10 mg to 25 mg, 25 mg to 50 mg, 50 mg to 100 mg, 100 mg to 150 mg, 150 mg to 200 mg, 10 mg to 35 mg, 35 mg to 70 mg, 70 mg to 105 mg, 105 mg to 140 mg, 140 mg to 175 mg, or 175 mg to 200 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg.

Combination Therapy

In embodiments, a pediatric subject is administered niraparib in combination with one or more of surgery, a radiotherapy, a chemotherapy, an immunotherapy, an anti-angiogenic agent, or an anti-inflammatory.

In embodiments, a pediatric subject has been further administered or will be further administered an immune checkpoint inhibitor.

Exemplary immune checkpoint inhibitors include inhibitors of PD-1, LAG-3, CTLA-4, TIM-3, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R. In embodiments, an immune checkpoint inhibitor is an agent that inhibits PD-1, LAG-3, TIM-3, CTLA-4, TIGIT, IDO, or CSF1R.

In embodiments, an immune checkpoint inhibitor is an agent that inhibits PD-1 (e.g., a small molecule, a nucleic acid, a polypeptide, a carbohydrate, a lipid, a metal, a toxin, a PD-1 binding agent, or a PD-L1 binding agent).

In embodiments, a PD-1 inhibitor is a PD-L1/L2 binding agent (e.g., an antibody, an antibody conjugate, or an antigen-binding fragment thereof such as durvalumab, atezolizumab, avelumab, BGB-A333, SHR-1316, FAZ-053, CK-301, or, PD-L1 millamolecule, or derivatives thereof).

In embodiments, a PD-1 inhibitor is a PD-1 binding agent (e.g., an antibody, an antibody conjugate, or an antigen-binding fragment thereof such as nivolumab, pembrolizumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, AMP-514/MEDI-0680, AGEN-2034, CS1001, TSR-042, Sym-021, PF-06801591, LZMO09, KN-035, AB122, genolimzumab (CBT-501), AK 104, or GLS-010, or derivatives thereof). In embodiments, a PD-1 inhibitor is TSR-042.

In embodiments, a PD-1 inhibitor is administered to the subject periodically at a dose of about 50 mg to about 2000 mg, about 50 mg to about 1000 mg, or about 100 mg to about 500 mg.

In embodiments, a PD-1 inhibitor (e.g., TSR-042) is administered to the subject periodically at a dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, or about 1700 mg.

In some embodiments, a PD-1 inhibitor (e.g., TSR-042) is administered to the subject periodically at a dose that is an amount relative to body weight. In some embodiments, a dose of a PD-1 inhibitor (e.g., TSR-042) is within a range of about 0.01 mg/kg to 100 mg/kg of animal or human body weight; however, doses below or above this exemplary range are within the scope of the invention. A dose can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values). In embodiments, a dose of a PD-1 inhibitor (e.g., TSR-042) is about 0.5 mg/kg to about 10 mg/kg, about 0.5 mg/kg to about 8 mg/kg, about 1 mg/kg to about 8 mg/kg, about 2 mg/kg to about 8 mg/kg, or about 3 mg/kg to about 8 mg/kg. In embodiments, a dose of a PD-1 inhibitor (e.g., TSR-042) is about 1 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10 mg/kg.

In embodiments, a dose of a PD-1 inhibitor (e.g., TSR-042) is about 0.5 mg/kg to 2.0 mg/kg (e.g., about 0.5 mg/kg, 1.0 mg/kg, or 1.5 mg/kg). In embodiments, a dose of a PD-1 inhibitor (e.g., TSR-042) is about 3.0 mg/kg to 5.0 mg/kg (e.g., about 3.0 mg/kg, 3.5 mg/kg, or 4.0 mg/kg). In embodiments, a dose of a PD-1 inhibitor (e.g., TSR-042) is about 6.0 mg/kg to 8.0 mg/kg (e.g., about 6.5 mg/kg, about 7.0 mg/kg, or about 7.5 mg/kg).

In embodiments, a PD-1 inhibitor (e.g., TSR-042) is administered to the subject once every week, once every two weeks, once every three weeks, once every four weeks, once every five weeks, once every six weeks, once every seven weeks, once every eight weeks, once every nine weeks, or once every ten weeks.

In embodiments, a PD-1 inhibitor (e.g., TSR-042) is administered to the subject once every three weeks.

In embodiments, a PD-1 inhibitor (e.g., TSR-042) is administered to the subject in a dose of about 500 mg once every three weeks.

In embodiments, a PD-1 inhibitor (e.g., TSR-042) is administered to the subject in a dose of about 1.0 mg/kg to 10 mg/kg once every three weeks. In embodiments, a dose of a PD-1 inhibitor (e.g., TSR-042) is about 0.5 mg/kg to 2.0 mg/kg (e.g., about 0.5 mg/kg, 1.0 mg/kg, or 1.5 mg/kg) is administered once every three weeks. In embodiments, a dose of a PD-1 inhibitor (e.g., TSR-042) is about 3.0 mg/kg to 5.0 mg/kg (e.g., about 3.0 mg/kg, 3.5 mg/kg, or 4.0 mg/kg) is administered once every three weeks. In embodiments, a dose of a PD-1 inhibitor (e.g., TSR-042) is about 6.0 mg/kg to 8.0 mg/kg (e.g., about 6.5 mg/kg, about 7.0 mg/kg, or about 7.5 mg/kg) is administered once every three weeks.

In embodiments, a PD-1 inhibitor (e.g., TSR-042) is administered as a first dose once every 3 weeks for 3, 4, or 5 cycles followed by a second dose administered once every six weeks. In embodiments, a first dose is about 500 mg of the PD-1 inhibitor (e.g., TSR-042). In embodiments, a second dose is about 1000 mg of the PD-1 inhibitor (e.g., TSR-042).

Frequency of Administration

In some embodiments, a composition disclosed herein is administered to an individual in need thereof once. In some embodiments, a composition disclosed herein is administered to an individual in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein. In some embodiments, a first administration of a composition disclosed herein is followed by a drug holiday.

The number of times a composition is administered to an individual in need thereof depends on the discretion of a medical professional, the disorder, the severity of the disorder, and the individual's response to the formulation. In some embodiments, a composition disclosed herein is administered once to an individual in need thereof with a mild acute condition. In some embodiments, a composition disclosed herein is administered more than once to an individual in need thereof with a moderate or severe acute condition. In the case wherein the patient's condition does not improve, upon the doctor's discretion the administration of niraparib may be administered chronically, that is, for an extended period of time, including throughout the duration of the patient's life in order to ameliorate or otherwise control or limit the symptoms of the patient's disease or condition.

In some embodiments, the composition is administered at predetermined time intervals over an extended period of time. In some embodiments, the niraparib composition is administered once every day. In some embodiments, the niraparib composition is administered every other day. In some embodiments, the niraparib composition is administered over 1 week, 2 weeks, 1 month, 2 months, 3 months, 6 months, 1 year, 2 years, 3 years, 4 years, 5 years, 6 years, 7 years, 8 years, 9 years, 10 years, 11 years, or 12-15 years.

In some embodiments, the niraparib composition is administered in doses having a dose-to-dose niraparib concentration variation of less than 50%, less than 40%, less than 30%, less than 20%, less than 10%, or less than 5%.

In the case wherein the patient's status does improve, upon the doctor's discretion the administration of the niraparib may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. A first or second dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. For example, a first or second dose reduction during a drug holiday may be a dose reduced from 5 mg to 1 mg, 10 mg to 5 mg, 20 mg to 10 mg, 25 mg to 10 mg, 50 mg to 25 mg, 75 mg to 50 mg, 75 mg to 25 mg, 100 mg to 50 mg, 150 mg to 75 mg, 100 mg to 25 mg, 200 mg to 100 mg, 200 to 50 mg, 250 mg to 100 mg, 300 mg to 50 mg, 300 mg to 100 mg, 300 mg to 200 mg, 400 mg to 50 mg, 400 mg to 100 mg, 400 mg to 200 mg, 500 mg to 50 mg, 500 mg to 100 mg, 500 mg to 250 mg, 1000 mg to 50 mg, 1000 mg to 100 mg, or 1000 mg to 500 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a first or second dose reduction during a drug holiday may be a dose reduced by 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

Once improvement of the patient's condition has occurred, a maintenance niraparib dose is administered if necessary. Subsequently, the dosage or the frequency of administration, or both, is optionally reduced, as a function of the symptoms, to a level at which the improved disease, disorder or condition is retained. In certain embodiments, patients require intermittent treatment on a long-term basis upon any recurrence of symptoms.

Niraparib Formulations

The present invention recognizes the need to provide improved dosage forms of niraparib having desirable disintegration profiles, pharmacokinetic characteristics, flow properties, and/or good storage stability. The present invention relates to a process for the preparation of a solid, orally administrable pharmaceutical composition, comprising a poly (adenosine diphosphate [ADP]-ribose) polymerase (PARP)-1 and -2 inhibitor, and its use for the prophylaxis and/or treatment of diseases. The present invention relates to solid dosage forms of niraparib and pharmaceutically acceptable salts thereof (e.g., niraparib tosylate monohydrate), having desirable pharmacokinetic characteristics which exhibit favorable storage stability and disintegration properties. Niraparib has the following structure:

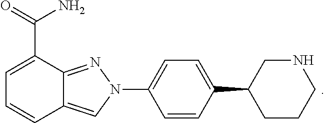 [SMILES: NC(=O)c1cccc2cn(-c3ccc([C@@H]4CCCNC4)cc3)nc12]

Niraparib is an orally available, selective poly(ADP-ribose) polymerase (PARP) 1 and 2 inhibitor. The chemical name for niraparib tosylate monohydrate is 2-{4-[(3S)-piperidin-3-yl]phenyl}-2H-indazole 7-carboxamide 4-methylbenzenesulfonate hydrate (1:1:1) and it has the following chemical structure:

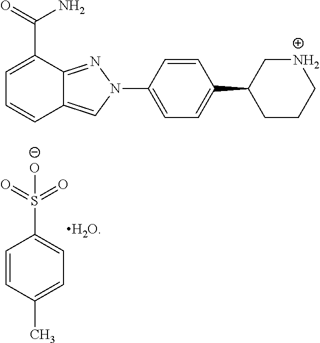

The empirical molecular formula for niraparib is C₂₆H₃₀N₄O₅S and its molecular weight is 510.61. Niraparib tosylate monohydrate drug substance is a white to off-white, non-hygroscopic crystalline solid. Niraparib solubility is pH independent below the pKa of 9.95, with an aqueous free base solubility of 0.7 mg/mL to 1.1 mg/mL across the physiological pH range.

Niraparib is a selective poly(ADP-ribose) polymerase (PARP) 1 and 2 inhibitor which selectively kills tumor cells in vitro and in mouse xenograft models. PARP inhibition leads to irreparable double strand breaks (DSBs), use of the error-prone DNA repair pathway, resultant genomic instability, and ultimately cell death. Additionally, PARP trapped at genetic lesions as a result of the suppression of autoparylation can contribute to cytotoxicity.

Niraparib, tradename ZEJULA®′ is indicated for the maintenance or treatment of adult patients with recurrent epithelial ovarian, fallopian tube, or primary peritoneal cancer following a complete or partial response to platinum-based chemotherapy. Each ZEJULA capsule contains 100 mg of niraparib (as tosylate monohydrate). The hard capsules may have a white body with “100 mg” printed in black ink, and a purple cap with “niraparib” printed in white ink. The current recommended dose of ZEJULA as monotherapy is three 100 mg capsules taken orally once daily, equivalent to a total daily dose of 300 mg.

Provided herein is an oral composition containing niraparib or its pharmaceutically acceptable salts.

In some embodiments, the oral composition includes from about 20 wt % to about 80 wt % of niraparib for treatment of a disorder or condition such as cancer; and a pharmaceutically acceptable carrier, wherein the niraparib is distributed with throughout the pharmaceutically acceptable carrier. In some embodiments, the oral composition includes from about 20 wt % to about 60 wt % of niraparib for treatment of a disorder or condition such as cancer; and a pharmaceutically acceptable carrier, wherein the niraparib is distributed with substantial uniformity throughout the pharmaceutically acceptable carrier. In some embodiments, the oral composition includes from about 35 wt % to about 55 wt % of niraparib for treatment of a disorder or condition such as cancer; and a pharmaceutically acceptable carrier, wherein the niraparib is distributed with substantial uniformity throughout the pharmaceutically acceptable carrier.

In some embodiments, the disorder or condition is cancer, for example, ovarian cancer. Other exemplary cancers are described herein.

In some embodiments, the niraparib is a pharmaceutically acceptable salt thereof. In some embodiments, the pharmaceutically acceptable salt is niraparib tosylate monohydrate.

In some embodiments, the pharmaceutical composition comprises about 10 mg to about 2000 mg of niraparib tosylate monohydrate. In some embodiments, the pharmaceutical composition comprises about 10 mg to about 1000 mg of niraparib tosylate monohydrate. In some embodiments, the pharmaceutical composition comprises about 10 mg to about 525 mg of niraparib tosylate monohydrate. In some embodiments, the pharmaceutical composition comprises about 425 mg to about 525 mg of niraparib tosylate monohydrate.

In some embodiments, the pharmaceutical composition comprises about 50 mg to about 300 mg of niraparib tosylate monohydrate. In some embodiments, the pharmaceutical composition comprises about 50 mg to about 525 mg of niraparib tosylate monohydrate. For example, the pharmaceutical composition can comprise about 100 mg to about 200 mg of niraparib tosylate monohydrate. For example, the pharmaceutical composition can comprise about 125 mg to about 175 mg of niraparib tosylate monohydrate.

The formulation can comprise one or more components, including niraparib. The components can be combined to create granules that are then compressed to form tablets.

The niraparib may be present in the formulation as a pharmaceutically acceptable salt. For example, the niraparib can be niraparib tosylate monohydrate. In some embodiments, the components can be combined to create a powder blend that is used to fill capsules. For example, the powder blend can be filled into gelatin capsules, such as size 0 gelatin capsules.

The niraparib may be present in the formulation as a pharmaceutically acceptable salt. For example, the niraparib can be niraparib tosylate monohydrate.

Exemplary formulations include those described in International Application Nos. PCT/US18/52979 and PCT/US2018/024603 (WO/2018/183354), each of which is incorporated by reference in its entirety.

The formulation can comprise one or more diluents. For example, the formulation can comprise lactose monohydrate.

The formulation can comprise one or more lubricants. For example, For example, the formulation can comprise magnesium stearate.

An exemplary niraparib formulation of the present invention comprises 100 mg of niraparib (based on free base, 1.000 mg niraparib anhydrous free base is equivalent to 1.594 mg niraparib tosylate monohydrate), lactose monohydrate and magnesium stearate. An exemplary niraparib formulation of the present invention comprises 100 mg of niraparib (based on free base, 1.000 mg niraparib anhydrous free base is equivalent to 1.594 mg niraparib tosylate monohydrate), lactose monohydrate, magnesium stearate and tartrazine.

In some embodiments, the pharmaceutical composition is formulated into solid oral pharmaceutical dosage forms. Solid oral pharmaceutical dosage forms include, but are not limited to, tablets, capsules, powders, granules and sachets. For example, the solid oral pharmaceutical dosage form can be a tablet or a capsule.

In embodiments, the pharmaceutical composition is formulated into liquid oral dosage forms. In embodiments, a liquid oral dosage form is a suspension. In embodiments, a liquid oral dosage form is a solution.

In certain embodiments, a solid dosage form can be further manipulated for use in any of the methods described herein. For example, a tablet can be crushed and administered with food or mixed with a liquid to form a solution or suspension. The contents of a capsule may be administered with food (e.g., soft food) as sprinkles.

In some embodiments, a therapeutically effective amount of niraparib or a pharmaceutically acceptable salt thereof administered to a subject via a solid dosage form is in the range of about 1 mg to about 2000 mg. In some embodiments, a therapeutically effective amount of niraparib or a pharmaceutically acceptable salt thereof administered to a subject via a solid dosage form is in the range of about 1 mg to about 1000 mg. In some embodiments, a therapeutically effective amount of niraparib or a pharmaceutically acceptable salt thereof administered to a subject via a solid dosage form is in the range of from about 50 mg to about 300 mg. In some embodiments, a niraparib formulation is administered as a solid dosage form at a concentration of about 50 mg to about 100 mg. In some embodiments, the niraparib formulation is administered as a solid dosage form at concentration of about 100 mg to about 300 mg. For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a therapeutically effective amount of niraparib tosylate monohydrate administered to a subject via a solid dosage form can be from about 1 mg to about 2000 mg, for example, from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a therapeutically effective amount of niraparib tosylate monohydrate administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a therapeutically effective amount of niraparib tosylate monohydrate administered to a subject via a solid dosage form can be about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some embodiments, a therapeutically effective amount of niraparib tosylate monohydrate administered to a subject via a solid dosage form is about 79.7 mg. In some embodiments, a therapeutically effective amount of niraparib tosylate monohydrate administered to a subject via a solid dosage form is about 159.4 mg. In some embodiments, a therapeutically effective amount of niraparib tosylate monohydrate administered to a subject via a solid dosage form is about 318.8 mg. In some embodiments, a therapeutically effective amount of niraparib tosylate monohydrate administered to a subject via a solid dosage form is about 478.0 mg. In some aspects, the solid oral dosage form can be administered one, two, or three times a day (b.i.d).

Contemplated compositions of the present invention provide a therapeutically effective amount of niraparib or a pharmaceutically acceptable salt thereof over an interval of about 30 minutes to about 8 hours after administration, enabling, for example, once-a-day, twice-a-day, three times a day, and etc. administration if desired.

Pharmacodynamics

Niraparib inhibits PARP-1 and PARP-2 enzymes in vitro with IC₅₀ of 3.8 nM (0.82 ng/mL) and 2.1 nM (0.67 ng/mL), respectively. Niraparib inhibits intracellular PARP activity, with an IC₅₀ of 4 nM (1.28 mg/mL) and an IC₉₀ of 50 nM (16 ng/mL). A single dose of 50 mg/kg niraparib in tumor models resulted in >90% PARP inhibition and with daily dosing, tumor regression. At a dose of 50 mg/kg, tumor concentrations of 4567 ng/mL were achieved at 6 hours, which exceeds the PARP IC₉₀ and resulted in tumor regression. In this same model, a dose of 75 mg/kg niraparib did not result in tumor regression; tumor regression was achieved when dosing was switched to a 50 mg/kg dose of niraparib.

As used herein, fasted human pharmacokinetic studies include both single dose, fasted, human pharmacokinetic studies and multiple dose, fasted, human pharmacokinetic studies. Multiple dose, fasted, human pharmacokinetic studies are performed in accordance to the FDA Guidance documents and/or analogous EMEA Guidelines. Pharmacokinetic parameters for steady state values may be determined directly from multiple dose, fasted, human pharmacokinetic studies or may be conveniently determined by extrapolation of single dose data using standard methods or industry standard software such as WinNonlin version 5.3 or higher.

In some embodiments, a once daily oral administration of a niraparib composition described herein to a human subject provides a mean peak plasma concentration (Cₘₐₓ) of 600 ng/mL to 1000 ng/mL. For example, a once daily oral administration of a niraparib composition described herein to a human subject can provide a mean peak plasma concentration (Cₘₐₓ) of 600 ng/mL, 625 ng/mL, 650 ng/mL, 675 ng/mL, 700 ng/mL, 725 ng/mL, 750 ng/mL, 775 ng/mL, 800 ng/mL, 825 ng/mL, 850 ng/mL, 875 ng/mL, 900 ng/mL, 925 ng/mL, 950 ng/mL, 975 ng/mL or 1000 ng/mL. For example, a once daily oral administration of a niraparib composition described herein to a human subject can provide a mean peak plasma concentration (Cₘₐₓ) of 804 ng/mL.

In some embodiments, a once daily oral administration of a niraparib composition described herein to a human subject provides a mean peak plasma concentration (Cₘₐₓ) in 0.5 to 6 hours. For example, a once daily oral administration of a niraparib composition described herein to a human subject can provide a mean peak plasma concentration (Cₘₐₓ) in about 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, or 6 hours.

In some embodiments, an absolute bioavailability of niraparib provided in a composition described herein is about 60-90%. For example, an absolute bioavailability of niraparib provided in a composition described herein can be about 60%, 65%, 70%, 75%, 80%, 85% or 90%. For example, an absolute bioavailability of niraparib provided in a composition described herein can be about 73%.

In some embodiments, concomitant administration of a high fat meal does not significantly affect the pharmacokinetics of a niraparib composition described herein after administration of a dose described herein. For example, concomitant administration of a high fat meal may not significantly affect the pharmacokinetics of a niraparib composition described herein after administration of a 50 mg, 100 mg, 150 mg, 200 mg, 250 mg, 300 mg, 350 mg or 400 mg dose of niraparib.

In some embodiments, niraparib is moderately protein bound to human plasma after administration to a human subject. For example, after administration to a human subject about 60%, 65%, 70%, 75%, 80%, 85% or 90% of the niraparib is protein bound to human plasma. For example, after administration to a human subject about 83% of the niraparib is protein bound to human plasma.

In some embodiments, an apparent volume of distribution (Vd/F) of niraparib is from about 500 L to about 2000 L after administration to a human subject. For example, an apparent volume of distribution (Vd/F) of niraparib can be about 500 L, 550 L, 600 L, 650 L, 700 L, 750 L, 800 L, 850 L, 900 L, 950 L, 1000 L, 1100 L, 1200 L, 1300 L, 1350 L, 1400 L, 1450 L, 1500 L, 1600 L, 1700 L, 1800 L, 1900 L or 2000 L after administration to a human subject. For example, an apparent volume of distribution (Vd/F) of niraparib can be about 1220 L after administration to a human subject. For example, an apparent volume of distribution (Vd/F) of niraparib can be about 1074 L after administration to a human subject with cancer.

In some embodiments, following administration of niraparib provided in a composition described herein, the mean terminal half-life (t_{1/2}) of niraparib is from about 40 to 60 hours. For example, following administration of niraparib provided in a composition described herein, the mean terminal half-life (t_{1/2}) of niraparib can be about 40 hours, 42 hours, 44 hours, 46 hours, 48 hours, 50 hours, 52 hours, 54 hours, 56 hours, 58 hours or 60 hours. For example, following administration of niraparib provided in a composition described herein, the mean terminal half-life (t_{1/2}) of niraparib can be about 48 to 51 hours. For example, following administration of niraparib provided in a composition described herein, the mean terminal half-life (tin) of niraparib can be about 48 hours, 49 hours, 50 hours or 51 hours.

In some embodiments, following administration of niraparib provided in a composition described herein, the apparent total clearance (CL/F) of niraparib is from about 10 L/hour to about 20 L/hour. For example, following administration of niraparib provided in a composition described herein, the apparent total clearance (CL/F) of niraparib can be about 10 L/hour, 11 L/hour, 12 L/hour, 13 L/hour, 14 L/hour, 15 L/hour, 16 L/hour, 17 L/hour, 18 L/hour, 19 L/hour or 20 L/hour. For example, following administration of niraparib provided in a composition described herein, the apparent total clearance (CL/F) of niraparib can be about 16.2 L/hour.

In some embodiments, the formulations disclosed herein provide a release of niraparib from the composition within 1 minute, or within 5 minutes, or within 10 minutes, or within 15 minutes, or within 30 minutes, or within 60 minutes or within 90 minutes. In other embodiments, a therapeutically effective amount of niraparib is released from the composition within 1 minute, or within 5 minutes, or within 10 minutes, or within 15 minutes, or within 30 minutes, or within 60 minutes or within 90 minutes. In some embodiments the composition comprises a niraparib tablet formulation providing immediate release of niraparib. In some embodiments the composition comprises a niraparib tablet formulation providing immediate release of niraparib within 1 minute, or within 5 minutes, or within 10 minutes, or within 15 minutes, or within 30 minutes, or within 60 minutes or within 90 minutes.

The niraparib formulations and dosage forms described herein display pharmacokinetic profiles that can result in Cₘᵢₙ niraparib blood plasma levels at steady state from about 10 ng/ml to about 100 ng/ml. In one embodiment, the niraparib formulations described herein provide blood plasma levels immediately prior to the next dose (Cₘᵢₙ) at steady state from about 25 ng/ml to about 100 ng/ml. In another embodiment, the niraparib formulations described herein provide Cₘᵢₙ blood plasma levels at steady state from about 40 ng/ml to about 75 ng/ml. In yet another embodiment, the niraparib formulations described herein provide Cₘᵢₙ blood plasma levels at steady state of about 50 ng/ml.

The niraparib formulations described herein are administered and dosed in accordance with good medical practice, taking into account the clinical condition of the individual patient, the site and method of administration, scheduling of administration, and other factors known to medical practitioners. In human therapy, the dosage forms described herein deliver niraparib formulations that maintain a therapeutically effective amount of niraparib of at least 10 ng/ml or typically at least about 100 ng/ml in plasma at steady state while reducing the side effects associated with an elevated Cₘₐₓ blood plasma level of niraparib.

In some embodiments, greater than about 95%; or greater than about 90%; or greater than about 80%; or greater than about 70% of the niraparib dosed by weight is absorbed into the bloodstream within 1, 2, 3, 4, 5, 6, 7, 8, 9, 10, 11, 12, 16, 18, or 24 hours after administration.

Niraparib Concentration/Amount

By means of methods and compositions described herein, formulations can be made that achieve the desired disintegration characteristics and target pharmacokinetic profiles described herein. For example, therapeutically effective doses of niraparib can be administered once, twice or three times daily in tablets using the manufacturing methods and compositions that have been described herein to achieve these results. In some embodiments, the niraparib or a pharmaceutically acceptable prodrug or salt thereof is present in an amount of from 20-80 wt %, 45-70 wt %, 40-50 wt %, 45-55 wt %, 50-60 wt %, 55-65 wt %, 60-70 wt %, 65-75 wt %, 70-80 wt %, or 40-60 wt %.

In some embodiments, the compositions described herein have a concentration of niraparib or a pharmaceutically acceptable prodrug or salt thereof of from about 1% to about 70%, from about 5% to about 70%, from about 10% to about 70%, from about 15% to about 70%, from about 20% to about 70%, from about 25% to about 70%, from about 30% to about 70%, from about 35% to about 70%, from about 40% to about 70%, from about 45% to about 70%, from about 50% to about 70%, from about 55% to about 70%, from about 60% to about 70%, from about 65% to about 70% by weight of the composition.

In some embodiments, the compositions described herein have a concentration of niraparib or a pharmaceutically acceptable prodrug or salt thereof of from about 1% to about 65%, from about 5% to about 65%, from about 10% to about 65%, from about 15% to about 65%, from about 20% to about 65%, from about 25% to about 65%, from about 30% to about 65%, from about 35% to about 65%, from about 40% to about 65%, from about 45% to about 65%, from about 50% to about 65%, from about 55% to about 65%, or from about 60% to about 65% by weight of the composition.

In some embodiments, the compositions described herein have a concentration of niraparib or a pharmaceutically acceptable prodrug or salt thereof of from about 1% to about 60%, from about 5% to about 60%, from about 10% to about 60%, from about 15% to about 60%, from about 20% to about 60%, from about 25% to about 60%, from about 30% to about 60%, from about 35% to about 60%, from about 40% to about 60%, from about 45% to about 60%, from about 50% to about 60%, or from about 55% to about 60% by weight of the composition.

In some embodiments, the compositions described herein have a concentration of niraparib or a pharmaceutically acceptable prodrug or salt thereof of from about 1% to about 55%, from about 5% to about 55%, from about 10% to about 55%, from about 15% to about 55%, from about 20% to about 55%, from about 25% to about 55%, from about 30% to about 55%, from about 35% to about 55%, from about 40% to about 55%, from about 45% to about 55%, or from about 50% to about 55% by weight of the composition.

In some embodiments, the compositions described herein have a concentration of niraparib or a pharmaceutically acceptable prodrug or salt thereof of from about 1% to about 50%, from about 5% to about 50%, from about 10% to about 50%, from about 15% to about 50%, from about 20% to about 50%, from about 25% to about 50%, from about 30% to about 50%, from about 35% to about 50%, from about 40% to about 50%, or from about 45% to about 50% by weight of the composition.

In some embodiments, the compositions described herein have a concentration of niraparib or a pharmaceutically acceptable prodrug or salt thereof of from about 1% to about 45%, from about 5% to about 45%, from about 10% to about 45%, from about 15% to about 45%, from about 20% to about 45%, from about 25% to about 45%, from about 30% to about 45%, from about 35% to about 45%, or from about 40% to about 45% by weight of the composition.

In some embodiments, the compositions described herein have a concentration of niraparib or a pharmaceutically acceptable prodrug or salt thereof of from about 1% to about 40%, from about 5% to about 40%, from about 10% to about 40%, from about 15% to about 40%, from about 20% to about 40%, from about 25% to about 40%, from about 30% to about 40%, from about 35% to about 40% by weight of the composition.

In some embodiments, the compositions described herein have a concentration of niraparib or a pharmaceutically acceptable prodrug or salt thereof of from about 1% to about 35%, from about 5% to about 35%, from about 10% to about 35%, from about 15% to about 35%, from about 20% to about 35%, from about 25% to about 35%, or from about 30% to about 35% by weight of the composition.

In some embodiments, the compositions described herein have a concentration of niraparib or a pharmaceutically acceptable prodrug or salt thereof of about 1%, 5%, 10%, 15%, 20%, 25%, 30%, 35%, 40%, 45%, 50%, 55%, or 60% by weight of the composition. In some embodiments, the compositions described herein have a concentration of niraparib tosylate monohydrate of about 19.16% by weight of the composition. In some embodiments, the compositions described herein have a concentration of niraparib tosylate monohydrate of about 38.32% by weight of the composition. In some embodiments, the compositions described herein have a concentration of niraparib tosylate monohydrate of about 47.8% by weight of the composition. In some embodiments, the compositions described herein have a concentration of niraparib tosylate monohydrate of about 57.48% by weight of the composition. In some embodiments, the compositions described herein have a concentration of niraparib tosylate monohydrate of about 76.64% by weight of the composition.

In some embodiments, the compositions described herein have an amount of niraparib or a pharmaceutically acceptable prodrug or salt thereof of from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg.

For example, the compositions described herein can have an amount of niraparib tosylate monohydrate of from about 1 mg to about 2000 mg, for example, from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg.

In some embodiments, the compositions described herein have an amount of niraparib or a pharmaceutically acceptable prodrug or salt thereof of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg. For example, the compositions described herein can have an amount of niraparib tosylate monohydrate of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg.

In some embodiments, the compositions described herein have an amount of niraparib or a pharmaceutically acceptable prodrug or salt thereof of about 25 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg. For example, the compositions described herein can have an amount of niraparib tosylate monohydrate of about 25 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg. In some embodiments, the compositions described herein have an amount of niraparib tosylate monohydrate of about 79.7 mg. In some embodiments, the compositions described herein have an amount of niraparib tosylate monohydrate of about 159.4 mg. In some embodiments, the compositions described herein have an amount of niraparib tosylate monohydrate of about 318.8 mg. In some embodiments, the compositions described herein have an amount of niraparib tosylate monohydrate of about 478.0 mg or about 478.2 mg.

Pharmaceutically Acceptable Salts

In some embodiments, the niraparib used in a composition disclosed herein is the form of a free base, pharmaceutically acceptable salt, prodrug, analog or complex. In some instances, the niraparib comprises the form of a pharmaceutically acceptable salt. In some embodiments, with respect to niraparib in a composition, a pharmaceutically acceptable salt includes, but is not limited to, 4-methylbenzenesulfonate salts, sulfate salts, benzenesulfate salts, fumarate salts, succinate salts, and stereoisomers or tautomers thereof. In some embodiments, with respect to niraparib in a composition, a pharmaceutically acceptable salt includes, but is not limited to, tosylate salts. In some embodiments, with respect to niraparib in a composition, a pharmaceutically acceptable salt includes, but is not limited to, tosylate monohydrate salts.

Pharmaceutically Acceptable Excipients

In some aspects, the pharmaceutical composition disclosed herein comprises one or more pharmaceutically acceptable excipients. In some aspects, the pharmaceutical composition disclosed herein further comprises one or more pharmaceutically acceptable excipients. In some embodiments, the one or more pharmaceutically acceptable excipient is present in an amount of about 0.1-99% by weight. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, stabilizers, suspending agents, adsorbents, granulating agents, preservatives, buffers, coloring agents and sweeteners or combinations thereof. Examples of binders include microcrystalline cellulose, hydroxypropyl methylcellulose, carboxyvinyl polymer, polyvinylpyrrolidone, polyvinylpolypyrrolidone, carboxymethylcellulose calcium, carboxymethylcellulose sodium, ceratonia, chitosan, cottonseed oil, dextrates, dextrin, ethylcellulose, gelatin, glucose, glyceryl behenate, galactomannan polysaccharide, hydroxyethyl cellulose, hydroxyethylmethyl cellulose, hydroxypropyl cellulose, hypromellose, inulin, lactose, magnesium aluminum silicate, maltodextrin, methylcellulose, poloxamer, polycarbophil, polydextrose, polyethylene glycol, polyethylene oxide, polymethacrylates, sodium alginate, sorbitol, starch, sucrose, sunflower oil, vegetable oil, tocofersolan, zein, or combinations thereof. Examples of disintegrants include hydroxypropyl methylcellulose (HPMC), low substituted hydroxypropyl cellulose (L-HPC), croscarmellose sodium, sodium starch glycolate, lactose, magnesium aluminum silicate, methylcellulose, polacrilin potassium, sodium alginate, starch, or combinations thereof. Examples of a lubricant include stearic acid, sodium stearyl fumarate, glyceryl behenate, calcium stearate, glycerin monostearate, glyceryl palmitostearate, magnesium lauryl sulfate, mineral oil, palmitic acid, myristic acid, poloxamer, polyethylene glycol, sodium benzoate, sodium chloride, sodium lauryl sulfate, talc, zinc stearate, potassium benzoate, magnesium stearate or combinations thereof. Examples of diluents include talc, ammonium alginate, calcium carbonate, calcium lactate, calcium phosphate, calcium silicate, calcium sulfate, cellulose, cellulose acetate, corn starch, dextrates, dextrin, dextrose, erythritol, ethylcellulose, fructose, fumaric acid, glyceryl palmitostearate, isomalt, kaolin, lactitol, lactose, magnesium carbonate, magnesium oxide, maltodextrin, maltose, mannitol, microcrystalline cellulose, polydextrose, polymethacrylates, simethicone, sodium alginate, sodium chloride, sorbitol, starch, sucrose, sulfobutylether O-cyclodextrin, tragacanth, trehalose, xylitol, or combinations thereof. In some embodiments, the pharmaceutically acceptable excipient is hydroxypropyl methylcellulose (HPMC). In some embodiments, the pharmaceutically acceptable excipient is low substituted hydroxypropyl cellulose (L-HPC). In some embodiments, the pharmaceutically acceptable excipient is lactose. In some embodiments, the pharmaceutically acceptable excipient is lactose monohydrate. In some embodiments, the pharmaceutically acceptable excipient is magnesium stearate. In some embodiments, the pharmaceutically acceptable excipient is lactose monohydrate and magnesium stearate.

Various useful fillers or diluents include, but are not limited to calcium carbonate (Barcroft™, MagGran™, Millicarb™, Pharma-Carb™, Precarb™, Sturcal™ Vivapres Ca™), calcium phosphate, dibasic anhydrous (Emcompress Anhydrous™ Fujicalin™), calcium phosphate, dibasic dihydrate (Calstar™, Di-Cafos™, Emcompress™) calcium phosphate tribasic (Tri-Cafos™, TRI-TAB™), calcium sulphate (Destab™, Drierite™, Snow White™, Cal-Tab™, Compactrol™), cellulose powdered (Arbocel™ Elcema™, Sanacet™), silicified microcrystalline cellulose (ProSolv® SMCC), cellulose acetate, compressible sugar (Di-Pac™), confectioner's sugar, dextrates (Candex™, Emdex™), dextrin (Avedex™, Caloreen™, Primogran W™), dextrose (Caridex™, Dextrofin™, Tab fine D-IOO™), fructose (Fructofin™, Krystar™), kaolin (Lion™, Sim 90TH), lactitol (Finlac DC™, Finlac MCX™), lactose (Anhydrox™, CapsuLac™, Fast-Flo™, FlowLac™, GranuLac™, InhaLac™, Lactochem™, Lactohaie™, Lactopress™, Microfine™, Microtose™, Pharmatose™, Prisma Lac™, Respitose™, SacheLac™, SorboLac™, Super-Tab™, Tablettose™, Wyndale™, Zeparox™), lactose monohydrate, magnesium carbonate, magnesium oxide (MagGran MOTH), maltodextrin (C*Dry MD™, Lycatab DSH™, Maldex™, Maitagran™, Maltrin™, Maltrin QD™, Paselli MD 10 PH™, Star-Dri™), maltose (Advantose 100TH), mannitol (Mannogem™, Pearlitol™) microcrystalline cellulose (Avicel PH™, Celex™, Celphere™, Ceolus KG™, Emcocel™, Pharmacel™, Tabulose™, Vivapur™), polydextrose (Litesse™), simethicone (Dow Corning Q7-2243 LVA™, Dow Corning Q7-2587TH, Sentry Simethicone™), sodium alginate (Keltone™, Protanal™), sodium chloride (Alberger™), sorbitol (Liponec 70-NC™, Liponic 76-NCv, Meritol™, Neosorb™, Sorbitol Instant™, Sorbogem™), starch (Flufiex W™, Instant Pure-Cote™, Melojel™, Meritena Paygel 55™, Perfectamyl D6PH™, Pure-Cote™, Pure-Dent™, Pure-Ge1™, Pure-Set™, Purity 21™, Purity 826™, Tablet White™) pregelatinized starch, sucrose, trehalose and xylitol, or mixtures thereof.

In some embodiments, a filler such as lactose monohydrate is present in an amount of about 5-90% by weight. In some embodiments, a filler such as lactose monohydrate is present in an amount of about 5-80% by weight. In some embodiments, a filler such as lactose monohydrate is present in an amount of about 5-70% by weight. In some embodiments, a filler such as lactose monohydrate is present in an amount of about 5-60% by weight. In some embodiments, a filler such as lactose monohydrate is present in an amount of about 5-50% by weight. In some embodiments, a filler such as lactose monohydrate is present in an amount of about 5-40% by weight. In some embodiments, a filler such as lactose monohydrate is present in an amount of about 5-30% by weight. In some embodiments, a filler such as lactose monohydrate is present in an amount of about 25-90% by weight. In some embodiments, a filler such as lactose monohydrate is present in an amount of about 25-80% by weight. In some embodiments, a filler such as lactose monohydrate is present in an amount of about 25-70% by weight. In some embodiments, a filler such as lactose monohydrate is present in an amount of about 25-60% by weight. In some embodiments, a filler such as lactose monohydrate is present in an amount of about 25-50% by weight. In some embodiments, a filler such as lactose monohydrate is present in an amount of about 25-40% by weight. In some embodiments, a filler such as lactose monohydrate is present in an amount of about 40-90% by weight. In some embodiments, a filler such as lactose monohydrate is present in an amount of about 40-80% by weight. In some embodiments, a filler such as lactose monohydrate is present in an amount of about 40-70% by weight. In some embodiments, a filler such as lactose monohydrate is present in an amount of about 40-60% by weight. In some embodiments, a filler such as lactose monohydrate is present in an amount of about 40-50% by weight. In some embodiments, a filler such as lactose monohydrate is present in an amount of about 40% by weight. In some embodiments, a filler such as lactose monohydrate is present in an amount of about 50% by weight. In some embodiments, a filler such as lactose monohydrate is present in an amount of about 60% by weight. In some embodiments, a filler such as lactose monohydrate is present in an amount of about 70% by weight. In some embodiments, a filler such as lactose monohydrate is present in an amount of about 80% by weight.

In some embodiments, a filler such as lactose monohydrate is present in an amount of from about 25 mg to about 1000 mg, from about 50 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, or from about 500 mg to about 1000 mg. For example, a filler such as lactose monohydrate can be present in an amount of from about 25 mg to about 1000 mg, from about 50 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, or from about 500 mg to about 1000 mg.

In some embodiments, a filler such as lactose monohydrate is present in an amount of from about 25 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, or from about 500 mg to about 550 mg. For example, a filler such as lactose monohydrate can be present in an amount of from about 25 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, or from about 500 mg to about 550 mg.

In some embodiments, a filler such as lactose monohydrate is present in an amount of about 15 mg, about 25 mg, about 50 mg, about 100 mg, about 150 mg, about 200 mg, about 250 mg, about 300 mg, about 350 mg, about 400 mg, about 450 mg, or about 500 mg. For example, a filler such as lactose monohydrate can be present in an amount of about 15 mg, about 25 mg, about 50 mg, about 100 mg, about 150 mg, about 200 mg, about 250 mg, about 300 mg, about 350 mg, about 400 mg, about 450 mg, or about 500 mg. In some embodiments, a filler such as lactose monohydrate is present in an amount of about 334.2 mg. In some embodiments, a filler such as lactose monohydrate is present in an amount of about 254.5 mg. In some embodiments, a filler such as lactose monohydrate is present in an amount of about 174.8 mg. In some embodiments, a filler such as lactose monohydrate is present in an amount of about 95.1 mg. In some embodiments, a filler such as lactose monohydrate is present in an amount of about 15.4 mg.

Various useful disintegrants include, but are not limited to, alginic acid (Protacid™, Satialgine H8™), calcium phosphate, tribasic (TRI-TAB™), carboxymethylcellulose calcium (ECG 505™), carboxymethylcellulose sodium (Akucell™ Finnfix™, Nymcel Tylose CB™), colloidal silicon dioxide (Aerosil™, Cab-O-Si1™, Wacker HDK™), croscarmellose sodium (Ac-Di-Sol™, Pharmacel XL™, Primellose™, Solutab™ Vivasol™), crospovidone (Collison CL™, Collison CL-M™, Polyplasdone XL™), docusate sodium, guar gum (Meyprodor™, Meyprofm™, Meyproguar™), low substituted hydroxypropyl cellulose, magnesium aluminum silicate (Magnabite™, Neusilin™ Pharmsorb™, Veegum™), methylcellulose (Methocel™, Metolose™), microcrystalline cellulose (Avicel PH™, Ceoius KG™, Emcoel™ Ethispheres™ Fibrocel™ Pharmacel™ Vivapur™), povidone (Collison™, Plasdone™) sodium alginate (Kelcosol™, Ketone™ Protanal™), sodium starch glycolate, polacrilin potassium (Amberlite IRP88™), silicified microcrystalline cellulose (ProSotv™), starch (Aytex P™, Fluftex W™, Melojel™ Meritena™, Paygel 55™, Perfectamyl D6PH™, Pure-Bind™, Pure-Cote™, Pure-Dent™ Purity 21™, Purity 826™, Tablet White™) or pre-gelatinized starch (Lycatab PGS™ Merigel™, National 78-1551™, Pharma-Ge1™, Prejel™, Sepistab ST200™, Spress B820™, Starch 1500 G™, Tablitz™, Unipure LD™), or mixtures thereof. In some embodiments, a disintegrant is optionally used in an amount of about 0.1-99% by weight. In some embodiments, a disintegrant is optionally used in an amount of about 0.1-50% by weight. In some embodiments, a disintegrant is optionally used in an amount of about 0.1-10% by weight. In some embodiments, a disintegrant is present in an amount of from about 0.1 mg to 0.5 mg, 0.5 mg to 1 mg, 1 mg to 2 mg, 2 mg to 2.5 mg, 2.5 mg to 5 mg, 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23.5 mg, 23 mg to 25.5 mg, 25 mg to 27.5 mg, 27 mg to 30 mg, 29 mg to 31.5 mg, 31 mg to 33.5 mg, 33 mg to 35.5 mg, 35 mg to 37.5 mg, 37 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg. In some embodiments, a disintegrant is present in an amount of about 0.1 mg, 0.5 mg, 1 mg, 2 mg, 2.5 mg, 5 mg, 7 mg, 9 mg, 11 mg, 13 mg, 15 mg, 17 mg, 19 mg, 21 mg, 23 mg, 25 mg, 27.5 mg, 30 mg, 31.5 mg, 33.5 mg, 35.5 mg, 37.5 mg, 40 mg, 45 mg, 50 mg, 55 mg, 60 mg, 65 mg, 70 mg, 75 mg, 80 mg, 85 mg, 90 mg, 95 mg, or 100 mg.

Various useful lubricants include, but are not limited to, calcium stearate (HyQual™), glycerine monostearate (Imwitor™ 191 and 900, Kessco GMSS™, 450 and 600, Myvaplex 600P™, Myvatex™, Rita GMS™, Stepan GMS™, Tegin™, Tegin™ 503 and 515, Tegin 4100™, Tegin M™, Unimate GMS™), glyceryl behenate (Compritol 888 ATO™), glyceryl palmitostearate (Precirol ATO 5™), hydrogenated castor oil (Castorwax MP80™, Croduret™, Cutina HR™, Fancol™, Simulsol 1293™), hydrogenated vegetable oil 0 type I (Sterotex™, Dynasan P60™, Hydrocote™, Lipovol HS-K™, Sterotex HM™), magnesium lauryl sulphate, magnesium stearate, medium-chain triglycerides (Captex 300™, Labrafac CC™, Miglyol 810™, Neobee MS™, Nesatol™, Waglinol 3/9280™), poloxamer (Pluronic™, Synperonic™), polyethylene 5 glycol (Carbowax Sentry™, Lipo™, Lipoxol™, Lutrol E™, Pluriol E™), sodium benzoate (Antimol™), sodium chloride, sodium lauryl sulphate (Elfan 240™, Texapon Kl 2P™), sodium stearyl fumarate (Pruv™), stearic acid (Hystrene™, Industrene™, Kortacid 1895™, Pristerene™), talc (Altaic™, Luzenac™, Luzenac Pharma™, Magsil Osmanthus™, 0 Magsil Star™, Superiore™), sucrose stearate (Surfhope SE Pharma D-1803 F™) and zinc stearate (HyQual™) or mixtures thereof. Examples of suitable lubricants include, but are not limited to, magnesium stearate, calcium stearate, zinc stearate, stearic acid, talc, glyceryl behenate, polyethylene glycol, polyethylene oxide polymers, sodium lauryl sulfate, magnesium lauryl sulfate, sodium oleate, sodium stearyl fumarate, DL-leucine, colloidal silica, and others as known in the art. In some embodiments a lubricant is magnesium stearate.

In some embodiments, a lubricant such as magnesium stearate is present in an amount of about 0.1-5% by weight. In some embodiments, a lubricant such as magnesium stearate is present in an amount of about 0.1-2% by weight. In some embodiments, a lubricant such as magnesium stearate is present in an amount of about 0.1-1% by weight. In some embodiments, a lubricant such as magnesium stearate is present in an amount of about 0.1-0.75% by weight. In some embodiments, a lubricant such as magnesium stearate is present in an amount of about 0.1-5% by weight. In some embodiments, a lubricant such as magnesium stearate is present in an amount of about 0.2-5% by weight. In some embodiments, a lubricant such as magnesium stearate is present in an amount of about 0.2-2% by weight. In some embodiments, a lubricant such as magnesium stearate is present in an amount of about 0.2-1% by weight. In some embodiments, a lubricant such as magnesium stearate is present in an amount of about 0.2-0.75% by weight. In some embodiments, a lubricant such as magnesium stearate is present in an amount of about 0.3% by weight. In some embodiments, a lubricant such as magnesium stearate is present in an amount of about 0.4% by weight. In some embodiments, a lubricant such as magnesium stearate is present in an amount of about 0.5% by weight. In some embodiments, a lubricant such as magnesium stearate is present in an amount of about 0.6% by weight. In some embodiments, a lubricant such as magnesium stearate is present in an amount of about 0.7% by weight. In some embodiments, a lubricant is present in an amount of from about 0.01 mg to 0.05 mg, 0.05 mg to 0.1 mg, 0.1 mg to 0.2 mg, 0.2 mg to 0.25 mg, 0.25 mg to 0.5 mg, 0.5 mg to 0.75 mg, 0.7 mg to 0.95 mg, 0.9 mg to 1.15 mg, 1.1 mg to 1.35 mg, 1.3 mg to 1.5 mg, 1.5 mg to 1.75 mg, 1.75 to 1.95 mg, 1.9 mg to 2.15 mg, 2.1 mg to 2.35 mg, 2.3 mg to 2.55 mg, 2.5 mg to 2.75 mg, 2.7 mg to 3.0 mg, 2.9 mg to 3.15 mg, 3.1 mg to 3.35 mg, 3.3 mg to 3.5 mg, 3.5 mg to 3.75 mg, 3.7 mg to 4.0 mg, 4.0 mg to 4.5 mg, 4.5 mg to 5.0 mg, 5.0 mg to 5.5 mg, 5.5 mg to 6.0 mg, 6.0 mg to 6.5 mg, 6.5 mg to 7.0 mg, 7.0 mg to 7.5 mg, 7.5 mg to 8.0 mg, 8.0 mg to 8.5 mg, 8.5 mg to 9.0 mg, 9.0 mg to 9.5 mg, or 9.5 mg to 10.0 mg. In some embodiments, a lubricant is present in an amount of about 0.01 mg, 0.05 mg, 0.1 mg, 0.2 mg, 0.25 mg, 0.5 mg, 0.7 mg, 0.9 mg, 1.1 mg, 1.3 mg, 1.5 mg, 1.7 mg, 1.9 mg, 2. mg, 2.3 mg, 2.5 mg, 2.75 mg, 3.0 mg, 3.1 mg, 3.3 mg, 3.5 mg, 3.7 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, or 10.0 mg.

Various useful glidants include, but are not limited to, tribasic calcium phosphate (TRI-TAB™), calcium silicate, cellulose, powdered (Sanacel™, Solka-Floe™) colloidal silicon dioxide (Aerosil™, Cab-O-Sil M-5P™, Wacker HDK™), magnesium silicate, magnesium trisilicate, starch (Melojel™, Meritena™, Paygel 55™, Perfectamyl D6PH™, Pure-Bind™, Pure-Cote™, Pure-Dent™, Pure-Gel™, Pure-Set™, Purity 21™ Purity 826™, Tablet White™) and talc (Luzenac Pharma™, Magsil Osmanthus™, Magsil Star™, Superiore™), or mixtures thereof. In some embodiments, a glidant is optionally used in an amount of about 0-15% by weight. In some embodiments, a glidant is present in an amount of from about 0.1 mg to 0.5 mg, 0.5 mg to 1 mg, 1 mg to 2 mg, 2 mg to 2.5 mg, 2.5 mg to 5 mg, 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23.5 mg, 23 mg to 25.5 mg, 25 mg to 27.5 mg, 27 mg to 30 mg, 29 mg to 31.5 mg, 31 mg to 33.5 mg, 33 mg to 35.5 mg, 35 mg to 37.5 mg, 37 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg. In some embodiments, a glidant is present in an amount of about 0.1 mg, 0.5 mg, 1 mg, 2 mg, 2.5 mg, 5 mg, 7 mg, 9 mg, 11 mg, 13 mg, 15 mg, 17 mg, 19 mg, 21 mg, 23 mg, 25 mg, 27.5 mg, 30 mg, 31.5 mg, 33.5 mg, 35.5 mg, 37.5 mg, 40 mg, 45 mg, 50 mg, 55 mg, 60 mg, 65 mg, 70 mg, 75 mg, 80 mg, 85 mg, 90 mg, 95 mg, or 100 mg.

Pharmaceutically acceptable surfactants include, but are limited to both non-ionic and ionic surfactants suitable for use in pharmaceutical dosage forms. Ionic surfactants may include one or more of anionic, cationic or zwitterionic surfactants. Various useful surfactants include, but are not limited to, sodium lauryl sulfate, monooleate, monolaurate, monopalmitate, monostearate or another ester of polyoxyethylene sorbitan, sodium dioctylsulfosuccinate (DOSS), lecithin, stearyl alcohol, cetostearylic alcohol, cholesterol, polyoxyethylene ricin oil, polyoxyethylene fatty acid glycerides, poloxamer, or any other commercially available co-processed surfactant like SEPITRAP® 80 or SEPITRAP® 4000 and mixtures thereof. In some embodiments, surfactant is optionally used in an amount of about 0-5% by weight. In some embodiments, a surfactant is present in an amount of from about 0.1 mg to 0.5 mg, 0.5 mg to 1 mg, 1 mg to 2 mg, 2 mg to 2.5 mg, 2.5 mg to 5 mg, 5 mg to 7.5 mg, 7 mg to 9.5 mg, 9 mg to 11.5 mg, 11 mg to 13.5 mg, 13 mg to 15.5 mg, 15 mg to 17.5 mg, 17 to 19.5 mg, 19 mg to 21.5 mg, 21 mg to 23.5 mg, 23 mg to 25.5 mg, 25 mg to 27.5 mg, 27 mg to 30 mg, 29 mg to 31.5 mg, 31 mg to 33.5 mg, 33 mg to 35.5 mg, 35 mg to 37.5 mg, 37 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, or 95 mg to 100 mg. In some embodiments, a surfactant is present in an amount of about 0.1 mg, 0.5 mg, 1 mg, 2 mg, 2.5 mg, 5 mg, 7 mg, 9 mg, 11 mg, 13 mg, 15 mg, 17 mg, 19 mg, 21 mg, 23 mg, 25 mg, 27.5 mg, 30 mg, 31.5 mg, 33.5 mg, 35.5 mg, 37.5 mg, 40 mg, 45 mg, 50 mg, 55 mg, 60 mg, 65 mg, 70 mg, 75 mg, 80 mg, 85 mg, 90 mg, 95 mg, or 100 mg.

Disintegration

Disintegration is a measure of the quality of the oral dosage forms, e.g. tablets. In general, pharmacopoeia (e.g. the US Pharmacopeia, British Pharmacopoeia, Indian Pharmacopoeia) have their own set of standards and specify disintegration tests. Pharmacopoeia of a number of international entities have been harmonised by the International conference on Harmonisation (ICH) and are interchangeable. A disintegration test is performed to find out the time it takes for a solid oral dosage form to completely disintegrate. The time of disintegration can be a measure of the quality. This is because, for example, the disintegration event is the rate limiting step to the release of the active material being carried by the tablet. If the disintegration time is too slow; it means that the active ingredient may in turn be released too slowly thus possibly impacting the rate of presentation of the active to the body once ingested. Vice versa, if disintegration is too fast the reverse may be true.

A disintegration test is conducted using a disintegration apparatus. Although there are slight variations in the different pharmacopoeias, the basic construction and the working of the apparatus in general remains the same. A typical test follows. The apparatus consists of a basket made of transparent polyvinyl or other plastic material. It typically has tubes set into the same basket with equal diameter and a wire mesh made of stainless steel with uniform mesh size is fixed to each of the tubes. Small metal discs may be used to enable immersion of the dosage form completely. The entire basket-rack assembly is movable by reciprocating motor which is fixed to the apex of the basket-rack assembly. The entire assembly is immersed in a vessel containing the medium in which the disintegration test is to be carried out. The vessel is provided with a thermostat to regulate the temperature of the fluid medium to the desired temperature.

The disintegration test for each dosage form is given in a pharmacopoeia. There are some general tests for typical types of dosage forms. Some of the types of dosage forms and their disintegration tests are: (1) Uncoated tablets—the test may use distilled water as medium at 37+/−2 C at 29-32 cycles per minute; test is completed after 15 minutes. It is acceptable when there is no palpable core at the end of the cycle (for at least 5 tablets or capsules) and if the mass does not stick to the immersion disc. (2) Coated tablets—the same test procedure may be adapted but the time of operation is 30 minutes. (3) Enteric coated/Gastric resistant tablets—the test may be carried out first in distilled water (at room temperature for 5 min.; USP and no distilled water per BP and IP), then it is tested in 0.1 M HCL (up to 2 hours; BP) or Stimulated gastric fluid (1 hour; USP) followed by Phosphate buffer, pH 6.8 (1 hour; BP) or Stimulated intestinal fluid without enzymes (1 hour; USP). (4) Chewable tablets—exempted from disintegration test (BP and IP), 4 hours (USP). These are a few examples for illustration.

An exemplary disintegration test uses a standard USP <701> test apparatus. One tablet each are placed in six of the disintegration tester slots, containing a stainless steel mesh at the bottom. A magnetic sensor is placed on top of the tablets. The basket containing the slots is immersed in a controlled temperature bath of water at 37 C. The basket moves up and down in the bath between 29-32 cycles per minute. Once the tablet completely disintegrates, the sensor on top of the tablet makes contact with the mesh. The sensor automatically will record the time at which the tablet has disintegrated.

In some embodiments, the tablet has a disintegration time of about 30 seconds to about 300 seconds. In some embodiments, the tablet has a disintegration time of about 30 seconds to about 200 seconds. In some embodiments, the tablet has a disintegration time of about 30 seconds to about 150 seconds. In some embodiments, the tablet has a disintegration time of about 30 seconds, about 40 seconds, about 50 seconds, about 60 seconds, about 70 seconds, about 80 seconds, about 90 seconds, about 100 seconds, about 110 seconds, about 120 seconds, about 130 seconds, about 140 seconds, about 150 seconds, about 160 seconds, about 170 seconds, about 180 seconds, about 190 seconds, about 200 seconds, about 210 seconds, about 220 seconds, about 230 seconds, about 240 seconds, about 250 seconds, about 260 seconds, about 270 seconds, about 280 seconds, about 290 seconds, or about 300 seconds.

Dissolution

Drug dissolution represents a critical factor affecting the rate of systemic absorption. A variety of in vitro methods have been developed for assessing the dissolution properties of pharmaceutical formulations, and dissolution testing is sometimes used as a surrogate for the direct evaluation of drug bioavailability. See, e.g., Emmanuel et al., Pharmaceutics (2010), 2:351-363, and references cited therein. Dissolution testing measures the percentage of the API that has been released from the drug product (i.e., tablet or capsule) and dissolved in the dissolution medium under controlled testing conditions over a defined period of time. To maintain sink conditions, the saturation solubility of the drug in the dissolution media should be at least three times the drug concentration. For low solubility compounds, dissolution may sometimes be determined under non-sink conditions. Dissolution is affected by the properties of the API (e.g., particle size, crystal form, bulk density), the composition of the drug product (e.g., drug loading, excipients), the manufacturing process (e.g., compression forces) and the stability under storage conditions (e.g., temperature, humidity). The capsule dosage form prepared by the processes described herein can be subjected to in vitro dissolution evaluation according to Test 711 “Dissolution” in the United States Pharmacopoeia 37, United States Pharmacopoeial Convention, Inc., Rockville, Md., 2014 (“USP 711”) to determine the rate at which the active substance is released from the dosage form, and the content of the active substance can be determined in solution by high performance liquid chromatography. This test is provided to determine compliance with the dissolution requirements where stated in the individual monograph for dosage forms administered orally. In this general chapter, a dosage unit is defined as 1 tablet or 1 capsule or the amount specified. Of the types of apparatus described herein, use the one specified in the individual monograph. Where the label states that an article is enteric-coated, and where a dissolution or disintegration test that does not specifically state that it is to be applied to delayed-release articles is included in the individual monograph, the procedure and interpretation given for Delayed-Release Dosage Forms is applied unless otherwise specified in the individual monograph. For hard or soft gelatin capsules and gelatin-coated tablets that do not conform to the Dissolution specification, repeat the test as follows. Where water or a medium with a pH of less than 6.8 is specified as the Medium in the individual monograph, the same Medium specified may be used with the addition of purified pepsin that results in an activity of 750,000 Units or less per 1000 mL. For media with a pH of 6.8 or greater, pancreatin can be added to produce not more than 1750 USP Units of protease activity per 1000 mL.

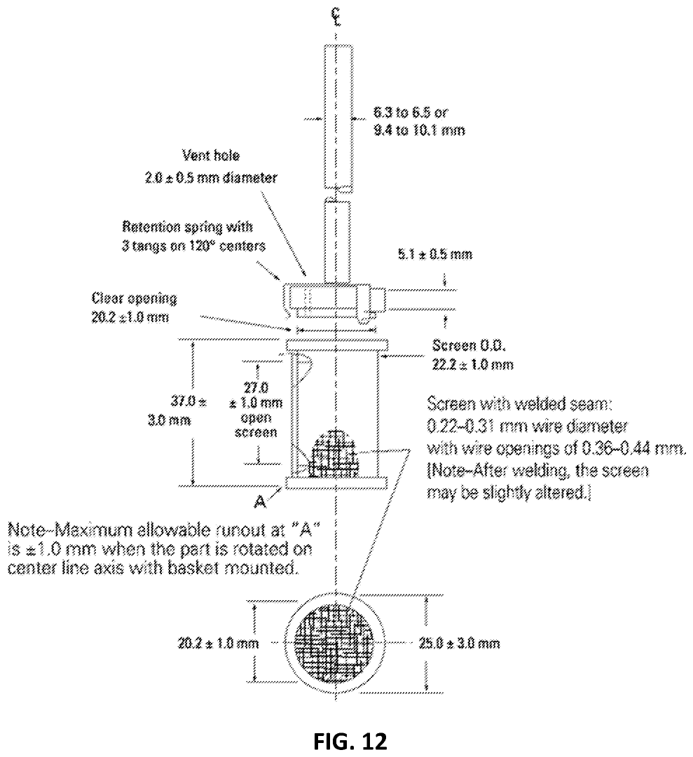

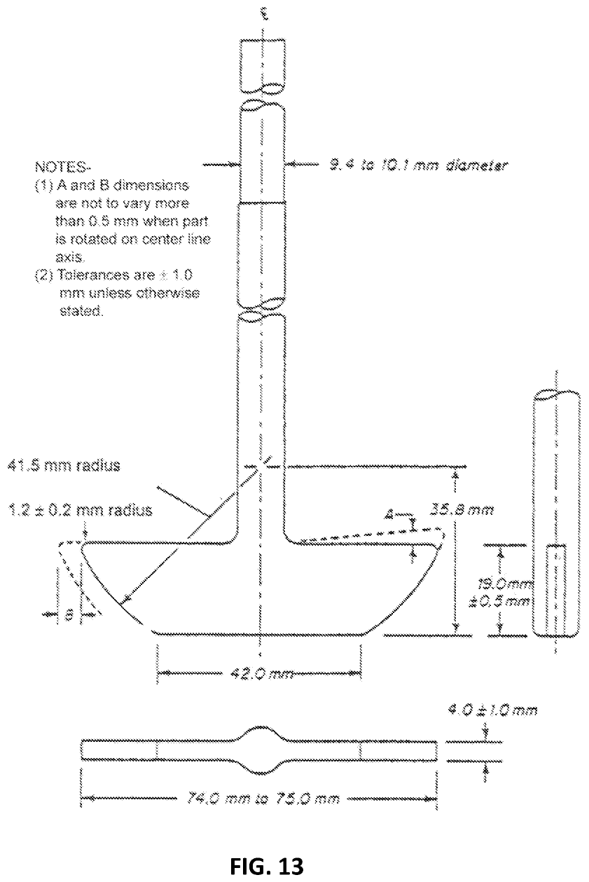

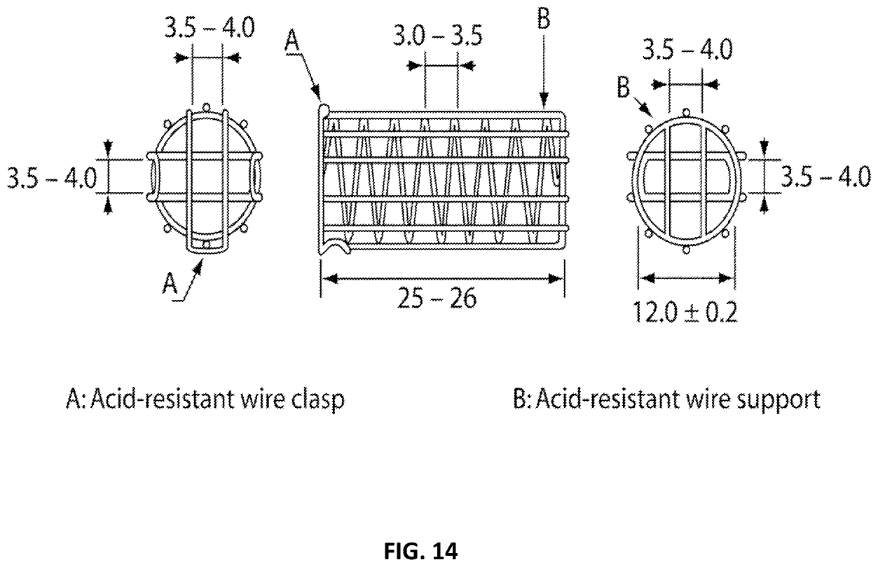

FIGS. 12-14 are exemplary illustrations of apparatuses used in an USP dissolution evaluation.

USP <711> Apparatus 1 (Basket Apparatus)

The assembly can comprise the following: a vessel, which may be covered, made of glass or other inert, transparent material; a motor; a metallic drive shaft; and a cylindrical basket. The vessel is partially immersed in a suitable water bath of any convenient size or heated by a suitable device such as a heating jacket. The water bath or heating device permits holding the temperature inside the vessel at 37±0.5 during the test and keeping the bath fluid in constant, smooth motion. No part of the assembly, including the environment in which the assembly is placed, contributes significant motion, agitation, or vibration beyond that due to the smoothly rotating stirring element. An apparatus that permits observation of the specimen and stirring element during the test is preferable. The vessel can be cylindrical, with a hemispherical bottom and with one of the following dimensions and capacities: for a nominal capacity of 1 L, the height can be 160 mm to 210 mm and its inside diameter can be 98 mm to 106 mm; for a nominal capacity of 2 L, the height can be 280 mm to 300 mm and its inside diameter can be 98 mm to 106 mm; and for a nominal capacity of 4 L, the height can be 280 mm to 300 mm and its inside diameter can be 145 mm to 155 mm. Its sides are flanged at the top. A fitted cover may be used to retard evaporation. The shaft can be positioned so that its axis is not more than 2 mm at any point from the vertical axis of the vessel and rotates smoothly and without significant wobble that could affect the results. A speed-regulating device can be used that allows the shaft rotation speed to be selected and maintained at the specified rate given in the individual monograph, within ±4%.

Shaft and basket components of the stirring element can be fabricated of stainless steel, type 316, or other inert material. A basket having a gold coating of about 0.0001 inch (2.5 μm) thick may be used. A dosage unit can be placed in a dry basket at the beginning of each test. The distance between the inside bottom of the vessel and the bottom of the basket can be maintained at 25±2 mm during the test.

USP <711> Apparatus 2 (Paddle Apparatus)

Use the assembly from Apparatus 1, except that a paddle formed from a blade and a shaft is used as the stirring element. The shaft is positioned so that its axis is not more than 2 mm from the vertical axis of the vessel at any point and rotates smoothly without significant wobble that could affect the results. The vertical center line of the blade passes through the axis of the shaft so that the bottom of the blade is flush with the bottom of the shaft. The paddle conforms to the specifications shown in FIG. 40. The distance of 25±2 mm between the bottom of the blade and the inside bottom of the vessel is maintained during the test. The metallic or suitably inert, rigid blade and shaft comprise a single entity. A suitable two-part detachable design may be used provided the assembly remains firmly engaged during the test. The paddle blade and shaft may be coated with a suitable coating so as to make them inert. The dosage unit is allowed to sink to the bottom of the vessel before rotation of the blade is started. A small, loose piece of nonreactive material, such as not more than a few turns of wire helix, may be attached to dosage units that would otherwise float. An alternative sinker device is shown in FIG. 41. Other validated sinker devices may be used.

When comparing the test and reference products, dissolution profiles can be compared using a similarity factor (f₂). The similarity factor is a logarithmic reciprocal square root transformation of the sum of squared error and is a measurement of the similarity in the percent (%) of dissolution between the two curves. Two dissolution profiles can be considered similar when the f₂ value is equal to or greater than 50.

f₂=50 log {[1+(l/n)Σₜ₌₁ⁿ(Rₜ−Tₜ)²]″⁰⁻⁵−100}

In some aspects, dissolution rates are measured by a standard USP 2 rotating paddle apparatus as disclosed in USP 711, Apparatus 2. In some embodiments, the dosage form is added to a solution containing a buffer, e.g., phosphate, HCl, acetate, borate, carbonate, or citrate buffer. In some embodiments, the dosage form is added to a solution containing a buffer, e.g., phosphate, HCl, acetate, borate, carbonate, or citrate buffer, with a quantity of enzyme that results in a desired protease activity of dissolution medium. In some embodiments, at appropriate times following test initiation (e.g., insertion of the dosage form into the apparatus), filtered aliquots from the test medium are analyzed for niraparib by high performance liquid chromatography (HPLC). Dissolution results are reported as the percent of the total dose of niraparib tested dissolved versus time.

In some aspects, dissolution rates are measured by a standard USP 2 rotating paddle apparatus as disclosed in USP 711, Apparatus 2. In some embodiments, the dosage form is added to a solution containing a buffer, e.g., phosphate, HCl, acetate, borate, carbonate, or citrate buffer. In some embodiments, the dosage form is added to a solution with a pH of from 2-13, 3-12, 4-10, 5-9, 6-8, 4.1-5.5, or 5.8-8.8, e.g., a solution with a pH of 2, 3, 3.5 4, 4.1, 5, 5.8, 6, 7, 7.2, 7.5, 8, 8.3, 8.8, 9, 10, 11, 12, or 13. In some embodiments, the dosage form is added to a solution containing a buffer, e.g., phosphate, HCl, acetate, borate, carbonate, or citrate buffer, with a quantity of enzyme that results in the desired protease activity. In some embodiments, at appropriate times following test initiation (e.g., insertion of the dosage form into the apparatus), filtered aliquots from the test medium are analyzed for niraparib by high performance liquid chromatography (HPLC). Dissolution results are reported as the percent of the total dose of niraparib tested dissolved versus time. Dissolution rates of the compositions described herein can be consistent, for example, the dissolution of the compositions can be at least 90%, 95%, 98%, 99%, or 100% in 5, 10, 15, 30, 45, 60, or 90 minutes.

In some embodiments, the solid dosage form of any of the embodiments described herein, under dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes. In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes.

In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes.

In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 45 minutes.

In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes.

In some embodiments, the solid dosage form of any of the embodiments described herein, under the conditions of dissolution evaluation, dissolves: not less than 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes.

In some embodiments, after being stored at 25° C./60% RH for 3 months, the solid dosage form of any of the embodiments described herein, under dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes.

In some embodiments, after being stored at 25° C./60% RH for 3 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes. In some embodiments, after being stored at 25° C./60% RH for 3 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes

In some embodiments, after being stored at 25° C./60% RH for 3 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes. In some embodiments, after being stored at 25° C./60% RH for 3 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, after being stored at 25° C./60% RH for 3 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes. In some embodiments, after being stored at 25° C./60% RH for 3 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes.

In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes. In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes. In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes.

In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes. In some embodiments, after being stored at 25° C./60% RH for 6 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes.

In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes. In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes. In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes.

In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes. In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes.

In some embodiments, after being stored at 25° C./60% RH for 9 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes. In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes.

In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes. In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes.

In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes. In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes. In some embodiments, after being stored at 25° C./60% RH for 12 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes.

In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes. In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes.

In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes. In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes. In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes. In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes. In some embodiments, after being stored at 25° C./60% RH for 24 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes. In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 5 minutes. In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes. In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 15 minutes. In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 30 minutes.

In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 10 minutes. In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 60 minutes.

In some embodiments, after being stored at 25° C./60% RH for 36 months, the solid dosage form of any of the embodiments described herein, under the conditions of dissolution evaluation, dissolves: not less than 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the niraparib in 90 minutes.

Stability

In some embodiments, the pharmaceutical composition disclosed herein is stable for at least about: 30 days, 60 days, 90 days, 6 months, 1 year, 18 months, 2 years, 3 years, 4 years, or 5 years, for example about 80%-100% such as about: 80%, 90%, 95%, or 100% of the active pharmaceutical agent in the pharmaceutical composition is stable, e.g., as measured by High Performance Liquid Chromatography (HPLC). In some embodiments, about 80%-100% (e.g., about: 90%-100% or 95-100%) of niraparib or a pharmaceutically acceptable salt thereof (e.g., niraparib tosylate monohydrate) in the pharmaceutical composition disclosed herein is stable for at least about: 30, 60, 90, 180, 360, 540, or 720 days, for example greater than 90 days, which can be measured by HPLC. In some embodiments, about: 80%, 85%, 90%, 95%, or 100% (e.g., about 95%) of the niraparib or a pharmaceutically acceptable salt thereof (e.g., niraparib tosylate monohydrate) is stable for 30 days or more, which can be measured by HPLC.

In some embodiments, the pharmaceutical composition disclosed herein is stable with respect to particle size distribution for at least about: 30 days, 60 days, 90 days, 6 months, 1 year, 18 months, 2 years, 3 years, 4 years, or 5 years, for example about 80%-100% such as about: 80%, 90%, 95%, or 100% of the pharmaceutical composition is stable with respect to particle size distribution. In some embodiments, the stable niraparib particles described herein in a solid oral dosage form will not show an increase in effective particle size of greater than 50% up to about 3, 6, 9, 12, 24 or 36 months storage at room temperature (about 15° C. to about 25° C.). In some embodiments, the stable niraparib particles described herein in a solid oral dosage form will not show an increase in effective particle size of greater than 60% up to about 3, 6, 9, 12, 24 or 36 months storage at room temperature (about 15° C. to about 25° C.). In some embodiments, the stable niraparib particles described herein in a solid oral dosage form will not show an increase in effective particle size of greater than 70% up to about 3, 6, 9, 12, 24 or 36 months storage at room temperature (about 15° C. to about 25° C.). In some embodiments, the stable niraparib particles described herein in a solid oral dosage form will not show an increase in effective particle size of greater than 80% up to about 3, 6, 9, 12, 24 or 36 months storage at room temperature (about 15° C. to about 25° C.). In some embodiments, the stable niraparib particles described herein in a solid oral dosage form will not show an increase in effective particle size of greater than 90% up to about 3, 6, 9, 12, 24 or 36 months storage at room temperature (about 15° C. to about 25° C.). In some embodiments, the stable niraparib particles described herein in a solid oral dosage form will not show an increase in effective particle size of greater than 95% up to about 3, 6, 9, 12, 24 or 36 months storage at room temperature (about 15° C. to about 25° C.).

In some embodiments, the stable niraparib particles described herein in a solid oral dosage form will not show an increase in effective particle size of greater than 50% up to 3, 6, 9, 12, 24 or 36 months storage at about 15° C. to 30° C., 15° C. to 40° C., or 15° C. to 50° C. In some embodiments, the stable niraparib particles described herein in a solid oral dosage form will not show an increase in effective particle size of greater than 60% up to 3, 6, 9, 12, 24 or 36 months storage at about 15° C. to 30° C., 15° C. to 40° C., or 15° C. to 50° C. In some embodiments, the stable niraparib particles described herein in a solid oral dosage form will not show an increase in effective particle size of greater than 70% up to 3, 6, 9, 12, 24 or 36 months storage at about 15° C. to 30° C., 15° C. to 40° C., or 15° C. to 50° C. In some embodiments, the stable niraparib particles described herein in a solid oral dosage form will not show an increase in effective particle size of greater than 80% up to 3, 6, 9, 12, 24 or 36 months storage at about 15° C. to 30° C., 15° C. to 40° C., or 15° C. to 50° C. In some embodiments, the stable niraparib particles described herein in a solid oral dosage form will not show an increase in effective particle size of greater than 90% up to 3, 6, 9, 12, 24 or 36 months storage at about 15° C. to 30° C., 15° C. to 40° C., or 15° C. to 50° C. In some embodiments, the stable niraparib particles described herein in a solid oral dosage form will not show an increase in effective particle size of greater than 95% up to 3, 6, 9, 12, 24 or 36 months storage at about 15° C. to 30° C., 15° C. to 40° C., or 15° C. to 50° C.

In some embodiments, the pharmaceutical composition disclosed herein is stable with respect to compound degeneration for at least about: 30 days, 60 days, 90 days, 6 months, 1 year, 18 months, 2 years, 3 years, 4 years, or 5 years, for example about 80%-100% such as about: 80%, 90%, 95%, or 100% of the active pharmaceutical agent in the pharmaceutical composition is stable. Stability may be measured by High Performance Liquid Chromatography (HPLC). In some embodiments, about 80%-100% (e.g., about: 90%-100% or 95-100%) of niraparib or a pharmaceutically acceptable salt thereof (e.g., niraparib tosylate monohydrate) in the pharmaceutical composition disclosed herein is stable for at least about: 30, 60, 90, 180, 360, 540, or 720 days, for example greater than 90 days. In some embodiments, about: 80%, 85%, 90%, 95%, or 100% (e.g., about 95%) of the niraparib or a pharmaceutically acceptable salt thereof (e.g., niraparib tosylate monohydrate) is stable with respect to compound degeneration for 30 days or more. In each case, stability may be measured by HPLC or another method known in the art. Methods for assessing the chemical storage stability of solid dosage forms are described in the literature. See, e.g., S. T. Colgan, T. J. Watson, R. D. Whipple, R. Nosal, J. V. Beaman, D. De Antonis, “The Application of Science and Risk Based Concepts to Drug Substance Stability Strategies” J. Pharm. Innov. 7:205-2013 (2012); Waterman K C, Carella A J, Gumkowski M J, et al. Improved protocol and data analysis for accelerated shelf-life estimation of solid dosage forms. Pharm Res 2007; 24(4):780-90; and S. T. Colgan, R. J. Timpano, D. Diaz, M. Roberts, R. Weaver, K. Ryan, K. Fields, G. Scrivens, Opportunities for Lean Stability Strategies” J. Pharm. Innov. 9:259-271 (2014).

In some embodiments, the pharmaceutical formulations described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 5% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, at least about 6 months, at least about 7 months, at least about 8 months, at least about 9 months, at least about 10 months, at least about 11 months, at least about 12 months, at least about 24 months, or at least about 36 months under storage conditions (e.g. room temperature). In some embodiments, the formulations described herein are stable with respect to compound degradation over a period of at least about 1 week. In some embodiments, the formulations described herein are stable with respect to compound degradation over a period of at least about 1 month. In some embodiments, the formulations described herein are stable with respect to compound degradation over a period of at least about 3 months. In some embodiments, the formulations described herein are stable with respect to compound degradation over a period of at least about 6 months. In some embodiments, the formulations described herein are stable with respect to compound degradation over a period of at least about 9 months. In some embodiments, the formulations described herein are stable with respect to compound degradation over a period of at least about 12 months.

Methods for assessing the chemical stability of solid dosage forms during storage, including under accelerated aging conditions are described in the literature. See, e.g., S. T. Colgan, T. J. Watson, R. D. Whipple, R. Nosal, J. V. Beaman, D. De Antonis, “The Application of Science and Risk Based Concepts to Drug Substance Stability Strategies” J. Pharm. Innov. 7:205-2013 (2012); Waterman K C, Carella A J, Gumkowski M J, et al. Improved protocol and data analysis for accelerated shelf-life estimation of solid dosage forms. Pharm Res 2007; 24(4):780-90; and S. T. Colgan, R. J. Timpano, D. Diaz, M. Roberts, R. Weaver, K. Ryan, K. Fields, G. Scrivens, Opportunities for Lean Stability Strategies” J. Pharm. Innov. 9:259-271 (2014). Chemical stability of solid dosage forms during storage may also be dictated by the International Council for Harmonisation of Technical Requirements for Pharmaceuticals for Human Use (ICH) or the World Health Organization (WHO).

Depending on the region of the world in which a pharmaceutical composition is intended to be used and/or stored, stability studies may be performed according to the climatic conditions of the country. The world is generally divided into five different zones: temperate, Mediterranean/subtropical, hot dry, hot humid/tropical zone, and hot/higher humidity. Those skilled in the relevant art may determine the appropriate conditions for testing in a specific climatic zone.

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 40° C. and 75% relative humidity (RH).

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of impurities (e.g. exemplary impurities described herein) after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of known impurities after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of known impurities after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of known impurities after storage for 1 month, 3 months, 6 months, 9 months, 12 months, 24 months, or 36 months at 40° C. and 75% relative humidity (RH).

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single unspecified degradation product, such as any single unspecified niraparib degradation products after storage for 1 month, 3 months, 6 months, 9 months, 12 months, 24 months, or 36 months at 40° C. and 75% relative humidity (RH).

In some embodiments, the invention provides an oral dosage form comprising niraparib and a pharmaceutically acceptable carrier, wherein the dosage form exhibits less than 3.0%, 2.5%, 2.0%, 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, such as total niraparib degradation products after storage for 1 month, 3 months, 6 months, 9 months, 12 months, 24 months, or 36 months at 40° C. and 70% relative humidity (RH).

In one aspect provided herein is composition comprising a tablet comprising: an effective amount of niraparib to inhibit polyadenosine diphosphate ribose polymerase (PARP) when administered to a subject in need thereof; wherein the tablet has at least one of the following: a) the tablet comprises less than 0.2% by weight of any single niraparib degradation product; b) the tablet comprises less than 0.2% by weight of any single niraparib degradation product after storage for 1 month at 40° C. and 75% relative humidity (RH); and c) the tablet comprises less than 0.2% by weight of any single niraparib degradation product after storage for 2 months at 40° C. and 75% relative humidity (RH).

In some embodiments, the tablet comprises less than 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product. In some embodiments, tablet comprises less than 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product after storage for 1 month at 40° C. and 75% relative humidity (RH). In some embodiments, the tablet comprises less than 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product after storage for 2 months at 40° C. and 75% relative humidity (RH).

In some embodiments, the tablet comprises about 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product. In some embodiments, tablet comprises about 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product after storage for 1 month at 40° C. and 75% relative humidity (RH). In some embodiments, the tablet comprises about 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product after storage for 2 months at 40° C. and 75% relative humidity (RH).

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products, such as one or more niraparib degradation products, after storage for 1 month, 3 months, 6 months, 9 months, 12 months, 24 months, or 36 months at 40° C. and 75% relative humidity (RH).

In some embodiments, the amount of one or more or total impurity or degradation products of niraparib is from about 0.01 mg to 0.05 mg, 0.05 mg to 0.1 mg, 0.1 mg to 0.2 mg, 0.2 mg to 0.25 mg, 0.25 mg to 0.5 mg, 0.5 mg to 0.75 mg, 0.7 mg to 0.95 mg, 0.9 mg to 1.15 mg, 1.1 mg to 1.35 mg, 1.3 mg to 1.5 mg, 1.5 mg to 1.75 mg, 1.75 to 1.95 mg, 1.9 mg to 2.15 mg, 2.1 mg to 2.35 mg, 2.3 mg to 2.55 mg, 2.5 mg to 2.75 mg, 2.7 mg to 3.0 mg, 2.9 mg to 3.15 mg, 3.1 mg to 3.35 mg, 3.3 mg to 3.5 mg, 3.5 mg to 3.75 mg, 3.7 mg to 4.0 mg, 4.0 mg to 4.5 mg, 4.5 mg to 5.0 mg, 5.0 mg to 5.5 mg, 5.5 mg to 6.0 mg, 6.0 mg to 6.5 mg, 6.5 mg to 7.0 mg, 7.0 mg to 7.5 mg, 7.5 mg to 8.0 mg, 8.0 mg to 8.5 mg, 8.5 mg to 9.0 mg, 9.0 mg to 9.5 mg, or 9.5 mg to 10.0 mg. In some embodiments, the amount of one or more or total impurity or degradation products of niraparib is less than about or about 0.01 mg, 0.05 mg, 0.1 mg, 0.2 mg, 0.25 mg, 0.5 mg, 0.7 mg, 0.9 mg, 1.1 mg, 1.3 mg, 1.5 mg, 1.7 mg, 1.9 mg, 2. mg, 2.3 mg, 2.5 mg, 2.75 mg, 3.0 mg, 3.1 mg, 3.3 mg, 3.5 mg, 3.7 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, or 10.0 mg.

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of formation of one or more degradation products after storage for 1 month, 3 months, 6 months, 9 months, 12 months, 24 months, or 36 months at 40° C. and 75% relative humidity (RH).

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single degradation product, after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single degradation product, after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single degradation product, after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of any single degradation product, after storage for 1 month, 3 months, 6 months, 9 months, 12 months, 24 months, or 36 months at 40° C. and 75% relative humidity (RH).

In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, including niraparib degradation products after storage for 1 month, 3 months, 6 months, 9 months, 12 months, 24 months, or 36 months at 5° C. In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, including niraparib degradation products after storage for 1 month, 3 months, 6 months, 9 months, 12 months, 24 months, or 36 months at 25° C. and 60% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, including total niraparib degradation products after storage for 1 month, 3 months, 6 months, 9 months, 12 months, 24 months, or 36 months at 30° C. and 65% relative humidity (RH). In some embodiments, the invention provides an oral dosage form comprising niraparib and a pharmaceutically acceptable carrier, wherein the dosage form exhibits less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of formation of total degradation products, including niraparib degradation products after storage for 1 month, 3 months, 6 months, 9 months, 12 months, 24 months, or 36 months at 40° C. and 70% relative humidity (RH).

In some embodiments, the composition comprises less than 10% by weight of water. In some embodiments, the composition comprises less than 10% by weight of water after storage for 1 month at 40° C. and 75% relative humidity (RH). In some embodiments, the composition comprises less than 10% by weight of water after storage for 2 months at 40° C. and 75% relative humidity (RH).

In some embodiments, the composition comprises less than 30%, 25%, 20%, 15%, 10%, 9%, 8%, 7%, 6%, 5%, 4%, 3%, 2%, 1%, 0.5%, or 0.1% by weight of water. In some embodiments, the composition comprises about 30%, 25%, 20%, 15%, 10%, 9%, 8%, 7%, 6%, 5%, 4%, 3%, 2%, 1%, 0.5%, or 0.1% by weight of water. In some embodiments, the composition comprises less than 30%, 25%, 20%, 15%, 10%, 9%, 8%, 7%, 6%, 5%, 4%, 3%, 2%, 1%, 0.5%, or 0.1% by weight of water after storage for 1 month at 40° C. and 75% relative humidity (RH). In some embodiments, the composition comprises about 30%, 25%, 20%, 15%, 10%, 9%, 8%, 7%, 6%, 5%, 4%, 3%, 2%, 1%, 0.5%, or 0.1% by weight of water after storage for 1 month at 40° C. and 75% relative humidity (RH). In some embodiments, the composition comprises less than 30%, 25%, 20%, 15%, 10%, 9%, 8%, 7%, 6%, 5%, 4%, 3%, 2%, 1%, 0.5%, 0.1% by weight of water after storage for 2 months at 40° C. and 75% relative humidity (RH). In some embodiments, the composition comprises about 30%, 25%, 20%, 15%, 10%, 9%, 8%, 7%, 6%, 5%, 4%, 3%, 2%, 1%, 0.5%, or 0.1% by weight of water after storage for 2 months at 40° C. and 75% relative humidity (RH). In some embodiments, the composition comprises less than 30%, 25%, 20%, 15%, 10%, 9%, 8%, 7%, 6%, 5%, 4%, 3%, 2%, 1%, 0.5%, or 0.1% by weight of water after storage for 3 months, 6 months, 9 months, 12 months, 24 months, or 36 months at 40° C. and 75% relative humidity (RH). In some embodiments, the composition comprises about 30%, 25%, 20%, 15%, 10%, 9%, 8%, 7%, 6%, 5%, 4%, 3%, 2%, 1%, 0.5%, or 0.1% by weight of water after storage for 3 months, 6 months, 9 months, 12 months, 24 months, or 36 months at 40° C. and 75% relative humidity (RH).

Particle Size

In some embodiments, the pharmaceutical composition disclosed herein comprises pluralities of particulates. In some embodiments, the pharmaceutical composition comprises a plurality of first particulates and a plurality of second particulates. In some embodiments, the plurality of first particulates comprises niraparib. In some embodiments, the plurality of second particulates comprises lactose monohydrate. In some embodiments, the pharmaceutical composition disclosed herein comprises a plurality of third particulates. In some embodiments the plurality of third particulates comprises magnesium stearate.

The particle size of niraparib particles can be an important factor which can effect bioavailability, blend uniformity, segregation, and flow properties. In general, smaller particle sizes of a drug increases the drug absorption rate of permeable drugs with substantially poor water solubility by increasing the surface area and kinetic dissolution rate. The particle size of niraparib can also affect the suspension or blend properties of the pharmaceutical formulation. For example, smaller particles are less likely to settle and therefore form better suspensions. In some embodiments, the niraparib may optionally be screened niraparib. In some embodiments, the niraparib is not screened.

The pharmaceutical compositions disclosed herein comprise niraparib particles. In various embodiments, the niraparib formulations, in aqueous dispersions or as dry powders (which can be administered directly, as a powder for suspension, or used in a solid dosage form), can comprise niraparib with compatible excipients.

Particle size reduction techniques include, by way of example, grinding, milling (e.g., air-attrition milling (jet milling), ball milling), coacervation, complex coacervation, high pressure homogenization, spray drying and/or supercritical fluid crystallization. In some instances, particles are sized by mechanical impact (e.g., by hammer mills, ball mill and/or pin mills). In some instances, particles are sized via fluid energy (e.g., by spiral jet mills, loop jet mills, and/or fluidized bed jet mills).

In some embodiments, target and maximum particle size, including particle size distribution, is determined through analytical sieving in accordance with USP <786> or other appropriately validated methods. Exemplary filters used in particulate size generation include, without limitation, #16, #18, #20, #25, #30 #40, #60, #80, #100, #120, #140, #160, #180, #200, #220, and #240 size mesh screens. Diameter of granules can be also determined using Retsch AS 200 magnetic sieve shaker at an amplitude of 30 to 90 Hz with time interval between 5 to 30 minutes {Refer: USP 29<786> Particle size distribution estimation by analytical sieving).

In some embodiments, the niraparib particles have a tap density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than 0. less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, the niraparib particles have a bulk density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than 0. less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, 10%, 50%, or 90% of the particles of an excipient by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, 10%, 50%, or 90% of the particles of an excipient by weight have a particle size of more than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, 10% of the lactose monohydrate particles by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm. In some embodiments, 50% of the lactose monohydrate particles by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm. In some embodiments, 90% of the lactose monohydrate particles by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, 10% of the lactose monohydrate particles by weight have a particle size of more than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, 50% of the lactose monohydrate particles by weight have a particle size of more than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, 90% of the lactose monohydrate particles by weight have a particle size of more than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, the lactose monohydrate particles have a tap density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than 0. less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, the lactose monohydrate particles have a bulk density of less than 0.99 mg/mL, less than 0.98 mg/mL, less than 0.97 mg/mL, less than 0.96 mg/mL, less than 0.95 mg/mL, less than 0.94 mg/mL, less than 0.93 mg/mL, less than 0.92 mg/mL, less than 0.91 mg/mL, less than 0.90 mg/mL, less than 0.89 mg/mL, less than 0.88 mg/mL, less than 0.87 mg/mL, less than 0.86 mg/mL, less than 0.85 mg/mL, less than 0.84 mg/mL, less than 0.83 mg/mL, less than 0.82 mg/mL, less than 0.81 mg/mL, less than 0.80 mg/mL, less than 0.79 mg/mL, less than 0.78 mg/mL, less than 0.77 mg/mL, less than 0.76 mg/mL, less than 0.75 mg/mL, less than 0.74 mg/mL, less than 0.73 mg/mL, less than 0.72 mg/mL, less than 0.71 mg/mL, less than 0.70 mg/mL, less than 0.69 mg/mL, less than 0.68 mg/mL, less than 0.67 mg/mL, less than 0.66 mg/mL, less than 0.65 mg/mL, less than 0.64 mg/mL, less than 0.63 mg/mL, less than 0.62 mg/mL, less than 0.61 mg/mL, less than 0.60 mg/mL, less than 0. less than 0.59 mg/mL, less than 0.58 mg/mL, less than 0.57 mg/mL, less than 0.56 mg/mL, less than 0.55 mg/mL, less than 0.54 mg/mL, less than 0.53 mg/mL, less than 0.52 mg/mL, less than 0.51 mg/mL, less than 0.50 mg/mL, less than 0.49 mg/mL, less than 0.48 mg/mL, less than 0.47 mg/mL, less than 0.46 mg/mL, less than 0.45 mg/mL, less than 0.44 mg/mL, less than 0.43 mg/mL, less than 0.42 mg/mL, less than 0.41 mg/mL, less than 0.40 mg/mL, less than 0.39 mg/mL, less than 0.38 mg/mL, less than 0.37 mg/mL, less than 0.36 mg/mL, less than 0.35 mg/mL, less than 0.34 mg/mL, less than 0.33 mg/mL, less than 0.32 mg/mL, less than 0.31 mg/mL, less than 0.30 mg/mL, less than 0.29 mg/mL, less than 0.28 mg/mL, less than 0.27 mg/mL, less than 0.26 mg/mL, less than 0.25 mg/mL, less than 0.24 mg/mL, less than 0.23 mg/mL, less than 0.22 mg/mL, less than 0.21 mg/mL, less than 0.20 mg/mL, less than 0.19 mg/mL, less than 0.18 mg/mL, less than 0.17 mg/mL, less than 0.16 mg/mL, less than 0.15 mg/mL, less than 0.14 mg/mL, less than 0.13 mg/mL, less than 0.12 mg/mL, less than 0.11 mg/mL, or less than 0.10 mg/mL.

In some embodiments, 10% of the magnesium stearate particles by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm. In some embodiments, 50% of the magnesium stearate particles by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm. In some embodiments, 90% of the magnesium stearate particles by weight have a particle size of less than 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, 1000 μm, 1050 μm, 1100 μm, 1150 μm or 1200 μm.

In some embodiments, 10% of the magnesium stearate particles by weight have a particle size of more than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, 50% of the magnesium stearate particles by weight have a particle size of more than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, 90% of the magnesium stearate particles by weight have a particle size of more than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, 10% of the lactose monohydrate particles by weight have a particle size of from 5 μm to 1000 μm, from 20 μm to 1000 μm, from 50 μm to 1000 μm, from 75 μm to 1000 μm, from 100 μm to 1000 μm, from 250 μm to 1000 μm, from 500 μm to 1000 μm, or from 750 μm to 1000 μm. In some embodiments, 50% of the lactose monohydrate particles by weight have a particle size of from 5 μm to 1000 μm, from 20 μm to 1000 μm, from 50 μm to 1000 μm, from 75 μm to 1000 μm, from 100 μm to 1000 μm, from 250 μm to 1000 μm, from 500 μm to 1000 μm, or from 750 μm to 1000 μm. In some embodiments, 90% of the lactose monohydrate particles by weight have a particle size of from 5 μm to 1000 μm, from 20 μm to 1000 μm, from 50 μm to 1000 μm, from 75 μm to 1000 μm, from 100 μm to 1000 μm, from 250 μm to 1000 μm, from 500 μm to 1000 μm, or from 750 μm to 1000 μm.

In some embodiments, 10% of the lactose monohydrate particles by weight have a particle size of from 5 μm to 500 μm, from 20 μm to 500 μm, from 50 μm to 500 μm, from 75 μm to 500 μm, from 100 μm to 500 μm, or from 250 μm to 500 μm. In some embodiments, 50% of the lactose monohydrate particles by weight have a particle size of from 5 μm to 500 μm, from 20 μm to 500 μm, from 50 μm to 500 μm, from 75 μm to 500 μm, from 100 μm to 500 μm, or from 250 μm to 500 μm. In some embodiments, 90% of the lactose monohydrate particles by weight have a particle size of from 5 μm to 500 μm, from 20 μm to 500 μm, from 50 μm to 500 μm, from 75 μm to 500 μm, from 100 μm to 500 μm, or from 250 μm to 500 μm.

In some embodiments, 10% of the lactose monohydrate particles by weight have a particle size of from 5 μm to 250 μm, from 20 μm to 250 μm, from 50 μm to 250 μm, from 75 μm to 250 μm, or from 100 μm to 250 μm. In some embodiments, 50% of the lactose monohydrate particles by weight have a particle size of from 5 μm to 250 μm, from 20 μm to 250 μm, from 50 μm to 250 μm, from 75 μm to 250 μm, or from 100 μm to 250 μm. In some embodiments, 90% of the lactose monohydrate particles by weight have a particle size of from 5 μm to 250 μm, from 20 μm to 250 μm, from 50 μm to 250 μm, from 75 μm to 250 μm, or from 100 μm to 250 μm.

In some embodiments, about 30%, 40%, 50%, 60%, 70%, or 80% of the lactose monohydrate particles by weight have a particle size of from 53 μm to 500 μm.

A method of making a formulation comprising niraparib can comprise obtaining niraparib; obtaining lactose monohydrate that has been screened with a screen; combining the niraparib with the screened lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. In some embodiments, obtaining niraparib comprises obtaining niraparib that has been screened. In some embodiments, combining the niraparib with the screened lactose monohydrate comprises combining unscreened niraparib with the screened lactose monohydrate.

Powder Characteristics

As used herein, “permeability” is a measure of the powder's resistance to air flow. The permeability test utilizes the vented piston to constrain the powder column under a range of applied normal stresses; while air is passed through the powder column. The relative difference in air pressure between the bottom and the top of the powder column is a function of the powder's permeability. Tests can be carried out under a range of normal stresses and air flow rates. Usually, a lower pressure drop is indicative of higher permeability and often, better flow properties.

As used herein, the “flow rate index” (or FRI) is a measure of a powder's sensitivity to variable flow rate and is obtained as the ratio of the total energy required to induce powder flow at 10 mm/s and 100 mm/s blade tip speed. A larger deviation from 1 indicates greater sensitivity of a powder to variable flow rate.

FRI=Flow Energy @ 10_{mm/s}/Flow Energy @100ₘₘₘ/*

As used herein, “specific energy” or SE is a measure of the powder flow in low stress environment and is derived from the shear forces acting on the blades as they rotate upward through the powder. The SE is recorded as the flow energy of the powder normalized by its weight in mJ/g during the upward spiral movement of the blades in a FT4 Powder Rheometer describe above. A lower SE is an indication of a less cohesive powder and better flow properties.

As used herein, “flow function” or FF is a parameter commonly used to rank powder's flowability and is determined using a shear test. The data produced in the shear test represents the relationship between shear stress and normal stress, which can be plotted to define the powder's Yield Locus. Fitting Mohr stress circles to the yield locus identifies the Major Principle Stress (MPS) and Unconfined Yield Strength (UYS). Flow function is the ratio of Major Principle Stress (MPS) to the Unconfined Yield Strength (UYS):

FF=MPS/UYS.

Flow characteristics can be evaluated by different tests such as angle of repose, Carr's index, Hausner ratio or flow rate through an orifice. Measures that may be taken to ensure that the compositions according to the invention have good flow and dispersion properties involve the preparation or processing of the powder particles.

In certain embodiments, powder characterization described herein can be determined using a FT4 Powder Rheometer (Freeman Technology), e.g., a FT4 Powder Rheometer with the 25 mm vessel assembly having 23.5 mm diameter blades, vented piston, a segmented rotational shear cell accessory and a 10 or 25 ml borosilicate vessel. The FT4 Powder Rheometer is capable of quantitatively measuring the flowability characteristics of particulate compositions, and these measurements can be utilized to predict the characteristics of the particulate composition when being pneumatically conveyed, e.g., in a dilute phase. The FT4 Powder Rheometer includes a container for holding a powder sample and a rotor having a plurality of blades that is configured to move in the axial direction (e.g., vertically) through the powder sample while rotating the blades relative to the container. See, for example, U.S. Pat. No. 6,065,330 by Freeman et al., which is incorporated herein by reference in its entirety. Powder testing can be generally divided into three categories: dynamic tests, permeability test and shear test.

For example, dynamic testing can use the 23.5 mm diameter blades and a 25 mL powder sample in the borosiliate test vessel. Powder is filled into the vessel and the blades are simultaneously rotated and moved axially into the powder sample as the axial and rotational forces are measure and used to calculate the dynamic flowability parameters, such as flow rate index (FRI) and Specific Energy (SE).

For example, using an FT4 Powder samples various manufactured blends can be subjected to the following tests as described in the FT4 user manual and/or associated Freeman Technology literature: The FT4 Aeration test determines Basic Flowability Energy, Specific Energy, Conditioned Bulk Density, Aerated Energy, Aeration Ratio and Normalised Aeration Sensitivity. The standard 25 mm Aeration program can be optimized to achieve improved reproducibility over the Freeman method. The FT4 Permeability test determines the Pressure Drop at compaction pressures from 0.6 kPa to 15 kPa. The standard 25 mm Permeability program can be optimized to achieve improved reproducibility over the Freeman method. The FT4 Shear test can be performed using the standard 25 mm Shear 3 kPa program which determines incipient shear stress up to a compaction pressure of 3 kPa. The FT4 Compressibility test can be performed using the standard 25 mm Compressibility 1-15 kPa which determines percentage compressibility up to a compaction pressure of 15 kPa. For example, powder can be filled into a vessel. The powder bed with a vested piston can be exposed to varying normal stress increased stepwise, e.g., from 1 kPa to 15 kPa. The pressure drop across the powder bed can be measured while air is flushed through the powder at a constant velocity, e.g., 2 mm/s.

Shear testing can be used measure powder shear properties which involves the stress limit required to initiate a powder flow. The shear testing uses a segmented rotational shear cell head and a 10 ml powder sample in the borosilicate test vessel. Powder is filled into the vessel. The shear cell head is simultaneously rotated and moved axially under the powder sample at pre-determined normal stresses as the shear stresses are measured to calculate several parameters, including the flow function (FF). Usually, powders of low cohesion have higher FF and that represents better flow properties. The permeability test can measure the ease of air transmission through a bulk powder which can be related to the powder's flowability. For example, a permeability testing can use a vented piston with an aeration base and 10 mL powder sample in the borosilicated test vessel.

BFE and SE are determined by the FT4 Powder Rheometer using the Stability and Variable Flow Rate method (“the SVFR method”). The SVFR method includes seven test cycles using a stability method and four test cycles using a variable flow rate method, where each test cycle includes a conditioning step before the measurement is taken. The conditioning step homogenizes the compositions by creating a uniform low stress packing of particles throughout the sample, which removes any stress history or excess entrained air prior to the measurement. The stability method includes maintaining the blade tip speed at about 100 millimeters per second (mm/s) during the test cycles, whereas the variable flow rate method involves four measurements using different blade tip speeds, namely about 100 mm/s, about 70 mm/s, about 40 mm/s and about 10 mm/s. The test measures the energy required to rotate the blade through the powder from the top of the vessel to the bottom and to rotate the blade through the powder from the bottom to the top of the vessel.

BFE is the total energy measured during the seventh cycle during the stability method measurements of the SVFR method described above (i.e., at a tip speed set at 100 mm/s) while the blade is rotating from the top of the vessel to the bottom. The BFE is a measurement of the energy required to establish a particular flow pattern in a (conditioned) powder, which is established by a downward counter-clockwise motion of the blade that puts the powder under a compressive stress. The BFE, when considered in conjunction with other powder characteristics, can be used to predict the pneumatic conveyance properties of the compositions described herein. For some particulate compositions, the lower the BFE, the more easily the compositions described herein can be made to flow in a regular and invariable manner, e.g., without significant variations in line pressure. However, for the compositions having a small volume of very fine particles, the composition may be relatively uncompressible due to a lack of entrained air that would otherwise surround the fine particles. That is, the compositions disclosed herein may begin in a relatively efficient packing state, and therefore blade movement in the rheometer is not accommodated by the air pockets that exist in more cohesive powders, i.e., powders containing higher levels of very fine particles. This may result in more contact stress, and therefore a higher BFE than powders that include many very fine particles.

The SE is the converse of the BFE, in the sense that the flow pattern is generated by an upward, clockwise motion of the blade in the powder rheometer, generating gentle lifting and low stress flow of the composition. Specifically, SE is the total energy measured during the seventh cycle during the stability method measurements of the SVFR method described above (i.e., at a tip speed set at 100 mm/s) while the blade is rotating from the bottom of the vessel to the top. As with the BFE, the reduced number of very fine particles in the compositions described herein may create an efficient particle packing state and the SE will be increased as compared to the same or similar powder that includes a larger volume of very fine particles.

Conditioned Bulk Density (“CBD”) may also be measured with the FT4 Powder Rheometer using the SVFR method. Bulk density may be measured at various packing conditions, and measuring the mass of a precise volume of conditioned powder provides the CBD. The CBD of a composition having a low percentage of very fine particles, e.g., that has been classified to remove very fine particles, may be higher than the CBD of the same powder that includes a higher percentage of very fine particles (e.g., that has not been classified to remove very fine particles). Thus, a higher CBD may indicate the presence of fewer very fine-sized particles (e.g., <5 μm) in the composition.

AE is a measure of how much energy is required for a powder to become aerated, which is directly related to the cohesive strength of the powder (i.e., the tendency for particles to “stick” together). AE may be determined in the FT4 Powder Rheometer using the aeration test, which provides a precise air velocity to the base of the vessel containing the powder and measures the change in energy required to rotate the blades through the powder sample as the air velocity changes. During the aeration test, the air velocity (e.g., in mm/s) is varied over a range of from about 0.2 millimeters per second (mm/s) to about 2.0 mm/s, e.g., in 0.2 mm/s increments. As a general rule, the less cohesive, and therefore more easily fluidized the composition, the lower the AE, and the more easily the powder composition can be pneumatically conveyed.

Another measure of cohesiveness is the AR, which is a unitless quantity expressing the ratio of AE at zero air velocity to the AE at a given air velocity. If the AR is 1, then there is very little change in AE as the air velocity increases, and the composition is said to be cohesive. Powders with ARs of 2 to 20 are said to have average sensitivity to aeration, and most powders fall within this range. At an AR above 20, powders are considered sensitive to aeration. As a general rule, the larger the AR and the lower the AE, the less cohesive and therefore more easily fluidized and pneumatically conveyed the powder.

The pressure drop, measured by the Permeability test, is a measure of the resistance to air flow between particles and through the powder bed. Pressure drop may be measured with the FT4 Powder Rheometer using a Permeability test which measures the pressure drop across the powder bed as a function of the applied normal stress (kinematic) in kPa. The less the pressure drop that is measured, the more likely the powder is to flow when pneumatically conveyed. Typically, a powder with low permeability will generate a pressure drop of over 50 mbar from at about 15 kPa and at an air velocity of 0.5 mm/s. In contrast, permeable powders will barely register a pressure drop at this air velocity. Powder permeability can be associated to its tendency towards bridging or segregation which are highly undesired occurrences during the manufacture of drug product. The permeability number measures relative ease for air to travel through a conditioned powder bed; low number indicates high permeability and therefore less chances for bridging/segregation

Compressibility is another characteristic that can affect flowability and may be measured by the FT4 Powder Rheometer using the compressibility test. Compressibility is a measure of how bulk density increases on compression. The less compressible a powder is, the more likely it is to flow when pneumatically conveyed because there are more paths for air. In other words, free flowing materials tend to be insensitive to compressibility. For example, a highly compressible composition with lower flowability would be characterized by a compressibility of about 40% at 15 kPa; and a more flowable sample would have a compressibility of less than 20% at 15 kPa.

Morphology

The three dimensional morphology can render the milled or annealed or screened niraparib particles or blended compositions of the present invention more suitable for drug product manufacturing, e.g., coating, mixing, compression, extrusion etc. than unmilled or unannealed or unscreened niraparib particles or blended compositions.

The niraparib particles or blended compositions of the present invention can be prepared by any suitable processes known in the art. In certain embodiments, the niraparib particles or blended compositions of the present invention are prepared by a process described herein. The niraparib particles can have a needle shape in some embodiments. The niraparib partices can have a rod shape in some embodiments. In some embodiments, the niraparib particles are shaped like fine rods and plates and are birefringent under cross-polarized light.

An “aspect ratio” is the ratio of width divided by length of a particle.

“Elongation” is defined as 1-aspect ratio. Shapes symmetrical in all axes, such as circles or squares, will tend to have an elongation close to 0, whereas needle-shaped particles will have values closer to 1. Elongation is more an indication of overall form than surface roughness.

“Convexity” is a measurement of the surface roughness of a particle and is calculated by dividing the perimeter of an imaginary elastic band around the particle by the true perimeter of the particle. A smooth shape, regardless of form, has a convexity of 1 while a very ‘spiky’ or irregular object has a convexity closer to 0.

“Circularity” or “high sensitivity circularity” is a measurement of the ratio of the actual perimeter of a particle to the perimeter of a circle of the same area. A perfect circle has a circularity of 1 while a very narrow rod has a High Sensitivity (HS) Circularity close to 0. The higher the HS Circularity value the closer it is to a circle. Intuitively, circularity is a measure of irregularity or the difference from a perfect circle.

Milling

In some embodiments, a composition described herein comprises unmilled, milled, or a mixture of milled and unmilled niraparib particles. In some embodiments, the niraparib particles of a composition described herein are unmilled niraparib particles. In some embodiments, the niraparib particles of a composition described herein are milled niraparib particles. In some embodiments, the niraparib particles of a composition described herein are wet milled particles.

In some embodiments, niraparib particles can be milled with a milling apparatus. Various milling apparatus are known in the art including for example wet mills, ball mills, rotary mills, and fluid air milling systems.

An embodiment of the inventive method comprises wet-milling niraparib to provide a wet-milled niraparib composition. “Wet-milling” can also be referred to as “media milling” or “wet-bead milling.” In an embodiment of the invention, the method comprises wet-milling the niraparib in any suitable manner. Exemplary mills that may be suitable for wet-milling include, but are not limited to, ball (or bead) mill, rod mill, hammer mill, colloid mill, fluid-energy mill, high-speed mechanical screen mill, and centrifugal classifier mill. The size and amount of milling media (e.g., beads) may be varied, as appropriate, depending on, e.g., the desired size of the niraparib particles and the duration of the milling. In some embodiments, the milling media (e.g., beads) may be from about 0.5 mm to about 10 mm. The method may comprise wet-milling using any suitable amount of milling media. In some embodiments, the milling media may comprise from about 30% to about 70% of the volume of the mill chamber.

The inventive method may comprise wet-milling the mixture for any suitable duration. The duration of the wet-milling may be varied, as appropriate, depending on, e.g., the desired size of the niraparib particles, the size and/or amount of beads, and/or batch size. In some embodiments of the invention, the duration of the wet-milling may be from about one minute or less to about 20 minutes or more. In some embodiments, the duration of the wet-milling may be from about 2 minutes to about 15 minutes. In an embodiment of the invention, a change in any one or more of milling speed (impeller/tip speed), size or amount of the milling media, rate the mixture is fed into the mill, the viscosity or temperature of the mixture, amount of niraparib in the mixture, and size or hardness of niraparib particles may change the duration of milling required to achieve the desired particle size.

In some embodiments which include wet-milling a mixture of niraparib and aqueous liquid carrier, the method comprises drying the wet-milled, niraparib composition having the desired niraparib particle size. The drying may be carried out in any suitable manner, including but not limited to, spray-drying. An embodiment of the method further comprises processing the wet-milled niraparib composition into any suitable pharmaceutical composition.

In some embodiments, a method may comprise reaerating the wet-milled niraparib composition. DE aerating is optional and in some embodiments, the method may lack a reaerating step. DE aerating may be performed in any suitable manner such as, e.g., by vacuuming the mixture.

In some embodiments, reaerating the wet-milled niraparib composition provides a first-pass, wet-milled niraparib composition. A “pass,” as used herein, comprises wet-milling once and reaerating once as described herein. The inventive methods may comprise any suitable number of passes. The number of passes is not limited and in some embodiments, the inventive methods may comprise one, two, three, four, five, six, seven, eight, nine, ten, or more passes. In this regard, the inventive method may comprise repeating the wet-milling and/or reaerating described herein one or more times. The number of passes may be varied, as appropriate, depending on the desired size of the niraparib particles, the starting size of the niraparib particles, the amount of niraparib in the mixture, the amount of liquid carrier, the rate at which the mixture is added to the mill, and/or the temperature of the milling chamber. In some embodiments, the method comprises sizing a sample of the wet-milled, niraparib composition following each pass to determine if the niraparib particles have the desired size range. If the niraparib particles are too large, the method may comprise repeating wet-milling for one or more additional passes. If the niraparib particles have an acceptable size, the method may comprise processing the wet-milled niraparib composition to provide a pharmaceutical composition.

The wet-milling of the inventive method, regardless of the number of passes, may provide niraparib particles having any suitable cumulative size distribution.

An embodiment of the inventive method comprises processing the wet-milled niraparib composition to provide a pharmaceutical composition. The processing of the inventive method may be in any suitable manner to provide any suitable dosage form. In some embodiments, processing the wet-milled niraparib composition comprises encapsulating the wet-milled niraparib composition to provide a capsule. The pharmaceutical compositions prepared by the methods of the present invention can be encapsulated using large-scale production methods. Suitable methods of encapsulation include plate processes, rotary die-processes, microencapsulation processes, and machine encapsulation processes as disclosed in Remington's.

Another embodiment of the invention provides a method of preparing a pharmaceutical composition comprising wet-milling niraparib particles in a liquid carrier to provide a wet-milled niraparib composition and processing the wet-milled niraparib composition to provide a pharmaceutical composition. The method comprises wet-milling and processing as described herein with respect to other aspects of the invention.

A ball mill is a cylindrical device used in grinding or mixing materials. Ball mills typically rotate around a horizontal axis, partially filled with the material to be ground in addition to any grinding medium if used. Different materials are used as media, including ceramic balls such as high density alumina media, flint pebbles and stainless steel balls. An internal cascading effect reduces the particulate material to a finer powder. Industrial ball mills can operate continuously, fed at one end and discharged at the other end. Large to medium-sized ball mills are mechanically rotated on their axis, but small ones normally consist of a cylindrical capped container that sits on two drive shafts with belts used to transmit rotary motion.

Rotary mills, are also referred to as burr mills, disk mills, and attrition mills, typically include two metal plates having small projections (i.e. burrs). Alternatively, abrasive stones may be employed as the grinding plates. One plate may be stationary while the other rotates, or both may rotate in opposite directions.

A fluid air milling system utilizes turbulent free jets in combination with a high efficiency centrifugal classifier in a common housing. A typical fluid air milling system includes an inlet, chamber with rotor, screen, and an outlet. Feed can be introduced into the common housing through either a double flapper valve or injector. Flooding the pulverizing zone to a level above the grinding nozzles forms the mill load. Turbulent free jets can be used to accelerate the particles for impact and breakage. After impact the fluid and size reduced particles leave the bed and travel upwards to the centrifugal classifier where rotor speed will define which size will continue with the fluid through the rotor and which will be rejected back to the particle bed for further size reduction. The high degree of particle dispersion leaving the pulverizing zone aids in the efficient removal of fine particles by the classifier. Operating parameters of rotor speed, nozzle pressure, and bed level allow for optimizing productivity, product size, and distribution shape (slope). A low-pressure air purge can be used to seal the gap between the rotor and the outlet plenum eliminating particles bypassing the rotor and allowing for close top size control.

As the particle size of a powder decreases, the surface area typically increases. However, as the particle size of a powder decreases, the tendency to form agglomerations can also increase. This tendency to form agglomerations can offset any benefits obtained by increasing the surface area.

In some embodiments, milled particles have a higher packing density (i.e. relative to the same particles unmilled). For example, the packing density can increase by 0.2, 0.4, 0.6, 0.8, 1.0 or 1.2 g/cc. An increase in packing density of even 5 or 10% can be particularly beneficial for reducing the volume of powdered materials for shipping. In some embodiments, the packing density of milled particles or particle blends is increased by at least 20% relative to the same particles or particle blends that are unmilled.

Annealing

In some embodiments, a method of making a composition described herein, such as a niraparib capsule formulation, comprises annealing the niraparib particles one or more times. For example, a method of making a niraparib capsule formulation can comprise heating and cooling the niraparib particles one, two, three, four, five, or more times. In some embodiments, the niraparib particles are annealed after milling, such as wet milling.

Annealing can comprise heating and cooling niraparib particles. For example, annealing can comprises heating niraparib particles to a temperature of about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C. for about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, or 14 hours, followed by cooling the niraparib particles.

For example, after heating the niraparib particles, the niraparib particles can be cooled to a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. over a period of time. For example, after heating the niraparib particles, the niraparib particles can be cooled to a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. over a period of about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 15 hours, 15 hours, 17 hours, 18 hours, 19 hours, 20 hours, 21 hours, 22 hours, 23 hours, or 24 hours or longer.

For example, annealing can comprises heating niraparib particles to a temperature of about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., or 90° C. followed by cooling the niraparib particles to a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. over a period of about 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 15 hours, 15 hours, 17 hours, 18 hours, 19 hours, 20 hours, 21 hours, 22 hours, 23 hours, or 24 hours or longer.

In some embodiments, particles of a composition described herein, such as niraparib particles, are annealed (e.g., heated and cooled) one or more times. For example, the niraparib particles of a composition described herein can be heated and cooled one, two, three, four, five, or more times.

In some embodiments, annealed particles exhibit a lower total energy of powder flow (i.e. relative to the same particles unannealed). In some embodiments, particles annealed two or more times, such as two or three or four or five or more times, exhibit a lower total energy of powder flow (i.e. relative to the same particles unannealed or annealed once). This equates to less energy expenditure for handing (e.g. conveying and mixing) powdered materials. Annealing two or more times can lower the total energy of powder flow by 5%, 10%, 20%, 30%, 40%, 50%, 60%, or greater.

The free-flowing powder can exhibit any one or combination of improved properties as just described. In some embodiments, the niraparib particles of the present invention have a three dimensional morphology.

Measurement of particle size for niraparib formulations described herein can use, for example, wet dispersion laser diffraction method for particle size determination using a Malvern Mastersizer 3000 Particle Size Analyzer equipped with the Hydro MV sample dispersion unit. The particle size analyzer can determine particle size using low-angle laser light scattering and calculates results in % volume based on equivalent spheres. Volume distributions for the D₁₀, D₅₀, D₉₀, D_{4,3}, and D_{3,2} can be determined. The suspension is added to the tank until the obscuration is in range, targeting a 10% obscuration. Measurements are taken once the obscuration remains consistent.

The percentage of thicker particles can be determined using an instrument that measures the size and shape of particles, such as by the technique of static image analysis, for example, a Malvern Instrument Morphologi G3. The intensity of light can be quantified by a grey scale factor which depends on the amount of light reaching the detector. The grey scale image of a particle ranges from 0 (black) to 255 (white) and it is related to the thickness of the particle. The lower the intensity value the darker the image therefore the thicker the particle. In certain embodiments, the niraparib particles or blended compositions of the present invention have greater than 30%, greater than 40%, greater than 45% or greater than 50% of the particles with intensity less than 80. In one embodiment, 30-100%, 30-90%, 30-80%, 30%-70%, 30-60%, 40-60% or 40-50% of the niraparib particles or blended compositions of the present invention have intensity less than 80.

Each of FIGS. 15A-15I depicts an exemplary scanning electron microscope (SEM) image of niraparib particles used in a batch.

In some embodiments, milled or annealed or screened niraparib particles in blended compositions of the present invention are slightly more elongated, less circular and more edgy, as indicated by lower aspect ratio, lower HS circularity and lower convexity values, respectively, than unmilled or unannealed or unscreened niraparib particles in blended compositions. In some embodiments, the niraparib particles in blended compositions of the present invention have a circularity value in the range of less than 0.8, 0.7, 0.6, 0.5, 0.4, 0.3, 0.2, or 0.1. In another embodiment, 40% of the niraparib particles in blended compositions by accumulated volume has a circularity value in the range of 0.1 to 0.6. In some embodiments, the niraparib particles in blended compositions of the present invention has an aspect ratio in the range of 0.55 to 1.0. In some embodiments, the niraparib particles in blended compositions of the present invention has a convexity value in the range 0.95 to 1.0.

Internal Friction Angle

In some embodiments, an angle of internal friction between niraparib particles or between particles of a blended composition described herein can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees.

In some embodiments, an angle of internal friction between niraparib particles can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees.

In some embodiments, an angle of internal friction between particles of a blend of niraparib particles and lactose monohydrate particles can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees. In some embodiments, an angle of internal friction between particles of a blend of niraparib particles and lactose monohydrate particles can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0.

In some embodiments, an angle of internal friction between particles of a blend of niraparib particles, lactose monohydrate particles and magnesium stearate particles, can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.4, 36.5, 36.6, 36.7, 36.8, 36.9, 37.0, 37.1, 37.2, 37.3, 37.4, 37.5, 37.6, 37.7, 37.8, 37.9, 38.0, 38.1, 38.2, 38.3, 38.4, 38.5, 38.6, 38.7, 38.8, 38.9, 39.0, 39.1, 39.2, 39.3, 39.4, 39.5, 39.6, 39.7, 39.8, 39.9, 40.0, 40.1, 40.2, 40.3, 40.4, 40.5, 40.6, 40.7, 40.8, 40.9 or 50.0 degrees. In some embodiments, an angle of internal friction between particles of a blend of niraparib particles, lactose monohydrate particles and magnesium stearate particles, can be at most about 28.0, 28.1, 28.2, 28.3, 28.4, 28.5, 28.6, 28.7, 28.8, 28.9, 30.0, 29.1, 29.2, 29.3, 29.4, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0.

Flow Function (FF) Ratio

In some embodiments, the Flow Function (FF) Ratio of niraparib particles or of particles of a blended composition described herein can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0.

In some embodiments, the Flow Function (FF) Ratio of niraparib particles can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In some embodiments, the Flow Function (FF) Ratio of particles of a blend of niraparib particles and lactose monohydrate particles can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0. In some embodiments, the Flow Function (FF) Ratio of particles of a blend of niraparib particles (e.g., milled niraparib particles) and lactose monohydrate particles can be at least about 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0.

In some embodiments, the Flow Function (FF) Ratio of particles of a blend of niraparib particles, lactose monohydrate particles and magnesium stearate particles, can be at least about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0. In some embodiments, the Flow Function (FF) Ratio of particles of a blend of niraparib particles, lactose monohydrate particles and magnesium stearate particles, can be at least about 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0.

Wall Friction

A Wall Friction test can be used to provide a measurement of the sliding resistance between a powder and the surface of process equipment, such as an encapsulator or blender or hopper. This can be important for understanding discharge behavior from hoppers, continuity of flow in transfer chutes and tablet ejection forces. It is also useful when investigating whether a powder will adhere to the wall of process equipment and various other surfaces, such as the inside of sachets, capsules and other packaging material. The measurement principle is very similar to the shear cell test, but rather than shearing powder against powder, in this test a coupon of material representing the process equipment wall is sheared against the powder in question. The FT4 Wall Friction accessory allows for a range of coupons to be investigated, and bespoke surfaces can be manufactured if required. Data is typically represented as a plot of shear stress against normal stress, allowing the determination of Wall Friction Angle (phi). The greater the wall friction angle, the higher the resistance between the powder and wall coupon.

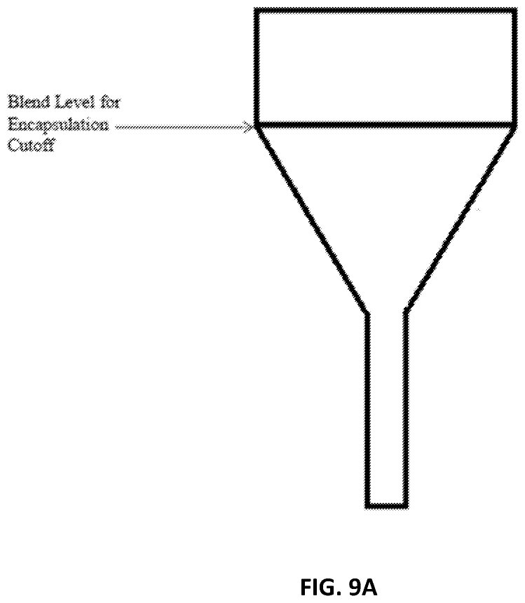

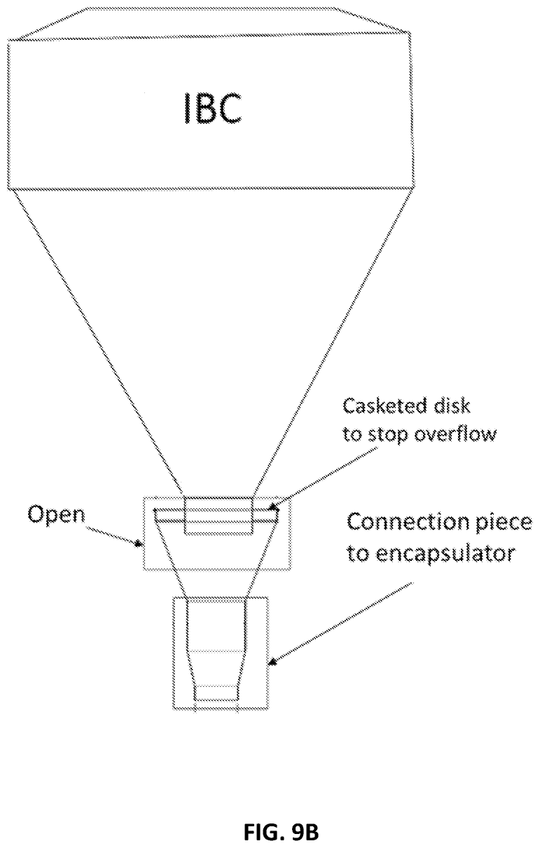

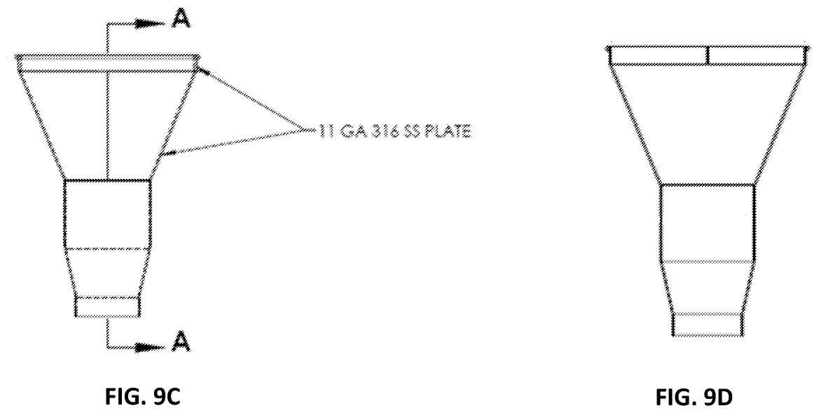

Exemplary diagrams relating to exemplary blenders and transfer chutes are provided in FIGS. 9A-9D.

Hoppers are used extensively throughout the processing environment and whilst they are often considered to be simple systems, they are responsible for causing a great deal of process interruption and product quality issues. If a powder possesses properties that are not optimized for the hopper geometry and equipment surface, then flow from the hopper may be variable or even none existent. Data from shear cell and wall friction tests can be used to calculate the critical hopper dimensions to ensure good flow.

A Wall Friction test can be used to measure the sliding resistance between the powder and the surface of the process equipment. This is particularly important for understanding discharge behavior from hoppers, continuity of flow in transfer chutes and tablet ejection forces. It is also useful when investigating whether a powder will adhere to the wall of process equipment and various other surfaces, such as the inside of sachets, capsules and other packaging material.

The measurement principle is very similar to the shear cell test, but rather than shearing powder against powder, in this test a coupon of material representing the process equipment wall is sheared against the powder in question. The FT4 Wall Friction accessory allows for a range of coupons to be investigated. Wall Friction is typically represented as a plot of shear stress against normal stress, allowing the determination of Wall Friction Angle (phi). The greater the wall friction angle, the higher the resistance between the powder and wall coupon.

In some embodiments, the wall friction angle of niraparib particles or of particles of a blended composition described herein can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0 degrees.

In some embodiments, the wall friction angle of niraparib particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0 degrees.

In some embodiments, the wall friction angle of particles of a blend of niraparib particles and lactose monohydrate particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0 degrees. In some embodiments, the wall friction angle of particles of a blend of niraparib particles (e.g., milled niraparib particles) and lactose monohydrate particles can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0.

In some embodiments, the wall friction angle of particles of a blend of niraparib particles, lactose monohydrate particles and magnesium stearate particles, can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0 degrees. In some embodiments, the wall friction angle of particles of a blend of niraparib particles, lactose monohydrate particles and magnesium stearate particles, can be at most about 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, or 26.0 degrees.

Compressibility

In some embodiments, the compressibility percentage measured at 15 kPa of particles of a composition, such as an unmilled or milled composition described herein, can be at most or at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%.

In some embodiments, the compressibility percentage measured at 15 kPa of milled or unmilled niraparib particles of a composition described herein can be at most or at least about 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%.

In some embodiments, the compressibility percentage measured at 15 kPa of unmilled or milled niraparib particles of a composition described herein that have been annealed once time can be at least about 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%. In some embodiments, the compressibility percentage measured at 15 kPa of unmilled or milled niraparib particles of a composition described herein that have been annealed once time can be at most about 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9%, 50.0%, or 60%.

In some embodiments, the compressibility percentage measured at 15 kPa of unmilled or milled niraparib particles of a composition described herein that have been annealed two or more times can be at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9% or 30.0%. In some embodiments, the compressibility percentage measured at 15 kPa of unmilled or milled niraparib particles of a composition described herein that have been annealed two or more times can be at most about 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9% or 30.0%.

In some embodiments, the compressibility percentage measured at 15 kPa of niraparib particles can be at most or at least about 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 30.0%, 29.1%, 29.2%, 29.3%, 29.4%, 29.5%, 29.6%, 29.7%, 29.8%, 29.9%, 30.0%, 30.1%, 30.2%, 30.3%, 30.4%, 30.5%, 30.6%, 30.7%, 30.8%, 30.9%, 31.0%, 31.1%, 31.2%, 31.3%, 31.4%, 31.5%, 31.6%, 31.7%, 31.8%, 31.9%, 32.0%, 32.1%, 32.2%, 32.3%, 32.4%, 32.5%, 32.6%, 32.7%, 32.8%, 32.9%, 33.0%, 33.1%, 33.2%, 33.3%, 33.4%, 33.5%, 33.6%, 33.7%, 33.8%, 33.9%, 34.0%, 34.1%, 34.2%, 34.3%, 34.4%, 34.5%, 34.6%, 34.7%, 34.8%, 34.9%, 35.0%, 35.1%, 35.2%, 35.3%, 35.4%, 35.5%, 35.6%, 35.7%, 35.8%, 35.9%, 36.0%, 36.1%, 36.2%, 36.3%, 36.4%, 36.5%, 36.6%, 36.7%, 36.8%, 36.9%, 37.0%, 37.1%, 37.2%, 37.3%, 37.4%, 37.5%, 37.6%, 37.7%, 37.8%, 37.9%, 38.0%, 38.1%, 38.2%, 38.3%, 38.4%, 38.5%, 38.6%, 38.7%, 38.8%, 38.9%, 39.0%, 39.1%, 39.2%, 39.3%, 39.4%, 39.5%, 39.6%, 39.7%, 39.8%, 39.9%, 40.0%, 40.1%, 40.2%, 40.3%, 40.4%, 40.5%, 40.6%, 40.7%, 40.8%, 40.9% or 50.0%.

In some embodiments, the compressibility percentage measured at 15 kPa of particles of a blend of niraparib particles and lactose monohydrate particles can be at most or at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9% or 20.0%. In some embodiments, the compressibility percentage measured at 15 kPa of a blend of niraparib particles (e.g., milled niraparib particles) and lactose monohydrate particles can be at most about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9% or 13.0%. In some embodiments, the compressibility percentage measured at 15 kPa of a blend of niraparib particles (e.g., milled niraparib particles) and lactose monohydrate particles can be at least about 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, or 17.0%.

In some embodiments, the compressibility percentage measured at 15 kPa of a blend of niraparib particles, lactose monohydrate particles and magnesium stearate particles can be at most or at least about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9% or 20.0%. In some embodiments, the compressibility percentage measured at 15 kPa of particles of a blend of niraparib particles, lactose monohydrate particles and magnesium stearate particles, can be at most about 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9% or 13.0%.

In some embodiments, the compressibility percentage measured at 15 kPa of particles of a blend of niraparib particles, lactose monohydrate particles and magnesium stearate particles, can be at least about 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, or 17.0%.

Dose-to-Dose Uniformity

The present disclosure further recognizes the challenges present in formulation of niraparib (e.g., the formulation of capsules), wherein each contains substantially similar concentrations of niraparib or its pharmaceutically acceptable salts. In particular, it is desirable to achieve dose-to-dose uniformity in each unit dosage form (e.g., each capsule) in term of niraparib content and/or distribution.

Typical capsules are packaged and administered orally. For example, a single administration (i.e. a single dose) of a niraparib capsule may include a single capsule, two capsules, three capsules or more taken orally by the subject.

Dose to dose variability can be a challenge. Specifically, it is not desirable for one or more capsules of a lot or batch of capsules to have significant variations of drug content from one capsule to another. For example, it is not desirable for one or more capsules of a lot or batch of capsules encapsulated at later times during the encapsulation process to include higher concentrations of niraparib than one or more or all of the capsules encapsulated during the earlier times during the encapsulation process. It is not desirable for one or more capsules of a lot or batch of capsules encapsulated at certain times during the encapsulation process to include higher concentrations of niraparib than one or more or all of the capsules encapsulated during other times during the encapsulation process.

Without being limited as to theory, there are at least two possibilities that could result in the variations of drug content from one capsule to another. Variation could result from niraparib segregation in the bulk container or result from niraparib segregation during the encapsulation process itself. Segregation of a physical blend can occur for many reasons, but typically involves two main and sometimes co-contributing attributes: the physical properties of the formulation components and the process of manufacturing.

In some embodiments, the composition has a dose-to-dose niraparib concentration variation of less than 50%. In some embodiments, the composition has a dose-to-dose niraparib concentration variation of less than 40%. In some embodiments, the composition has a dose-to-dose niraparib concentration variation of less than 30%. In some embodiments, the composition has a dose-to-dose niraparib concentration variation of less than 20%. In some embodiments, the composition has a dose-to-dose niraparib concentration variation of less than 10%. In some embodiments, the composition has a dose-to-dose niraparib concentration variation of less than 5%. Specific standards for dosage uniformity may be found at: 1) Ph. Eur. 2.9.40. Uniformity of Dosage Units, 2) JP 6.02 Uniformity of Dosage Units, and 3) USP General Chapter Uniformity of Dosage Units each of which is incorporated by reference herein.

In some embodiments, the dose-to-dose niraparib concentration variation is based on 10 consecutive doses. In some embodiments, the dose-to-dose niraparib concentration variation is based on 8 consecutive doses. In some embodiments, the dose-to-dose niraparib concentration variation is based on 5 consecutive doses. In some embodiments, the dose-to-dose niraparib concentration variation is based on 3 consecutive doses. In some embodiments, the dose-to-dose niraparib concentration variation is based on 2 consecutive doses.

Capsules

In some embodiments, the pharmaceutical composition is formulated into a solid oral pharmaceutical dosage form that is a capsule.

In embodiments, a capsule is any described in WO 2018/183349, which is incorporate herein by reference.

The term capsule is intended to encompass any encapsulated shell filled with medicines in powder, pellet, semisolid or liquid form. Generally, capsules are made of liquid solutions of gelling agents like as gelatin (animal protein) and plant polysaccharides. These include modified forms of starch and cellulose and other derivatives like carrageenans as well as polymers such as PVA. Capsule ingredients may be broadly classified as: (1) Gelatin Capsules: Gelatin capsules are made of gelatin manufactured from the collagen of animal skin or bone. Also known as gel caps or gelcaps, succinated gelatin is also suitable. In gelatin capsules, other ingredients can also be added for their shape, color and hardness like as plasticizers, sorbitol to decrease or increase the capsule's hardness, preservatives, coloring agents, lubricants and disintegrants; (2) Vegetable or non-gelatin capsules: They are made of starch, HPMC, carrageenan, PVA, or hypromellose, a polymer formulated from cellulose.

In some embodiments, a therapeutically effective amount of niraparib or a pharmaceutically acceptable salt thereof administered to a subject via a solid dosage form is in the range of about 1 mg to about 1000 mg. In some embodiments, a therapeutically effective amount of niraparib or a pharmaceutically acceptable salt thereof administered to a subject via a solid dosage form is in the range of from about 50 mg to about 300 mg. In some embodiments, a niraparib formulation is administered as a solid dosage form at a concentration of about 50 mg to about 100 mg. In some embodiments, the niraparib formulation is administered as a solid dosage form at concentration of about 100 mg to about 300 mg. For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a therapeutically effective amount of niraparib tosylate monohydrate administered to a subject via a solid dosage form can be from about 1 mg to about 1000 mg, for example, from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a therapeutically effective amount of niraparib tosylate monohydrate administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a therapeutically effective amount of niraparib tosylate monohydrate administered to a subject via a solid dosage form can be about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some embodiments, a therapeutically effective amount of niraparib tosylate monohydrate administered to a subject via a solid dosage form is about 79.7 mg. In some embodiments, a therapeutically effective amount of niraparib tosylate monohydrate administered to a subject via a solid dosage form is about 159.4 mg. In some embodiments, a therapeutically effective amount of niraparib tosylate monohydrate administered to a subject via a solid dosage form is about 318.8 mg. In some embodiments, a therapeutically effective amount of niraparib tosylate monohydrate administered to a subject via a solid dosage form is about 478.2 mg. In some aspects, the solid oral dosage form can be administered one, two, or three times a day (b.i.d).

Contemplated compositions of the present invention provide a therapeutically effective amount of niraparib or a pharmaceutically acceptable salt thereof over an interval of about 30 minutes to about 8 hours after administration, enabling, for example, once-a-day, twice-a-day, three times a day, and etc. administration if desired.

The formulations described herein may be introduced into a suitable capsule by using an encapsulator, e.g., an encapsulator equipped with pellet dosing chamber. The capsule sizes may be 00, 00EL, 0, 0EL, 1, 1EL, 2, 2EL, 3, 4 or 5. In some embodiments, the particles in the capsule are in a size 0 or smaller, for example, a size 1 or smaller capsule.

In some aspects, the pharmaceutical composition disclosed herein is encapsulated into discrete units. In some embodiments, the discrete units are capsules or packets. In some embodiments, the pharmaceutical composition disclosed herein is enclosed in a capsule.

In some embodiments, the capsule is formed using materials which include, but are not limited to, natural or synthetic gelatin, pectin, casein, collagen, protein, starch, modified starch, polyvinylpyrrolidone, polyvinyl alcohol, acrylic polymers, cellulose derivatives, or combinations thereof. In some embodiments, the capsule is formed using preservatives, coloring and opacifying agents, flavorings and sweeteners, sugars, gastroresistant substances, or combinations thereof. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the capsule is broken such that the particulates are sprinkled on soft foods such as apple sauce, dispersed or dissolved in a liquid (water, juice (such as apple, orange, grape), milk, formula) and swallowed without chewing or administered through a nasogastric or gastric tube. In some embodiments, the shape and size of the capsule also vary. Examples of capsule shapes include, but are not limited to, round, oval, tubular, oblong, twist off, or a non-standard shape. The size of the capsule may vary according to the volume of the particulates. In some embodiments, the size of the capsule is adjusted based on the volume of the particulates and powders. Hard or soft gelatin capsules may be manufactured in accordance with conventional methods as a single body unit comprising the standard capsule shape. A single-body soft gelatin capsule typically may be provided, for example, in sizes from 1 to 24 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size. In some embodiments, the pharmaceutical composition disclosed herein (e.g., capsule) is swallowed as a whole. Other suitable capsules also include chewable capsules; seamless capsules (e.g., suitable for sprinkling onto food or administered via tube); or capsules suitable as lozenges. In some embodiments, the pharmaceutical composition disclosed herein (e.g., capsule) does not completely disintegrate in mouth within about: 2, 3, 4, 5, 6, 7, 8, 9, 10, 11, 12, 13, 14, 15, 16, 17, 18, 19, or 20 minutes. In some embodiments, the pharmaceutical composition disclosed herein is not a film. In some embodiments, the pharmaceutical composition disclosed herein is not for buccal administration. In some embodiments, the pharmaceutical composition disclosed herein (e.g., capsule) dissolves in stomach or intestine.

In some embodiments, a capsule disclosed herein has a net weight ranging from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. For example, a capsule disclosed herein can have a net weight ranging from about 50 mg to 150 mg, from about 75 mg to about 125 mg, about 90 mg to about 110 mg, about 93 mg to about 107 mg, about 94 mg to about 106 mg, or about 95 mg to about 105 mg.

In some embodiments, a capsule disclosed herein has a net weight of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg. For example, a capsule disclosed herein can have a net weight of about 100 mg, about 98 mg, about 96 mg, about 94 mg, about 92 mg, about 90 mg, about 80 mg, about 70 mg, about 60 mg, or about 50 mg.

In some cases, a capsule has a volume ranging from about 0.1 to 0.9 ml, e.g., about 0.6 ml to about 0.8 ml, about 0.4 ml to about 0.6 ml, about 0.3 ml to about 0.5 ml, about 0.2 ml to about 0.4 ml, or about 0.1 ml to about 0.3 ml. In some cases, the capsule has a volume of about 0.9 ml, about 0.8 ml, about 0.7 ml, about 0.6 ml, about 0.5 ml, about 0.4 ml, about 0.35 ml, about 0.3 ml, about 0.25 ml, about 0.2 ml, about 0.15 ml, or about 0.1 ml. In some cases, a body of the capsule ranges from about 9 mm to about 20 mm long, e.g., about 17 mm to about 20 mm long, about 17 mm to about 19 mm long, about 16 mm to about 20 mm long, about 15 mm to about 19 mm long, about 14 mm to about 18 mm long, about 13 mm to about 17 mm long, about 12 mm to about 16 mm long, about 11 mm to about 15 mm long, about 10 mm to about 14 mm long, about 9 mm to about 13 mm long, about 9 mm to about 12 mm long, about 9 mm to about 11 mm long, or about 9 mm to about 10 mm long. In some cases, the body of the capsule is about 18 mm long, about 17 mm long, about 16 mm long, about 15 mm long, about 14 mm long, about 13 mm long, about 12 mm long, about 11 mm long, about 10 mm long, or about 9 mm long. In some cases, a cap of the capsule ranges from about 6 mm to about 12 mm long, e.g., about 10 mm to 12 mm long, about 9 mm to about 11 mm long, about 8 mm to about 10 mm long, about 7 mm to about 9 mm long, or about 6 mm to about 8 mm long. In some cases, the cap of the capsule is about 11 mm long, about 10 mm long, about 9 mm long, about 8 mm long, about 7 mm long, or about 6 mm long. In some cases, the body of the capsule has an external diameter ranging from about 4 mm to about 9 mm, e.g., about 6 mm to about 8 mm, about 7 mm to about 9 mm, about 7 mm to about 8 mm, about 5 mm to about 7 mm, or about 4 mm to about 6 mm. In some cases, the body of the capsule has an external diameter of about 9 mm, about 8 mm, about 7 mm, about 6 mm, about 5 mm, or about 4 mm. In some cases, a cap of the capsule has an external diameter ranging from about 4 mm to about 9 mm, e.g., about 7 mm to about 9 mm, about 6 mm to about 9 mm, about 7 mm to about 8 mm, about 5 mm to about 7 mm, or about 4 mm to about 6 mm. In some cases, the cap of the capsule has an external diameter of about 9 mm, about 8 mm, about 7 mm, about 6 mm, about 5 mm, or about 4 mm. In some cases, an overall closed length of the capsule ranges from about 10 mm to about 24 mm, e.g., about 20 mm to about 24 mm, or about 21 mm to about 23 mm, about 20 mm to about 22 mm, about 19 mm to about 21 mm, about 18 mm to about 20 mm, about 17 mm to about 19 mm, about 16 mm to about 18 mm, about 15 mm to about 17 mm, about 14 mm to about 16 mm, about 13 mm to about 15 mm, about 12 mm to about 14 mm, about 11 mm to about 13 mm, or about 10 mm to about 12 mm. In some cases, the overall closed length of the capsule is about 22 mm, about 24 mm, about 23 mm, about 21 mm, about 20 mm, about 19 mm, about 18 mm, about 17 mm, about 16 mm, about 15 mm, about 14 mm, about 13 mm, about 12 mm, about 11 mm, or about 10 mm.

In some cases, the capsule has a capacity of from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg. In some cases, the capsule has a capacity of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg.

For example, the capsule can have a capacity of from about 50 mg to about 800 mg, e.g., about 400 mg to about 800 mg, about 350 mg to about 450 mg, about 300 mg to about 500 mg, about 300 mg to about 400 mg, about 250 mg to about 350 mg, about 200 mg to about 300 mg, about 200 mg to about 250 mg, about 150 mg to about 200 mg, about 100 mg to about 200 mg, about 100 mg to about 150 mg, about 50 mg to about 100 mg, about 600 g, about 500 mg, about 450 mg, about 425 mg, about 400 mg, about 375 mg, about 350 mg, about 325 mg, about 300 mg, about 275 mg, about 250 mg, about 225 mg, about 200 mg, about 175 mg, about 150 mg, about 125 mg, about 100 mg, or about 75 mg. In some cases, the capsule comprises a powder with a powder density of about 0.4 g/ml to about 1.6 g/ml, e.g., about 0.4 g/ml, g/ml 1.2 g/ml, g/ml 1 g/ml, or g/ml 0.8 g/ml. In some cases, the capsule is oblong.

The method can comprise administration of a niraparib composition in 1, 2, 3, or 4 capsules once, twice, or three times daily; for example 1 or 2 or 3 capsules.

In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate) is from about 1:10 to about 10:1, respectively, for example about 1:2, about 1:3, about 1:4, about 1:5, about 1:6, about 1:7, about 1:8, about 1:9, about 1:10, about 10:1, about 9:1, about 8:1, about 7:1, about 6:1, about 5:1, about 4:1, about 3:1, or about 2:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., magnesium stearate) is from about 10:1 to about 100:1, respectively, for example about 10:1, about 20:1, about 30:1, about 40:1, about 50:1, about 60:1, about 70:1, about 80:1, or about 90:1. In some embodiments, the weight ratio of a non-active pharmaceutical ingredient (e.g., lactose monohydrate or magnesium stearate) to an active pharmaceutical ingredient (e.g., niraparib or a pharmaceutically acceptable salt thereof such as niraparib tosylate monohydrate) to is from about 3:2 to about 11:1, from about 3:1 to about 7:1, from about 1:1 to about 5:1, from about 9:2 to about 11:2, from about 4:2 to about 6:2, about 5:1, or about 2.5:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate or magnesium stearate) is about 1:1.6. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate or magnesium stearate) is about 1:2. In some embodiments, the weight ratio of niraparib or a pharmaceutically acceptable salt thereof such as niraparib tosylate monohydrate to lactose monohydrate is about 38:61, for example, 38.32:61.18. In some embodiments, the weight ratio of niraparib or a pharmaceutically acceptable salt thereof such as niraparib tosylate monohydrate to magnesium stearate is about 77:1, for example, 76.64:1.

In some embodiments, the weight ratio of a first non-active pharmaceutical ingredient to a second non-active pharmaceutical ingredient is from about 5:1 to about 200:1, respectively, for example about 5:1, about 10:1, about 20:1, about 40:1, about 50:1, about 75:1, about 100:1, about 110:1, about 120:1, about 130:1, about 140:1, about 150:1, about 160:1, about 170:1, about 180:1, about 190:1, or about 200:1. In some embodiments, the weight ratio of lactose monohydrate to magnesium stearate is about 120:1 to about 125:1. In some embodiments, the weight ratio of lactose monohydrate to magnesium stearate is about 122.36:1.

Tablets

In some embodiments, the pharmaceutical composition is formulated into a solid oral pharmaceutical dosage form that is a tablet.

In embodiments, a tablet is any described in International Application No. PCT/US18/52979, which is incorporated herein by reference.

In some embodiments, a therapeutically effective amount of niraparib or a pharmaceutically acceptable salt thereof administered to a subject via a solid dosage form is in the range of about 1 mg to about 2000 mg. In some embodiments, a therapeutically effective amount of niraparib or a pharmaceutically acceptable salt thereof administered to a subject via a solid dosage form is in the range of about 1 mg to about 1000 mg. In some embodiments, a therapeutically effective amount of niraparib or a pharmaceutically acceptable salt thereof administered to a subject via a solid dosage form is in the range of from about 50 mg to about 300 mg. In some embodiments, a niraparib formulation is administered as a solid dosage form at a concentration of about 50 mg to about 100 mg. In some embodiments, the niraparib formulation is administered as a solid dosage form at concentration of about 100 mg to about 300 mg. For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a therapeutically effective amount of niraparib tosylate monohydrate administered to a subject via a solid dosage form can be from about 1 mg to about 2000 mg, for example, from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a therapeutically effective amount of niraparib tosylate monohydrate administered to a subject via a solid dosage form can be from about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some aspects, the solid oral dosage form can be administered one, two, or three times a day (b.i.d).

For example, a therapeutically effective amount of niraparib or a pharmaceutically acceptable salt thereof administered to a subject via a solid dosage form can be about 1 mg to 5 mg, 5 mg to 10 mg, 10 mg to 20 mg, 20 mg to 25 mg, 25 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a therapeutically effective amount of niraparib tosylate monohydrate administered to a subject via a solid dosage form can be about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. In some embodiments, a therapeutically effective amount of niraparib tosylate monohydrate administered to a subject via a solid dosage form is about 79.7 mg. In some embodiments, a therapeutically effective amount of niraparib tosylate monohydrate administered to a subject via a solid dosage form is about 159.4 mg. In some embodiments, a therapeutically effective amount of niraparib tosylate monohydrate administered to a subject via a solid dosage form is about 318.8 mg. In some embodiments, a therapeutically effective amount of niraparib tosylate monohydrate administered to a subject via a solid dosage form is about 478.0 mg. In some aspects, the solid oral dosage form can be administered one, two, or three times a day (b.i.d).

Contemplated compositions of the present invention provide a therapeutically effective amount of niraparib or a pharmaceutically acceptable salt thereof over an interval of about 30 minutes to about 8 hours after administration, enabling, for example, once-a-day, twice-a-day, three times a day, and etc. administration if desired.

In some embodiments, the tablet is formed using materials which include, but are not limited to, natural or synthetic gelatin, pectin, casein, collagen, protein, modified starch, polyvinylpyrrolidone, acrylic polymers, cellulose derivatives, or combinations thereof. In some embodiments, the tablet is formed using preservatives, coloring and opacifying agents, flavorings and sweeteners, sugars, gastroresistant substances, or combinations thereof. In some embodiments, the tablet is coated. In some embodiments, the coating covering the tablet includes, but is not limited to, immediate release coatings, protective coatings, enteric or delayed release coatings, sustained release coatings, barrier coatings, seal coatings, or combinations thereof. The term “coating” means a process by which an outer layer of coating material is applied to the surface of a dosage form in order to confer specific benefits over uncoated variety. It involves application of a coat, including sugar or polymeric coats, on the dosage form. The advantages of tablet coating are taste masking, odor masking, physical and chemical protection, protection of the drug in the stomach, and to control its release profile. Coating may be applied to a wide range of oral solid dosage form, such as particles, powders, granules, crystals, pellets and tablets. When coating composition is applied to a batch of tablets in a coating pan, the tablet surfaces become covered with a polymeric film.

In some embodiments, the tablet is broken or crushed such that the particulates are sprinkled on soft foods and swallowed without chewing or can be suitable for administration via a feeding tube. In some embodiments, the shape and size of the tablet also vary. In some embodiments, the pharmaceutical composition disclosed herein (e.g., tablet) is swallowed as a whole. In some embodiments, the pharmaceutical composition disclosed herein is not a film. In some embodiments, the pharmaceutical composition disclosed herein is not for buccal administration. In some embodiments, the pharmaceutical composition disclosed herein (e.g., tablet) dissolves in stomach or intestine.

In one aspect provided herein is a composition comprising a tablet comprising: an effective amount of niraparib to inhibit polyadenosine diphosphate ribose polymerase (PARP) when administered to a subject in need thereof wherein the tablet has at least one of the following: a) a net weight of at least 200, 500, or 800 mg; b) a thickness of at least 4.0 mm; and c) a friability of less than 2%; wherein the effective amount of niraparib is from about 50 mg to about 350 mg based on the niraparib free base.

In some embodiments, the effective amount of niraparib is from about 75 mg to about 125 mg based on the niraparib free base. In some embodiments, the effective amount of niraparib is about 50 mg, about 100 mg, or about 150 mg based on the niraparib free base. In some embodiments, the effective amount of niraparib is about 100 mg based on the niraparib free base.

In some embodiments, the tablet disclosed herein has a net weight of at least 200 mg, at least 210 mg, at least 220 mg, at least 230 mg, at least 240 mg, at least 250 mg, at least 260 mg, at least 270 mg, at least 280 mg, at least 290 mg, at least 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, or at least 500 mg. In some embodiments, the tablet disclosed herein has a net weight of at least 300 mg.

In some embodiments, the effective amount of niraparib is from about 175 mg to about 225 mg based on the niraparib free base. In some embodiments, the effective amount of niraparib is about 150 mg, about 200 mg, or about 250 mg based on the niraparib free base. In some embodiments, the effective amount of niraparib is about 200 mg based on the niraparib free base.

In some embodiments, the tablet disclosed herein has a net weight of at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, at least 710 mg, at least 720 mg, at least 730 mg, at least 740 mg, at least 750 mg, at least 760 mg, at least 770 mg, at least 780 mg, at least 790 mg, or at least 800 mg. In some embodiments, the tablet has a net weight of at least 600 mg.

In some embodiments, the effective amount of niraparib is from about 275 mg to about 325 mg based on the niraparib free base. In some embodiments, the effective amount of niraparib is about 250 mg, about 300 mg, or about 350 mg based on the niraparib free base. In some embodiments, the effective amount of niraparib is about 300 mg based on the niraparib free base.

In some embodiments, the tablet disclosed herein has a net weight of at least 800 mg, at least 810 mg, at least 820 mg, at least 830 mg, at least 840 mg, at least 850 mg, at least 860 mg, at least 870 mg, at least 880 mg, at least 890 mg, at least 900 mg, at least 910 mg, at least 920 mg, at least 930 mg, at least 940 mg, at least 950 mg, at least 960 mg, at least 970 mg, at least 980 mg, at least 990 mg, at least 1000 mg, at least 1010 mg, at least 1020 mg, at least 1030 mg, at least 1040 mg, at least 1050 mg, at least 1060 mg, at least 1070 mg, at least 1080 mg, at least 1090 mg, at least 1100 mg, at least 1110 mg, at least 1120 mg, at least 1130 mg, at least 1140 mg, at least 1150 mg, at least 1160 mg, at least 1170 mg, at least 1180 mg, at least 1190 mg, or at least 1200 mg. In some embodiments, the tablet disclosed herein has a net weight of about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, or about 1200 mg. In some embodiments, the tablet has a net weight of at least 1000.

In some embodiments, the tablet disclosed herein has a thickness of at least 4.0 mm, at least 4.1 mm, at least 4.2 mm, at least 4.3 mm, at least 4.4 mm, at least 4.5 mm, at least 4.6 mm, at least 4.7 mm, at least 4.8 mm, at least 4.9 mm, at least 5.0 mm, at least 5.1 mm, at least 5.2 mm, at least 5.3 mm, at least 5.4 mm, at least 5.5 mm, at least 5.6 mm, at least 5.7 mm, at least 5.8 mm, at least 5.9 mm, at least 6.0 mm, at least 6.1 mm, at least 6.2 mm, at least 6.3 mm, at least 6.4 mm, at least 6.5 mm, at least 6.6 mm, at least 6.7 mm, at least 6.8, at least 6.9 mm, at least 7.0 mm, at least 7.1 mm, at least 7.2 mm, at least 7.3 mm, at least 7.4 mm, at least 7.5 mm, at least 7.6 mm, at least 7.7 mm, at least 7.8 mm, at least 7.9 mm, at least 8.0 mm, at least 8.5 mm, at least 9.0 mm, at least 9.5 mm, or at least 10 mm. In some embodiments, the tablet disclosed herein has a thickness of about 4.5 mm, about 4.6 mm, about 4.7 mm, about 4.8 mm, about 4.9 mm, about 5.0 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, about 6.1 mm, about 6.2 mm, about 6.3 mm, about 6.4 mm, about 6.5 mm, about 6.6 mm, about 6.7 mm, about 6.8, about 6.9 mm, about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 8.0 mm, about 8.5 mm, about 9.0 mm, about 9.5 mm, or about 10 mm.

In some embodiments, the tablet disclosed herein has a friability of less than 2%, less than 1.9%, less than 1.8%, less than 1.7%, less than 1.6%, less than 1.5%, less than 1.4%, less than 1.3%, less than 1.2%, less than 1.1%, less than 1.0%, less than 0.9%, less than 0.8%, less than 0.7%, less than 0.6%, less than 0.5%, less than 0.4%, less than 0.3%, less than 0.2%, or less than 0.1%.

In some embodiments, a tablet disclosed herein has a net weight ranging from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg, 1000 mg to 1050 mg, 1050 mg to 1100 mg, 1100 mg to 1150 mg, 1150 mg to 1200 mg, 1200 mg to 1250 mg, 1250 mg to 1300 mg, 1300 mg to 1350 mg, 1350 mg to 1400 mg, 1400 mg to 1450 mg, 1450 mg to 1500 mg, 1500 mg to 1550 mg, 1550 mg to 1600 mg, 1600 mg to 1650 mg, 1650 mg to 1700 mg, 1700 mg to 1750 mg, 1750 mg to 1800 mg, 1800 mg to 1850 mg, 1850 mg to 1900 mg, 1900 mg to 1950 mg, or 1950 mg to 2000 mg. For example, a tablet disclosed herein can have a net weight ranging from about 50 mg to 150 mg, from about 75 mg to about 125 mg, about 90 mg to about 110 mg, about 93 mg to about 107 mg, about 94 mg to about 106 mg, or about 95 mg to about 105 mg. In other instances, a tablet disclosed herein has a net weight ranging from about 850 mg to 900 mg, from about 900 mg to about 950 mg, from about 950 mg to 1000 mg, from about 1000 mg to about 1050 mg, from about 1050 mg to about 1100 mg, from about 1100 mg to 1150 mg, from about 1150 mg to 1200 mg, from about 1200 mg to 1250 mg, from about 1250 mg to 1300 mg, from about 1300 mg to 1350 mg, from about 1350 mg to 1400 mg, from about 1400 mg to 1450 mg, from about 1450 mg to 1500 mg, from about 1500 mg to 1550 mg, from about 1550 mg to 1600 mg, from about 1600 mg to 1650 mg, from about 1650 mg to 1700 mg, from about 1700 to about 1750 mg, from about 1750 mg to 1800 mg, from about 1800 mg to about 1850 mg, from about 1850 mg to 1900 mg, from about 1900 mg to about 1950 mg, or from about 1950 mg to 2000 mg.

In some embodiments, a tablet disclosed herein has a net weight of about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, or 2000 mg. For example, a tablet disclosed herein can have a net weight of about 100 mg, about 98 mg, about 96 mg, about 94 mg, about 92 mg, about 90 mg, about 80 mg, about 70 mg, about 60 mg, or about 50 mg. In other instances, a tablet disclosed herein has a net weight ranging from about 1050 mg, 1040 mg, 1030 mg, 1020 mg, 1010 mg, about 1000 mg, about 990 mg, about 980 mg, about 970 mg, about 960 mg, about 950 mg, or about 940 mg.

In some embodiments, the niraparib comprises niraparib free base or a pharmaceutically acceptable salt thereof. In some embodiments, the pharmaceutically acceptable salt of niraparib is niraparib tosylate.

The method can comprise administration of a niraparib composition in 1, 2, 3, or 4 tablets once, twice, or three times daily; for example 1 or 2 or 3 tablets.

In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., lactose monohydrate, lactose anhydrous, mannitol, or calcium phosphate dibasic) is from about 1:10 to about 10:1, respectively, for example about 1:2, about 1:3, about 1:4, about 1:5, about 1:6, about 1:7, about 1:8, about 1:9, about 1:10, about 10:1, about 9:1, about 8:1, about 7:1, about 6:1, about 5:1, about 4:1, about 3:1, or about 2:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., microcrystalline cellulose, starch, polyethylene oxide, or hydroxypropyl methylcellulose (HPMC)) is from about 1:10 to about 10:1, respectively, for example about 1:2, about 1:3, about 1:4, about 1:5, about 1:6, about 1:7, about 1:8, about 1:9, about 1:10, about 10:1, about 9:1, about 8:1, about 7:1, about 6:1, about 5:1, about 4:1, about 3:1, or about 2:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., povidone, hydroxylpropyl cellulose, or hydroxypropyl methylcellulose) is from about 10:1 to about 100:1, respectively, for example about 10:1, about 20:1, about 30:1, about 40:1, about 50:1, about 60:1, about 70:1, about 80:1, or about 90:1. In some embodiments, the weight ratio of an active pharmaceutical ingredient (e.g., niraparib or a pharmaceutically acceptable salt thereof such as niraparib tosylate monohydrate) to a non-active pharmaceutical ingredient (e.g., magnesium stearate) is from about 10:1 to about 100:1, respectively, for example about 10:1, about 20:1, about 30:1, about 40:1, about 50:1, about 60:1, about 70:1, about 80:1, or about 90:1. In some embodiments, the weight ratio of a non-active pharmaceutical ingredient to an active pharmaceutical ingredient (e.g., niraparib or a pharmaceutically acceptable salt thereof such as niraparib tosylate monohydrate) to is from about 3:2 to about 11:1, from about 3:1 to about 7:1, from about 1:1 to about 5:1, from about 9:2 to about 11:2, from about 4:2 to about 6:2, about 5:1, or about 2.5:1. In some embodiments, the weight ratio of an active pharmaceutical ingredient (e.g., niraparib or a pharmaceutically acceptable salt thereof such as niraparib tosylate monohydrate) to a non-active pharmaceutical ingredient is about 1:1.6. In some embodiments, the weight ratio of an active pharmaceutical ingredient (e.g., niraparib or a pharmaceutically acceptable salt thereof such as niraparib tosylate monohydrate) to a non-active pharmaceutical ingredient is about 1:2. In some embodiments, the weight ratio of an active pharmaceutical ingredient (e.g., niraparib or a pharmaceutically acceptable salt thereof such as niraparib tosylate monohydrate) to a non-active pharmaceutical ingredient is about 1:1.1. In some embodiments, the weight ratio of an active pharmaceutical ingredient (e.g., niraparib or a pharmaceutically acceptable salt thereof such as niraparib tosylate monohydrate) to a non-active pharmaceutical ingredient is about 1:1. In some embodiments, the weight ratio of niraparib or a pharmaceutically acceptable salt thereof such as niraparib tosylate monohydrate to lactose monohydrate is about 48:20, for example, 47.8:20.4 In some embodiments, the weight ratio of niraparib or a pharmaceutically acceptable salt thereof such as niraparib tosylate monohydrate to lactose monohydrate is about 48:19, for example, 47.8:19.4. In some embodiments, the weight ratio of niraparib or a pharmaceutically acceptable salt thereof such as niraparib tosylate monohydrate to lactose monohydrate is about 48:18, for example, 47.8:17.9. In some embodiments, the weight ratio of niraparib or a pharmaceutically acceptable salt thereof such as niraparib tosylate monohydrate to magnesium stearate is about 48:1, for example, 47.8:1.

In some embodiments, the weight ratio of a first non-active pharmaceutical ingredient to a second non-active pharmaceutical ingredient is from about 1:1 to about 200:1, respectively, for example about 1:1, about 2:1, about 3:1, about 4:1, about 5:1, about 10:1, about 15:1, about 20:1, about 25:1, about 30:1, about 40:1, about 50:1, about 60:1, about 70:1, about 75:1, about 80:1, about 90:1, about 100:1, about 110:1, about 120:1, about 130:1, about 140:1, about 150:1, about 160:1, about 170:1, about 180:1, about 190:1, or about 200:1. In some embodiments, the weight ratio of lactose monohydrate to magnesium stearate is about 120:1 to about 125:1. In some embodiments, the weight ratio of lactose monohydrate to magnesium stearate is about 122.36:1. In some embodiments, the weight ratio of lactose monohydrate to magnesium stearate is about 20:1. In some embodiments, the weight ratio of lactose monohydrate to magnesium stearate is about 10:1.

In one embodiment, an exemplary niraparib formulation comprises 478.0 mg of niraparib tosylate monohydrate, 203.5 mg of lactose monohydrate, 203.5 mg of microcrystalline cellulose, 40.0 mg of crospovidone, and 20.0 mg of povidone for the intragranular phase; and 40.0 mg of crospovidone, 5.0 mg of silicon dioxide, and 10.0 mg of magnesium stearate for the extragranular phase. In one embodiment, an exemplary niraparib formulation comprises 47.8% by weight of niraparib tosylate monohydrate, 20.4% by weight of lactose monohydrate, 20.4% by weight of microcrystalline cellulose, 4.0% by weight of crospovidone, and 2.0% by weight of povidone for the intragranular phase; and 4.0% by weight of crospovidone, 0.5% by weight of silicon dioxide, and 1.0% by weight of magnesium stearate for the extragranular phase.

In one embodiment, an exemplary niraparib formulation comprises 478.0 mg of niraparib tosylate monohydrate, 193.5 mg of lactose monohydrate, 193.5 mg of microcrystalline cellulose, 40.0 mg of croscarmellose, and 40.0 mg of hydroxypropyl cellulose for the intragranular phase; and 40.0 mg of croscarmellose sodium, 5.0 mg of silicon dioxide, and 10.0 mg of magnesium stearate for the extragranular phase. In one embodiment, an exemplary niraparib formulation comprises 47.8% by weight of niraparib tosylate monohydrate, 19.4% by weight of lactose monohydrate, 19.4% by weight of microcrystalline cellulose, 4.0% by weight of croscarmellose, and 4.0% by weight of hydroxypropyl cellulose for the intragranular phase; and 4.0% by weight of croscarmellose sodium, 0.5% by weight of silicon dioxide, and 1.0% by weight of magnesium stearate for the extragranular phase.

In one embodiment, an exemplary niraparib formulation comprises 478.0 mg of niraparib tosylate monohydrate, 178.5 mg of lactose monohydrate, 178.5 mg of microcrystalline cellulose, 40.0 mg of crospovidone, 40.0 mg of povidone, and 25.0 mg of silicon dioxide for the intragranular phase; and 40.0 mg of crospovidone, 10.0 mg of silicon dioxide, and 10.0 mg of magnesium stearate for the extragranular phase. In one embodiment, an exemplary niraparib formulation comprises 47.8% by weight of niraparib tosylate monohydrate, 17.9% by weight of lactose monohydrate, 17.9% by weight of microcrystalline cellulose, 4.0% by weight of crospovidone, 4.0% by weight of povidone, and 2.5% by weight of silicon dioxide for the intragranular phase; and 4.0% by weight of crospovidone, 1.0% by weight of silicon dioxide, and 1.0% by weight of magnesium stearate for the extragranular phase.

In one embodiment, an exemplary niraparib formulation comprises 478.0 mg of niraparib tosylate monohydrate, 201.0 mg of microcrystalline cellulose, 201.0 mg of calcium phosphate dibasic, 40.0 mg of crospovidone, 20.0 mg of povidone, and 5.0 mg magnesium stearate for the intragranular phase; and 40.0 mg of crospovidone, 5.0 mg of silicon dioxide, and 10.0 mg of magnesium stearate for the extragranular phase. In one embodiment, an exemplary niraparib formulation comprises 47.8% by weight of niraparib tosylate monohydrate, 20.1% by weight of microcrystalline cellulose, 20.1% by weight of calcium phosphate dibasic, 4.0% by weight of crospovidone, 2.0% by weight of povidone, and 0.5% by weight magnesium stearate for the intragranular phase; and 4.0% by weight of crospovidone, 0.5% by weight of silicon dioxide, and 1.0% by weight of magnesium stearate for the extragranular phase.

In one embodiment, an exemplary niraparib formulation comprises 478.0 mg of niraparib tosylate monohydrate, 201.0 mg of microcrystalline cellulose, 201.0 mg of mannitol, 40.0 mg of croscarmellose sodium, 20.0 mg of hydroxylpropyl cellulose, and 5.0 mg magnesium stearate for the intragranular phase; and 40.0 mg of croscarmellose sodium, 5.0 mg of silicon dioxide, and 10.0 mg of magnesium stearate for the extragranular phase. In one embodiment, an exemplary niraparib formulation comprises 47.8% by weight of niraparib tosylate monohydrate, 20.1% by weight of microcrystalline cellulose, 20.1% by weight of mannitol, 4.0% by weight of croscarmellose sodium, 2.0% by weight of hydroxylpropyl cellulose, and 0.5% by weight magnesium stearate for the intragranular phase; and 4.0% by weight of croscarmellose sodium, 0.5% by weight of silicon dioxide, and 1.0% by weight of magnesium stearate for the extragranular phase.

In one embodiment, an exemplary niraparib formulation comprises 478.0 mg of niraparib tosylate monohydrate, 201.0 mg of microcrystalline cellulose, 201.0 mg of mannitol, 40.0 mg of crospovidone, 20.0 mg of povidone, and 5.0 mg magnesium stearate for the intragranular phase; and 40.0 mg of crospovidone, 5.0 mg of silicon dioxide, and 10.0 mg of magnesium stearate for the extragranular phase. In one embodiment, an exemplary niraparib formulation comprises 47.8% by weight of niraparib tosylate monohydrate, 20.1% by weight of microcrystalline cellulose, 20.1% by weight of mannitol, 4.0% by weight of crospovidone, 2.0% by weight of povidone, and 0.5% by weight of magnesium stearate for the intragranular phase; and 4.0% by weight of crospovidone, 0.5% by weight of silicon dioxide, and 1.0% by weight of magnesium stearate for the extragranular phase.

In one aspect disclosed herein is tablet composition comprising: a) an effective amount of niraparib to inhibit polyadenosine diphosphate ribose polymerase (PARP) when administered to a subject in need thereof; b) a first diluent selected from lactose monohydrate, lactose anhydrous, mannitol, and calcium phosphate dibasic; c) magnesium stearate; d) a second diluent selected from microcrystalline cellulose, starch, polyethylene oxide, and hydroxypropyl methylcellulose (HPMC); and e) a binder selected from povidone (PVP), hydroxypropyl cellulose (HPC), and hydroxypropyl methylcellulose (HPMC).

In another aspect disclosed herein is tablet composition comprising the following components on a weight percentage basis:

(a) in an intragranular portion:
(i) 40-50% niraparib tosylate monohydrate;
(ii) 9-11% of a first diluent;
(iii) 30-40% of a second diluent;
(iv) 1-3% of a binder;
(v) 0.1-2% of a disintegrant;
(vi) 2-4% of a glidant or adsorbant or absorbant; and
(vii) 0.1-2% of a lubricant;

(b) in an extragranular portion:
0.1-2% of a disintegrant;
(ii) 0.1-2% of a glidant or adsorbant or absorbant; and
(iii) 0.1-2% of a lubricant.

In another aspect provided herein is a composition comprising a tablet comprising the following components on a weight percentage basis:

(a) in an intragranular portion:
(i) 40-50% niraparib tosylate monohydrate;
(ii) 9-40% of a diluent;
(iii) 1-3% of a binder;
(iv) 0.1-2% of a disintegrant;
(v) 2-4% of a glidant or adsorbant or absorbant; and
(vi) 0.1-2% of a lubricant;

(b) in an extragranular portion:
(vii) 0.1-2% of a disintegrant;
(viii) 0.1-2% of a glidant or adsorbant or absorbant; and
(ix) 0.1-2% of a lubricant.

In some embodiments, the lubricant is magnesium stearate.

In some embodiments, the diluent is lactose, mannitol, calcium phosphate dibasic, microcrystalline cellulose, starch, polyethylene oxide, or hydroxypropyl methylcellulose (HPMC). In some embodiments, the lactose is anhydrous, monohydrate, crystalline, or spray-dried. In some embodiments, the mannitol is spray dried or crystalline.

In some embodiments, the first diluent is lactose monohydrate. In some embodiments, the lactose monohydrate is spray dried or crystalline. In some embodiments, the first diluent is mannitol. In some embodiments, the mannitol is spray dried or crystalline. In some embodiments, the first diluent is calcium phosphate dibasic.

In some embodiments, the second diluent is microcrystalline cellulose. In some embodiments, the second diluent is starch, polyethylene oxide, or hydroxypropyl methylcellulose (HPMC).

In some embodiments, the binder is povidone (PVP). In some embodiments, the binder is hydroxypropyl cellulose (HPC). In some embodiments, the binder is hydroxypropyl methylcellulose (HPMC).

In some embodiments, composition further comprises a disintegrant. In some embodiments, the disintegrant is crospovidone or croscarmellose. In some embodiments, the croscarmellose is croscarmellose sodium. In some embodiments, the composition further comprises a large meso-porous silica excipient as an adsorbant. In some embodiments, the large meso-porous silica excipient absorbs water. In some embodiments, the composition further comprises an intermediate meso-porous silica excipient as a glidant. In some embodiments, the intermediate meso-porous silica comprises syloid FP-244. In some embodiments, the composition further comprises an additional excipient as an adsorbant such as bentonite, talc, microcrystalline cellulose, charcoal, fumed silica, magnesium carbonate, or similar excipients.

In some embodiments, the composition further comprises silicon dioxide. In some embodiments, the silicon dioxide is present in an amount of about 0.1% to about 10% by weight. In some embodiments, the silicon dioxide is present in an amount of about 0.1% to about 5% by weight. In some embodiments, the silicon dioxide is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, or about 5% by weight.

In some embodiments, the composition further comprises an intragranular phase. In some embodiments, the intragranular phase comprises silicon dioxide. In some embodiments, the silicon dioxide in the intragranular phase is present in an amount of about 0.1% to about 10% by weight. In some embodiments, the silicon dioxide in the intragranular phase is present in an amount of about 0.1% to about 5% by weight. In some embodiments, the silicon dioxide in the intragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, or about 5% by weight.

In some embodiments, wherein the intragranular phase does not comprise magnesium stearate. In some embodiments, the intragranular phase comprises niraparib, lactose monohydrate, microcrystalline cellulose, crospovidone, and povidone. In some embodiments, the intragranular phase comprises niraparib, lactose monohydrate, microcrystalline cellulose, croscarmellose, and hydroxypropyl cellulose (HPC). In some embodiments, the intragranular phase comprises niraparib, lactose monohydrate, microcrystalline cellulose, croscarmellose, and hydroxypropyl methylcellulose (HMPC). In some embodiments, the intragranular phase comprises niraparib, lactose monohydrate, microcrystalline cellulose, crospovidone, povidone, and a large meso-porous silica excipient as an adsorbant or absorbant or an intermediate meso-porous silica excipient as a glidant. In some embodiments, the intragranular phase comprises niraparib, lactose monohydrate, microcrystalline cellulose, crospovidone, povidone, and a large meso-porous silica excipient as an adsorbant or absorbant. In some embodiments, the intragranular phase comprises niraparib, lactose monohydrate, microcrystalline cellulose, crospovidone, povidone, and an intermediate meso-porous silica excipient as a glidant.

In some embodiments, the intragranular phase comprises magnesium stearate. In some embodiments, the intragranular phase comprises niraparib, microcrystalline cellulose, calcium phosphate dibasic, crospovidone, povidone, and magnesium stearate. In some embodiments, the intragranular phase comprises niraparib, microcrystalline cellulose, mannitol, croscarmellose, hydroxypropyl cellulose (HPC), and magnesium stearate. In some embodiments, the intragranular phase comprises niraparib, microcrystalline cellulose, mannitol, croscarmellose, hydroxypropyl methylcellulose (HPMC), and magnesium stearate. In some embodiments, the intragranular phase comprises niraparib, microcrystalline cellulose, mannitol, crospovidone, povidone, and magnesium stearate.

In some embodiments, the composition further comprises an extragranular phase. In some embodiments, the extragranular phase comprises magnesium stearate. In some embodiments, the extragranular phase comprises crospovidone. In some embodiments, the extragranular phase comprises croscarmellose.

In some embodiments, the extragranular phase comprises silicon dioxide. In some embodiments, the silicon dioxide in the extragranular phase is present in an amount of about 0.1% to about 10% by weight. In some embodiments, the silicon dioxide in the extragranular phase is present in an amount of about 0.1% to about 5% by weight. In some embodiments, the silicon dioxide in the extragranular phase is present in an amount of about 0.1% to about 2.5% by weight. In some embodiments, the silicon dioxide in the extragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, or about 5% by weight.

In some embodiments, the niraparib is present in an amount of about 5-90% by weight. In some embodiments, the niraparib is present in an amount of about 5-80% by weight. In some embodiments, the niraparib is present in an amount of about 5-70% by weight. In some embodiments, the niraparib is present in an amount of about 5-60% by weight. In some embodiments, the niraparib is present in an amount of about 5-50% by weight. In some embodiments, the niraparib is present in an amount of about 5-40% by weight. In some embodiments, the niraparib is present in an amount of about 5-30% by weight. In some embodiments, the niraparib is present in an amount of about 5-20% by weight. In some embodiments, the niraparib is present in an amount of about 5-10% by weight. In some embodiments, the niraparib is present in an amount of about 10-90% by weight. In some embodiments, the niraparib is present in an amount of about 10-80% by weight. In some embodiments, the niraparib is present in an amount of about 10-70% by weight. In some embodiments, the niraparib is present in an amount of about 10-60% by weight. In some embodiments, the niraparib is present in an amount of about 10-50% by weight. In some embodiments, the niraparib is present in an amount of about 10-40% by weight. In some embodiments, the niraparib is present in an amount of about 10-30% by weight. In some embodiments, the niraparib is present in an amount of about 10-20% by weight. In some embodiments, the niraparib is present in an amount of about 20-90% by weight. In some embodiments, the niraparib is present in an amount of about 20-80% by weight. In some embodiments, the niraparib is present in an amount of about 20-70% by weight. In some embodiments, the niraparib is present in an amount of about 20-60% by weight. In some embodiments, the niraparib is present in an amount of about 20-50% by weight. In some embodiments, the niraparib is present in an amount of about 20-40% by weight. In some embodiments, the niraparib is present in an amount of about 20-30% by weight. In some embodiments, the niraparib is present in an amount of about 30-90% by weight. In some embodiments, the niraparib is present in an amount of about 30-80% by weight. In some embodiments, the niraparib is present in an amount of about 30-70% by weight. In some embodiments, the niraparib is present in an amount of about 30-60% by weight. In some embodiments, the niraparib is present in an amount of about 30-50% by weight. In some embodiments, the niraparib is present in an amount of about 30-40% by weight. In some embodiments, the niraparib is present in an amount of about 40-90% by weight. In some embodiments, the niraparib is present in an amount of about 40-80% by weight. In some embodiments, the niraparib is present in an amount of about 40-70% by weight. In some embodiments, the niraparib is present in an amount of about 40-60% by weight. In some embodiments, the niraparib is present in an amount of about 40-50% by weight. In some embodiments, the niraparib is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% by weight. In some embodiments, the niraparib is the pharmaceutically acceptable salt of niraparib. In some embodiments, the niraparib is niraparib tosylate monohydrate.

In some embodiments, the second diluent (e.g., microcrystalline cellulose, starch, polyethylene oxide, and hydroxypropyl methylcellulose (HPMC)) is present in an amount of about 5-90% by weight. In some embodiments, the second diluent (e.g., microcrystalline cellulose, starch, polyethylene oxide, and hydroxypropyl methylcellulose (HPMC)) is present in an amount of about 5-80% by weight. In some embodiments, the second diluent (e.g., microcrystalline cellulose, starch, polyethylene oxide, and hydroxypropyl methylcellulose (HPMC)) is present in an amount of about 5-70% by weight. In some embodiments, the second diluent (e.g., microcrystalline cellulose, starch, polyethylene oxide, and hydroxypropyl methylcellulose (HPMC)) is present in an amount of about 5-60% by weight. In some embodiments, the second diluent (e.g., microcrystalline cellulose, starch, polyethylene oxide, and hydroxypropyl methylcellulose (HPMC)) is present in an amount of about 5-50% by weight. In some embodiments, the second diluent (e.g., microcrystalline cellulose, starch, polyethylene oxide, and hydroxypropyl methylcellulose (HPMC)) is present in an amount of about 5-40% by weight. In some embodiments, the second diluent (e.g., microcrystalline cellulose, starch, polyethylene oxide, and hydroxypropyl methylcellulose (HPMC)) is present in an amount of about 5-30% by weight. In some embodiments, the second diluent (e.g., microcrystalline cellulose, starch, polyethylene oxide, and hydroxypropyl methylcellulose (HPMC)) is present in an amount of about 5-20% by weight. In some embodiments, the second diluent (e.g., microcrystalline cellulose, starch, polyethylene oxide, and hydroxypropyl methylcellulose (HPMC)) is present in an amount of about 5-10% by weight. In some embodiments, the second diluent (e.g., microcrystalline cellulose, starch, polyethylene oxide, and hydroxypropyl methylcellulose (HPMC)) is present in an amount of about 10-90% by weight. In some embodiments, the second diluent (e.g., microcrystalline cellulose, starch, polyethylene oxide, and hydroxypropyl methylcellulose (HPMC)) is present in an amount of about 10-80% by weight. In some embodiments, the second diluent (e.g., microcrystalline cellulose, starch, polyethylene oxide, and hydroxypropyl methylcellulose (HPMC)) is present in an amount of about 10-70% by weight. In some embodiments, the second diluent (e.g., microcrystalline cellulose, starch, polyethylene oxide, and hydroxypropyl methylcellulose (HPMC)) is present in an amount of about 10-60% by weight. In some embodiments, the second diluent (e.g., microcrystalline cellulose, starch, polyethylene oxide, and hydroxypropyl methylcellulose (HPMC)) is present in an amount of about 10-50% by weight. In some embodiments, the second diluent (e.g., microcrystalline cellulose, starch, polyethylene oxide, and hydroxypropyl methylcellulose (HPMC)) is present in an amount of about 10-40% by weight. In some embodiments, the second diluent (e.g., microcrystalline cellulose, starch, polyethylene oxide, and hydroxypropyl methylcellulose (HPMC)) is present in an amount of about 10-30% by weight. In some embodiments, the second diluent (e.g., microcrystalline cellulose, starch, polyethylene oxide, and hydroxypropyl methylcellulose (HPMC)) is present in an amount of about 10-20% by weight. In some embodiments, the second diluent (e.g., microcrystalline cellulose, starch, polyethylene oxide, and hydroxypropyl methylcellulose (HPMC)) is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 5′7%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 7′7%, about 78%, about 79%, about 80%, about 81%, about 82%, about 83%, about 84%, about 85%, about 86%, about 87%, about 88%, about 89%, or about 90% by weight.

In some embodiments, the microcrystalline cellulose is present in an amount of about 5-90% by weight. In some embodiments, the microcrystalline cellulose is present in an amount of about 5-80% by weight. In some embodiments, the microcrystalline cellulose is present in an amount of about 5-70% by weight. In some embodiments, the microcrystalline cellulose is present in an amount of about 5-60% by weight. In some embodiments, the microcrystalline cellulose is present in an amount of about 5-50% by weight. In some embodiments, the microcrystalline cellulose is present in an amount of about 5-40% by weight. In some embodiments, the microcrystalline cellulose is present in an amount of about 5-30% by weight. In some embodiments, the microcrystalline cellulose is present in an amount of about 5-20% by weight. In some embodiments, the microcrystalline cellulose is present in an amount of about 5-10% by weight. In some embodiments, the microcrystalline cellulose is present in an amount of about 10-90% by weight. In some embodiments, the microcrystalline cellulose is present in an amount of about 10-80% by weight. In some embodiments, the microcrystalline cellulose is present in an amount of about 10-70% by weight. In some embodiments, the microcrystalline cellulose is present in an amount of about 10-60% by weight. In some embodiments, the microcrystalline cellulose is present in an amount of about 10-50% by weight. In some embodiments, the microcrystalline cellulose is present in an amount of about 10-40% by weight. In some embodiments, the microcrystalline cellulose is present in an amount of about 10-30% by weight. In some embodiments, the microcrystalline cellulose is present in an amount of about 10-20% by weight. In some embodiments, the microcrystalline cellulose is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 6′7%, about 68%, about 69%, about 70%, about 71%, about 72%, about 73%, about 74%, about 75%, about 76%, about 7′7%, about 78%, about 79%, about 80%, about 81%, about 82%, about 83%, about 84%, about 85%, about 86%, about 87%, about 88%, about 89%, or about 90% by weight.

In some embodiments, the first diluent, such as lactose monohydrate, lactose anhydrous, mannitol, and calcium phosphate dibasic, is present in an amount of about 5-90% by weight. In some embodiments, the first diluent, such as lactose monohydrate, lactose anhydrous, mannitol, and calcium phosphate dibasic, is present in an amount of about 5-80% by weight. In some embodiments, the first diluent, such as lactose monohydrate, lactose anhydrous, mannitol, and calcium phosphate dibasic, is present in an amount of about 5-70% by weight. In some embodiments, the first diluent, such as lactose monohydrate, lactose anhydrous, mannitol, and calcium phosphate dibasic, is present in an amount of about 5-60% by weight. In some embodiments, the first diluent, such as lactose monohydrate, lactose anhydrous, mannitol, and calcium phosphate dibasic, is present in an amount of about 5-50% by weight. In some embodiments, the first diluent, such as lactose monohydrate, lactose anhydrous, mannitol, and calcium phosphate dibasic, is present in an amount of about 5-40% by weight. In some embodiments, the first diluent, such as lactose monohydrate, lactose anhydrous, mannitol, and calcium phosphate dibasic, is present in an amount of about 5-30% by weight. In some embodiments, the first diluent, such as lactose monohydrate, lactose anhydrous, mannitol, and calcium phosphate dibasic, is present in an amount of about 5-20% by weight. In some embodiments, the first diluent, such as lactose monohydrate, lactose anhydrous, mannitol, and calcium phosphate dibasic, is present in an amount of about 5-10% by weight. In some embodiments, the first diluent, such as lactose monohydrate, lactose anhydrous, mannitol, and calcium phosphate dibasic, is present in an amount of about 10-90% by weight. In some embodiments, the first diluent, such as lactose monohydrate, lactose anhydrous, mannitol, and calcium phosphate dibasic, is present in an amount of about 10-80% by weight. In some embodiments, the first diluent, such as lactose monohydrate, lactose anhydrous, mannitol, and calcium phosphate dibasic, is present in an amount of about 10-70% by weight. In some embodiments, the first diluent, such as lactose monohydrate, lactose anhydrous, mannitol, and calcium phosphate dibasic, is present in an amount of about 10-60% by weight. In some embodiments, the first diluent, such as lactose monohydrate, lactose anhydrous, mannitol, and calcium phosphate dibasic, is present in an amount of about 10-50% by weight. In some embodiments, the first diluent, such as lactose monohydrate, lactose anhydrous, mannitol, and calcium phosphate dibasic, is present in an amount of about 10-40% by weight. In some embodiments, the first diluent, such as lactose monohydrate, lactose anhydrous, mannitol, and calcium phosphate dibasic is present in an amount of about 10-30% by weight. In some embodiments, the first diluent, such as lactose monohydrate, lactose anhydrous, mannitol, and calcium phosphate dibasic is present in an amount of about 10-20% by weight. In some embodiments, the first diluent, such as lactose monohydrate, lactose anhydrous, mannitol, and calcium phosphate dibasic, is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% by weight.

In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-90% by weight. In some embodiments, the lactose is anhydrous, monohydrate, crystalline, or spray-dried. In some embodiments, the mannitol is spray dried or crystalline. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-80% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-70% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-60% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-50% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-40% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-30% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-20% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5-10% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-90% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-80% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-70% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-60% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-50% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-40% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-30% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 10-20% by weight. In some embodiments, the diluent, such as lactose, mannitol, calcium phosphate dibasic, microcrystalline cellulose, starch, polyethylene oxide, or hydroxypropyl methylcellulose (HPMC), is present in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% by weight.

In some embodiments, the binder, such as povidone, hydroxylpropyl cellulose, or hydroxypropyl methylcellulose, is present in an amount of about 1-40% by weight. In some embodiments, the binder, such as povidone, hydroxylpropyl cellulose, or hydroxypropyl methylcellulose, is present in an amount of about 1-30% by weight. In some embodiments, the binder, such as povidone, hydroxylpropyl cellulose, or hydroxypropyl methylcellulose, is present in an amount of about 1-20% by weight. In some embodiments, the binder, such as povidone, hydroxylpropyl cellulose, or hydroxypropyl methylcellulose, is present in an amount of about 1-10% by weight. In some embodiments, the binder, such as povidone, hydroxylpropyl cellulose, or hydroxypropyl methylcellulose, is present in an amount of about 1-5% by weight. In some embodiments, the binder, such as povidone, hydroxylpropyl cellulose, or hydroxypropyl methylcellulose, is present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the disintegrant, such as crospovidone or croscarmellose, is present in an amount of about 0.1-40% by weight. In some embodiments, the disintegrant, such as crospovidone or croscarmellose, is present in an amount of about 0.1-30% by weight. In some embodiments, the disintegrant, such as crospovidone and croscarmellose, is present in an amount of about 0.1-20% by weight. In some embodiments, the disintegrant, such as crospovidone or croscarmellose, is present in an amount of about 0.1-10% by weight. In some embodiments, the disintegrant, such as crospovidone and croscarmellose, is present in an amount of about 0.1-5% by weight. In some embodiments, the disintegrant, such as crospovidone or croscarmellose, is present in an amount of about 0.1%, about 0.2%, about 0.3%, about 0.4%, about 0.5%, about 0.6, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the crospovidone is present in an amount of about 0.1-40% by weight. In some embodiments, the crospovidone is present in an amount of about 0.1-30% by weight. In some embodiments, the crospovidone is present in an amount of about 0.1-20% by weight. In some embodiments, the crospovidone is present in an amount of about 0.1-10% by weight. In some embodiments, the crospovidone is present in an amount of about 0.1-5% by weight. In some embodiments, the crospovidone is present in an amount of about 0.1%, about 0.2%, about 0.3%, about 0.4%, about 0.5%, about 0.6, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the croscarmellose is present in an amount of about 0.1-40% by weight. In some embodiments, the croscarmellose is present in an amount of about 0.1-30% by weight. In some embodiments, the croscarmellose is present in an amount of about 0.1-20% by weight. In some embodiments, the croscarmellose is present in an amount of about 0.1-10% by weight. In some embodiments, the croscarmellose is present in an amount of about 0.1-5% by weight. In some embodiments, the croscarmellose is present in an amount of about 0.1%, about 0.2%, about 0.3%, about 0.4%, about 0.5%, about 0.6, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight. In some embodiments, the croscarmellose is croscarmellose sodium.

In some embodiments, the glidant, such as silicon dioxide, is present in an amount of about 0.1-40% by weight. In some embodiments, the glidant, such as silicon dioxide, is present in an amount of about 0.1-30% by weight. In some embodiments, the glidant, such as silicon dioxide, is present in an amount of about 0.1-20% by weight. In some embodiments, the glidant, such as silicon dioxide, is present in an amount of about 0.1-10% by weight. In some embodiments, the glidant, such as silicon dioxide, is present in an amount of about 0.1-5% by weight. In some embodiments, the glidant, such as silicon dioxide, is present in an amount of about 0.1%, about 0.2%, about 0.3%, about 0.4%, about 0.5%, about 0.6, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the silicon dioxide, is present in an amount of about 0.1-40% by weight. In some embodiments, the silicon dioxide is present in an amount of about 0.1-30% by weight. In some embodiments, the silicon dioxide is present in an amount of about 0.1-20% by weight. In some embodiments, the silicon dioxide is present in an amount of about 0.1-10% by weight. In some embodiments, the silicon dioxide is present in an amount of about 0.1-5% by weight. In some embodiments, the silicon dioxide is present in an amount of about 0.1%, about 0.2%, about 0.3%, about 0.4%, about 0.5%, about 0.6, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the lubricant, such as magnesium stearate, in the intragranular phase or extragranular phase is present in an amount of about 0.1-40% by weight. In some embodiments, the lubricant, such as magnesium stearate, in the intragranular phase or extragranular phase is present in an amount of about 0.1-30% by weight. In some embodiments, the lubricant, such as magnesium stearate, in the intragranular phase or extragranular phase is present in an amount of about 0.1-20% by weight. In some embodiments, the lubricant, such as magnesium stearate, in the intragranular phase or extragranular phase is present in an amount of about 0.1-10% by weight. In some embodiments, the lubricant, such as magnesium stearate, in the intragranular phase or extragranular phase is present in an amount of about 0.1-5% by weight. In some embodiments, the lubricant, such as magnesium stearate, in the intragranular phase or extragranular phase is present in an amount of about 0.1-2.5% by weight. In some embodiments, the lubricant, such as magnesium stearate, in the intragranular phase or extragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the magnesium stearate in the intragranular phase is present in an amount of about 0.1-40% by weight. In some embodiments, the magnesium stearate in the intragranular phase is present in an amount of about 0.1-30% by weight. In some embodiments, magnesium stearate in the intragranular phase is present in an amount of about 0.1-20% by weight. In some embodiments, magnesium stearate in the intragranular phase is present in an amount of about 0.1-10% by weight. In some embodiments, the magnesium stearate in the intragranular phase is present in an amount of about 0.1-5% by weight. In some embodiments, the magnesium stearate in the intragranular phase is present in an amount of about 0.1-2.5% by weight. In some embodiments, the magnesium stearate in the intragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the magnesium stearate in the extragranular phase is present in an amount of about 0.1-40% by weight. In some embodiments, the magnesium stearate in the extragranular phase is present in an amount of about 0.1-30% by weight. In some embodiments, magnesium stearate in the extragranular phase is present in an amount of about 0.1-20% by weight. In some embodiments, magnesium stearate in the extragranular phase is present in an amount of about 0.1-10% by weight. In some embodiments, the magnesium stearate in the extragranular phase is present in an amount of about 0.1-5% by weight. In some embodiments, the magnesium stearate in the extragranular phase is present in an amount of about 0.1-2.5% by weight. In some embodiments, the magnesium stearate in the extragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

Also provided in another aspect is a composition comprising a tablet comprising a) an effective amount of niraparib to inhibit polyadenosine diphosphate ribose polymerase (PARP) when administered to a subject in need thereof; and b) silicon dioxide; wherein the effective amount of niraparib is from about 50 mg to about 350 mg based on the niraparib free base.

In some embodiments, the effective amount of niraparib is from about 75 mg to about 125 mg based on the niraparib free base. In some embodiments, the effective amount of niraparib is about 50 mg, 100 mg, or about 150 mg based on the niraparib free base. In some embodiments, the effective amount of niraparib is about 100 mg based on the niraparib free base. In some embodiments, the effective amount of niraparib is from about 175 mg to about 225 mg based on the niraparib free base. In some embodiments, the effective amount of niraparib is about 150 mg, 200 mg, or about 250 mg based on the niraparib free base. In some embodiments, the effective amount of niraparib is about 200 mg based on the niraparib free base. In some embodiments, the effective amount of niraparib is from about 275 mg to about 325 mg based on the niraparib free base. In some embodiments, the effective amount of niraparib is about 250 mg, about 300 mg, or about 350 mg based on the niraparib free base. In some embodiments, the effective amount of niraparib is about 300 mg based on the niraparib free base. In some embodiments, the niraparib comprises niraparib free base or a pharmaceutically acceptable salt thereof. In some embodiments, the pharmaceutically acceptable salt of niraparib is niraparib tosylate.

In some embodiments, silicon dioxide provides improved flow properties. In some embodiments, silicon dioxide improves tensile strength, hardness, and/or bonding of intragranular materials. In some embodiments, silicon dioxide improves the properties of the composition comprising niraparib that is directly compressed to form the tablet, such as reducing the adherence or stickiness of the composition.

In some embodiments, the silicon dioxide is present in the intragranular phase. In some embodiments, the silicon dioxide in the intragranular phase is present in an amount of about 0.1-40% by weight. In some embodiments, the silicon dioxide in the intragranular phase is present in an amount of about 0.1-30% by weight. In some embodiments, silicon dioxide in the intragranular phase is present in an amount of about 0.1-20% by weight. In some embodiments, silicon dioxide in the intragranular phase is present in an amount of about 0.1-10% by weight. In some embodiments, the silicon dioxide in the intragranular phase is present in an amount of about 0.1-5% by weight. In some embodiments, the silicon dioxide in the intragranular phase is present in an amount of about 0.1-2.5% by weight. In some embodiments, the silicon dioxide in the intragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

In some embodiments, the silicon dioxide is present in the extragranular phase. In some embodiments, the silicon dioxide in the extragranular phase is present in an amount of about 0.1-40% by weight. In some embodiments, the silicon dioxide in the extragranular phase is present in an amount of about 0.1-30% by weight. In some embodiments, silicon dioxide in the extragranular phase is present in an amount of about 0.1-20% by weight. In some embodiments, silicon dioxide in the extragranular phase is present in an amount of about 0.1-10% by weight. In some embodiments, the silicon dioxide in the extragranular phase is present in an amount of about 0.1-5% by weight. In some embodiments, the silicon dioxide in the extragranular phase is present in an amount of about 0.1-2.5% by weight. In some embodiments, the silicon dioxide in the extragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% by weight.

Intragranular Phase/Extragranular Phase Distribution

In some embodiments, the distribution of the intragranular phase components and extragranular phase components provide desirable disintegration profiles. In another aspect, provided herein is a composition comprising a tablet comprising: an effective amount of niraparib to inhibit polyadenosine diphosphate ribose polymerase (PARP) when administered to a subject in need thereof; wherein the tablet further comprises an intragranular phase and an extragranular phase; and the tablet has at least one of the following: a) the amount of components used to form the intragranular phase is about 50% to about 98% by weight of the tablet composition; and b) the amount of components used to form the extragranular phase is about 2% to about 50% by weight of the tablet composition.

In some embodiments, the amount of components used to form the intragranular phase is about 50% to about 98% by weight of the tablet composition. In some embodiments, the amount of components used to form the intragranular phase is about 55% to about 98% by weight of the tablet composition. In some embodiments, the amount of components used to form the intragranular phase is about 60% to about 98% by weight of the tablet composition. In some embodiments, the amount of components used to form the intragranular phase is about 65% to about 98% by weight of the tablet composition. In some embodiments, the amount of components used to form the intragranular phase is about 70% to about 98% by weight of the tablet composition. In some embodiments, the amount of components used to form the intragranular phase is about 75% to about 98% by weight of the tablet composition. In some embodiments, the amount of components used to form the intragranular phase is about 80% to about 98% by weight of the tablet composition. In some embodiments, the amount of components used to form the intragranular phase is about 85% to about 98% by weight of the tablet composition. In some embodiments, the amount of components used to form the intragranular phase is about 90% to about 98% by weight of the tablet composition. In some embodiments, the amount of components used to form the intragranular phase is about 92.5% to about 97.5% by weight of the tablet composition. In some embodiments, the amount of components used to form the intragranular phase is about 95% by weight of the tablet composition. In some embodiments, the amount of components used to form the intragranular phase is about 50%, about 55%, about 60%, about 65%, about 70%, about 75%, about 80%, about 85%, about 90%, about 91%, about 92%, about 93%, about 94%, about 95%, about 96%, about 97%, or about 98% by weight of the tablet composition.

In some embodiments, the amount of components used to form the extragranular phase is about 2% to about 50% by weight of the tablet composition. In some embodiments, the amount of components used to form the extragranular phase is about 2% to about 45% by weight of the tablet composition. In some embodiments, the amount of components used to form the extragranular phase is about 2% to about 40% by weight of the tablet composition. In some embodiments, the amount of components used to form the extragranular phase is about 2% to about 35% by weight of the tablet composition. In some embodiments, the amount of components used to form the extragranular phase is about 2% to about 30% by weight of the tablet composition. In some embodiments, the amount of components used to form the extragranular phase is about 2% to about 25% by weight of the tablet composition. In some embodiments, the amount of components used to form the extragranular phase is about 2% to about 20% by weight of the tablet composition. In some embodiments, the amount of components used to form the extragranular phase is about 2% to about 15% by weight of the tablet composition. In some embodiments, the amount of components used to form the extragranular phase is about 2% to about 10% by weight of the tablet composition. In some embodiments, the amount of components used to form the extragranular phase is about 2% to about 5% by weight of the tablet composition. In some embodiments, the amount of components used to form the extragranular phase is about 2.5% to about 7.5% by weight of the tablet composition. In some embodiments, the amount of components used to form the extragranular phase is about 5% by weight of the tablet composition. In some embodiments, the amount of components used to form the extragranular phase is about 2%, about 2.5%, about 3%, about 3.5%, about 4%, about 4.5%, about 5%, about 5.5%, about 6.0%, about 6.5%, about 7.0%, about 7.5%, about 8.0%, about 8.5%, about 9.0%, about 9.5%, about 10.0%, about 15%, about 20%, about 25%, about 30%, about 35%, about 40%, about 45%, or about 50% by weight of the tablet composition.

Methods of Making Niraparib Formulations

Provided herein are methods of manufacturing niraparib compositions (e.g., suitable for methods described herein).

Methods of Making Niraparib Capsule Formulations

Provided herein are methods of manufacturing niraparib capsule compositions for treating cancers. Also described herein are niraparib capsule formulations containing niraparib tosylate monohydrate, lactose monohydrate and magnesium stearate formed by disclosed methods, and the therapeutic use of such formulation orally. The disclosed formulation can be a dry powder blend in a capsule containing niraparib as an active pharmaceutical ingredient (API), an excipient such as lactose monohydrate, and lubricant such as magnesium stearate. The niraparib capsule composition can contain 19.2˜38.3% w/w niraparib, 61.2˜80.3% w/w lactose, and at least 0.5% w/w magnesium stearate.

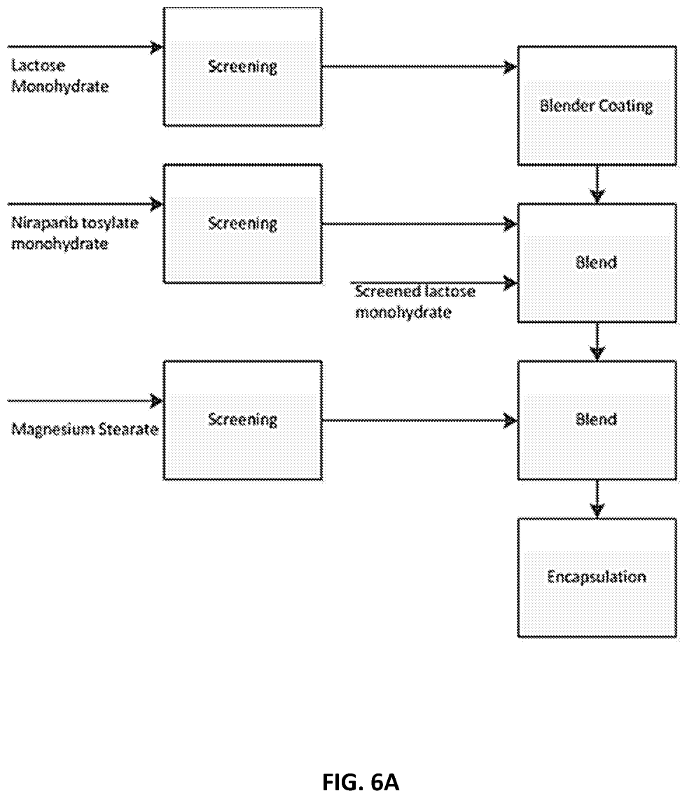

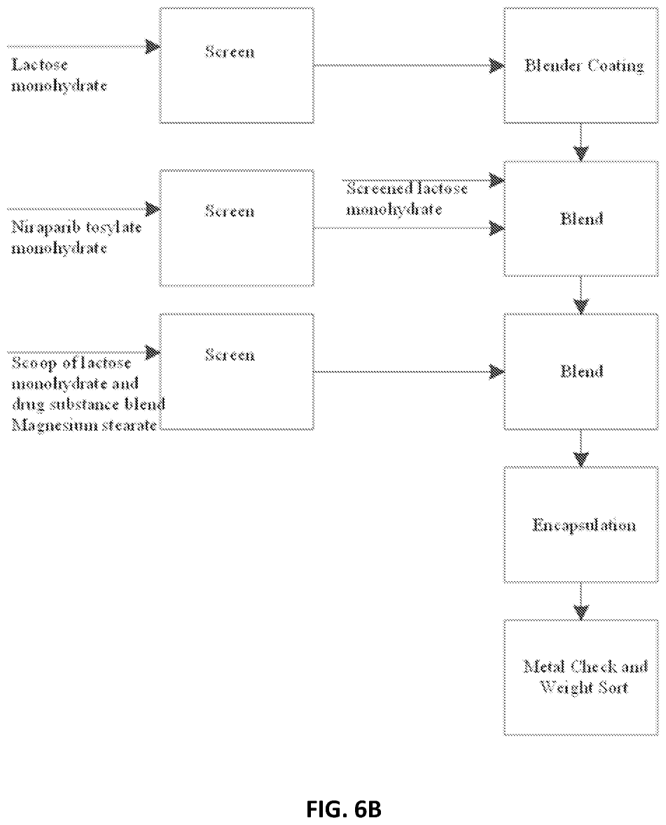

The manufacturing process can comprise blending screened lactose with niraparib followed by mixing and blending with screened magnesium stearate and further followed by encapsulation, wherein lactose is screened through a mesh screen, for example, having a mesh size of at most 600 microns, and magnesium stearate is screened through a mesh screen, for example, having a size of greater than 250 microns. The manufacturing process can comprise blending screened lactose with screened niraparib followed by mixing and blending with screened magnesium stearate and further followed by encapsulation, wherein lactose is screened through a mesh screen, for example, having a mesh size of at most 600 microns, and niraparib is screened through a mesh screen, for example, having a size of greater than 425 microns, and magnesium stearate is screened through a mesh screen, for example, having a size of greater than 250 microns. In some embodiments, the manufacturing process comprises obtaining screened lactose that has been screened through a mesh screen, for example, with a size of about 600 microns, and obtaining screened niraparib that has been screened through a mesh screen, for example, with a size of about 1180 microns, and obtaining screened magnesium stearate that has been screened through a mesh screen, for example, with a size of about 600 microns. An exemplary diagram showing the manufacturing process is shown in FIG. 6.

Different screening methods can be used for screening niraparib, for example, a conical mill, a vibratory sifter, or an oscillating screen where manufacturing process utilizes screened niraparib.

Various blenders can be used for blending the mixed compositions, for example, V-blender and double cone blender. Different blending conditions may be used for blenders having different sizes, including variations in size, speed, and time of blending.

In some embodiments, hold times between blending and encapsulation are about 1, 2, 3 or 4 days. In some embodiments, hold times between blending and encapsulation are less than 1, 2, 3 or 4 days.

A variety of encapsulators are used including manual, semi-automatic and full automatic encapsulators. In some embodiments, a manual encapsulation machine is used. And in some other embodiments, an automated encapsulator is used. In some embodiments, a Profill (Torpac, Fairfield, N.J.) manual encapsulation machine is used. And in some other embodiments, an automated Bosch GKF 330 powder filling encapsulator is used. The speed of the encapsulator can be adjusted to aid non-ideal powder flow. The encapsulator relies upon centrifugal force to move the powder from the hopper across the dosing bowl, where the powder then fills the holes in the dosing disc. Increasing the speed of the encapsulator increases the rotational velocity of the bowl and the associated centrifugal force. The increased force has the potential to improve the powder flow and reduce segregation.

In some embodiments, the speed of the encapsulator is greater than 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 75,000, 100,000, 150,000 or 200,000 capsules/hour. In some embodiments, the speed of the encapsulator ranges from 12,000 to 18,000 capsules/hour.

The height of the dosing disc can be set at a height lower than 17.5 mm to prevent overfill. During manufacturing, sticking on the tamping pins and the dosing disc was noted in certain batches. To mitigate the sticking potential, a coating can added to the tamping pins and dosing disc and screening of the drug substance can performed. The tamping pin and dosing disc can be coated with nickel and chrome coating which helps eliminate build-up and possible stickiness during encapsulation. To eliminate or reduce non-ideal powder flow and sticking during encapsulation that may have been the result of static charge, screening can be introduced to de-lump the drug substance. Due to the reduced mechanical agitation, the screening may reduce the potential for triboelectrification of the drug substance.

In some embodiments, the pharmaceutical composition of the present invention is prepared by blending the niraparib with excipients. The blending of above components can preferably be carried out in a mixer, for example in a tumble blender. Bulk density and tapped density can be determined according to USP 24, Test 616 “Bulk Density and Tapped Density”.

In some embodiments, the solid dosage forms of the present invention may be in the form of a powder (including a sterile packaged powder, a dispensable powder, or an effervescent powder), or a capsule (including both soft or hard capsules, e.g., capsules made from animal-derived gelatin or plant-derived HPMC, or “sprinkle capsules”). In some embodiments, the pharmaceutical formulation is in the form of a powder. Additionally, pharmaceutical formulations of the present invention may be administered as a single capsule or in multiple capsule dosage form. In some embodiments, the pharmaceutical formulation is administered in one, or two, or three, or four, capsules.

In some embodiments, solid dosage forms, e.g., capsules, are prepared by mixing niraparib particles with one or more pharmaceutical excipients to form a bulk blend composition. When referring to these bulk blend compositions as homogeneous, it is meant that the niraparib particles are dispersed evenly throughout the composition so that the composition may be readily subdivided into equally effective unit dosage forms, such as capsules. The individual unit dosages may also comprise film coatings, which disintegrate upon oral ingestion or upon contact with diluents.

Non-limiting pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The invention should not be considered limited to these particular conditions for combining the components and it will be understood, based on this disclosure that the advantageous properties can be achieved through other conditions provided the components retain their basic properties and substantial homogeneity of the blended formulation components of the formulation is otherwise achieved without any significant segregation.

In one embodiment for preparing the blend, the components are weighed and placed into a blending container. Blending is performed for a period of time to produce a homogenous blend using suitable mixing equipment. Optionally, the blend is passed through a mesh screen to delump the blend. The screened blend may be returned to the blending container and blended for an additional period of time. Lubricant may then be added and the blend mixed for an additional period of time.

In the pharmaceutical industry, milling is often used to reduce the particle size of solid materials. Many types of mills are available including cone mills, pin mills, hammer mills and jet mills. One of the most commonly used types of mill is the hammer mill. The hammer mill utilizes a high-speed rotor to which a number of fixed or swinging hammers are attached. The hammers can be attached such that either the knife face or the hammer face contacts the material. As material is fed into the mill, it impacts on the rotating hammers and breaks up into smaller particles. A screen is located below the hammers, which allows the smaller particles to pass through the openings in the screen. Larger particles are retained in the mill and continue to be broken up by the hammers until the particles are fine enough to flow through the screen. The material may optionally be screened. In screening, material is placed through a mesh screen or series of mesh screens to obtain the desired particle size.

A capsule may be prepared, e.g., by placing the bulk blend niraparib formulation, described above, inside of a capsule. In some embodiments, the niraparib formulations (non-aqueous suspensions and solutions) are placed in a soft gelatin capsule. In other embodiments, the niraparib formulations are placed in standard gelatin capsules or non-gelatin capsules. In other embodiments, the niraparib formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the niraparib formulation is delivered in a capsule form. For example, the capsule may comprise between about 1 mg to about 1000 mg of niraparib or a pharmaceutically acceptable salt thereof. In some embodiments, the capsule comprises from about 1 mg to 5 mg, 5 mg to 10 mg, 10 mg to 20 mg, 20 mg to 25 mg, 35 mg to 50 mg, 50 mg to 75 mg, 70 mg to 95 mg, 90 mg to 115 mg, 110 mg to 135 mg, 130 mg to 155 mg, 150 mg to 175 mg, 170 to 195 mg, 190 mg to 215 mg, 210 mg to 235 mg, 230 mg to 255 mg, 250 mg to 275 mg, or 270 mg to 300 mg, 290 mg to 315 mg, 310 mg to 335 mg, 330 mg to 355 mg, 350 mg to 375 mg, 370 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, or 950 mg to 1000 mg of niraparib or a pharmaceutically acceptable salt thereof. In some embodiments, the capsule comprises from about 1 to about 300 mg of niraparib or a pharmaceutically acceptable salt thereof. In some embodiments, the capsule comprises from about 300 mg to about 1000 mg of niraparib or a pharmaceutically acceptable salt thereof. In some embodiments, the capsule comprises about 1 mg, 5 mg, 10 mg, 20 mg, 25 mg, 35 mg, 50 mg, 75 mg, 100 mg, 125 mg, 150 mg, 175 mg, 200 mg, 225 mg, 250 mg to 275 mg, 300 mg, 325 mg, 350 mg 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, or 1000 mg of niraparib or a pharmaceutically acceptable salt thereof.

Another embodiment of the present invention also provides a process for the preparation of pharmaceutical composition of niraparib or a pharmaceutically acceptable salt thereof (e.g., niraparib tosylate monohydrate), comprising the steps of obtaining niraparib that has been screened; obtaining lactose monohydrate that has been screened with a screen; combining the screened niraparib with the screened lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. The method can further comprise encapsulating the composition comprising niraparib, lactose monohydrate and magnesium stearate.

Another embodiment of the present invention also provides a process for the preparation of pharmaceutical composition of niraparib or a pharmaceutically acceptable salt thereof (e.g., niraparib tosylate monohydrate), comprising the steps of obtaining niraparib that has been screened with a screen having a mesh size of greater than 425 microns; combining the screened niraparib with lactose monohydrate to form a composition comprising niraparib and lactose monohydrate; blending the composition comprising niraparib and lactose monohydrate; combining the blended composition comprising niraparib and lactose monohydrate with magnesium stearate to form a composition comprising niraparib, lactose monohydrate and magnesium stearate; and blending the composition comprising niraparib, lactose monohydrate and magnesium stearate. The method can further comprise encapsulating the composition comprising niraparib, lactose monohydrate and magnesium stearate.

Another embodiment of the present invention also provides a process for the preparation of pharmaceutical composition of niraparib or a pharmaceutically acceptable salt thereof (e.g., niraparib tosylate monohydrate), comprising the steps of obtaining niraparib that has been screened; combining the screened niraparib with the screened lactose monohydrate to form a composition comprising niraparib and lactose monohydrate, blending the composition comprising niraparib and lactose monohydrate, combining the blended composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than 250 microns, and blending the composition comprising niraparib, lactose monohydrate and magnesium stearate.

In some embodiments, obtaining niraparib that has been screened comprises obtaining niraparib that has been screened with a screen having a mesh size of greater than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining niraparib that has been screened comprises obtaining niraparib that has been screened with a screen having a mesh size of greater than 425 μm.

In some embodiments, obtaining niraparib that has been screened comprises obtaining niraparib that has been screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining niraparib that has been screened comprises obtaining niraparib that has been screened with a screen having a mesh size of about 1180 microns.

In some embodiments, obtaining screened lactose monohydrate that has been screened with a screen comprises obtaining screened lactose monohydrate that has been screened with a screen having a mesh size of at most about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining screened lactose monohydrate that has been screened with a screen comprises obtaining screened lactose monohydrate that has been screened with a screen having a mesh size of at most about 600 microns.

In some embodiments, obtaining screened lactose monohydrate that has been screened with a screen comprises obtaining screened lactose monohydrate that has been screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, obtaining screened lactose monohydrate that has been screened with a screen comprises obtaining screened lactose monohydrate that has been screened with a screen having a mesh size of about 600 microns. In some embodiments, over 50% of the screened lactose monohydrate is present as particles with a diameter of between 53 microns and 500 microns.

In some embodiments, the magnesium stearate is magnesium stearate screened with a screen having a mesh size of greater than 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, the magnesium stearate is magnesium stearate screened with a screen having a mesh size of greater than 250 microns.

In some embodiments, the magnesium stearate is magnesium stearate screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm. In some embodiments, the magnesium stearate is magnesium stearate screened with a screen having a mesh size of about 600 microns.

In some embodiments, the method further comprises obtaining lactose monohydrate that has been screened before combining the screened niraparib with the screened lactose monohydrate to form a composition comprising niraparib and lactose monohydrate. In some embodiments, the particle size of the lactose monohydrate is about the same as the particle size of the niraparib.

In some embodiments, the composition comprising niraparib and lactose monohydrate is screened with a screen having a mesh size of at most about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, the composition comprising niraparib and lactose monohydrate is screened with a screen having a mesh size of about 5 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 550 μm, 600 μm, 650 μm, 700 μm, 750 μm, 800 μm, 850 μm, 900 μm, 950 μm, or 1000 μm.

In some embodiments, the screened niraparib is screened with a conical mill, a vibratory sifter, or an oscillating screen.

In some embodiments, the method further comprises encapsulating the blended the composition comprising niraparib, lactose monohydrate and magnesium stearate.

In some embodiments, the encapsulating comprises encapsulating the blended the composition comprising niraparib, lactose monohydrate and magnesium stearate into a capsule comprising gelatin.

In some embodiments, the number of blending revolutions for blending niraparib and an excipient is about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

In some embodiments, the number of blending revolutions for blending niraparib and lactose monohydrate is about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

In some embodiments, the number of blending revolutions for blending a composition comprising niraparib and lactose monohydrate with magnesium stearate is about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.

Methods of Making Niraparib Tablet Formulations

Provided herein are methods of manufacturing niraparib tablet compositions for treating cancers. Also described herein are niraparib tablet formulations containing niraparib tosylate monohydrate and at least one pharmaceutically acceptable excipient formed by disclosed methods, and the therapeutic use of such formulation orally. In some embodiments, the formulation comprises niraparib; a first diluent selected from lactose monohydrate, lactose anhydrous, mannitol, and calcium phosphate dibasic, magnesium stearate; a second diluent selected from microcrystalline cellulose, starch, polyethylene oxide, and hydroxypropyl methylcellulose (HPMC); and a binder selected from povidone, hydroxypropyl cellulose, and hydroxypropyl methylcellulose. In some embodiments, the formulation comprises the active niraparib tosylate (monohydrate) at about 35% w/w to about 60% w/w. In some embodiments, the formulation comprises the active niraparib tosylate (monohydrate) at about 40% w/w to about 55% w/w. In some embodiments, the formulation comprises the active niraparib tosylate (monohydrate) at about 45% w/w to about 50% w/w. In some embodiments, the formulation comprises the active niraparib tosylate (monohydrate) at about 47.8% w/w.

In some embodiments, the pharmaceutical composition of the present invention is prepared by blending the niraparib with excipients. The blending of above components can preferably be carried out in a mixer, for example in a tumble blender. Bulk density and tapped density can be determined according to USP 24, Test 616 “Bulk Density and Tapped Density”.

In some embodiments, the solid dosage forms of the present invention may be in the form of a powder (including a sterile packaged powder, a dispensable powder, or an effervescent powder), a capsule (including both soft or hard capsules, e.g., capsules made from animal-derived gelatin or plant-derived HPMC, or “sprinkle capsules”), or a tablet. In some embodiments, the pharmaceutical formulation is in the form of a powder. Additionally, pharmaceutical formulations of the present invention may be administered as a single capsule or in multiple capsule dosage form. In some embodiments, the pharmaceutical formulation is administered in one, or two, or three, or four, capsules. In some embodiments, the solid dosage forms disclosed herein are in the form of tablet. In some embodiments, the pharmaceutical formulations disclosed herein are administered as a single tablet or in multiple tablet dosage forms. In some embodiments, the pharmaceutical formulation is administered in one, or two, or three, or four tablets.

In some embodiments, solid dosage forms, are prepared by mixing niraparib particles with one or more pharmaceutical excipients to form a bulk blend composition. When referring to these bulk blend compositions as homogeneous, it is meant that the niraparib particles are dispersed evenly throughout the composition so that the composition may be readily subdivided into equally effective unit dosage forms, such as capsules or tablets. The individual unit dosages may also comprise film coatings, which disintegrate upon oral ingestion or upon contact with diluents.

Non-limiting pharmaceutical techniques for preparation of solid dosage forms include, e.g., one or a combination of methods: (1) dry mixing, (2) direct compression, (3) milling, (4) dry or non-aqueous granulation, (5) wet or dry granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

The invention should not be considered limited to these particular conditions for combining the components and it will be understood, based on this disclosure that the advantageous properties can be achieved through other conditions provided the components retain their basic properties and substantial homogeneity of the blended formulation components of the formulation is otherwise achieved without any significant segregation.

In one embodiment for preparing the blend, the components are weighed and placed into a blending container. Blending is performed for a period of time to produce a homogenous blend using suitable mixing equipment. Optionally, the blend is passed through a mesh screen to delump the blend. The screened blend may be returned to the blending container and blended for an additional period of time. Lubricant may then be added and the blend mixed for an additional period of time.

In the pharmaceutical industry, milling is often used to reduce the particle size of solid materials. Many types of mills are available including pin mills, hammer mills and jet mills. One of the most commonly used types of mill is the hammer mill. The hammer mill utilizes a high-speed rotor to which a number of fixed or swinging hammers are attached. The hammers can be attached such that either the knife face or the hammer face contacts the material. As material is fed into the mill, it impacts on the rotating hammers and breaks up into smaller particles. A screen is located below the hammers, which allows the smaller particles to pass through the openings in the screen. Larger particles are retained in the mill and continue to be broken up by the hammers until the particles are fine enough to flow through the screen. The material may optionally be screened. In screening, material is placed through a mesh screen or series of mesh screens to obtain the desired particle size.

Wet Granulation

In some embodiments, wet granulation is used to prepare the formulations disclosed herein.

Disclosed herein in one aspect is a method of making a composition comprising a tablet from wet granulation comprising niraparib comprising: a) forming an intragranular phase comprising i) combining niraparib, a first diluent (e.g., lactose monohydrate, lactose anhydrous, mannitol, and calcium phosphate dibasic), and a second diluent (e.g., microcrystalline cellulose-microcrystalline cellulose, starch, polyethylene oxide, and hydroxypropyl methylcellulose (HPMC)) to form a composition comprising niraparib, the first diluent, and the second diluent; and ii) wet granulating the composition comprising niraparib, the first diluent, and second diluent to form granules; b) forming an extragranular phase comprising iii) combining the granules with at least one pharmaceutically acceptable excipient to form a mixture; and c) forming a tablet by compressing the mixture obtained from step iii).

Also disclosed herein is a method of making a composition comprising a tablet from wet granulation comprising niraparib comprising: a) forming an intragranular phase comprising i) combining niraparib, lactose monohydrate, and microcrystalline cellulose to form a composition comprising niraparib, lactose monohydrate, and microcrystalline cellulose; and ii) wet granulating the composition comprising niraparib, lactose monohydrate, and microcrystalline cellulose to form granules; b) forming an extragranular phase comprising iii) combining the granules with at least one pharmaceutically acceptable excipient to form a mixture; and c) forming a tablet by compressing the mixture obtained from step iii).

In some embodiments, the wet granulating from step ii) further comprises adding a binder. In some embodiments, the binder is a liquid binder. In some embodiments, the liquid binder is dissolved povidone. In some embodiments, the liquid binder is dissolved starch, dissolved hydroxypropyl cellulose (HPC), dissolved hydroxypropyl methylcellulose (HPMC), or liquid polyethylene glycol (PEG). In some embodiments, the liquid binder is a melted binder. In some embodiments, the melted binder is a hydrophilic polyethylene glycol (PEG), poloxamer, hydrophobic fatty acid, fatty alcohol, wax, hydrogenated vegetable oil, or glyceride. In some embodiments, the binder is a dry binder. In some embodiments, the dry binder is hydroxypropyl cellulose (HPC). In some embodiments, the dry binder is hydroxypropyl methylcellulose (HPMC). In some embodiments, the dry binder is povidone (PVP) or starch. In some embodiments, the wet granulating from step ii) further comprises wet-sieving. In some embodiments, the wet granulating from step ii) further comprises drying and dry sieving.

Moisture-Activated Dry Granulation

In some embodiments, moisture-activated dry granulation is used to prepare the formulation described herein.

Provided herein in another aspect is a method of making a composition comprising a tablet from moisture-activated dry granulation comprising niraparib comprising: (a) forming an intragranular phase comprising i) combining niraparib, a first diluent (e.g., lactose monohydrate, lactose anhydrous, mannitol, and calcium phosphate dibasic), and a second diluent (e.g., microcrystalline cellulose microcrystalline cellulose, starch, polyethylene oxide, and hydroxypropyl methylcellulose (HPMC)) to form a composition comprising niraparib, the first diluent, and the second diluent; ii) granulating the composition comprising niraparib, the first diluent, and the second diluent to form granules; and (b) forming an extragranular phase comprising iii) combining the granules with at least one pharmaceutically acceptable excipient to form a mixture; and (c) forming a tablet by compressing the mixture obtained from step iii). A method as provided herein where the combining step i) further comprises combining with an adsorbant or absorbant.

Provided herein in another aspect is a method of making a composition comprising a tablet from moisture-activated dry granulation comprising niraparib comprising: (a) forming an intragranular phase comprising i) combining niraparib, lactose monohydrate, and microcrystalline cellulose to form a composition comprising niraparib, lactose monohydrate, and microcrystalline cellulose; ii) granulating the composition comprising niraparib, lactose monohydrate, and microcrystalline cellulose to form granules; and (b) forming an extragranular phase comprising iii) combining the granules with at least one pharmaceutically acceptable excipient to form a mixture; and (c) forming a tablet by compressing the mixture obtained from step iii).

In some embodiments, the granulating from step ii) further comprises adding a binder. In some embodiments, the binder is a liquid binder. In some embodiments, the liquid binder is dissolved povidone. In some embodiments, the liquid binder is water, dissolved starch, dissolved hydroxypropyl cellulose (HPC), dissolved hydroxypropyl methylcellulose (HPMC), or liquid polyethylene glycol (PEG). In some embodiments, the composition further comprises a dry binder. In some embodiments, water is added to the composition comprising the dry binder. In some embodiments, the granulating from step ii) further comprises drying and dry sieving. In some embodiments, drying comprises the addition of a glidant. In some embodiments, the glidant is silicon dioxide. In some embodiments, the glidant is silicon dioxide, tribasic calcium phosphate, calcium silicate, cellulose, magnesium silicate, magnesium trisilicate, starch, talc, or mixtures thereof.

Dry Granulation

In some embodiments, dry granulation is used to prepare the formulations described herein.

Provided in another aspect is a method of making a composition comprising a tablet from dry granulation comprising niraparib comprising: a) forming an intragranular phase comprising i) combining niraparib, a first diluent (e.g., lactose monohydrate, lactose anhydrous, mannitol, and calcium phosphate dibasic), a second diluent (e.g., microcrystalline cellulose microcrystalline cellulose, starch, polyethylene oxide, and hydroxypropyl methylcellulose (HPMC)), and a lubricant (e.g., magnesium stearate) to form a composition comprising niraparib, the first diluent, the second diluent, and the lubricant; and ii) dry granulating the composition comprising niraparib, the first diluent, the second diluent, and the lubricant to form granules; b) forming an extragranular phase comprising iii) combining the granules with at least one pharmaceutically acceptable excipient to form a mixture; and c) forming a tablet by compressing the mixture obtained from step iii).

In some embodiments, the composition further comprises a dry binder. In some embodiments, water is added to the composition comprising the dry binder. In some embodiments, combining niraparib, the first diluent, the second diluent, and the lubricant to form a composition comprising niraparib, the first diluent, the second diluent, and the lubricant from step i) further comprises blending the niraparib, the first diluent, the second diluent, and the lubricant. In some embodiments, dry granulating from step ii) comprises slugging and milling. In some embodiments, the ribbon thickness is from about 0.1 mm to about 2 mm. In some embodiments, the ribbon thickness is about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3, about 1.4 mm, about 1.5 mm, about 1.6 mm, about 1.7 mm, about 1.8 mm, about 1.9 mm, or about 2.0 mm.

Provided in another aspect is a method of making a composition comprising a tablet from dry granulation comprising niraparib comprising: a) forming an intragranular phase comprising i) combining niraparib, a diluent selected from mannitol and calcium phosphate dibasic, microcrystalline cellulose, and magnesium stearate to form a composition comprising niraparib, the diluent selected from mannitol and calcium phosphate dibasic, microcrystalline cellulose, and magnesium stearate; and ii) dry granulating the composition comprising niraparib, the diluent selected from mannitol and calcium phosphate dibasic, microcrystalline cellulose, and magnesium stearate to form granules; b) forming an extragranular phase comprising iii) combining the granules with at least one pharmaceutically acceptable excipient to form a mixture; and c) forming a tablet by compressing the mixture obtained from step iii).

In some embodiments, the composition further comprises a dry binder. In some embodiments, water is added to the composition comprising the dry binder. In some embodiments, combining niraparib, a diluent selected from mannitol and calcium phosphate dibasic, microcrystalline cellulose, and magnesium stearate to form a composition comprising niraparib, the diluent selected from mannitol and calcium phosphate dibasic, microcrystalline cellulose, and magnesium stearate from step i) further comprises blending the niraparib, a diluent selected from mannitol and calcium phosphate dibasic, microcrystalline cellulose, and magnesium stearate. In some embodiments, dry granulating from step ii) comprises slugging and milling. In some embodiments, the ribbon thickness is from about 0.1 mm to about 2 mm.

In some embodiments, the composition from step i) further comprises a glidant (e.g., silicon dioxide). In some embodiments, the at least one pharmaceutically acceptable excipient for combining the granules with at least one pharmaceutically acceptable excipient to form a mixture from step iii) is a glidant (e.g., silicon dioxide). In some embodiments, the at least one pharmaceutically acceptable excipient for combining the granules with at least one pharmaceutically acceptable excipient to form a mixture from step iii) is a lubricant (e.g., magnesium stearate). In some embodiments, combining the granules with at least one pharmaceutically acceptable excipient to form a mixture from step iii) comprises blending the granules with at least one pharmaceutically acceptable excipient. In some embodiments, the composition from step i) is a blend composition.

In some embodiments, the composition from step i) further comprises silicon dioxide. In some embodiments, the at least one pharmaceutically acceptable excipient for combining the granules with at least one pharmaceutically acceptable excipient to form a mixture from step iii) is silicon dioxide. In some embodiments, the at least one pharmaceutically acceptable excipient for combining the granules with at least one pharmaceutically acceptable excipient to form a mixture from step iii) is magnesium stearate. In some embodiments, combining the granules with at least one pharmaceutically acceptable excipient to form a mixture from step iii) comprises blending the granules with at least one pharmaceutically acceptable excipient. In some embodiments, the composition from step i) is a blend composition.

In some embodiments, the amount of components used to form the intragranular phase is about 50% to about 98% by weight of the tablet composition. In some embodiments, the amount of components used to form the intragranular phase is about 85% to about 98% by weight of the tablet composition. In some embodiments, the amount of components used to form the intragranular phase is about 90% to about 98% by weight of the tablet composition. In some embodiments, the amount of components used to form the intragranular phase is about 92.5% to about 97.5% by weight of the tablet composition. In some embodiments, the amount of components used to form the intragranular phase is about 95% by weight of the tablet composition.

In some embodiments, the amount of components used to form the extragranular phase is about 2% to about 50% by weight of the tablet composition. In some embodiments, the amount of components used to form the extragranular phase is about 2% to about 15% by weight of the tablet composition. In some embodiments, the amount of components used to form the extragranular phase is about 2% to about 10% by weight of the tablet composition. In some embodiments, the amount of components used to form the extragranular phase is about 2.5% to about 7.5% by weight of the tablet composition. In some embodiments, the amount of components used to form the extragranular phase is about 5% by weight of the tablet composition.

In some embodiments, the granules have a bulk density of about 0.10 to about 0.99 g/cm³. In some embodiments, the granules have a bulk density of about 0.10 to about 0.90 g/cm³. In some embodiments, the granules have a bulk density of about 0.10 to about 0.80 g/cm³. In some embodiments, the granules have a bulk density of about 0.10 to about 0.70 g/cm³. In some embodiments, the granules have a bulk density of about 0.10 to about 0.60 g/cm³. In some embodiments, the granules have a bulk density of about 0.10 to about 0.50 g/cm³. In some embodiments, the granules have a bulk density of about 0.10 to about 0.40 g/cm³. In some embodiments, the granules have a bulk density of about 0.10 to about 0.30 g/cm³. In some embodiments, the granules have a bulk density of about 0.10 to about 0.20 g/cm³. In some embodiments, the granules have a bulk density of about 0.20 to about 0.99 g/cm³. In some embodiments, the granules have a bulk density of about 0.20 to about 0.90 g/cm³. In some embodiments, the granules have a bulk density of about 0.20 to about 0.80 g/cm³. In some embodiments, the granules have a bulk density of about 0.20 to about 0.70 g/cm³. In some embodiments, the granules have a bulk density of about 0.20 to about 0.60 g/cm³. In some embodiments, the granules have a bulk density of about 0.20 to about 0.50 g/cm³. In some embodiments, the granules have a bulk density of about 0.20 to about 0.40 g/cm³. In some embodiments, the granules have a bulk density of about 0.20 to about 0.30 g/cm³.

In some embodiments, the granules have a bulk density of about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, or about 0.99 g/cm³.

In some embodiments, the granules have a tapped density of about 0.10 to about 0.99 g/cm³. In some embodiments, the granules have a tapped density of about 0.10 to about 0.90 g/cm³. In some embodiments, the granules have a tapped density of about 0.10 to about 0.80 g/cm³. In some embodiments, the granules have a tapped density of about 0.10 to about 0.70 g/cm³. In some embodiments, the granules have a tapped density of about 0.10 to about 0.60 g/cm³. In some embodiments, the granules have a tapped density of about 0.10 to about 0.50 g/cm³. In some embodiments, the granules have a tapped density of about 0.10 to about 0.40 g/cm³. In some embodiments, the granules have a tapped density of about 0.10 to about 0.30 g/cm³. In some embodiments, the granules have a tapped density of about 0.10 to about 0.20 g/cm³. In some embodiments, the granules have a tapped density of about 0.20 to about 0.99 g/cm³. In some embodiments, the granules have a tapped density of about 0.20 to about 0.90 g/cm³. In some embodiments, the granules have a tapped density of about 0.20 to about 0.80 g/cm³. In some embodiments, the granules have a tapped density of about 0.20 to about 0.70 g/cm³. In some embodiments, the granules have a tapped density of about 0.20 to about 0.60 g/cm³. In some embodiments, the granules have a tapped density of about 0.20 to about 0.50 g/cm³. In some embodiments, the granules have a tapped density of about 0.20 to about 0.40 g/cm³. In some embodiments, the granules have a tapped density of about 0.20 to about 0.30 g/cm³. In some embodiments, the granules have a tapped density of about 0.30 to about 0.99 g/cm³. In some embodiments, the granules have a tapped density of about 0.30 to about 0.90 g/cm³. In some embodiments, the granules have a tapped density of about 0.30 to about 0.80 g/cm³. In some embodiments, the granules have a tapped density of about 0.30 to about 0.70 g/cm³. In some embodiments, the granules have a tapped density of about 0.30 to about 0.60 g/cm³. In some embodiments, the granules have a tapped density of about 0.30 to about 0.50 g/cm³. In some embodiments, the granules have a tapped density of about 0.30 to about 0.40 g/cm³.

In some embodiments, the granules have a tapped density of about 0.10, about 0.11, about 0.12, about 0.13, about 0.14, about 0.15, about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, or about 0.99 g/cm³.

Intragranular Phase/Extragranular Phase Distribution

In another aspect, provided herein is method of preparing formulations with specific distributions of intragranular phase and extragranular phase components. Provided in one aspect is a method of making a composition comprising a tablet comprising niraparib comprising: a) forming an intragranular phase comprising i) combining niraparib and at least one pharmaceutically acceptable excipient to form a composition comprising niraparib and at least one pharmaceutically acceptable excipient; and ii) granulating the composition comprising niraparib and at least one pharmaceutically acceptable excipient to form granules; b) forming an extragranular phase comprising iii) combining the granules with at least one pharmaceutically acceptable excipient to form a mixture; and c) forming a tablet by compressing the mixture obtained from step iii); wherein the tablet has at least one of the following: (1) the amount of components used to form the intragranular phase is about 50% to about 98% by weight of the tablet composition; and (2) the amount of components used to form the extragranular phase is about 2% to about 50% by weight of the tablet composition.

In some embodiments, the amount of components used to form the intragranular phase is about 50% to about 98% by weight of the tablet composition. In some embodiments, the amount of components used to form the intragranular phase is about 85% to about 98% by weight of the tablet composition. In some embodiments, the amount of components used to form the intragranular phase is about 90% to about 98% by weight of the tablet composition. In some embodiments, the amount of components used to form the intragranular phase is about 92.5% to about 97.5% by weight of the tablet composition. In some embodiments, the amount of components used to form the intragranular phase is about 95% by weight of the tablet composition. In some embodiments, the amount of components used to form the extragranular phase is about 2% to about 50% by weight of the tablet composition. In some embodiments, the amount of components used to form the extragranular phase is about 2% to about 15% by weight of the tablet composition. In some embodiments, the amount of components used to form the extragranular phase is about 2% to about 10% by weight of the tablet composition. In some embodiments, the amount of components used to form the extragranular phase is about 2.5% to about 7.5% by weight of the tablet composition. In some embodiments, the amount of components used to form the extragranular phase is about 5% by weight of the tablet composition.

In some embodiments, the at least one pharmaceutically acceptable excipient from step i) is a second diluent (e.g., microcrystalline cellulose, starch, polyethylene oxide, and hydroxylpropyl methylcellulose (HPMC). In some embodiments, the at least one pharmaceutically acceptable excipient from step i) is a first diluent (e.g., lactose monohydrate, lactose anhydrous, mannitol, and calcium phosphate dibasic). In some embodiments, the at least one pharmaceutically acceptable excipient from step i) is a lubricant (e.g., magnesium stearate). In some embodiments, the at least one pharmaceutically acceptable excipient is a glidant (e.g., silicon dioxide).

In some embodiments, the at least one pharmaceutically acceptable excipient from step i) is microcrystalline cellulose. In some embodiments, the at least one pharmaceutically acceptable excipient from step i) is lactose monohydrate, lactose anhydrous, mannitol, or calcium phosphate dibasic. In some embodiments, the at least one pharmaceutically acceptable excipient from step i) is magnesium stearate. In some embodiments, the at least one pharmaceutically acceptable excipient from step i) is silicon dioxide.

In some embodiments, the granulating from step ii) is wet granulating. In some embodiments, the wet granulating further comprises adding a binder. In some embodiments, the binder is a liquid binder. In some embodiments, the liquid binder is dissolved povidone. In some embodiments, the liquid binder is dissolved starch, dissolved hydroxypropyl cellulose (HPC), dissolved hydroxypropyl methylcellulose (HPMC), or liquid polyethylene glycol (PEG). In some embodiments, the liquid binder is a melted binder. In some embodiments, the melted binder is a hydrophilic polyethylene glycol (PEG), poloxamer, hydrophobic fatty acid, fatty alcohol, wax, hydrogenated vegetable oil, or glyceride. In some embodiments, the binder is a dry binder. In some embodiments, the dry binder is hydroxypropyl cellulose (HPC). In some embodiments, the dry binder is hydroxypropyl methylcellulose (HPMC). In some embodiments, the dry binder is povidone (PVP) or starch. In some embodiments, the wet-granulating from step ii) further comprises wet-sieving. In some embodiments, the wet granulating from step ii) further comprises drying and dry sieving. In some embodiments, wherein drying comprises the addition of a glidant. In some embodiments, the glidant is silicon dioxide.

In some embodiments, the granulating from step ii) is dry-granulating. In some embodiments, dry-granulating comprises slugging and milling.

In some embodiments, the at least one pharmaceutically acceptable excipient for combining the granules with at least one pharmaceutically acceptable excipient to form a mixture from step iii) is silicon dioxide. In some embodiments, the at least one pharmaceutically acceptable excipient for combining the granules with at least one pharmaceutically acceptable excipient to form a mixture from step iii) is magnesium stearate.

Dosage Form Coating

The term “coating” means a process by which an outer layer of coating material is applied to the surface of a dosage form in order to confer specific benefits over uncoated variety. It involves application of a coat, including sugar or polymeric coats, on the dosage form. The advantages of tablet coating are taste masking, odor masking, physical and chemical protection, enhancing safety of dosage form handling, protection of the drug in chemically challenging environments (e.g. stomach), and to control its release profile. Coating may be applied to a wide range of oral solid dosage form, such as particles, powders, granules, crystals, pellets and tablets. When coating composition is applied to a batch of tablets in a coating pan, the tablet surfaces become covered with a polymeric film. In some embodiments, a solid dosage form may comprise a coating systems of polyvinyl alcohol (PVA) with polyethylene glycol (PEG/Macrogol) as a plasticizer. In some embodiments, coating systems may comprise: i) PVA, ii) HPMC with glycerol triacetate (triacetin) as a plasticizer, iii) ethylcellulose with a plasticizer agent, iv) Eudragit with a plasticizer agent and v) acrylates. Commercial coating systems are also available in the art and may be used with any of the solid dosage forms disclosed herein.

Kits/Articles of Manufacture

If desired, the niraparib may be provided in a kit. The kits include a therapeutically effective dose of niraparib for treating diseases and conditions, such as cancer. The dosage forms may be packaged on blister cards for daily administration convenience and to improve adherence.

The disclosure also provides kits for preventing, treating or ameliorating the symptoms of a disease or disorder in a mammal. Such kits generally will comprise one or more of niraparib compositions or devices disclosed herein, and instructions for using the kit. The disclosure also contemplates the use of one or more of niraparib compositions, in the manufacture of medicaments for treating, abating, reducing, or ameliorating the symptoms of a disease, dysfunction, or disorder in a mammal, such as a human that has, is suspected of having, or at risk for developing cancer.

In some embodiments, a kit includes one or more additional containers, each with one or more of various materials (such as reagents, optionally in concentrated form, and/or devices) desirable from a commercial and user standpoint for use of a formulation described herein. Non-limiting examples of such materials include, but not limited to, buffers, diluents, filters, needles, syringes; carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the pharmaceutical compositions are presented in a pack or dispenser device which contains one or more unit dosage forms containing a compound provided herein. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed with invention as defined in the claims which follow. The invention disclosed herein is further illustrated by the following examples which in no way should be construed as being limiting.

### Example 1 Clinical Studies

The safety and efficacy of niraparib as maintenance therapy was studied in a Phase 3 randomized, double-blind, placebo-controlled trial (NOVA) in patients with platinum-sensitive recurrent epithelial ovarian, fallopian tube, or primary peritoneal cancer. All patients had received at least two prior platinum-containing regimens and were in response (complete or partial) to their most recent platinum-based regimen.

Eligible patients were assigned to one of two cohorts based on the results of a germline BRCA mutation test. Women who were hereditary germline BRCA mutation carriers were assigned to the germline BRCA mutated (gBRCAmut) cohort (n=203) and women who did not carry a hereditary germline BRCA mutation were assigned to the non-gBRCAmut cohort (n=350). Within each cohort, patients were randomized using a 2:1 allocation of niraparib to placebo. Randomization occurred within 8 weeks of the last dose of the most recent platinum-containing regimen.

Randomization within each cohort was stratified by time to progression after the penultimate platinum therapy (6 to <12 months and ≥12 months); use of bevacizumab in conjunction with the penultimate or last platinum regimen (yes/no); and best response during the most recent platinum regimen (complete response and partial response).

Patients began treatment on Cycle 1/Day 1 with niraparib 300 mg or matched placebo administered QD in continuous 28-day cycles. Clinic visits occurred each cycle (4 weeks ±3 days). Patients randomized to placebo were not allowed to cross over to niraparib treatment at any time.

The primary endpoint, PFS (progression-free survival), was determined by central independent assessment per RECIST (Response Evaluation Criteria in Solid Tumors, version 1.1) or clinical signs and symptoms and increased CA-125. PFS as defined in the NOVA study was measured from the time of randomization (which occurred up to 2 months after completion of the most recent chemotherapy regimen) to disease progression or death.

Prior to unblinding of the study, tumors of patients randomized to the non-gBRCAmut cohort were tested for the presence of homologous recombination deficiency (HRD) using the Myriad myChoice® HRD test, which evaluates three independent biomarkers of tumor genome instability: loss of heterozygosity, telomeric allelic imbalance, and large-scale state transitions. Tumors with homologous recombination deficiencies and those with somatic BRCA mutations were defined as HRD positive (HRDpos).

The primary efficacy analysis for PFS was prospectively defined and assessed for the gBRCAmut cohort. The primary efficacy analysis for PFS was prospectively defined and assessed for the non-gBRCAmut cohort with a hierarchical testing schema. In the first step, PFS was assessed in the group of patients with HRDpos tumors and if significant, PFS was assessed in the overall non-gBRCAmut cohort.

Secondary efficacy endpoints included chemotherapy-free interval (CFI), time to first subsequent therapy (TFST), PFS after the first subsequent therapy (PFS2), time to second subsequent therapy (TSST) and OS (overall survival).

Table 1 shows the results for the PFS primary endpoint for each of the primary efficacy populations (gBRCAmut cohort, the overall non-gBRCAmut cohort and the HRDpos group in the non-gBRCAmut cohort).

PFS was significantly longer for patients who received niraparib compared to those who received placebo for all three primary efficacy populations.

Within the gBRCAmut cohort, the median PFS from time of randomization was 21.0 months with niraparib versus 5.5 months with placebo.

In the overall non-gBRCAmut cohort, the median PFS from time of randomization was 9.3 months with niraparib versus 3.9 months with placebo.

PFS was also significantly longer with niraparib than with placebo in the HRDpos group of the non-gBRCAmut cohort: 12.9 months versus 3.8 months.

TABLE 1
PFS Primary Endpoints

| | | | non-gBRCAmut | | | |
|---|---|---|---|---|---|---|
| | gBRCAmut Cohort | | Cohort | | HRDpos* | |
| | Niraparib | Placebo | Niraparib | Placebo | Niraparib | Placebo |
| | (N = 138) | (N = 65) | (N = 234) | (N = 116) | (N = 106) | (N = 56) |
| PFS Median | 21.0 | 5.5 | 9.3 | 3.9 | 12.9 | 3.8 |
| (95% CI)^{†} | (12.9, NR) | (3.8, 7.2) | (7.2, 11.2) | (3.7, 5.5) | (8.1, 15.9) | (3.5, 5.7) |

| | | | |
|---|---|---|---|
| p-value | <0.0001 | <0.0001 | <0.0001 |
| Hazard Ratio (HR) | 0.27 | 0.45 | 0.38 |
| (95% CI) | (0.173, 0.410) | (0.338, 0.607) | (0.243, 0.586) |

*HRDpos represents a prospectively defined subgroup of the non-gBRCAmut cohort.
^{†}Progression-free survival is defined as the time in months from the date of randomization to disease progression or death.

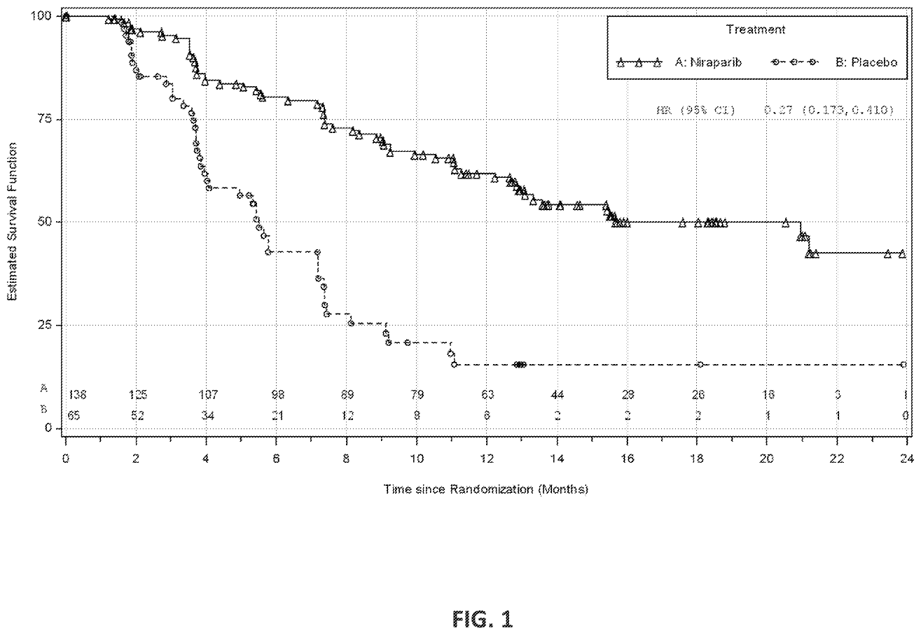

The Kaplan-Meier curves for the 2 treatment arms in the gBRCAmut cohort show early divergence of the curves with the niraparib curve consistently above that of placebo and sustained separation in the curves throughout the observation period (FIG. 1).

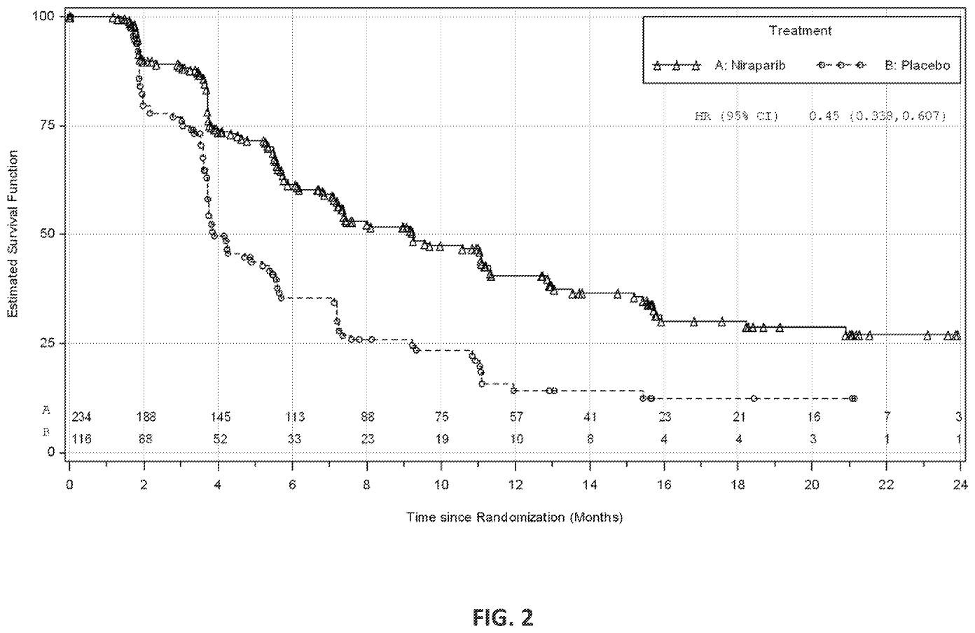

The Kaplan-Meier curves for the 2 treatment arms in the overall non-gBRCAmut cohort show early divergence of the curves with the niraparib curve consistently above that of placebo and sustained separation in the curves throughout the observation period (FIG. 2).

The secondary endpoints CFI and TFST demonstrated a persistent treatment effect in favor of the niraparib treatment arm in the gBRCAmut cohort: Median CFI was 22.8 months (95% CI: 17.9, NE) in the niraparib arm compared to 9.4 months (95% CI: 7.9, 10.6) in the placebo arm with a HR of 0.26 (95% CI: 0.166, 0.409) (p<0.0001). Median TFST was 21.0 months (95% CI: 17.5, NE) in the niraparib arm compared to 8.4 months (95% CI: 6.6, 10.6) in the placebo arm with a HR of 0.31 (95% CI: 0.205, 0.481) (p<0.0001).

In the non-gBRCAmut cohort: Median CFI was 12.7 months (95% CI: 11.0, 14.7) in the niraparib arm compared to 8.6 months (95% CI: 6.9, 10.0) in the placebo arm with an HR of 0.50 (95% CI: 0.370, 0.666) (p<0.0001). Median TFST was 11.8 months (95% CI: 9.7, 13.1) in the niraparib arm compared to 7.2 months (95% CI: 5.7, 8.5) in the placebo arm with an HR of 0.55 (95% CI: 0.412, 0.721) (p<0.0001).

At the time of the analysis, the secondary endpoint results for PFS2, OS and TSST were not mature enough to evaluate. However, no detrimental effect was observed at the time of data cutoff for any of the endpoints.

### Example 2—Pediatric Studies

Niraparib is an orally available, potent, and highly selective PARP1 and PARP2 inhibitor which is authorized for the treatment of certain cancers in adult patients. The effect of niraparib in pediatric subjects having cancer is studied in a two-part trial.

The trial assesses the effects of niraparib in combination with TSR-042, a humanised monoclonal antibody that binds with high affinity to programmed cell death protein-1 (PD-1), resulting in inhibition of binding to programmed cell death-ligand 1 (PD-L1) and programmed cell death-ligand 2. Methods for the preparation of TSR-042 are described in, e.g., International Publication Number WO 2014/179664.

The trial employs patients between 6 months and 18 years in age who are diagnosed with recurrent solid tumors that exhibit a breast cancer susceptibility gene (BRCA)ness mutational signature. A “BRCAness” mutational signature can serve as an indicator of homologous recombination deficiency (HRD), and it is especially prevalent among certain paediatric solid tumours. The definition of BRCAness is based on mutational signature 3 from COSMIC (Catalogue of Somatic Mutations in Cancer 2015). A tumour is defined as BRCAness positive if the lower limit of the 95% confidence interval (CI) for signature 3 is greater than zero. Pediatric cancers having a high prevalence of BRCAness mutation include osteosarcoma (having a median age of initial diagnosis between 10 and 19 years), neuroblastoma (having median age of initial diagnosis of 26 months), and adrenocortical carcinoma (rare, but having a median age of initial diagnosis of 4 years).

In the first part, the trial assesses an initial cohort of up to 32 patients who have a baseline body weight of at least 20 kg. Patients are eligible to enrol regardless of their biomarker status if they have one of the solid tumours with higher prevalence of BRCAness mutational signature or known BRCAness mutational signature tested and confirmed from recurrent disease. In this part, TSR-042 is administered in doses ranging from 1 to 7.5 mg/kg (starting dosage 3 mg/kg). Niraparib is administered orally in a daily amount of either 100 mg (patients up to 50 kg) or 200 mg (patients of more than 50 kg). Based on the dosage response obtained in this first cohort of patients, the study is expanded to include patients with a baseline body weight of less than 20 kg (cohort of up to 16 patients). The need for dose escalation or de-escalation during the study is determined based on observed dose-limiting toxicities (DLTs) on a 21-day cycle. The dose escalation and de-escalation is guided by the modified toxicity probability interval-2 (mTPI-2) design. The target probability of DLT is chosen as 0.3, and the proper dosing toxicity interval is defined as [0.26, 0.34]. That is, any dose with a true probability of DLT falling in the proper dosing interval is considered as a candidate for the maximum tolerated dose (MTD). This first part of the study confirms a recommended dosage for use in the second part. The initial dosage for niraparib in this part of the study is calculated based on toxicology studies which have been carried out in juvenile rats (about 5 weeks old at start of trial) and beagle dogs (about 8 months old at start of trial). In those studies, administration of niraparib for 1 month determined a no-observed-adverse-effect-level of 10 mg/kg/day in juvenile rats and 6 mg/kg/day in juvenile dogs.

In the second part of the trial, a phase 2, multi-centre, single-arm, open-label basket study is conducted to evaluate the efficacy and safety of the treatment in a population of about 40 paediatric patients. Patients are eligible on diagnosis of any of the following recurrent solid tumors (osteosarcoma, medulloblastoma, high-grade glioma, neuroblastoma, adrenocortical carcinoma, Ewing sarcoma, or rhabdomyosarcoma), or of any tumor histology with a documented positivity for BRCAness mutational signature 3 obtained from whole DNA sequencing of the tumor tissue. In this part of the study, biomarker-negative patients are capped at 30% of the total number of patients. Patients are not stratified by tumor type. All eligible patients begin treatment on cycle 1 day 1, and niraparib is dosed orally following a continuous daily dosing regimen. The starting dose is as determined from the first part of the study. TSR-042 is dosed IV every 3 weeks. Patients continue receiving treatment on study until disease progression or unacceptable toxicity, with an expected median time on treatment of 3 months. Radiographic evaluations to assess the tumour response to study treatment are conducted every 9 weeks while on study treatment or at any time when progression of disease is suspected.

The primary objective of the study is to evaluate the efficacy of the treatment (objective response rate) in the pediatric population. Secondary objectives include the evaluation of disease control rate (complete response, partial response, or stable disease), progression-free survival, duration of response, and overall survival in the pediatric population.

The results of the study indicate that niraparib can be used successfully in the treatment of pediatric cancer patients. This result may be contrasted with earlier studies, which have shown that little or no significant clinical response is obtained when a pediatric population receives a monotherapeutic treatment (e.g. Blumenthal et al., “Pembrolizumab: first experience with recurrent primary central nervous system (CNS) tumors” J Neurooncol. (2016) 129(3):453-460).

Pediatric Age Ranges

A pediatric subject is a subject from their day of birth to about 21 years of age or to about 18 years of age. A pediatric subject is a subject that is about six months of age to about 21 years of age. A pediatric subject is about six months of age to about 18 years of age, about one year of age to about 18 years of age, about 1 year of age to about 6 years of age, or about 6 years of age to about 18 years of age.

In embodiments, niraparib is administered to a pediatric subject of about six years of age to about 18 years of age.

Cancers

The exemplary methods described herein can be used to treat a pediatric subject having any type of cancer that is responsive to niraparib, either alone or in combination with one or more further therapeutic agents or treatments (e.g., as described herein).

In embodiments, a cancer is cancer is characterized by a homologous recombination repair (HRR) gene deletion, a mutation in the DNA damage repair (DDR) pathway, homologous recombination deficiency (HRD), BRCA deficiency (e.g., characterized by a BRCAness mutational signature), isocitrate dehydrogenase (IDH) mutation, high tumor mutation burden (TMB), and/or a chromosomal translocation. In embodiments, a cancer is a hypermutant cancer, a MSI-H cancer, a MSI-L cancer, or a MSS cancer. In embodiments, a cancer is characterized by one or more of these characteristics.

In embodiments, a cancer is a solid tumor.

In embodiments, a cancer is a non-CNS cancer (e.g., a non-CNS solid tumor). In embodiments, a cancer is neuroblastoma, hepatoblastoma, hepatocellular carcinoma, Wilms tumor, renal cell carcinoma, melanoma, adrenocortical carcinoma, adenocarcinoma of the colon, myoepithelial carcinoma, thymic cell carcinoma, nasopharyngeal carcinoma, squamous cell carcinoma, mesothelioma, or clivus chordoma. In embodiments, a cancer is extracranial embryonal neuroblastoma.

In embodiments, a cancer is a CNS cancer (e.g., a primary CNS malignancy). In embodiments, a cancer is ependymoma. In embodiments, a cancer is a brain cancer (e.g., glioblastoma multiforme, gliosarcoma, astrocytoma, glioblastoma, medulloblastoma, glioma, supratentorial primitive neuroectodermal tumor, atypical teratoid rhabdoid tumor, choroid plexus carcinoma, malignant ganglioma, gliomatosis cerebri, meningioma, or paraganglioma). In embodiments, a cancer is high-grade astrocytoma, low-grade astrocytoma, anaplastic astrocytoma, fibrillary astrocytoma, pilocytic astrocytoma, a high-grade glioma, low-grade glioma, diffuse intrinsic pontine glioma (DIPG), or anaplastic mixed glioma.

In embodiments, a cancer is a carcinoma.

In embodiments a cancer is a gonadal tumor.

In embodiments, a cancer is a hematological cancer. In embodiments, a cancer is a lymphoma (e.g., Hodgkin's lymphoma (e.g., relapsed or refractory classic Hodgkin's Lymphoma (cHL)), non-Hodgkin's lymphoma, diffuse large B-cell lymphoma, precursor T-lymphoblastic lymphoma, lymphoepithelial carcinoma, or malignant histiocytosis).

In embodiments, a cancer is a sarcoma (e.g., Ewings sarcoma, osteosarcoma, rhabdomyosarcoma, embryonal rhabdomyosarcoma, synovial sarcoma, alveolar rhabdomyosarcoma, alveolar soft part sarcoma, spindle cell sarcoma, angiosarcoma, epithelialoid sarcoma, inflammatory myofibroblastic tumor, or malignant rhadoid tumor).

In embodiments, a cancer is Ewing's sarcoma, osteosarcoma, ERS, a CNS tumor, or neuroblastoma.

In embodiments, a cancer is recurrent.

In embodiments, a subject is a pediatric subject with a solid tumor (e.g., a recurrent solid tumor). In embodiments, a solid tumor is characterized by a biomarker (e.g., BRCA deficiency, high TMB, and/or PD-L1 expression). In embodiments, a solid tumor (e.g., a recurrent solid tumor) is Ewing's sarcoma, osteosarcoma, rhabdomyosarcoma, neuroblastoma, medulloblastoma, high-grade glioma, or adrenocortical carcinoma.

In embodiments, a pediatric subject has not received at least one other line of treatment (LOT).

In embodiments, a pediatric subject has previously received at least one other line of treatment (LOT). In embodiments, a previous line of treatment is immunotherapy. In embodiments, a previous line of treatment is not immunotherapy. In embodiments, a pediatric subject is refractory to a previously-received line of treatment (e.g., a previously-administered chemotherapy). In embodiments, a pediatric subject is resistant to a previously-received line of treatment (e.g., a previously-administered chemotherapy).

Exemplary Dosage Regimens of Niraparib

Niraparib can be administered according to a dosage regimen that is determined by a subject body weight, by a subject's body surface area (BSA), or according to a flat dose.

Exemplary dosage amounts of niraparib based on niraparib freebase are described herein. In embodiments, niraparib is administered as niraparib tosylate monohydrate.

For example, niraparib can be administered in an amount that is about 25 mg/m² to about 300 mg/m², about 25 mg/m² to about 275 mg/m², about 25 mg/m² to about 250 mg/m², about 25 mg/m² to about 200 mg/m², about 50 mg/m² to about 300 mg/m², about 50 mg/m² to about 275 mg/m², about 50 mg/m² to about 250 mg/m², about 50 mg/m² to about 200 mg/m², about 75 mg/m² to about 300 mg/m², about 75 mg/m² to about 275 mg/m², about 75 mg/m² to about 250 mg/m², about 75 mg/m² to about 200 mg/m², about 100 mg/m² to about 300 mg/m², about 100 mg/m² to about 275 mg/m², about 100 mg/m² to about 250 mg/m², about 100 mg/m² to about 200 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², or about 200 mg/m².

Niraparib can be orally administered in an amount that is about 25 mg to about 300 mg or about 25 mg to about 500 mg.

In embodiments, niraparib is administered in an amount that is about 25 mg, about 50 mg, about 75 mg, about 100 mg, about 125 mg, about 150 mg, about 175 mg, or about 200 mg.

In embodiments, niraparib is administered in an amount that is about 75 mg, about 100 mg, about 130 mg, or about 160 mg. In embodiments, niraparib is administered in an amount that is about 100 mg.

In embodiments, niraparib is administered in an amount that is about 150 mg, about 200 mg, about 260 mg, or about 320 mg. In embodiments, niraparib is administered in an amount that is about 200 mg.

In embodiments, niraparib is administered in an amount that is about 225 mg, about 300 mg, about 390 mg, or about 480 mg. In embodiments, niraparib is administered in an amount that is about 300 mg.

In embodiments, niraparib is administered as a unit dose form that is a capsule comprising about 50 mg of niraparib.

Niraparib is administered periodically to a pediatric subject. In embodiments, niraparib is administered once daily. In embodiments, niraparib is once every two days, once every three days, once every four days, once every five days, once every six days, or once every seven days.

In embodiments, two different amounts of niraparib are administered to the subject on alternating days on which dosages are administered to said subject.

In embodiments, a dosage of niraparib as described herein (e.g., a unit dose that is a tablet comprising about 50 mg niraparib) is administered with food (e.g., a dose is mixed with food).

Exemplary Combination Therapies

Niraparib also can be administered in combination with another therapeutic agent or treatment. In embodiments, a pediatric subject is administered niraparib in combination with one or more of surgery, a radiotherapy, a chemotherapy, an immunotherapy, an anti-angiogenic agent, or an anti-inflammatory.

In embodiments of combination therapy, niraparib is orally administered to a subject at a daily dose of about 50 mg based on free base.

In embodiments of combination therapy, niraparib is orally administered to a subject at a daily dose of about 100 mg based on free base.

In embodiments of combination therapy, niraparib is orally administered to a subject at a daily dose of about 200 mg based on free base.

In embodiments, a pediatric subject has been further administered or will be further administered an immune checkpoint inhibitor.

Exemplary immune checkpoint inhibitors include inhibitors of PD-1, LAG-3, CTLA-4, TIM-3, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, DO, or CSF1R. In embodiments, an immune checkpoint inhibitor is an agent that inhibits PD-1, LAG-3, TIM-3, CTLA-4, TIGIT, DO, or CSF1R.

In embodiments, an immune checkpoint inhibitor is an agent that inhibits PD-1 (e.g., a small molecule, a nucleic acid, a polypeptide, a carbohydrate, a lipid, a metal, a toxin, a PD-1 binding agent, or a PD-L1 binding agent).

In embodiments, a PD-1 inhibitor is a PD-L1/L2 binding agent (e.g., an antibody, an antibody conjugate, or an antigen-binding fragment thereof such as durvalumab, atezolizumab, avelumab, BGB-A333, SHR-1316, FAZ-053, CK-301, or, PD-L1 millamolecule, or derivatives thereof).

In embodiments, a PD-1 inhibitor is a PD-1 binding agent (e.g., an antibody, an antibody conjugate, or an antigen-binding fragment thereof such as nivolumab, pembrolizumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, AMP-514/MEDI-0680, AGEN-2034, CS1001, TSR-042, Sym-021, PF-06801591, LZMO09, KN-035, AB122, genolimzumab (CBT-501), AK 104, or GLS-010, or derivatives thereof). In embodiments, a PD-1 inhibitor is TSR-042.

In embodiments, a PD-1 inhibitor is administered intravenously.

In embodiments, a PD-1 inhibitor is administered to the subject periodically at a dose of about 50 mg to about 2000 mg, about 50 mg to about 1000 mg, or about 100 mg to about 500 mg.

In embodiments, a PD-1 inhibitor is administered to the subject periodically at a dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, or about 1700 mg.

In some embodiments, a dose of a PD-1 inhibitor (e.g., TSR-042) is in an amount relative to body weight. In some embodiments, a dose of a PD-1 inhibitor (e.g., TSR-042) agent is within a range of about 0.01 mg/kg to 100 mg/kg of animal or human body weight; however, doses below or above this exemplary range are within the scope of the invention. A dose of a PD-1 inhibitor (e.g., TSR-042) can be about 0.01 mg/kg to about 50 mg/kg of total body weight (e.g., about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, or a range defined by any two of the foregoing values).

In embodiments, a PD-1 inhibitor is administered to the subject once every week, once every two weeks, once every three weeks, once every four weeks, once every five weeks, once every six weeks, once every seven weeks, once every eight weeks, once every nine weeks, or once every ten weeks. In embodiments, a PD-1 inhibitor (e.g., TSR-042) is administered to the subject once every three weeks.

In embodiments, a PD-1 inhibitor is administered as a first dose once every 3 weeks for 3, 4, or 5 cycles followed by a second dose administered once every six weeks. In embodiments, a first dose is about 500 mg of the PD-1 inhibitor. In embodiments, a second dose is about 1000 mg of the PD-1 inhibitor.

In embodiments, a PD-1 inhibitor (e.g., TSR-042) is administered as a dose of about 500 mg once every about three weeks in combination with daily oral administration of 50 mg niraparib based on free base. In embodiments, niraparib is administered as a solid oral dosage form (e.g., a tablet or capsule). In embodiments, niraparib is administered as a liquid oral dosage form (e.g., a solution or suspension).

In embodiments, a PD-1 inhibitor (e.g., TSR-042) is administered as a dose of about 500 mg once every about three weeks in combination with daily oral administration of 100 mg niraparib based on free base. In embodiments, niraparib is administered as a solid oral dosage form (e.g., a tablet or capsule). In embodiments, niraparib is administered as a liquid oral dosage form (e.g., a solution or suspension).

In embodiments, a PD-1 inhibitor (e.g., TSR-042) is administered as a dose of about 500 mg once every about three weeks in combination with daily oral administration of 200 mg niraparib based on free base. In embodiments, niraparib is administered as a solid oral dosage form (e.g., a tablet or capsule). In embodiments, niraparib is administered as a liquid oral dosage form (e.g., a solution or suspension).

In embodiments, a PD-1 inhibitor (e.g., TSR-042) is administered as a weight-based dose of about 0.5 mg/kg to 10 mg/kg once every about three weeks in combination with daily oral administration of 50 mg niraparib based on free base. In embodiments, a dose of a PD-1 inhibitor (e.g., TSR-042) administered every three weeks is about 0.5 mg/kg to 2 mg/kg (e.g., 0.5 mg/kg, 1.0 mg/kg, or 1.5 mg/kg). In embodiments, a dose of a PD-1 inhibitor (e.g., TSR-042) administered every three weeks is about 3.0 mg/kg to 5.0 mg/kg (e.g., 3.0 mg/kg, 3.5 mg/kg, or 4.0 mg/kg). In embodiments, a dose of a PD-1 inhibitor (e.g., TSR-042) administered every three weeks is about 6.0 mg/kg to 8.0 mg/kg (e.g., 6.5 mg/kg, 7.0 mg/kg, or 7.5 mg/kg). In embodiments, niraparib is administered as a solid oral dosage form (e.g., a tablet or capsule). In embodiments, niraparib is administered as a liquid oral dosage form (e.g., a solution or suspension).

In embodiments, a PD-1 inhibitor (e.g., TSR-042) is administered as a weight-based dose of about 0.5 mg/kg to 10 mg/kg once every about three weeks in combination with daily oral administration of 100 mg niraparib based on free base. In embodiments, a dose of a PD-1 inhibitor (e.g., TSR-042) administered every three weeks is about 0.5 mg/kg to 2 mg/kg (e.g., 0.5 mg/kg, 1.0 mg/kg, or 1.5 mg/kg). In embodiments, a dose of a PD-1 inhibitor (e.g., TSR-042) administered every three weeks is about 3.0 mg/kg to 5.0 mg/kg (e.g., 3.0 mg/kg, 3.5 mg/kg, or 4.0 mg/kg). In embodiments, a dose of a PD-1 inhibitor (e.g., TSR-042) administered every three weeks is about 6.0 mg/kg to 8.0 mg/kg (e.g., 6.5 mg/kg, 7.0 mg/kg, or 7.5 mg/kg). In embodiments, niraparib is administered as a solid oral dosage form (e.g., a tablet or capsule). In embodiments, niraparib is administered as a liquid oral dosage form (e.g., a solution or suspension).

In embodiments, a PD-1 inhibitor (e.g., TSR-042) is administered as a weight-based dose of about 0.5 mg/kg to 10 mg/kg once every about three weeks in combination with daily oral administration of 200 mg niraparib based on free base. In embodiments, a dose of a PD-1 inhibitor (e.g., TSR-042) administered every three weeks is about 0.5 mg/kg to 2 mg/kg (e.g., 0.5 mg/kg, 1.0 mg/kg, or 1.5 mg/kg). In embodiments, a dose of a PD-1 inhibitor (e.g., TSR-042) administered every three weeks is about 3.0 mg/kg to 5.0 mg/kg (e.g., 3.0 mg/kg, 3.5 mg/kg, or 4.0 mg/kg). In embodiments, a dose of a PD-1 inhibitor (e.g., TSR-042) administered every three weeks is about 6.0 mg/kg to 8.0 mg/kg (e.g., 6.5 mg/kg, 7.0 mg/kg, or 7.5 mg/kg). In embodiments, niraparib is administered as a solid oral dosage form (e.g., a tablet or capsule). In embodiments, niraparib is administered as a liquid oral dosage form (e.g., a solution or suspension).

### Example 3 Tablet Formulations Prepared from Wet Granulation

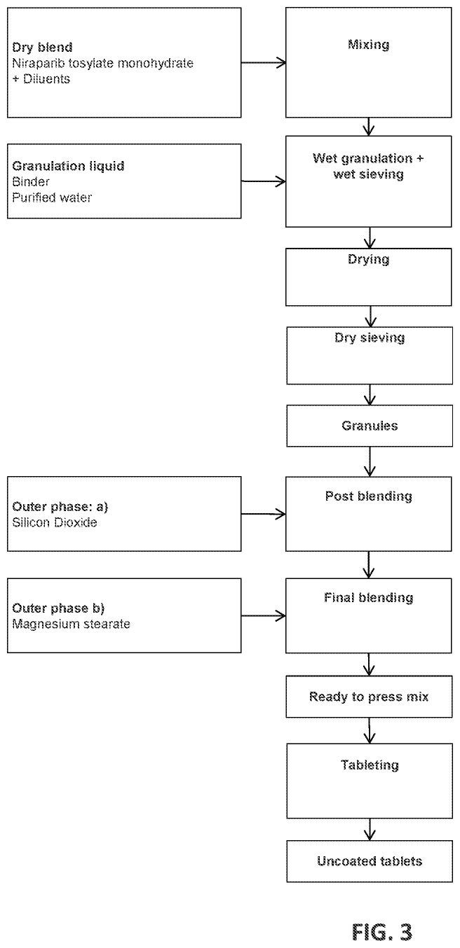

The following formulations shown in Tables 2-3 were prepared through wet granulation as shown in FIG. 3.

TABLE 2
Formulation 1 (300 mg Niraparib)

| | | |
|---|---|---|
| Component | Amount (mg) | % |

Intragranular Phase

| | | |
|---|---|---|
| Niraparib | 478.0 | 47.8 |
| Tosylate | | |
| Monohydrate | | |
| Lactose | 203.5 | 20.4 |
| Monohydrate | | |
| Microcrystalline | 203.5 | 20.4 |
| Cellulose | | |
| Crospovidone | 40.0 | 4.0 |
| Povidone | 20.0 | 2.0 |
| Purified water | | N/A |
| Total | 945.0 | 94.5 |
| (intragranular | | |
| phase) | | |

Extragranular Phase

| | | |
|---|---|---|
| Crospovidone | 40.0 | 4.0 |
| Silicon Dioxide | 5.0 | 0.5 |
| Magnesium | 10.00 | 1.0 |
| Stearate | | |
| Total | 55.0 | 5.5 |
| (extragranular | | |
| phase) | | |

TABLE 3
Formulation 2 (300 mg Niraparib)

| | | |
|---|---|---|
| Component | Amount (mg) | % |

Intragranular Phase

| | | |
|---|---|---|
| Niraparib | 478.0 | 47.8 |
| Tosylate | | |
| Monohydrate | | |
| Lactose | 193.5 | 19.4 |
| Monohydrate | | |
| Microcrystalline | 193.50 | 19.4 |
| Cellulose | | |
| Croscarmellose | 40.0 | 4.0 |
| Hydroxypropyl | 40.0 | 4.0 |
| cellulose | | |
| Purified water | | N/A |
| Total | 945.0 | 94.5 |
| (intragranular | | |
| phase) | | |

Extragranular Phase

| | | |
|---|---|---|
| Croscarmellose | 40.0 | 4.0 |
| Sodium | | |
| Silicon Dioxide | 5.00 | 0.5 |
| Magnesium | 10.00 | 1.0 |
| Stearate | | |
| Total | 55.00 | 5.5 |
| (extragranular | | |
| phase) | | |

### Example 4 Tablet Formulations Prepared from Moisture-Activated Dry Granulation

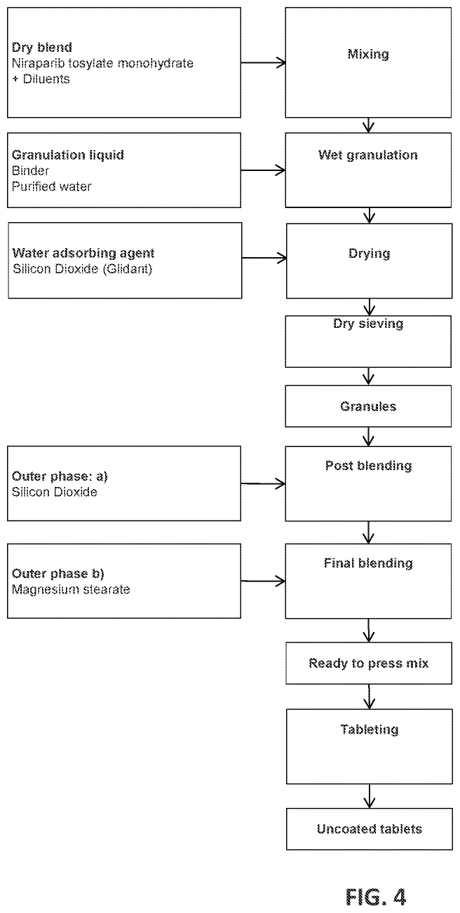

The following formulations shown in Table 4 were prepared through moisture-activated dry granulation as shown in FIG. 4.

TABLE 4
Formulation 3 (300 mg Niraparib)

| | | |
|---|---|---|
| Component | Amount (mg) | % |

Intragranular Phase

| | | |
|---|---|---|
| Niraparib | 478.0 | 47.8 |
| Tosylate | | |
| Monohydrate | | |
| Lactose | 178.5 | 17.9 |
| Monohydrate | | |
| Microcrystalline | 178.5 | 17.9 |
| Cellulose | | |
| Crospovidone | 40.0 | 4.0 |
| Povidone | 40.0 | 4.0 |
| Purified water | | N/A |
| Silicon Dioxide | 25.0 | 2.5 |
| Total | 940.0 | 94.0 |
| (intragranular | | |
| phase) | | |

Extragranular Phase

| | | |
|---|---|---|
| Crospovidone | 40.0 | 4.0 |
| Silicon Dioxide | 10.0 | 1.0 |
| Magnesium | 10.00 | 1.0 |
| Stearate | | |
| Total | 60.00 | 6.00 |
| (extragranular | | |
| phase) | | |

### Example 5 Tablet Formulations Prepared from Dry Granulation

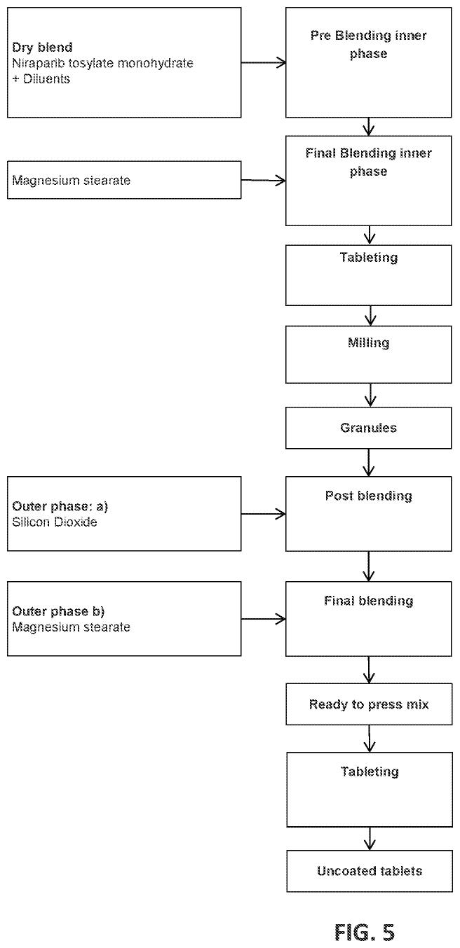

The following formulations shown in Tables 5-7 were prepared through dry granulation as shown in FIG. 5.

TABLE 5
Formulation 4 (300 mg Niraparib)

| | | |
|---|---|---|
| Component | Amount (mg) | % |

Intragranular Phase

| | | |
|---|---|---|
| Niraparib | 478.0 | 47.8 |
| Tosylate | | |
| Monohydrate | | |
| Microcrystalline | 201.0 | 20.1 |
| Cellulose | | |
| Calcium | 201.0 | 20.1 |
| phosphate dibasic | | |
| Crospovidone | 40.0 | 4.0 |
| Povidone | 20.0 | 2.0 |
| Magnesium | 5.0 | 0.5 |
| Stearate | | |
| Total | 945.0 | 94.5 |
| (intragranular | | |
| phase) | | |

Extragranular Phase

| | | |
|---|---|---|
| Crospovidone | 40.0 | 4.0 |
| Silicon Dioxide | 5.0 | 0.5 |
| Magnesium | 10.00 | 1.0 |
| Stearate | | |
| Total | 55.0 | 5.5 |
| (extragranular | | |
| phase) | | |

TABLE 6
Formulation 5 (300 mg Niraparib)

| | | |
|---|---|---|
| Component | Amount (mg) | % |

Intragranular Phase

| | | |
|---|---|---|
| Niraparib | 478.0 | 47.8 |
| Tosylate | | |
| Monohydrate | | |
| Microcrystalline | 201.0 | 20.1 |
| Cellulose | | |
| Mannitol | 201.0 | 20.1 |
| Croscarmellose | 40.0 | 4.0 |
| Sodium | | |
| Hydroxypropyl | 20.0 | 2.0 |
| cellulose | | |
| Magnesium | 5.0 | 0.5 |
| Stearate | | |
| Total | 945.0 | 94.5 |
| (intragranular | | |
| phase) | | |

Extragranular Phase

| | | |
|---|---|---|
| Croscarmellose | 40.0 | 4.0 |
| Sodium | | |
| Silicon Dioxide | 5.0 | 0.5 |
| Magnesium | 10.00 | 1.0 |
| Stearate | | |
| Total | 55.0 | 5.5 |
| (extragranular | | |
| phase) | | |

TABLE 7
Formulation 6 (300 mg Niraparib)

| | | |
|---|---|---|
| Component | Amount (mg) | % |

Intragranular Phase

| | | |
|---|---|---|
| Niraparib | 478.0 | 47.8 |
| Tosylate | | |
| Monohydrate | | |
| Microcrystalline | 201.0 | 20.1 |
| Cellulose | | |
| Mannitol | 201.0 | 20.1 |
| Crospovidone | 40.0 | 4.0 |
| Povidone | 20.0 | 2.0 |
| Magnesium | 5.0 | 0.5 |
| Stearate | | |
| Total | 945.0 | 94.5 |
| (intragranular | | |
| phase) | | |

Extragranular Phase

| | | |
|---|---|---|
| Crospovidone | 40.0 | 4.0 |
| Silicon Dioxide | 5.0 | 0.5 |
| Magnesium | 10.00 | 1.0 |
| Stearate | | |
| Total | 55.0 | 5.5 |
| (extragranular | | |
| phase) | | |

### Example 6 Tablet Stability Under Storage Conditions

The stability of the tablets disclosed herein, such as those disclosed in Examples 1-3, is evaluated under storage in HDPE bottles ‘open dish’ under accelerated conditions, such as at 40° C. and 75% relative humidity (RH). Stability may be evaluated, for example, for 1 3, 6, 9, 12, 24 or 36 months.

The tablets corresponding to Formulations 1-6 were evaluated for the amount of total impurities at 40° C. and 75% relative humidity (RH) after storage for 0, 1, and 2 months, and the total impurity measured for each of the tablets was less than 0.2%.

The tablets corresponding to Formulations 1-6 were also evaluated for water content at 40° C. and 75% relative humidity (RH) after storage for 0, 1, and 2 months, and the results are summarized in Table 8.

TABLE 8
Water Content (%)

| | | Water Content | Water Content |
|---|---|---|---|
| | Water Content | at 1 month | at 2 months |
| | at 0 months | (40° C./75 RH) | (40° C./75 RH) |
| Tablet | [%] | [%] | [%] |
| Formulation 1 | 5.0 | 7.2 | 6.0 |
| Formulation 2 | 4.3 | 6.4 | 5.7 |
| Formulation 3 | 7.7 | 7.6 | 7.0 |
| Formulation 4 | 4.3 | 6.4 | 6.0 |
| Formulation 5 | 3.4 | 5.1 | 4.1 |
| Formulation 6 | 4.2 | 6.0 | 4.9 |

### Example

Different batches of niraparib 100 mg capsules with various batch sizes were generated by the processes described herein. The batch size ranged from about 10,000 capsules to about 300,000 capsules using V-blenders or double cone blenders. For all batches, all components (API, lactose, and magnesium stearate) were screened. Both manual and automated encapsulators were used. Different batches produced herein are summarized in Table 9.

TABLE 9
Batches of 100 mg niraparib capsules produced

| | Batch | | | |
|---|---|---|---|---|
| Batch | Size | | | |
| Number | (capsules) | Screening Process | Blender | Encapsulator |
| A | 108,000 | API - screened with | double cone | manual |
| | | a mesh screen | | encapsulator |
| M | 115,000 | Lactose - screened | double cone | automated |
| B | 250,000 | or used a round | V-blender | encapsulator |
| C | 185,000 | separator | V-blender | |
| H | 18,750 | Magnesium stearate- | V-blender | |
| I | 55,000 | screened with mesh | V-blender | |
| | | screen | | |

### Example 8

A blend uniformity test was performed on a bulk hold drum at two time points. The samples were taken from the top, middle, and bottom of the drum. The results of the uniformity test are summarized in Table 10. It can be seen that the results in the % recovery column range over 5.9% for the three samples taken.

TABLE 10
Blend uniformity results of bulk hold drum

| Sample location | Sample weight (mg) | % Recovery |
|---|---|---|
| Top | 884.45 | 100.9 |
| Middle | 821.17 | 98.7 |
| Bottom | 504.30 | 95.0 |
| Average | NA | 98.2 |
| Standard Deviation | NA | 2.98 |

### Example 9

Assay and uniformity testing are described in Table 11.

TABLE 11
Assay and content uniformity of two batches

| | Assay | |
|---|---|---|
| Batch Number | (% Label Claim) | Content Uniformity |
| A | 98.0 | 6.3 |
| M | 99.7 | 2.6 |

### Example 10

Two larger scale batches were produced. With the increased scale, sampling of the blended material was conducted to confirm the process parameters used resulted in a uniform blend. The additional sampling included blend uniformity in the V-blender and in the bulk receiving container. Bulk density and tapped density were measured and used to calculate the Hausner Ratio and Carr Index. The resultant data demonstrate a bulk density of 0.525-0.590 g/cc, a tapped density of 0.820-0.900 g/cc, a Hausner's ratio of 1.52-1.67 and a Carr's index of 34-40. Prelubrication blend uniformity after addition of magnesium stearate was uniform

### Example 11

After the blending and sampling steps, the bulk blend for batches B and C were each separated into several containers and sampled for blend uniformity before encapsulation. All containers demonstrate a similar uniformity around 100% with a low standard deviation. Both batches exhibited similar dissolution profiles.

### Example 12

Blend uniformity was taken after initial blending and after the lubricant was added. The discharged blend was then tested in the bulk container for uniformity. Encapsulation was cutoff at a pre-specified point to ensure uniform assay in capsules during the encapsulation run. FIGS. 6A and 6B illustrate the basic manufacturing process. The blend was uniformly blended both before and after the lubricant was added. The contents were discharged into a single container for both batches to prepare for encapsulation. The single container was sampled for uniformity and results indicated that the bulk blend was uniform after transferring to the final bulk container. Bulk density and tapped density were measured and used to calculate the Hausner Ratio and Carr Index. Bulk density and tapped density were measured and used to calculate the Hausner Ratio and Carr Index. The resultant data demonstrate a bulk density of 0.516-0.582 g/cc, a tapped density of 0.831-0.0.846 g/cc, a Hausner's ratio of 1.43-1.64, a Carr's index of 20-22, and a Flowdex of 20-22 mm.

### Example 13

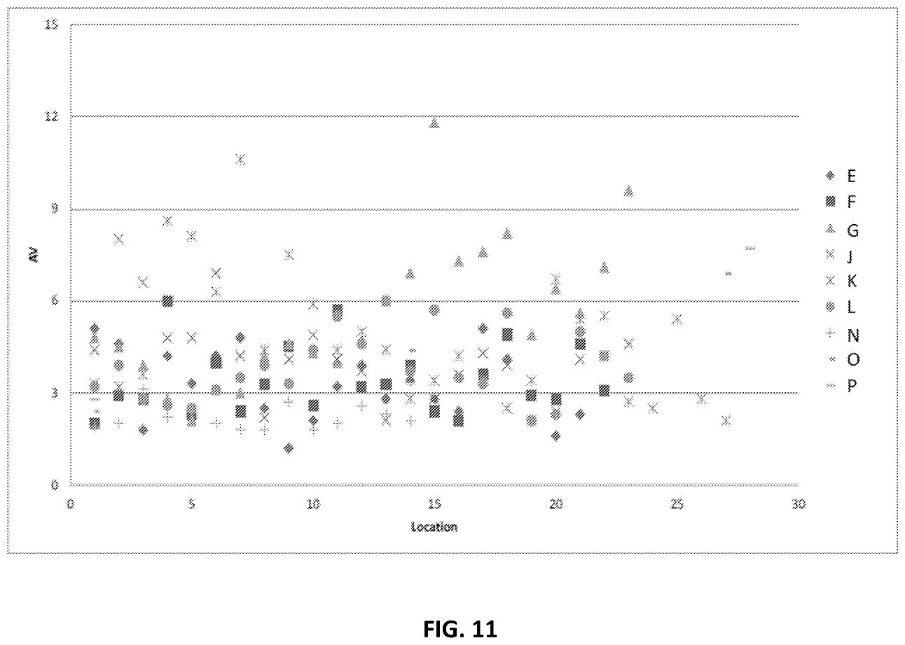

In preparing certain drug product batches, segregation of the blend occurred during capsule filling, particularly during the end of the filling of the powder blend. Therefore, measurement of the stratified content uniformity (SCU) of the capsules and sampling from the dosing bowl were performed at the end of the run. Sampling results demonstrated that the niraparib content throughout the setup and encapsulation was uniform. The niraparib content from the stratified content uniformity (SCU) measurements was from 98.7% to 105.6% throughout the setup and encapsulation. Results from the dosing bowl at the end of the run demonstrated a slightly higher niraparib content as compared to the bulk container blend uniformity test results (104.9% to 105.1%). The dissolution of these batches was uniform. FIG. 11 is an exemplary graph of sampling location of the encapsulator dosing bowl for batches E, F, G, J, K, and L.

### Example 14

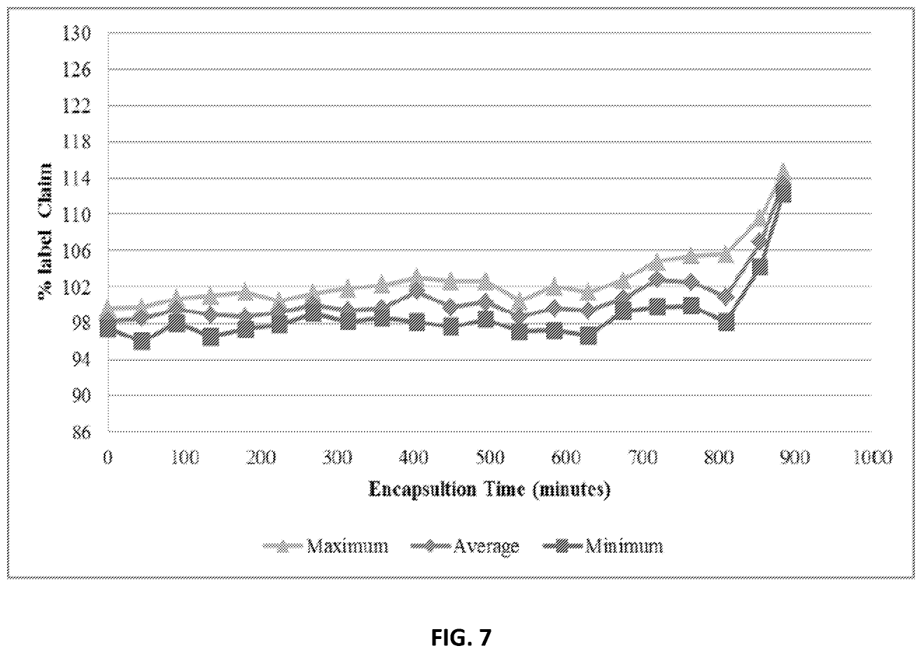

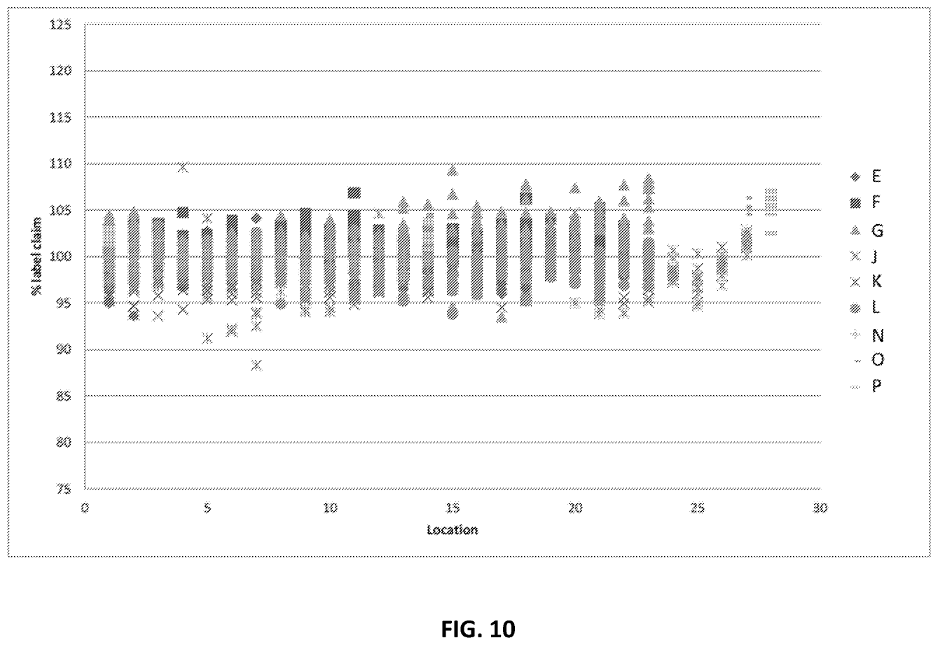

One or more batches were produced at the 185,000 capsule scale using a V-blender and an automated encapsulator. In-process sampling was performed to evaluate the uniformity of the capsules throughout the encapsulation process. Not less than twenty stratified content uniformity (SCU) in-process samples were taken over the encapsulation process of batch D. Blend uniformity testing was performed results demonstrated blend uniformity in the prelubrication blend and the final blend with a relatively low standard deviation at all sampling times. Powder characteristics of the powder blend were measured and calculated. The resultant data demonstrate a bulk density of 0.525-0.590 g/cc, a tapped density of 0.8086-0.900 g/cc, a Hausner's ratio of 1.41-1.67 and a Carr's index of 29-40, and a Flowdex of 20-22 mm. During the manufacture of the one or more batches, stratified content uniformity (SCU) was consistent throughout the run(s) until the later time points and in particular the last two time points (855 and 885 minutes). FIG. 7 illustrates the average, minimum, and maximum percent label claim values across the encapsulation process for a batch. FIG. 10 is an exemplary graph of individual stratified content uniformity data from different batches tested. One capsule (from batch K) tested at 170 minutes resulted in an assay value of 88.3%, but this capsule would have been rejected during weight sorting because it was outside of the in-process range. Stratified content uniformity (SCU) samples are not weight sorted.

### Example 15

Additional batches were produced to minimize blend segregation. These batches were divided into sub-lots at various time intervals and each sub-lot was analyzed for content uniformity. The batches used are described in Table 12. The niraparib tosylate monohydrate had a volume mean diameter of about 34.4 microns to about 58.4 microns, a D(3,2) of about 14.9 microns to about 23.4 microns, a bulk density of 0.34-0.45 g/cc, and/or a tapped density of 0.53-0.66 g/cc.

TABLE 12
Examples of batches manufactured

| Batch | Batch Size | Screening | | |
|---|---|---|---|---|
| Number | (capsules) | Process | Blender | Encapsulator |
| E | 185,000 | Drug substance - | V-blender | Automated |
| F | 185,000 | screened with | | Encapsulator |
| G | 185,000 | mesh screen | | (200 capsules/ |
| J | 55,000 | Lactose - screened | V-blender | minute) |
| K | 185,000 | or used round | V-blender | |
| L | 185,000 | separator | | |
| | | Magnesium | | |
| | | Stearate (screened | | |
| | | with mesh) | | |

### Example 16

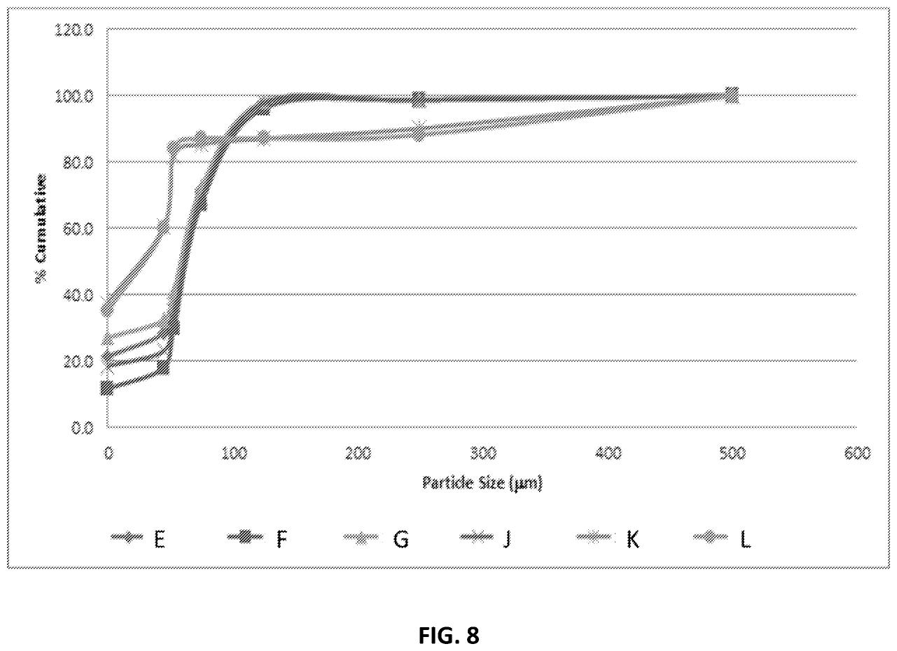

After initial mixing of the pre-lubricated blend with API and lactose (before magnesium stearate), samples were removed for blend uniformity analysis. All results demonstrated a uniform blend before the lubricant, magnesium stearate, is added. In any batch exhibiting lumps, the whole blend is removed from the V-blender, screened through a mesh screen and placed back in the V-blender for additional blending. Any changes in moisture content, if observed during blend storage, did not impact encapsulation or the final drug product. Following acceptance of the pre-lubrication blend, magnesium stearate was added and blended in V-blenders. The V-blender was sampled from various positions within the blender for final blend uniformity and the results demonstrated that the final blend was uniformly mixed. After final blending, samples are taken for analysis and demonstrate that the density of the batches were very similar. Particle size is presented graphically in FIG. 8. The final blend is discharged into bulk containers after the final blend samples are taken and show that the blend remains uniform after discharge into the bulk containers prior to encapsulation. The average % recovery for all samples taken for the batches was from 96.8% to 101.7%, indicating a reasonably uniform blend.

### Example 17

Stratified uniformity of the above sample batches was tested. To address potential segregation observed during the encapsulation, the capsules were divided into sub-lots. Once the blend hopper reached a defined level, collection of the capsules were stopped. The pre-defined cutoff point was where the powder blend reaches the end of the cylindrical portion of the blend hopper. All capsules tested prior to the cutoff passed the in-process acceptance criteria. Segregation was not observed in any of the batches.

### Example 18

Bulk hold stability was conducted on certain batches in a packaging configuration representative of commercial packaging. The capsules were tested for assay, degradation products, and dissolution at regular interval for bulk stability evaluation. Bulk hold study measurements from batches stored at 5° C., 25° C./60% RH, 30° C./65% RH, 40° C./75% RH were taken. The results demonstrated that less than 0.05% wt/wt of impurities were present initially and less than 0.05% wt/wt was present after storage for 1 and 3 months, and 0.1% after storage for 6, 9, and 12 months at 5° C., 25° C./60% RH, 30° C./65% RH, 40° C./75% RH for all samples tested. Less than or about 0.06% wt/wt of any single degradation product was present initially and less than 0.1% wt/wt of any single degradation product was present after storage for 1, 3, 6, 9, and 12 months at 5° C., 25° C./60% RH, 30° C./65% RH, 40° C./75% RH for all samples tested. Less than or about 0.06% wt/wt of total degradation product was present initially and less than 0.1% wt/wt of total degradation product was present after storage for 1, 3, 6, 9, and 12 months at 5° C., 25° C./60% RH, 30° C./65% RH, 40° C./75% RH for all samples tested. All dissolution passed the acceptance criteria.

### Example 19: Dissolution Data

100 mg niraparib capsules were manufactured. At the time of manufacture, the capsules were tested and released by USP 711 Apparatus 2 using a buffered solution. The dissolution profiles for niraparib capsules were obtained at bulk release, after packaging in the designated commercial packaging, and during stability storage at designated testing intervals. All dissolution passed the acceptance criteria.

### Example 20: Determination of Powder Composition Characteristics

Samples of powder compositions were prepared to evaluate the powder compositions disclosed herein. The following tests/measurements were made using a FT-4 powder rheometer from Freeman technology. See Table 13.

TABLE 13
Tests/measurements made using a FT-4 powder rheometer

| Test | Required output |
|---|---|
| Stability and | Stability profile and stability index |
| variable flow rate | Basic flowability energy |
| | Conditioned bulk density |
| | Flow rate index |
| | Specific energy |
| Wall Friction | Force vs.torque and wall friction |
| | angle, performed using extreme |
| | roughness attachments(most |
| | polished and roughest) |
| Permeability | Normal stress vs. pressure drop |
| | plots |
| Aeration | Air velocity vs. energy plots |
| | Aeration ratio |
| | Aerated energy |
| Compressibility | Normal stress vs. compressibility |
| | plots |
| | Compressibility index |
| Shear cell | Full mohr circle analysis |

The cohesion (kPa), Unconfined Yield Strength (UYS) (kPa), Major Principle Stress (MPS) (kPa), flow function (FF) (MPS/UYS), Angle of internal friction (AIF), and bulk density (BD) (g/cm³) were determined by carrying out shear cell tests using a FT-4 powder rheometer and the results can be seen in the tables below:

TABLE 14
Results from shear cell tests for indicated niraparib

| | Cohesion, | UYS, | MPS, | | AIF, | BD, |
|---|---|---|---|---|---|---|
| Material | kPa | kPa | kPa | FF | ° | g/cm³ |
| Milled, Annealed | 0.87 | 3.32 | 17.83 | 5.37 | 34.60 | 0.33 |
| Milled, Annealed | 0.82 | 3.04 | 17.24 | 5.67 | 33.26 | 0.40 |
| Non Milled, Annealed A | 1.02 | 3.97 | 18.50 | 4.66 | 35.80 | 0.37 |
| Non Milled, Annealed A | 1.10 | 4.36 | 18.64 | 4.27 | 36.54 | 0.38 |
| Milled, Non Annealed | 1.44 | 6.09 | 20.76 | 3.41 | 39.51 | 0.82 |
| Milled, Non Annealed | 1.14 | 5.07 | 21.68 | 4.27 | 41.44 | 0.54 |
| Non Milled, Non | 2.84 | 10.46 | 19.48 | 1.86 | 32.94 | 0.53 |
| Annealed | | | | | | |
| Non Milled, Non | 2.67 | 10.20 | 20.05 | 1.96 | 34.74 | 0.55 |
| Annealed | | | | | | |
| Milled, Annealed | 0.75 | 2.98 | 18.81 | 6.31 | 36.91 | 0.54 |
| Milled, Annealed | 0.84 | 3.30 | 19.12 | 5.79 | 36.11 | 0.54 |
| Non Milled, Annealed | 0.65 | 2.70 | 18.87 | 6.99 | 38.33 | 0.51 |
| Non Milled, Annealed | 0.61 | 2.54 | 19.35 | 7.62 | 38.91 | 0.50 |
| Non Milled, Annealed C | 0.97 | 3.44 | 15.95 | 4.63 | 31.07 | 0.50 |
| Non Milled, Annealed C | 0.98 | 3.44 | 15.66 | 4.56 | 30.37 | 0.50 |
| Non Milled, Annealed D | 1.14 | 3.99 | 16.44 | 4.12 | 30.49 | 0.44 |
| Non Milled, Annealed D | 1.06 | 3.76 | 16.24 | 4.32 | 31.30 | 0.46 |
| Non Milled, Annealed B | 1.26 | 4.56 | 16.70 | 3.66 | 31.99 | 0.50 |
| Non Milled, Annealed B | 1.13 | 4.10 | 16.62 | 4.05 | 32.24 | 0.50 |

AIF = Angle of internal friction;
BD = bulk density;
UYS = Unconfined Yield Strength;
MPS = Major Principle Stress;
FF = flow function (MPS/UYS)

TABLE 15
Results from shear cell tests for the blends
made with the indicated niraparib

| | Cohesion, | UYS, | MPS, | | AIF, | BD, |
|---|---|---|---|---|---|---|
| Material | kPa | kPa | kPa | FF | ° | g/cm³ |
| Non Milled, Annealed | 0.37 | 1.34 | 14.99 | 11.15 | 32.49 | 0.59 |
| Non Milled, Annealed | 0.32 | 1.15 | 14.61 | 12.67 | 31.43 | 0.57 |
| Milled, Annealed | 0.19 | 0.67 | 13.82 | 20.63 | 30.52 | 0.63 |
| Milled, Annealed | 0.21 | 0.73 | 14.27 | 19.45 | 30.55 | 0.65 |
| Milled, Annealed | 0.51 | 1.91 | 15.46 | 8.11 | 33.71 | 0.50 |
| Milled, Annealed | 0.41 | 1.56 | 15.49 | 9.96 | 34.98 | 0.52 |
| Non Milled, Annealed A | 0.40 | 1.54 | 15.64 | 10.14 | 35.25 | 0.49 |
| Non Milled, Annealed A | 0.32 | 1.27 | 15.61 | 12.32 | 36.25 | 0.51 |
| Non Milled, Non | 0.72 | 2.80 | 16.73 | 5.98 | 35.31 | 0.62 |
| Annealed | | | | | | |
| Non Milled, Non | 0.75 | 2.86 | 16.89 | 5.91 | 34.53 | 0.61 |
| Annealed | | | | | | |
| Milled, Non Annealed | 0.33 | 1.32 | 16.29 | 12.34 | 36.29 | 0.59 |
| Milled, Non Annealed | 0.56 | 2.17 | 16.27 | 7.50 | 35.00 | 0.60 |
| Non Milled, Annealed B | 0.58 | 2.18 | 14.99 | 6.88 | 33.93 | 0.59 |
| Non Milled, Annealed B | 0.57 | 2.17 | 15.11 | 6.97 | 34.60 | 0.60 |
| Non Milled, Annealed C | 0.55 | 2.05 | 14.94 | 7.28 | 33.38 | 0.61 |
| Non Milled, Annealed C | 0.32 | 1.16 | 14.41 | 12.40 | 32.84 | 0.62 |
| Non Milled, Annealed D | 0.37 | 1.34 | 14.36 | 10.69 | 32.49 | 0.58 |
| Non Milled, Annealed D | 0.27 | 1.01 | 14.51 | 14.35 | 33.85 | 0.58 |

AIF = Angle of internal friction;
BD = bulk density;
UYS = Unconfined Yield Strength;
MPS = Major Principle Stress;
FF = flow function (MPS/UYS)

### Example 21: Wall Friction Tests

A wall friction test method was developed to assess the interaction between the drug substance and stainless steel. The apparatus used is a FT-4 powder rheometer from Freeman technology. Various niraparib particles and niraparib blends obtained by the processes of the present invention were placed in a vessel containing the sample and a wall friction head to induce both vertical and rotational stresses. The powder sample was prepared by conditioning and then pre-consolidation using the standard FT4 blade and vented piston.

The wall friction head equipped with 1.2 microns average roughness of 316 Stainless Steel discs moves downwards to the surface of the sample and induces a normal stress as the disc contacts the top of the sample. The head continues to move downwards until the required normal stress is established. Slow rotation of the wall friction head then begins, inducing a shear stress. A shear plane is established between the disc and sample surfaces. As the powder bed resists the rotation of the wall friction head, the torque increases until the resistance is eventually overcome. At this point, a maximum torque is observed. The wall friction head continues to rotate at 18 degrees/min for 5 minutes. The torque required to maintain this rotational is measured which enables a “steady-state” shear stress to be calculated. The normal stress is maintained constant at the target applied stress for each step throughout that step. A series of shear stress values is measured for a range of target applied stresses. Due to the nature of the samples and the fact that an exact constant rotational torque is unlikely to be achieved, the software determines an average value during 10% of the shearing time. The wall friction angle is then calculated by drawing a best fit line through the data points on the graph, and measuring the angle subtended between this best fit line and the horizontal. The results were plotted.

These results suggest that the particles of the invention exhibit less sticky behavior to metal surfaces and have thus improved processability, e.g., for automated encapsulation of niraparib formulations described herein.

TABLE 16
Results from wall friction tests for the indicated niraparib batches

| Material | Ra | WFA, ° | BD, g/cm³ |
|---|---|---|---|
| Non Milled, Annealed | 0.05 μm | 24.32 | 0.51 |
| Non Milled, Annealed | 0.05 μm | 22.60 | 0.50 |
| Non Milled, Annealed | 0.05 μm | 21.91 | 0.49 |
| Milled, Annealed | 0.05 μm | 25.26 | 0.33 |
| Milled, Annealed | 0.05 μm | 29.53 | 0.65 |
| Milled, Annealed | 0.05 μm | 28.57 | 0.33 |
| Non Milled, Annealed A | 0.05 μm | 0.56 | 0.37 |
| Non Milled, Annealed A | 0.05 μm | 25.19 | 0.38 |
| Non Milled, Annealed A | 0.05 μm | 33.40 | 0.39 |
| Non Milled, Non Annealed | 0.05 μm | 37.05 | 0.53 |
| Non Milled, Non Annealed | 0.05 μm | 38.17 | 0.55 |
| Non Milled, Non Annealed | 0.05 μm | 38.86 | −0.73 |
| Milled, Non Annealed | 0.05 μm | 32.16 | 0.48 |
| Milled, Non Annealed | 0.05 μm | 34.29 | 0.51 |
| Milled, Non Annealed | 0.05 μm | 31.26 | 0.50 |
| Milled, Annealed | 0.05 μm | 15.77 | 0.53 |
| Milled, Annealed | 0.05 μm | 17.30 | 0.54 |
| Milled, Annealed | 0.05 μm | 19.94 | 0.53 |
| Non Milled Annealed B | 0.05 μm | 16.71 | 0.50 |
| Non Milled Annealed B | 0.05 μm | 29.20 | 0.49 |
| Non Milled Annealed B | 0.05 μm | 30.86 | 0.48 |
| Non Milled Annealed C | 0.05 μm | 29.60 | 0.50 |
| Non Milled Annealed C | 0.05 μm | 29.83 | 0.50 |
| Non Milled Annealed C | 0.05 μm | 30.54 | 0.49 |
| Non Milled Annealed D | 0.05 μm | 27.29 | 0.44 |
| Non Milled Annealed D | 0.05 μm | 31.10 | 0.46 |
| Non Milled Annealed D | 0.05 μm | 30.98 | 0.45 |

WFA = Wall friction angle;
BD = bulk density

TABLE 17
Results from wall friction tests for powder
blends made with indicated niraparib batches.

| Material | Ra | | WFA, ° | BD, g/cm³ |
|---|---|---|---|---|
| Non Milled, Annealed B | 0.05 | μm | 8.15 | 0.59 |
| Non Milled, Annealed B | 0.05 | μm | 14.09 | 0.60 |
| Non Milled, Annealed B | 0.05 | μm | 11.63 | 0.59 |
| Non Milled, Annealed B | 1.2 | μm | 24.39 | 0.59 |
| Non Milled, Annealed B | 1.2 | μm | 24.25 | 0.59 |
| Non Milled, Annealed B | 1.2 | μm | 24.15 | 0.61 |
| Non Milled, Annealed C | 0.05 | μm | 11.00 | 0.58 |
| Non Milled, Annealed C | 0.05 | μm | 13.05 | 0.63 |
| Non Milled, Annealed C | 0.05 | μm | 15.52 | 0.62 |
| Non Milled, Annealed C | 1.2 | μm | 25.21 | 0.62 |
| Non Milled, Annealed C | 1.2 | μm | 25.72 | 0.63 |
| Non Milled, Annealed C | 1.2 | μm | 24.38 | 0.62 |
| Milled, Annealed | 0.05 | μm | 8.79 | 0.65 |
| Milled, Annealed | 0.05 | μm | 17.36 | 0.65 |
| Milled, Annealed | 1.2 | μm | 24.03 | 0.66 |
| Milled, Annealed | 1.2 | μm | 25.02 | 0.65 |
| Non Milled, Annealed | 0.05 | um | 13.22 | 0.64 |
| Non Milled, Annealed | 0.05 | um | 16.37 | 0.63 |
| Non Milled, Annealed | 1.2 | μm | 24.80 | 0.62 |
| Non Milled, Annealed | 1.2 | μm | 24.70 | 0.63 |
| Milled, Annealed | 0.05 | μm | 19.00 | 0.51 |
| Milled, Annealed | 0.05 | μm | 22.77 | 0.54 |
| Milled, Annealed | 1.2 | μm | 26.65 | 0.50 |
| Milled, Annealed | 1.2 | μm | 27.23 | 0.87 |
| Non Milled, Annealed | 0.05 | μm | 14.17 | 0.49 |
| Non Milled, Annealed | 0.05 | μm | 22.72 | 0.52 |
| Non Milled, Annealed | 1.2 | μm | 26.96 | 0.50 |
| Non Milled, Annealed | 1.2 | μm | 27.78 | 0.54 |
| Non Milled, Non Annealed | 0.05 | μm | 15.90 | 0.61 |
| Non Milled, Non Annealed | 0.05 | μm | 21.46 | 0.62 |
| Non Milled, Non Annealed | 1.2 | μm | 25.27 | 0.60 |
| Non Milled, Non Annealed | 1.2 | μm | 25.57 | 0.59 |
| Milled, Non Annealed | 0.05 | μm | 13.40 | 0.60 |
| Milled, Non Annealed | 0.05 | μm | 15.66 | 0.60 |
| Milled, Non Annealed | 1.2 | μm | 27.17 | 0.60 |
| Milled, Non Annealed | 1.2 | μm | 26.86 | 0.61 |

WFA = Wall friction angle;
BD = bulk density

TABLE 18
Results from wall friction tests for smooth finish powder
blends made with indicated niraparib batches.

| Series Name | Ra | WFA, ° | BD, g/cm³ |
|---|---|---|---|
| Milled, Annealed | 0.05 μm | 8.79 | 0.65 |
| Milled, Annealed | 0.05 μm | 17.21 | 0.64 |
| Milled, Annealed | 0.05 μm | 17.36 | 0.65 |
| Non Milled, Non Annealed | 0.05 μm | 19.00 | 0.51 |
| Non Milled, Non Annealed | 0.05 μm | 22.77 | 0.54 |
| Non Milled, Non Annealed | 0.05 μm | 19.52 | 0.50 |
| Non Milled, Annealed | 0.05 μm | 14.17 | 0.49 |
| Non Milled, Annealed | 0.05 μm | 22.72 | 0.52 |
| Non Milled, Annealed | 0.05 μm | 18.84 | 0.53 |
| Non Milled, Non Annealed | 0.05 μm | 24.11 | 0.59 |
| Non Milled, Non Annealed | 0.05 μm | 15.90 | 0.61 |
| Non Milled, Non Annealed | 0.05 μm | 21.46 | 0.62 |
| Milled, Non Annealed | 0.05 μm | 13.40 | 0.60 |
| Milled, Non Annealed | 0.05 μm | 14.95 | 0.60 |
| Milled, Non Annealed | 0.05 μm | 15.66 | 0.60 |
| Non Milled, Annealed | 0.05 μm | 13.22 | 0.64 |
| Non Milled, Annealed | 0.05 μm | 16.37 | 0.63 |
| Non Milled, Annealed | 0.05 μm | 17.73 | 0.63 |

WFA = Wall friction angle;
BD = bulk density

### Example 22: Compressibility Determination

Compressibility is a measure of how density changes as a function of applied normal stress. By definition, compressibility is the percent change in volume after compression (%). The measurements were made using a FT-4 powder rheometer from Freeman technology.

Niraparib particles and blends thereof were placed in a vessel and a vented piston was used to compress the particles. The vented piston is designed such that the compression face is constructed from a woven stainless steel mesh and allows the entrained air in the powder to escape uniformly across the surface of the powder bed. A normal stress was applied in 8 sequential compression steps beginning at 0.5 kPa and ending at 15 kPa. In each step, the normal stress was held constant for 60 seconds and the compressibility was automatically calculated as a percentage change in volume. The results were plotted and the compressibility percentage measured at 15 kPa for various niraparib powder compositions.

As is illustrated by the above data in Examples 20-22, it has been found that using the methods described herein to produce powder compositions significantly increases flowability as evidenced by favorable changes in characteristics identified above, especially niraparib powders.

### First Set of Embodiments

1. A composition comprising a tablet comprising:
   an effective amount of niraparib to inhibit polyadenosine diphosphate ribose polymerase (PARP) when administered to a subject in need thereof;
   wherein the tablet has at least one of the following:
   (a) the tablet comprises less than 0.2% by weight of any single niraparib degradation product;
   (b) the tablet comprises less than 0.2% by weight of any single niraparib degradation product after storage for 1 month at 40° C. and 75% relative humidity (RH); and
   (c) the tablet comprises less than 0.2% by weight of any single niraparib degradation product after storage for 2 months at 40° C. and 75% relative humidity (RH).
2. The composition of embodiment 1, wherein the tablet comprises less than 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product.
3. The composition of embodiment 1, wherein the tablet comprises less than 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product after storage for 1 month at 40° C. and 75% relative humidity (RH).
4. The composition of embodiment 1, wherein the tablet comprises less than 0.2%, 0.1%, 0.09%, 0.08%, 0.07%, 0.06%, 0.05%, 0.04%, 0.03%, 0.02%, 0.01%, 0.005%, or 0.001% by weight of any single niraparib degradation product after storage for 2 months at 40° C. and 75% relative humidity (RH).
5. A composition comprising a tablet comprising:
   an effective amount of niraparib to inhibit polyadenosine diphosphate ribose polymerase (PARP) when administered to a subject in need thereof;
   wherein the tablet has at least one of the following:
   (a) a weight of at least 200, 500, or 800 mg;
   (b) a thickness of at least 4.0 mm; and
   (c) a friability of less than 2%;
   wherein the effective amount of niraparib is from about 50 mg to about 350 mg based on the niraparib free base.
6. The composition of embodiment 5, wherein the effective amount of niraparib is from about 75 mg to about 125 mg based on the niraparib free base.
7. The composition of embodiment 5, wherein the effective amount of niraparib is about 50 mg, 100 mg, or about 150 mg based on the niraparib free base.
8. The composition of embodiment 5, wherein the effective amount of niraparib is about 100 mg based on the niraparib free base.
9. The composition of any one of embodiments 5-8, wherein the tablet has a net weight of at least 200 mg, at least 210 mg, at least 220 mg, at least 230 mg, at least 240 mg, at least 250 mg, at least 260 mg, at least 270 mg, at least 280 mg, at least 290 mg, 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, or at least 500 mg.
10. The composition of any one of embodiments 5-8, wherein the tablet has a net weight of at least 300 mg.
11. The composition of embodiment 5, wherein the effective amount of niraparib is from about 175 mg to about 225 mg based on the niraparib free base.
12. The composition of embodiment 5, wherein the effective amount of niraparib is about 150 mg, 200 mg, or about 250 mg based on the niraparib free base.
13. The composition of embodiment 5, wherein the effective amount of niraparib is about 200 mg based on the niraparib free base.
14. The composition of any one of embodiments 11-13, wherein the tablet has a net weight of at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, at least 710 mg, at least 720 mg, at least 730 mg, at least 740 mg, at least 750 mg, at least 760 mg, at least 770 mg, at least 780 mg, at least 790 mg, or at least 800 mg.
15. The composition of any one of embodiments 11-13, wherein the tablet has a net weight of at least 600 mg.
16. The composition of embodiment 5, wherein the effective amount of niraparib is from about 275 mg to about 325 mg based on the niraparib free base.
17. The composition of embodiment 5, wherein the effective amount of niraparib is about 250 mg, about 300 mg, or about 350 mg based on the niraparib free base.
18. The composition of embodiment 5, wherein the effective amount of niraparib is about 300 mg based on the niraparib free base.
19. The composition of any one of embodiments 16-18, wherein the tablet has a net weight of at least 800 mg, at least 810 mg, at least 820 mg, at least 830 mg, at least 840 mg, at least 850 mg, at least 860 mg, at least 870 mg, at least 880 mg, at least 890 mg, at least 900 mg, at least 910 mg, at least 920 mg, at least 930 mg, at least 940 mg, at least 950 mg, at least 960 mg, at least 970 mg, at least 980 mg, at least 990 mg, at least 1000 mg, at least 1010 mg, at least 1020 mg, at least 1030 mg, at least 1040 mg, at least 1050 mg, at least 1060 mg, at least 1070 mg, at least 1080 mg, at least 1090 mg, at least 1100 mg, at least 1110 mg, at least 1120 mg, at least 1130 mg, at least 1140 mg, at least 1150 mg, at least 1160 mg, at least 1170 mg, at least 1180 mg, at least 1190 mg, or at least 1200 mg.
20. The composition of any one of embodiments 16-18, wherein the tablet has a net weight of at least 1000 mg.
21. The composition of any one of embodiments 5-20, wherein the tablet has a thickness of at least 4.0 mm, at least 4.1 mm, at least 4.2 mm, at least 4.3 mm, at least 4.4, at least 4.5 mm, at least 4.6 mm, at least 4.7 mm, at least 4.8 mm, at least 4.9 mm, at least 5.0 mm, at least 5.1 mm, at least 5.2 mm, at least 5.3 mm, at least 5.4 mm, at least 5.5 mm, at least 5.6 mm, at least 5.7 mm, at least 5.8 mm, at least 5.9 mm, at least 6.0 mm, at least 6.1 mm, at least 6.2 mm, at least 6.3 mm, at least 6.4 mm, at least 6.5 mm, at least 6.6 mm, at least 6.7 mm, at least 6.8, at least 6.9 mm, at least 7.0 mm, at least 7.1 mm, at least 7.2 mm, at least 7.3 mm, at least 7.4 mm, at least 7.5 mm, at least 7.6 mm, at least 7.7 mm, at least 7.8 mm, at least 7.9 mm, at least 8.0 mm, at least 8.5 mm, at least 9.0 mm, at least 9.5 mm, or at least 10 mm.
22. The composition of any one of embodiments 5-21, wherein the tablet has a friability of less than 2%, less than 1.9%, less than 1.8%, less than 1.7%, less than 1.6%, less than 1.5%, less than 1.4%, less than 1.3%, less than 1.2%, less than 1.1%, less than 1.0%, less than 0.9%, less than 0.8%, less than 0.7%, less than 0.6%, less than 0.5%, less than 0.4%, less than 0.3%, less than 0.2%, or less than 0.1%.
23. The composition of any one of embodiments 5-22, wherein the niraparib comprises niraparib free base or a pharmaceutically acceptable salt thereof.
24. The composition of embodiment 23, wherein the pharmaceutically acceptable salt of niraparib is niraparib tosylate.
25. A composition comprising a tablet comprising
   (a) an effective amount of niraparib to inhibit polyadenosine diphosphate ribose polymerase (PARP) when administered to a subject in need thereof; and
   (b) silicon dioxide;
      wherein the effective amount of niraparib is from about 50 mg to about 350 mg based on the niraparib free base.
26. The composition of embodiment 25, wherein the effective amount of niraparib is from about 75 mg to about 125 mg based on the niraparib free base.
27. The composition of embodiment 25, wherein the effective amount of niraparib is about 50 mg, 100 mg, or about 150 mg based on the niraparib free base.
28. The composition of embodiment 25, wherein the effective amount of niraparib is about 100 mg based on the niraparib free base.
29. The composition of embodiment 25, wherein the effective amount of niraparib is from about 175 mg to about 225 mg based on the niraparib free base.
30. The composition of embodiment 25, wherein the effective amount of niraparib is about 150 mg, 200 mg, or about 250 mg based on the niraparib free base.
31. The composition of embodiment 25, wherein the effective amount of niraparib is about 200 mg based on the niraparib free base.
32. The composition of embodiment 25, wherein the effective amount of niraparib is from about 275 mg to about 325 mg based on the niraparib free base.
33. The composition of embodiment 25, wherein the effective amount of niraparib is about 250 mg, about 300 mg, or about 350 mg based on the niraparib free base.
34. The composition of embodiment 25, wherein the effective amount of niraparib is about 300 mg based on the niraparib free base.
35. The composition of any one of embodiments 25-34, wherein the niraparib comprises niraparib free base or a pharmaceutically acceptable salt thereof.
36. The composition of embodiment 35, wherein the pharmaceutically acceptable salt of niraparib is niraparib tosylate.
37. A composition comprising a tablet comprising:
   an effective amount of niraparib to inhibit polyadenosine diphosphate ribose polymerase (PARP) when administered to a subject in need thereof;
   wherein the tablet further comprises an intragranular phase and an extragranular phase; and
      the tablet has at least one of the following:
   (a) the amount of components used to form the intragranular phase is about 50% to about 98% by weight of the tablet composition; and
   (b) the amount of components used to form the extragranular phase is about 2% to about 50% by weight of the tablet composition.
38. The composition of embodiment 37, wherein the amount of components used to form the intragranular phase is about 50% to about 98% by weight of the tablet composition.
39. The composition of embodiment 37, wherein the amount of components used to form the intragranular phase is about 85% to about 98% by weight of the tablet composition.
40. The composition of embodiment 37 wherein the amount of components used to form the intragranular phase is about 90% to about 98% by weight of the tablet composition.
41. The composition of embodiment 37, wherein the amount of components used to form the intragranular phase is about 92.5% to about 97.5% by weight of the tablet composition.
42. The composition of embodiment 37, wherein the amount of components used to form the intragranular phase is about 95% by weight of the tablet composition.
43. The composition of any one of embodiments 37-42, wherein the amount of components used to form the extragranular phase is about 2% to about 50% by weight of the tablet composition.
44. The composition of any one of embodiments 37-42, wherein the amount of components used to form the extragranular phase is about 2% to about 15% by weight of the tablet composition.
45. The composition of any one of embodiments 37-42, wherein the amount of components used to form the extragranular phase is about 2% to about 10% by weight of the tablet composition.
46. The composition of any one of embodiments 37-42, wherein the amount of components used to form the extragranular phase is about 2.5% to about 7.5% by weight of the tablet composition.
47. The composition of any one of embodiments 37-42, wherein the amount of components used to form the extragranular phase is about 5% by weight of the tablet composition.
48. The composition of any one of embodiments 1-47, further comprising a first diluent.
49. The composition of any one of embodiments 1-48, further comprising a second diluent.
50. The composition of any one of embodiments 1-49, further comprising a lubricant.
51. The composition of any one of embodiments 1-50, further comprising a binder.
52. A composition comprising a tablet comprising the following components on a weight percentage basis:
   (a) in an intragranular portion:
      (i) 40-50% niraparib tosylate monohydrate;
      (ii) 9-11% of a first diluent;
      (iii) 30-40% of a second diluent;
      (iv) 1-3% of a binder;
      (v) 0.1-2% of a disintegrant;
      (vi) 2-4% of a glidant or adsorbant or absorbant; and
      (vii) 0.1-2% of a lubricant;
   (b) in an extragranular portion:
      (i) 0.1-2% of a disintegrant;
      (ii) 0.1-2% of a glidant or adsorbant or absorbant; and
      (iii) 0.1-2% of a lubricant.
53. The composition of embodiment 52, wherein the lubricant is magnesium stearate.
54. A composition comprising a tablet comprising
   (a) an effective amount of niraparib to inhibit polyadenosine diphosphate ribose polymerase (PARP) when administered to a subject in need thereof;
   (b) a first diluent selected from lactose monohydrate, lactose anhydrous, mannitol, and calcium phosphate dibasic;
   (c) magnesium stearate;
   (d) a second diluent selected from microcrystalline cellulose, starch, polyethylene oxide, and hydroxypropyl methylcellulose (HPMC); and
   (e) a binder selected from povidone (PVP), hydroxypropyl cellulose (HPC), and hydroxypropyl methylcellulose (HPMC).
55. The composition of any one of embodiments 48-54, wherein the first diluent is lactose monohydrate.
56. The composition of embodiment 55, wherein the lactose monohydrate is spray dried or crystalline.
57. The composition of any one of embodiments 48-54, wherein the first diluent is mannitol.
58. The composition of embodiment 57, wherein the mannitol is spray dried or crystalline.
59. The composition of any one of embodiments 48-54, wherein the first diluent is calcium phosphate dibasic.
60. The composition of any one of embodiments 49-59, wherein the second diluent is microcrystalline cellulose.
61. The composition of any one of embodiments 49-59, wherein the second diluent is starch, polyethylene oxide, or hydroxypropyl methylcellulose (HPMC).
62. The composition of any one of embodiments 51-61, wherein the binder is povidone (PVP).
63. The composition of any one of embodiments 51-61, wherein the binder is hydroxypropyl cellulose (HPC).
64. The composition of any one of embodiments 51-61, wherein the binder is hydroxypropyl methylcellulose (HPMC).
65. The composition of any one of embodiments 1-64, wherein the composition further comprises a disintegrant.
66. The composition of embodiment 65, wherein the disintegrant is crospovidone or croscarmellose.
67. The composition of embodiment 66, wherein the croscarmellose is croscarmellose sodium.
68. The composition of any one of embodiments 1-67, wherein the composition further comprises a large meso-porous silica excipient as an adsorbant or absorbant.
69. The composition of embodiment 68, wherein the large meso-porous silica excipient absorbs water.
70. The composition of any one of embodiments 1-67, wherein the composition further comprises an intermediate meso-porous silica excipient as a glidant.
71. The composition of any one of embodiment 70, wherein the intermediate meso-porous silica comprises syloid FP-244.
72. The composition of any one of embodiments 1-71, wherein the composition further comprises silicon dioxide.
73. The composition of embodiment 72, wherein the silicon dioxide is present in an amount of about 0.1% to about 10% by weight.
74. The composition of embodiment 72, wherein the silicon dioxide is present in an amount of about 0.1% to about 5% by weight.
75. The composition of embodiment 72, wherein the silicon dioxide is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, or about 5% by weight.
76. The composition of any one of embodiments 1-75, wherein the composition further comprises an intragranular phase.
77. The composition of embodiment 76, wherein the intragranular phase comprises silicon dioxide.
78. The composition of embodiment 77, wherein the silicon dioxide in the intragranular phase is present in an amount of about 0.1% to about 10% by weight.
79. The composition of embodiment 77, wherein the silicon dioxide in the intragranular phase is present in an amount of about 0.1% to about 5% by weight.
80. The composition of embodiment 77, wherein the silicon dioxide in the intragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, or about 5% by weight.
81. The composition of embodiment 76, wherein the intragranular phase does not comprise magnesium stearate.
82. The composition of embodiment 81, wherein the intragranular phase comprises niraparib, lactose monohydrate, microcrystalline cellulose, crospovidone, and povidone.
83. The composition of embodiment 81, wherein the intragranular phase comprises niraparib, lactose monohydrate, microcrystalline cellulose, croscarmellose, and hydroxypropyl cellulose (HPC).
84. The composition of embodiment 81, wherein the intragranular phase comprises niraparib, lactose monohydrate, microcrystalline cellulose, croscarmellose, and hydroxypropyl methylcellulose (HMPC).
85. The composition of embodiment 81, wherein the intragranular phase comprises niraparib, lactose monohydrate, microcrystalline cellulose, crospovidone, povidone, and a large meso-porous silica excipient as an adsorbant or absorbant or an intermediate meso-porous silica excipient as a glidant.
86. The composition of embodiment 81, wherein the intragranular phase comprises niraparib, lactose monohydrate, microcrystalline cellulose, crospovidone, povidone, and a large meso-porous silica excipient as an adsorbant or absorbant.
87. The composition of embodiment 81, wherein the intragranular phase comprises niraparib, lactose monohydrate, microcrystalline cellulose, crospovidone, povidone, and an intermediate meso-porous silica excipient as a glidant.
88. The composition of embodiment 76, wherein the intragranular phase comprises magnesium stearate.
89. The composition of embodiment 88, wherein the intragranular phase comprises niraparib, microcrystalline cellulose, calcium phosphate dibasic, crospovidone, povidone, and magnesium stearate.
90. The composition of embodiment 88, wherein the intragranular phase comprises niraparib, microcrystalline cellulose, mannitol, croscarmellose, hydroxypropyl cellulose (HPC), and magnesium stearate.
91. The composition of embodiment 88, wherein the intragranular phase comprises niraparib, microcrystalline cellulose, mannitol, croscarmellose, hydroxypropyl methylcellulose (HPMC), and magnesium stearate.
92. The composition of embodiment 88, wherein the intragranular phase comprises niraparib, microcrystalline cellulose, mannitol, crospovidone, povidone, and magnesium stearate.
93. The composition of any one of embodiments 1-92, wherein the composition further comprises an extragranular phase.
94. The composition of embodiment 93, wherein the extragranular phase comprises magnesium stearate.
95. The composition of embodiments 93 or 94, wherein the extragranular phase comprises crospovidone.
96. The composition of embodiments 93 or 94, wherein the extragranular phase comprises croscarmellose.
97. The composition of any one of embodiments 93-96, wherein the extragranular phase comprises silicon dioxide.
98. The composition of embodiment 97, wherein the silicon dioxide in the extragranular phase is present in an amount of about 0.1% to about 10% by weight.
99. The composition of embodiment 97, wherein the silicon dioxide in the extragranular phase is present in an amount of about 0.1% to about 5% by weight.
100. The composition of embodiment 97, wherein the silicon dioxide in the extragranular phase is present in an amount of about 0.1% to about 2.5% by weight.
101. The composition of embodiment 97, wherein the silicon dioxide in the extragranular phase is present in an amount of about 0.1%, about 0.2%, about 0.3%, about 0.4%, about 0.5%, about 0.6, about 0.7%, about 0.8%, about 0.9%, about 1%, about 1.5%, about 2%, about 2.5%, about 3%, about 3.5%, about 4%, about 4.5%, or about 5% by weight.
102. The composition of any one of embodiments 1-101, wherein the tablet has a disintegration time of about 30 seconds to about 300 seconds.
103. The composition of any one of embodiments 1-101, wherein the tablet has a disintegration time of about 30 seconds to about 200 seconds.
104. The composition of any one of embodiments 1-101, wherein the tablet has a disintegration time of about 30 seconds to about 150 seconds.
105. The composition of any one of embodiments 1-101 wherein the tablet has a disintegration time of about 30 seconds, about 40 seconds, about 50 seconds, about 60 seconds, about 70 seconds, about 80 seconds, about 90 seconds, about 100 seconds, about 110 seconds, about 120 seconds, about 130 seconds, about 140 seconds, about 150 seconds, about 160 seconds, about 170 seconds, about 180 seconds, about 190 seconds, about 200 seconds, about 210 seconds, about 220 seconds, about 230 seconds, about 240 seconds, about 250 seconds, about 260 seconds, about 270 seconds, about 280 seconds, about 290 seconds, or about 300 seconds.
106. The composition of any one of embodiments 1-105, wherein the composition comprises less than 10% by weight of water.
107. The composition of any one of embodiments 1-106, wherein the composition comprises less than 10% by weight of water after storage for 1 month at 40° C. and 75% relative humidity (RH).
108. The composition of any one of embodiments 1-107, wherein the composition comprises less than 10% by weight of water after storage for 2 months at 40° C. and 75% relative humidity (RH).
109. A method of making a composition comprising a tablet from wet granulation comprising niraparib comprising:
   (a) forming an intragranular phase comprising
      i) combining niraparib, lactose monohydrate, and microcrystalline cellulose to form a composition comprising niraparib, lactose monohydrate, and microcrystalline cellulose; and
      ii) wet granulating the composition comprising niraparib, lactose monohydrate, and microcrystalline cellulose to form granules;
   (b) forming an extragranular phase comprising
      iii) combining the granules with at least one pharmaceutically acceptable excipient to form a mixture; and
   (c) forming a tablet by compressing the mixture obtained from step iii).
110. The method of embodiment 109, wherein the wet granulating from step ii) further comprises adding a binder.
111. The method of embodiment 110, wherein the binder is a liquid binder.
112. The method of embodiment 111, wherein the liquid binder is dissolved povidone.
113. The method of embodiment 111, wherein the liquid binder is dissolved starch, dissolved hydroxypropyl cellulose (HPC), dissolved hydroxypropyl methylcellulose (HPMC), or liquid polyethylene glycol (PEG).
114. The method of embodiment 111, wherein the liquid binder is a melted binder.
115. The method of embodiment 114, wherein the melted binder is a hydrophilic polyethylene glycol (PEG), poloxamer, hydrophobic fatty acid, fatty alcohol, wax, hydrogenated vegetable oil, or glyceride.
116. The method of embodiment 110, wherein the binder is a dry binder.
117. The method of embodiment 116, wherein the dry binder is hydroxypropyl cellulose (HPC).
118. The method of embodiment 116, wherein the dry binder is hydroxypropyl methylcellulose (HPMC).
119. The method of embodiment 116, wherein the dry binder is povidone (PVP) or starch.
120. The method of any one of embodiments 109-119, wherein the wet granulating from step ii) further comprises wet-sieving.
121. The method of any one of embodiments 109-120, wherein the wet granulating from step ii) further comprises drying and dry sieving.
122. A method of making a composition comprising a tablet from moisture-activated dry granulation comprising niraparib comprising:
   (a) forming an intragranular phase comprising
      i) combining niraparib, lactose monohydrate, and microcrystalline cellulose to form a composition comprising niraparib, lactose monohydrate, and microcrystalline cellulose; and
      ii) granulating the composition comprising niraparib, lactose monohydrate, and microcrystalline cellulose to form granules;
   (b) forming an extragranular phase comprising
      iii) combining the granules with at least one pharmaceutically acceptable excipient to form a mixture; and
   (c) forming a tablet by compressing the mixture obtained from step iii).
123. The method of embodiment 122, wherein the granulating from step ii) further comprises adding a binder.
124. The method of embodiment 123, wherein the binder is a liquid binder.
125. The method of embodiment 124, wherein the liquid binder is dissolved povidone.
126. The method of embodiment 124, wherein the liquid binder is water, dissolved starch, dissolved hydroxypropyl cellulose (HPC), dissolved hydroxypropyl methylcellulose (HPMC), or liquid polyethylene glycol (PEG).
127. The method of embodiment 122, wherein the composition further comprises a dry binder.
128. The method of embodiment 127, wherein water is added to the composition comprising the dry binder.
129. The method of any one of embodiments 122-128, wherein the granulating from step ii) further comprises drying and dry sieving.
130. The method of embodiment 129, wherein drying comprises the addition of a glidant.
131. The method of embodiment 130, wherein the glidant is silicon dioxide.
132. The method of embodiment 130, wherein the glidant is silicon dioxide, tribasic calcium phosphate, calcium silicate, cellulose, magnesium silicate, magnesium trisilicate, starch, talc, or mixtures thereof
133. A method of making a composition comprising a tablet from dry granulation comprising niraparib comprising:
   (a) forming an intragranular phase comprising
      i) combining niraparib, a diluent selected from mannitol and calcium phosphate dibasic, microcrystalline cellulose, and magnesium stearate to form a composition comprising niraparib, the diluent selected from mannitol and calcium phosphate dibasic, microcrystalline cellulose, and magnesium stearate; and
      ii) dry granulating the composition comprising niraparib, the diluent selected from mannitol and calcium phosphate dibasic, microcrystalline cellulose, and magnesium stearate to form granules;
   (b) forming an extragranular phase comprising
      iii) combining the granules with at least one pharmaceutically acceptable excipient to form a mixture; and
   (c) forming a tablet by compressing the mixture obtained from step iii).
134. The method of embodiment 133, wherein the composition further comprises a dry binder.
135. The method of embodiment 134, wherein water is added to the composition comprising the dry binder.
136. The method of any one of embodiments 133-135, wherein combining niraparib, a diluent selected from mannitol and calcium phosphate dibasic, microcrystalline cellulose, and magnesium stearate to form a composition comprising niraparib, the diluent selected from mannitol and calcium phosphate dibasic, microcrystalline cellulose, and magnesium stearate from step i) further comprises blending the niraparib, a diluent selected from mannitol and calcium phosphate dibasic, microcrystalline cellulose, and magnesium stearate.
137. The method of any one of embodiments 133-136, wherein dry granulating from step ii) comprises slugging and milling.
138. The method of any one of embodiments 133-136, wherein the ribbon thickness is from about 0.1 mm to about 2 mm.
139. The method of any one of embodiments 109-138, wherein the composition from step i) further comprises silicon dioxide.
140. The method of any one of embodiments 109-139, wherein the at least one pharmaceutically acceptable excipient for combining the granules with at least one pharmaceutically acceptable excipient to form a mixture from step iii) is silicon dioxide.
141. The method of any one of embodiments 109-140, wherein the at least one pharmaceutically acceptable excipient for combining the granules with at least one pharmaceutically acceptable excipient to form a mixture from step iii) is magnesium stearate.
142. The method of any one of embodiments 109-141, wherein combining the granules with at least one pharmaceutically acceptable excipient to form a mixture from step iii) comprises blending the granules with at least one pharmaceutically acceptable excipient.
143. The method of any one of embodiments 109-142, wherein the composition from step i) is a blend composition.
144. The method of any one of embodiments 109-143, wherein the amount of components used to form the intragranular phase is about 50% to about 98% by weight of the tablet composition.
145. The method of any one of embodiments 109-143, wherein the amount of components used to form the intragranular phase is about 85% to about 98% by weight of the tablet composition.
146. The method of any one of embodiments 109-143, wherein the amount of components used to form the intragranular phase is about 90% to about 98% by weight of the tablet composition.
147. The method of any one of embodiments 109-143, wherein the amount of components used to form the intragranular phase is about 92.5% to about 97.5% by weight of the tablet composition.
148. The method of any one of embodiments 109-143, wherein the amount of components used to form the intragranular phase is about 95% by weight of the tablet composition.
149. The method of any one of embodiments 109-148, wherein the amount of components used to form the extragranular phase is about 2% to about 50% by weight of the tablet composition.
150. The method of any one of embodiments 109-148, wherein the amount of components used to form the extragranular phase is about 2% to about 15% by weight of the tablet composition.
151. The method of any one of embodiments 109-148, wherein the amount of components used to form the extragranular phase is about 2% to about 10% by weight of the tablet composition.
152. The method of any one of embodiments 109-148, wherein the amount of components used to form the extragranular phase is about 2.5% to about 7.5% by weight of the tablet composition.
153. The method of any one of embodiments 109-148, wherein the amount of components used to form the extragranular phase is about 5% by weight of the tablet composition.
154. The method of any one of embodiments 109-153, wherein the granules have a bulk density of about 0.2 to about 0.7 g/cm³.
155. The method of any one of embodiments 109-154, wherein the granules have a tapped density of about 0.3 to about 0.9 g/cm³.
156. A method of making a composition comprising a tablet comprising niraparib comprising:
   (a) forming an intragranular phase comprising
      i) combining niraparib and at least one pharmaceutically acceptable excipient to form a composition comprising niraparib and at least one pharmaceutically acceptable excipient; and
      ii) granulating the composition comprising niraparib and at least one pharmaceutically acceptable excipient to form granules;
   (b) forming an extragranular phase comprising
      iii) combining the granules with at least one pharmaceutically acceptable excipient to form a mixture; and
   (c) forming a tablet by compressing the mixture obtained from step iii);
      wherein the tablet has at least one of the following:
   (1) the amount of components used to form the intragranular phase is about 50% to about 98% by weight of the tablet composition; and
   (2) the amount of components used to form the extragranular phase is about 2% to about 50% by weight of the tablet composition.
157. The method of embodiment 156, wherein the amount of components used to form the intragranular phase is about 50% to about 98% by weight of the tablet composition.
158. The method of embodiment 156, wherein the amount of components used to form the intragranular phase is about 85% to about 98% by weight of the tablet composition.
159. The method of embodiment 156, wherein the amount of components used to form the intragranular phase is about 90% to about 98% by weight of the tablet composition.
160. The method of embodiment 156, wherein the amount of components used to form the intragranular phase is about 92.5% to about 97.5% by weight of the tablet composition.
161. The method of embodiment 156, wherein the amount of components used to form the intragranular phase is about 95% by weight of the tablet composition.
162. The method of any one of embodiments 156-161, wherein the amount of components used to form the extragranular phase is about 2% to about 50% by weight of the tablet composition.
163. The method of any one of embodiments 156-161, wherein the amount of components used to form the extragranular phase is about 2% to about 15% by weight of the tablet composition.
164. The method of any one of embodiments 156-161, wherein the amount of components used to form the extragranular phase is about 2% to about 10% by weight of the tablet composition.
165. The method of any one of embodiments 156-161, wherein the amount of components used to form the extragranular phase is about 2.5% to about 7.5% by weight of the tablet composition.
166. The method of any one of embodiments 156-161, wherein the amount of components used to form the extragranular phase is about 5% by weight of the tablet composition.
167. The method of any one of embodiments 156-166, wherein the at least one pharmaceutically acceptable excipient from step i) is microcrystalline cellulose.
168. The method of any one of embodiments 156-167, wherein the at least one pharmaceutically acceptable excipient from step i) is lactose monohydrate, lactose anhydrous, mannitol, or calcium phosphate dibasic.
169. The method of any one of embodiments 156-168, wherein the at least one pharmaceutically acceptable excipient from step i) is magnesium stearate.
170. The method of any one of embodiments 156-169, wherein the at least one pharmaceutically acceptable excipient from step i) is silicon dioxide
171. The method of any one of embodiments 156-170, wherein the granulating from step ii) is wet granulating.
172. The method of embodiment 171, wherein the wet granulating further comprises adding a binder.
173. The method of embodiment 172, wherein the binder is a liquid binder.
174. The method of embodiment 173, wherein the liquid binder is dissolved povidone.
175. The method of embodiment 173, wherein the liquid binder is dissolved starch, dissolved hydroxypropyl cellulose (HPC), dissolved hydroxypropyl methylcellulose (HPMC), or liquid polyethylene glycol (PEG).
176. The method of embodiment 173, wherein the liquid binder is a melted binder.
177. The method of embodiment 176, wherein the melted binder is a hydrophilic polyethylene glycol (PEG), poloxamer, hydrophobic fatty acid, fatty alcohol, wax, hydrogenated vegetable oil, or glyceride.
178. The method of embodiment 172, wherein the binder is a dry binder.
179. The method of embodiment 178, wherein the dry binder is hydroxypropyl cellulose (HPC).
180. The method of embodiment 178, wherein the dry binder is hydroxypropyl methylcellulose (HPMC).
181. The method of embodiment 178, wherein the dry binder is povidone (PVP) or starch.
182. The method of any one of embodiments 171-181, wherein the wet-granulating from step ii) further comprises wet-sieving.
183. The method of any one of embodiments 171-182, wherein the wet granulating from step ii) further comprises drying and dry sieving.
184. The method of embodiment 183, wherein drying comprises the addition of a glidant.
185. The method of any one of embodiments 156-170, wherein the granulating from step ii) is dry granulating.
186. The method of embodiment 185, wherein the dry granulating comprises slugging and milling.
187. The method of any one of embodiments 156-186, wherein the at least one pharmaceutically acceptable excipient for combining the granules with at least one pharmaceutically acceptable excipient to form a mixture from step iii) is silicon dioxide.
188. The method of any one of embodiments 156-187, wherein the at least one pharmaceutically acceptable excipient for combining the granules with at least one pharmaceutically acceptable excipient to form a mixture from step iii) is magnesium stearate.
189. A method of treating cancer, comprising administering to a subject in need thereof an effective amount of a composition according to any one of embodiments 1-108.
190. The method of embodiment 189, wherein the cancer is selected from the group consisting of ovarian cancer, breast cancer, cervical cancer, endometrial cancer, prostate cancer, testicular cancer, pancreatic cancer, esophageal cancer, head and neck cancer, gastric cancer, bladder cancer, lung cancer, bone cancer, colon cancer, rectal cancer, thyroid cancer, brain and central nervous system cancers, glioblastoma, neuroblastoma, neuroendocrine cancer, rhabdoid cancer, keratoacanthoma, epidermoid carcinoma, seminoma, melanoma, sarcoma, bladder cancer, liver cancer, kidney cancer, myeloma, lymphoma, and combinations thereof.
191. The method of embodiments 189 or 190, wherein the cancer is selected from the group consisting of ovarian cancer, fallopian tube cancer, primary peritoneal cancer, and combinations thereof.
192. The method of embodiment 189, wherein said subject is a pediatric subject.
193. A method of treating cancer, comprising administering to a pediatric subject in need thereof an effective amount of niraparib.
194. The method of embodiment 192 or 193, wherein said cancer is characterized by a homologous recombination repair (HRR) gene deletion.
195. The method of any one of embodiments 192-194, wherein said cancer is characterized by a mutation in the DNA damage repair (DDR) pathway.
196. The method of any one of embodiments 192-195, wherein said cancer is characterized by homologous recombination deficiency (HRD).
197. The method of any one of embodiments 192-196, wherein said cancer is characterized by BRCA deficiency.
198. The method of any one of embodiments 192-197, wherein said cancer is characterized by an isocitrate dehydrogenase (IDH) mutation.
199. The method of any one of embodiments 192-198, wherein said cancer is characterized by a chromosomal translocation.
200. The method of any one of embodiments 192-199, wherein said cancer is a hypermutant cancer.
201. The method of any one of embodiments 192-200, wherein said cancer is a MSI-H or a MSI-L cancer.
202. The method of any one of embodiments 192-200, wherein said cancer is a MSS cancer.
203. The method of any one of embodiments 192-202, wherein said cancer is a non-CNS cancer.
204. The method of embodiment 203, wherein said cancer is a solid tumor.
205. The method of embodiment 203 or 204, wherein said cancer is neuroblastoma, hepatoblastoma, hepatocellular carcinoma, Wilms tumor, renal cell carcinoma, melanoma, adrenocortical carcinoma, adenocarcinoma of the colon, myoepithelial carcinoma, thymic cell carcinoma, nasopharyngeal carcinoma, squamous cell carcinoma, mesothelioma, clivus chordoma.
206. The method of embodiment 205, wherein said cancer is extracranial embryonal neuroblastoma.
207. The method of any one of embodiments 192-202, wherein said cancer is a CNS cancer.
208. The method of embodiment 207, wherein said cancer is a primary CNS malignancy.
209. The method of embodiment 207, wherein said cancer is ependymoma.
210. The method of embodiment 207, wherein said cancer is a brain cancer.
211. The method of embodiment 210, wherein said cancer is glioblastoma multiforme, gliosarcoma, astrocytoma, glioblastoma, medulloblastoma, glioma, supratentorial primitive neuroectodermal tumor, atypical teratoid rhabdoid tumor, choroid plexus carcinoma, malignant ganglioma, gliomatosis cerebri, meningioma, or paraganglioma.
212. The method of embodiment 211, wherein said cancer is high-grade astrocytoma, low-grade astrocytoma, anaplastic astrocytoma, fibrillary astrocytoma, or pilocytic astrocytoma.
213. The method of embodiment 211, wherein said cancer is a high-grade glioma, low-grade glioma, diffuse intrinsic pontine glioma (DIPG), or anaplastic mixed glioma.
214. The method of any one of embodiments 192-202, wherein said cancer is a carcinoma.
215. The method of any one of embodiments 192-202, wherein said cancer is a gonadal tumor.
216. The method of any one of embodiments 192-202, wherein said cancer is a hematological cancer.
217. The method of embodiment 216, wherein said cancer is lymphoma.
218. The method of embodiment 217, wherein said cancer is Hodgkin's lymphoma (e.g., relapsed or refractory classic Hodgkin's Lymphoma (cHL)), non-Hodgkin's lymphoma, diffuse large B-cell lymphoma, precursor T-lymphoblastic lymphoma, lymphoepithelial carcinoma, or malignant histiocytosis.
219. The method of any one of embodiments 192-202, wherein said cancer is a sarcoma.
220. The method of embodiment 219, wherein said cancer is Ewings sarcoma, osteosarcoma, rhabdomyosarcoma, embryonal rhabdomyosarcoma, synovial sarcoma, alveolar rhabdomyosarcoma, alveolar soft part sarcoma, spindle cell sarcoma, angiosarcoma, epithelialoid sarcoma, inflammatory myofibroblastic tumor, malignant rhadoid tumor
221. The method of any one of embodiments 192-202, wherein said cancer is Ewing's sarcoma, osteosarcoma, ERS, a CNS tumor, or neuroblastoma.
222. The method of any one of embodiments 192-221, wherein said cancer is recurrent.
223. The method of any one of embodiments 192-222, wherein said subject has not received at least one other line of treatment (LOT).
224. The method of any one of embodiments 192-222, wherein said subject has previously received at least one line of treatment (LOT).
225. The method of embodiment 224, wherein said at least one line of treatment is not an immunotherapy treatment
226. The method of embodiment 224 or 225, wherein said cancer is refractory to a previous line of treatment (LOT).
227. The method of any one of embodiments 192-226, wherein the pediatric patient is of about six months to about 18 years of age, about one year to about six years of age, or about six years to about 18 years of age.
228. The method of any one of embodiments 192-227, wherein the administered amount of niraparib is determined by said subject's weight.
229. The method of any one of embodiments 192-227, wherein the administered amount of niraparib is determined by said subject's body surface area (B S A).
230. The method of embodiment 229, wherein the administered amount of niraparib is about 25 mg/m² to about 300 mg/m², about 25 mg/m² to about 275 mg/m², about 25 mg/m² to about 250 mg/m², about 25 mg/m² to about 200 mg/m², about 50 mg/m² to about 300 mg/m², about 50 mg/m² to about 275 mg/m², about 50 mg/m² to about 250 mg/m², about 50 mg/m² to about 200 mg/m², about 75 mg/m² to about 300 mg/m², about 75 mg/m² to about 275 mg/m², about 75 mg/m² to about 250 mg/m², about 75 mg/m² to about 200 mg/m², about 100 mg/m² to about 300 mg/m², about 100 mg/m² to about 275 mg/m², about 100 mg/m² to about 250 mg/m², about 100 mg/m² to about 200 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², or about 200 mg/m².
231. The method of any one of embodiments 192-227, wherein the administered amount of niraparib is a flat dose.
232. The method of any one of embodiments 192-231, wherein niraparib is orally administered once daily.
233. The method of any one of embodiments 192-231, wherein niraparib is orally administered once every two days, once every three days, once every four days, once every five days, once every six days, or once every seven days.
234. The method of any one of embodiments 192-233, wherein niraparib is orally administered in an amount that is about 25 mg to about 300 mg or about 25 mg to about 500 mg.
235. The method of embodiment 234, wherein said niraparib is orally administered in an amount that is:
   about 25 mg, about 50 mg, about 75 mg, about 100 mg, about 125 mg, about 150 mg, about 175 mg, or about 200 mg;
   about 75 mg, about 100 mg, about 130 mg, or about 160 mg;
   about 150 mg, about 200 mg, about 260 mg, or about 320 mg; or
   about 225 mg, about 300 mg, about 390 mg, or about 480 mg.
236. The method of any one of embodiments 192-235, wherein two different amounts of niraparib are administered to the subject on alternating days on which dosages are administered to said subject.
237. The method of any one of embodiments 192-236, wherein said niraparib is administered as a unit dose form that is a tablet comprising about 50 mg niraparib.
238. The method of any one of embodiments 192-237, wherein the method further comprises administering another therapeutic agent or treatment.
239. The method of embodiment 238, wherein the method further comprises administering one or more of surgery, a radiotherapy, a chemotherapy, an immunotherapy, an anti-angiogenic agent, or an anti-inflammatory.
240. The method of embodiment 238 or 239, wherein the subject has been further administered or will be administered an immune checkpoint inhibitor.
241. The method of embodiment 240, wherein the immune checkpoint inhibitor is selected from an inhibitor of PD-1, LAG-3, CTLA-4, TIM-3, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R.
242. The method of embodiment 241, wherein the immune checkpoint inhibitor is an agent that inhibits PD-1, LAG-3, TIM-3, CTLA-4, TIGIT, IDO, or CSF1R.
243. The method of embodiment 242, wherein the immune checkpoint inhibitor is an agent that inhibits PD-1.
244. The method of embodiment 243, wherein the PD-1 inhibitor is a small molecule, a nucleic acid, a polypeptide, a carbohydrate, a lipid, a metal, a toxin, or a PD-1 binding agent.
245. The method of embodiment 243, wherein the PD-1 inhibitor is a PD-L1/L2 binding agent.
246. The method of embodiment 245, wherein the PD-L1/L2 binding agent is an antibody, an antibody conjugate, or an antigen-binding fragment thereof
247. The method of embodiment 246, wherein the PD-L1/L2 binding agent durvalumab, atezolizumab, avelumab, BGB-A333, SHR-1316, FAZ-053, CK-301, or, PD-L1 millamolecule, or derivatives thereof.
248. The method of embodiment 243 or 244, wherein the PD-1 inhibitor is a PD-1 binding agent.
249. The method of embodiment 248, wherein the PD-1 binding agent is an antibody, an antibody conjugate, or an antigen-binding fragment thereof
250. The method of embodiment 249, wherein the PD-1 inhibitor is nivolumab, pembrolizumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, AMP-514/MEDI-0680, AGEN-2034, CS1001, TSR-042, Sym-021, PF-06801591, LZMO09, KN-035, AB122, genolimzumab (CBT-501), AK 104, or GLS-010, or derivatives thereof
251. The method of embodiment 250, wherein the PD-1 inhibitor is TSR-042.
252. The method of any one of embodiments 243-251, wherein the PD-1 inhibitor is administered to the subject periodically at a dose of about 50 mg to about 2000 mg, about 50 mg to about 1000 mg, or about 100 mg to about 500 mg.
253. The method of embodiment 252, wherein the PD-1 inhibitor is administered to the subject periodically at a dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, or about 1700 mg.
254. The method of embodiment 252 or 253, wherein the PD-1 inhibitor is administered periodically to the subject at an administration interval that is once every week, once every two weeks, once every three weeks, once every four weeks, once every five weeks, once every six weeks, once every seven weeks, once every eight weeks, once every nine weeks, or once every ten weeks.
255. The method of embodiment 252 or 253, wherein the PD-1 inhibitor is administered as a first dose once every 3 weeks for 3, 4, or 5 cycles followed by a second dose administered once every six weeks.
256. The method of embodiment 255, wherein the first dose is about 500 mg of the PD-1 inhibitor.
257. The method of embodiment 255 or 256, wherein the second dose is about 1000 mg of the PD-1 inhibitor.
258. The method of any one of embodiments 192-257, wherein niraparib is administered with food.

### Second Set of Embodiments

1. A method of making a formulation comprising niraparib comprising:
   (a) obtaining niraparib;
   (b) obtaining lactose monohydrate that has been screened with a screen;
   (c) combining the niraparib with the screened lactose monohydrate to form a composition comprising niraparib and lactose monohydrate;
   (d) blending the composition comprising niraparib and lactose monohydrate;
   (e) combining the blended composition comprising niraparib and lactose monohydrate with magnesium stearate to form a composition comprising niraparib, lactose monohydrate and magnesium stearate; and
   (f) blending the composition comprising niraparib, lactose monohydrate and magnesium stearate.
2. The method of embodiment 1, wherein obtaining niraparib comprises obtaining niraparib that has been screened.
3. The method of embodiment 1, wherein combining the niraparib with the screened lactose monohydrate comprises combining unscreened niraparib with the screened lactose monohydrate.
4. A method of making a formulation comprising niraparib comprising:
   (a) obtaining niraparib, wherein the niraparib is optionally niraparib that has been screened;
   (b) obtaining lactose monohydrate that has been screened with a screen;
   (c) combining the screened niraparib with the screened lactose monohydrate to form a composition comprising niraparib and lactose monohydrate;
   (d) blending the composition comprising niraparib and lactose monohydrate;
   (e) combining the blended composition comprising niraparib and lactose monohydrate with magnesium stearate to form a composition comprising niraparib, lactose monohydrate and magnesium stearate; and
   (f) blending the composition comprising niraparib, lactose monohydrate and magnesium stearate.
5. The method of embodiment 4, wherein obtaining niraparib comprises obtaining niraparib that has been screened.
6. The method of embodiment 5, wherein obtaining niraparib that has been screened comprises obtaining niraparib that has been screened with a screen having a mesh size of greater than 425 microns.
7. The method of embodiment 6, wherein obtaining niraparib that has been screened with a screen having a mesh size of greater than 425 microns comprises obtaining niraparib that has been screened with a screen having a mesh size of about 850 microns or about 1180 microns.
8. The method of any one of embodiments 1-7, wherein obtaining lactose monohydrate that has been screened with a screen comprises obtaining screened lactose monohydrate that has been screened with a screen having a mesh size of at most about 600 microns.
9. The method of embodiment 8, wherein over 50% of the screened lactose monohydrate is present as particles with a diameter of between 53 microns and 500 microns.
10. The method of any one of embodiments 1-9, wherein the magnesium stearate is magnesium stearate screened with a screen having a mesh size of greater than 250 microns.
11. The method of embodiment 10, wherein the magnesium stearate is magnesium stearate screened with a screen having a mesh size of about 600 microns.
12. The method of any one of embodiments 1-11, wherein the method further comprises screening the blended composition comprising niraparib and lactose monohydrate before combining the blended composition comprising niraparib and lactose monohydrate with magnesium stearate.
13. The method of embodiment 12, wherein the blended composition comprising niraparib and lactose monohydrate is screened with a screen having a mesh size of about 600 microns.
14. A method of making a formulation comprising niraparib comprising:
   (a) obtaining niraparib, wherein the niraparib is optionally niraparib that has been screened with a screen having a mesh size of greater than 425 microns;
   (b) combining the niraparib with lactose monohydrate to form a composition comprising niraparib and lactose monohydrate;
   (c) blending the composition comprising niraparib and lactose monohydrate;
   (d) combining the blended composition comprising niraparib and lactose monohydrate with magnesium stearate to form a composition comprising niraparib, lactose monohydrate and magnesium stearate; and
   (e) blending the composition comprising niraparib, lactose monohydrate and magnesium stearate.
15. The method of embodiment 14, wherein the lactose monohydrate has been screened before combining the screened niraparib with the lactose monohydrate to form a composition comprising niraparib and lactose monohydrate.
16. The method of embodiment 15, wherein the lactose monohydrate that has been screened has been screened with a screen having a mesh size of at most about 600 microns.
17. The method of embodiment 15 or 16, wherein over 50% of the screened lactose monohydrate is present as particles with a diameter of between 53 microns and 500 microns.
18. The method of any one of embodiments 14-17, wherein obtaining niraparib that has been screened with a screen having a mesh size of greater than 425 microns comprises obtaining niraparib that has been screened with a screen having a mesh size of about 850 microns or about 1180 microns.
19. The method of any one of embodiments 14-18, wherein the magnesium stearate is magnesium stearate screened with a screen having a mesh size of greater than 250 microns.
20. The method of embodiment 19, wherein the magnesium stearate is magnesium stearate screened with a screen having a mesh size of about 600 microns.
21. The method of any one of embodiments 14-20, wherein the method further comprises screening the blended composition comprising niraparib and lactose monohydrate before combining the blended composition comprising niraparib and lactose monohydrate with magnesium stearate.
22. The method of embodiment 21, wherein the blended composition comprising niraparib and lactose monohydrate is screened with a screen having a mesh size of about 600 microns.
23. A method of making a formulation comprising niraparib comprising:
   (a) obtaining niraparib, wherein optionally niraparib is niraparib that has been screened;
   (b) combining the niraparib with lactose monohydrate to form a composition comprising niraparib and lactose monohydrate,
   (c) blending the composition comprising niraparib and lactose monohydrate,
   (d) combining the blended composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than 250 microns, and
   (e) blending the composition comprising niraparib, lactose monohydrate and magnesium stearate.
24. The method of embodiment 23, wherein the magnesium stearate is magnesium stearate screened with a screen having a mesh size of about 600 microns.
25. The method of embodiment 23 or 24, wherein the lactose monohydrate has been screened before combining the screened niraparib with the lactose monohydrate to form a composition comprising niraparib and lactose monohydrate.
26. The method of embodiment 25, wherein the lactose monohydrate has been screened with a screen having a mesh size of at most about 600 microns.
27. The method of embodiment 25 or 26, wherein over 50% of the screened lactose monohydrate is present as particles with a diameter of between 53 microns and 500 microns.
28. The method of any one of embodiments 23-27, wherein obtaining niraparib that has been screened comprises obtaining niraparib that has been screened with a screen having a mesh size of greater than 425 microns.
29. The method of embodiment 28, wherein obtaining niraparib that has been screened with a screen having a mesh size of greater than 425 microns comprises obtaining niraparib that has been screened with a screen having a mesh size of about 850 microns or about 1180 microns.
30. The method of any one of embodiments 23-29, wherein the method further comprises screening the blended composition comprising niraparib and lactose monohydrate before combining the blended composition comprising niraparib and lactose monohydrate with magnesium stearate.
31. The method of embodiment 30, wherein the blended composition comprising niraparib and lactose monohydrate is screened with a screen having a mesh size of about 600 microns.
32. A method of making a formulation comprising niraparib comprising:
   (a) obtaining niraparib, wherein optionally niraparib is niraparib that has been screened;
   (b) combining the niraparib with lactose monohydrate to form a composition comprising niraparib and lactose monohydrate;
   (c) blending the composition comprising niraparib and lactose monohydrate;
   (d) screening the blended composition comprising niraparib and lactose monohydrate;
   (e) combining the screened composition comprising niraparib and lactose monohydrate with magnesium stearate to form a composition comprising niraparib, lactose monohydrate and magnesium stearate; and
   (f) blending the composition comprising niraparib, lactose monohydrate and magnesium stearate.
33. The method of embodiment 32, wherein the blended composition comprising niraparib and lactose monohydrate is screened with a screen having a mesh size of about 600 microns.
34. The method of embodiment 32 or 33, wherein the lactose monohydrate has been screened before combining the screened niraparib with the lactose monohydrate to form a composition comprising niraparib and lactose monohydrate.
35. The method of embodiment 34, wherein the lactose monohydrate has been screened with a screen having a mesh size of at most about 600 microns.
36. The method of embodiment 34 or 35, wherein over 50% of the screened lactose monohydrate is present as particles with a diameter of between 53 microns and 500 microns.
37. The method of any one of embodiments 32-36, wherein obtaining niraparib that has been screened comprises obtaining niraparib that has been screened with a screen having a mesh size of greater than 425 microns.
38. The method of embodiment 37, wherein obtaining niraparib that has been screened with a screen having a mesh size of greater than 425 microns comprises obtaining niraparib that has been screened with a screen having a mesh size of about 850 microns or about 1180 microns.
39. The method of any one of embodiments 32-38, wherein the magnesium stearate is magnesium stearate screened with a screen having a mesh size of greater than 250 microns.
40. The method of embodiment 39, wherein the magnesium stearate is magnesium stearate screened with a screen having a mesh size of about 600 microns.
41. The method of any one of embodiments 1-40, wherein the screened niraparib has been annealed one or more times.
42. A method of making a formulation comprising niraparib comprising:
   (a) obtaining niraparib, wherein optionally niraparib is niraparib that has been screened, wherein the niraparib has been annealed two or more times;
   (b) combining the niraparib with lactose monohydrate to form a composition comprising niraparib and lactose monohydrate;
   (c) blending the composition comprising niraparib and lactose monohydrate;
   (d) combining the blended composition comprising niraparib and lactose monohydrate with magnesium stearate to form a composition comprising niraparib, lactose monohydrate and magnesium stearate; and
   (e) blending the composition comprising niraparib, lactose monohydrate and magnesium stearate.
43. The method of embodiment 42, wherein the blended composition comprising niraparib and lactose monohydrate is screened with a screen having a mesh size of about 600 microns.
44. The method of embodiment 42 or 43, wherein the lactose monohydrate has been screened before combining the screened niraparib with the lactose monohydrate to form a composition comprising niraparib and lactose monohydrate.
45. The method of embodiment 44, wherein the lactose monohydrate has been screened with a screen having a mesh size of at most about 600 microns.
46. The method of embodiment 44 or 45, wherein over 50% of the screened lactose monohydrate is present as particles with a diameter of between 53 microns and 500 microns.
47. The method of any one of embodiments 42-46, wherein obtaining niraparib that has been screened comprises obtaining niraparib that has been screened with a screen having a mesh size of greater than 425 microns.
48. The method of embodiment 47, wherein obtaining niraparib that has been screened with a screen having a mesh size of greater than 425 microns comprises obtaining niraparib that has been screened with a screen having a mesh size of 850 microns or about 1180 microns.
49. The method of any one of embodiments 42-48, wherein the magnesium stearate is magnesium stearate screened with a screen having a mesh size of greater than 250 microns.
50. The method of embodiment 49, wherein the magnesium stearate is magnesium stearate screened with a screen having a mesh size of about 600 microns.
51. The method of any one of embodiments 42-50, wherein the method further comprises screening the blended composition comprising niraparib and lactose monohydrate before combining the blended composition comprising niraparib and lactose monohydrate with magnesium stearate.
52. The method of embodiment 51, wherein the blended composition comprising niraparib and lactose monohydrate is screened with a screen having a mesh size of about 600 microns.
53. A method of making a formulation comprising niraparib comprising:
   (a) obtaining niraparib that has been screened with a screen having a mesh size of greater than 425 microns;
   (b) obtaining lactose monohydrate that has been screened with a screen;
   (c) combining the screened niraparib with lactose monohydrate to form a composition comprising niraparib and lactose monohydrate;
   (d) blending the composition comprising niraparib and lactose monohydrate;
   (e) screening the blended composition comprising niraparib and lactose monohydrate;
   (f) combining the screened composition comprising niraparib and lactose monohydrate with magnesium stearate to form a composition comprising niraparib, lactose monohydrate and magnesium stearate, wherein the magnesium stearate is magnesium stearate screened with a screen having a mesh size of greater than 250 microns; and
   (g) blending the composition comprising niraparib, lactose monohydrate and magnesium stearate.
54. The method of embodiment 53, wherein the niraparib has been annealed one or more times.
55. The method of any one of embodiments 1-54, wherein the niraparib has been milled.
56. The method of embodiment 55, wherein the niraparib has been wet milled.
57. The method of any one of embodiments 1-56, wherein the niraparib is screened, wherein the screening may be delumping or other such powder handling manually or mechanically.
58. The method of any one of embodiments 1-57, wherein the method further comprises encapsulating the blended the composition comprising niraparib, lactose monohydrate and magnesium stearate into one or more capsules.
59. The method of embodiment 58, wherein the one or more capsules are gelatin capsules.
60. The method of embodiment 58 or 59, wherein the encapsulating comprises using an encapsulator.
61. The method of any one of embodiments 58-60, wherein the encapsulating comprises encapsulating at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 100,000, 150,000, 200,000, 300,000, 400,000, or 500,000 of the one or more capsules.
62. The method of any one of embodiments 58-61, wherein the encapsulating comprises encapsulating at a rate of at least about 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 75,000, 100,000, 150,000 or 200,000 of the one or more capsules/hour.
63. The method of any one of embodiments 58-62, wherein the encapsulating comprises encapsulating the one or more capsules from a batch comprising the composition comprising niraparib, lactose monohydrate and magnesium stearate that is in the encapsulator.
64. The method of embodiment 63, wherein a portion of the volume of the batch in the encapsulator is used to encapsulate the one or more capsules.
65. The method of embodiment 64, the portion of the volume of the batch in the encapsulator used to encapsulate the one or more capsules is less than 100%, 99%, 98%, 97%, 96%, 95%, 90%, 85%, 80%, or 75% of a total initial volume of the batch.
66. The method of any one of embodiments 58-65, wherein one or more parts of the encapsulator are coated with a coating.
67. The method of embodiment 66, wherein the one or more coated parts comprises a tamping pin, a dosing disc, or both.
68. The method of embodiment 66 or 67, wherein the coating comprises nickel, chrome, or a combination thereof.
69. The method of any one of embodiments 58-68, wherein the encapsulating comprises automatic encapsulation.
70. The method of any one of embodiments 58-69, wherein adherence of the composition to one or more encapsulating components is reduced or prevented.
71. The method of any one of embodiments 58-70, wherein jamming of the encapsulator is reduced or prevented.
72. The method of any one of embodiments 1-71, wherein blending the composition comprising niraparib and lactose monohydrate comprises blending for about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.
73. The method of any one of embodiments 1-72, wherein blending the composition comprising niraparib, lactose monohydrate and magnesium stearate comprises blending for about 5 revolutions, 10 revolutions, 15 revolutions, 20 revolutions, 25 revolutions, 30 revolutions, 35 revolutions, 40 revolutions, 45 revolutions, 50 revolutions, 55 revolutions, 60 revolutions, 65 revolutions, 70 revolutions, 75 revolutions, 80 revolutions, 85 revolutions, 90 revolutions, 95 revolutions, 100 revolutions, 125 revolutions, 150 revolutions, 175 revolutions, 200 revolutions, 225 revolutions, 250 revolutions, 275 revolutions, 300 revolutions, 325 revolutions, 350 revolutions, 375 revolutions, 400 revolutions, 425 revolutions, 450 revolutions, 475 revolutions, 500 revolutions, 550 revolutions, 600 revolutions, 650 revolutions, 700 revolutions, 750 revolutions, 800 revolutions, 850 revolutions, 900 revolutions, 950 revolutions, or 1000 revolutions.
74. The method of any one of embodiments 1-73, wherein the blending comprises using a blender, and wherein the niraparib is distributed with substantial uniformity throughout the blender.
75. The method of any one of embodiments 58-74, wherein a dose-to-dose niraparib concentration variation in the one or more capsules is less than 50%.
76. The method of embodiment 75, wherein the dose-to-dose niraparib concentration variation in the one or more capsules is less than 40%.
77. The method of embodiment 75, wherein the dose-to-dose niraparib concentration variation in the one or more capsules is less than 30%.
78. The method of embodiment 75, wherein the dose-to-dose niraparib concentration variation in the one or more capsules is less than 20%.
79. The method of embodiment 75, wherein the dose-to-dose niraparib concentration variation in the one or more capsules is less than 10%.
80. The method of embodiment 75, wherein the dose-to-dose niraparib concentration variation in the one or more capsules is less than 5%.
81. The method of any one of embodiments 75-80, wherein the dose-to-dose niraparib concentration variation is based on 10 consecutive doses.
82. The method of embodiment 81, wherein the dose-to-dose niraparib concentration variation is based on 8 consecutive doses.
83. The method of embodiment 81, wherein the dose-to-dose niraparib concentration variation is based on 5 consecutive doses.
84. The method of embodiment 81, wherein the dose-to-dose niraparib concentration variation is based on 3 consecutive doses.
85. The method of embodiment 81, wherein the dose-to-dose niraparib concentration variation is based on 2 consecutive doses.
86. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate;
   wherein the capsule comprises the composition comprising niraparib, lactose monohydrate and magnesium stearate produced according the method of any one of embodiments 1-85.
87. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate.
88. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate;
   wherein the niraparib has been annealed two or more times.
89. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate;
   wherein the niraparib in the capsule has a Hausner's ratio of less than 1.7.
90. The composition of embodiment 89, wherein the niraparib in the capsule has a Hausner's ratio of about 1.48 or less.
91. The composition of embodiment 89, wherein the niraparib in the capsule has a Hausner's ratio of about 1.38 or less.
92. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate;
   wherein the formulation in the capsule has a Hausner's ratio of about 1.7 or less.
93. The composition of embodiment 92, wherein the formulation in the capsule has a Hausner's ratio of about 1.64 or less.
94. The composition of embodiment 92, wherein the formulation in the capsule has a Hausner's ratio of about 1.52 or less.
95. The composition of embodiment 92, wherein the formulation in the capsule has a Hausner's ratio of about 1.47 or less.
96. The composition of embodiment 92, wherein the formulation in the capsule has a Hausner's ratio of about 1.43 or less.
   The composition of embodiment 92, wherein the formulation in the capsule has a Hausner's ratio of about 1.41 or less.
97. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate;
   wherein
      (i) the niraparib in the capsule has an internal friction angle of 33.1 degrees or higher,
      (ii) the formulation in the capsule has an internal friction angle of less than 34 degrees,
      (iii) the niraparib in the capsule has flow function ratio value of more than 6.4, (iv) the formulation in the capsule has a flow function ratio value of more than 14.4,
      (v) the niraparib in the capsule has a wall friction angle of less than 29 at an Ra of 0.05,
      (vi) the formulation in the capsule has a wall friction angle of less than 15 degrees at an Ra of 0.05, and/or
      (vii) the formulation in the capsule has a wall friction angle of less than 26 degrees at an Ra of 1.2.
98. A composition comprising a capsule comprising a formulation comprising
   (a) an effective amount of niraparib to inhibit polyadenosine diphosphate ribose polymerase (PARP) when administered to a human,
   (b) lactose monohydrate, and
   (c) magnesium stearate;
   wherein the lactose monohydrate in the capsule has (i) a bulk density of about 0.2-0.8 mg/cm³ and/or (ii) a tapped density of about 0.3-0.9 mg/cm³.
99. A composition comprising a capsule comprising a formulation comprising
   (a) an effective amount of niraparib to inhibit polyadenosine diphosphate ribose polymerase (PARP) when administered to a human,
   (b) lactose monohydrate particles, and
   (c) magnesium stearate;
   wherein 50% or more of the lactose monohydrate particles in the capsule has a diameter of at least about 53 microns to about 500 microns, and/or 50% or more of the lactose monohydrate particles in the capsule has a diameter of at most about 250 microns.
100. The composition of any one of embodiments 86-99, wherein the composition is stable with respect to niraparib degradation after storage for 1 month, 3 months, 6 months, 9 months, 12 months, 24 months, or 36 months at 5° C.
101. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for 1 month, 3 months, 6 months, 9 months, 12 months, 24 months, or 36 months at 5° C.
102. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for 1 month, 3 months, 6 months, 9 months, 12 months, 24 months, or 36 months at 25° C. and 60% relative humidity (RH).
103. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for 1 month, 3 months, 6 months, 9 months, 12 months, 24 months, or 36 months at 30° C. and 65% relative humidity (RH).
104. The composition of embodiment 100, wherein the wherein composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of one or more niraparib degradation products after storage for 1 month, 3 months, 6 months, 9 months, 12 months, 24 months, or 36 months at 40° C. and 75% relative humidity (RH)
105. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for 1 month, 3 months, 6 months, 9 months, 12 months, 24 months, or 36 months at 5° C.
106. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for 1 month, 3 months, 6 months, 9 months, 12 months, 24 months, or 36 months at 25° C. and 60% relative humidity (RH).
107. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for 1 month, 3 months, 6 months, 9 months, 12 months, 24 months, or 36 months at 30° C. and 65% relative humidity (RH).
108. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of impurity after storage for 1 month, 3 months, 6 months, 9 months, 12 months, 24 months, or 36 months at 40° C. and 75% relative humidity (RH).
109. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for 1 month, 3 months, 6 months, 9 months, 12 months, 24 months, or 36 months at 5° C.
110. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for 1 month, 3 months, 6 months, 9 months, 12 months, 24 months, or 36 months at 25° C. and 60% relative humidity (RH).
111. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for 1 month, 3 months, 6 months, 9 months, 12 months, 24 months, or 36 months at 30° C. and 65% relative humidity (RH).
112. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01% 0.005%, or 0.001% by weight of any single unspecified niraparib degradation product after storage for 1 month, 3 months, 6 months, 9 months, 12 months, 24 months, or 36 months at 40° C. and 75% relative humidity (RH).
113. The composition of embodiment 100, wherein the composition comprises less than 3.0%, 2.5%, 2.0%, 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for 1 month, 3 months, 6 months, 9 months, 12 months, 24 months, or 36 months at 5° C.
114. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for 1 month, 3 months, 6 months, 9 months, 12 months, 24 months, or 36 months wherein composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for 1 month, 3 months, 6 months, 9 months, 12 months, 24 months, or 36 months at 30° C. and 65% relative humidity (RH).
115. The composition of embodiment 100, wherein the composition comprises less than 1.5%, 1.4%, 1.3%, 1.2% 1.1%, 1.0%, 0.9%, 0.8%, 0.7%, 0.6%, 0.5%, 0.4%, 0.3%, 0.2%, 0.1%, 0.05%, 0.025%, or 0.001% by weight of total niraparib degradation products after storage for 1 month, 3 months, 6 months, 9 months, 12 months, 24 months, or 36 months at 40° C. and 70% relative humidity (RH).
116. The composition of any one of embodiments 86-115, wherein the composition has an absolute bioavailability of niraparib of about 60 to about 90%.
117. The composition of any one of embodiments 86-116, wherein not less than 30%, 35%, 40%, 45%, 55%, 60%, 65% 70%, 75%, 80%, 85%, 90%, 95%, or 100% of the niraparib dissolves in 5, 10, 15, 20, 30, 45, 60, 90, or 120 minutes under dissolution evaluation.
118. The composition of embodiment 117 or 118, wherein not less than 30%, 35%, 40%, 45%, 55%, 60%, 65% 70%, 75%, 80%, 85%, 90%, 95%, or 100% of the niraparib dissolves in 5, 10, 15, 20, 30, 45, 60, 90, or 120 minutes under dissolution evaluation after storage of the composition for 1 month, 3 months, 6 months, 9 months, 12 months, 24 months, or 36 months at 25° C. and 60% relative humidity (RH).
119. The composition of any one of embodiments 86-118, wherein the composition comprises two or more capsules each comprising the formulation.
120. The composition of embodiment 119, wherein the two or more capsules comprises at least about 100, 500, 1,000, 5,000, 6,000, 7,000, 8,000, 9,000, 10,000, 11,000, 12,000, 13,000, 124,000, 15,000, 16,000, 17,000, 18,000, 19,000, 20,000, 21,000, 22,000, 23,000, 24,000, 25,000, 50,000, 100,000, 150,000, 200,000, 300,000, 400,000, or 500,000 capsules.
121. A method of treating cancer, comprising administering to a subject in need thereof an effective amount of a composition according to any one of embodiments 86-120.
122. The method of embodiment 121, wherein the composition is administered in doses having a dose-to-dose niraparib concentration variation of less than 50%, less than 40%, less than 30%, less than 20%, less than 10%, or less than 5%.
123. The method of embodiment 121 or 122, wherein the cancer is selected from the group consisting of adenocarcinoma, endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anus, squamous cell carcinoma of the penis, squamous cell carcinoma of the cervix, squamous cell carcinoma of the vagina, squamous cell carcinoma of the vulva, soft tissue sarcoma, melanoma, renal cell carcinoma, lung cancer, non-small cell lung cancer, adenocarcinoma of the lung, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, a hematological cancer, multiple myeloma, B-cell lymphoma, T-cell lymphoma, Hodgkin's lymphoma/primary mediastinal B-cell lymphoma, chronic myelogenous leukemia, acute myeloid leukemia, acute lymphoblastic leukemia, non-Hodgkin's lymphoma, neuroblastoma, a CNS tumor, diffuse intrinsic pontine glioma (DIPG), Ewing's sarcoma, embryonal rhabdomyosarcoma, osteosarcoma, or Wilms tumor, and combinations thereof.
124. The method of any one of embodiments 121-123, wherein the cancer is selected from the group consisting of ovarian cancer, fallopian tube cancer, primary peritoneal cancer, and combinations thereof.
125. The method of any one of embodiments 121-124, wherein the cancer is a recurrent cancer.
126. The method of any one of embodiments 121-125, wherein the subject is a human subject.
127. The method of embodiment 126, wherein the human subject was previously treated with a chemotherapy.
128. The method of embodiment 127, wherein the a chemotherapy is a platinum-based chemotherapy.
129. The method of embodiment 127 or 128, wherein the human subject had a complete or partial response to the chemotherapy.
130. The method of any one of embodiments 121-129, wherein the subject has a mean peak plasma concentration (Cₘₐₓ) of 600 ng/mL to 1000 ng/mL of the niraparib.
131. The method of embodiment 130, wherein the subject has the mean peak plasma concentration (Cₘₐₓ) within 0.5 to 6 hours after the administering.
132. The method of any one of embodiments 121-131, wherein about 60%, 65%, 70%, 75%, 80%, 85% or 90% of the niraparib is bound to human plasma protein of the subject after the administering.
133. The method of any one of embodiments 121-132, wherein an apparent volume of distribution (Vd/F) of the niraparib is from about 500 L to about 2000 L after administration to a human subject.
134. The method of any one of embodiments 121-133, wherein the niraparib has a mean terminal half-life (tin) of from about 30 to about 60 hours after the administering.
135. The method of any one of embodiments 121-134, wherein the niraparib has an apparent total clearance (CL/F) of from about 10 L/hour to about 20 L/hour after the administering.
136. The method of any one of embodiments 121-135, wherein at least about 60%, 65%, 70%, 75%, 80%, 85%, 90%, 95%, 96%, 97%, 98%, 99%, or 100% of the niraparib is released from the composition within 1 minute, or within 5 minutes, or within 10 minutes, or within 15 minutes, or within 30 minutes, or within 60 minutes or within 90 minutes after the administering.
137. The method of any one of embodiments 121-136, wherein the subject has a Cₘᵢₙ niraparib blood plasma level at steady state of from about 10 ng/ml to about 100 ng/ml after the administering.
138. The method of any one of embodiments 121-137, wherein at least about 70%, 80%, 90%, or 95% of the niraparib is absorbed into the bloodstream of the subject within 1, 2, 3, 4, 5, 6, 7, 8, 9, 10, 11, 12, 16, 18, or 24 hours after administering.
139. The method of any one of embodiments 121-138, wherein said subject is a pediatric subject.
140. A method of treating cancer, comprising administering to a pediatric subject in need thereof an effective amount of niraparib.
141. The method of any one of embodiments 121-140, wherein said cancer is characterized by a homologous recombination repair (HRR) gene deletion.
142. The method of any one of embodiments 121-141, wherein said cancer is characterized by a mutation in the DNA damage repair (DDR) pathway.
143. The method of any one of embodiments 121-143, wherein said cancer is characterized by homologous recombination deficiency (HRD).
144. The method of any one of embodiments 121-144, wherein said cancer is characterized by BRCA deficiency.
145. The method of any one of embodiments 121-141, wherein said cancer is characterized by an isocitrate dehydrogenase (IDH) mutation.
146. The method of any one of embodiments 121-142, wherein said cancer is characterized by a chromosomal translocation.
147. The method of any one of embodiments 121-146, wherein said cancer is a hypermutant cancer.
148. The method of any one of embodiments 121-147, wherein said cancer is a MSI-H or a MSI-L cancer.
149. The method of any one of embodiments 121-147, wherein said cancer is a MSS cancer.
150. The method of any one of embodiments 121-149, wherein said cancer is a non-CNS cancer.
151. The method of embodiment 150, wherein said cancer is a solid tumor.
152. The method of embodiment 150 or 151, wherein said cancer is neuroblastoma, hepatoblastoma, hepatocellular carcinoma, Wilms tumor, renal cell carcinoma, melanoma, adrenocortical carcinoma, adenocarcinoma of the colon, myoepithelial carcinoma, thymic cell carcinoma, nasopharyngeal carcinoma, squamous cell carcinoma, mesothelioma, clivus chordoma.
153. The method of embodiment 152, wherein said cancer is extracranial embryonal neuroblastoma.
154. The method of any one of embodiments 121-149, wherein said cancer is a CNS cancer.
155. The method of embodiment 154, wherein said cancer is a primary CNS malignancy.
156. The method of embodiment 155, wherein said cancer is ependymoma.
157. The method of embodiment 154, wherein said cancer is a brain cancer.
158. The method of embodiment 157, wherein said cancer is glioblastoma multiforme, gliosarcoma, astrocytoma, glioblastoma, medulloblastoma, glioma, supratentorial primitive neuroectodermal tumor, atypical teratoid rhabdoid tumor, choroid plexus carcinoma, malignant ganglioma, gliomatosis cerebri, meningioma, or paraganglioma.
159. The method of embodiment 158, wherein said cancer is high-grade astrocytoma, low-grade astrocytoma, anaplastic astrocytoma, fibrillary astrocytoma, pilocytic astrocytoma.
160. The method of embodiment 158, wherein said cancer is a high-grade glioma, low-grade glioma, diffuse intrinsic pontine glioma (DIPG), anaplastic mixed glioma.
161. The method of any one of embodiments 121-149, wherein said cancer is a carcinoma.
162. The method of any one of embodiments 121-149, wherein said cancer is a gonadal tumor.
163. The method of any one of embodiments 121-149, wherein said cancer is a hematological cancer.
164. The method of embodiment 163, wherein said cancer is lymphoma.
165. The method of embodiment 164, wherein said cancer is Hodgkin's lymphoma (e.g., relapsed or refractory classic Hodgkin's Lymphoma (cHL)), non-Hodgkin's lymphoma, diffuse large B-cell lymphoma, precursor T-lymphoblastic lymphoma, lymphoepithelial carcinoma, or malignant histiocytosis.
166. The method of any one of embodiments 121-149, wherein said cancer is a sarcoma.
167. The method of embodiment 166, wherein said cancer is Ewings sarcoma, osteosarcoma, rhabdomyosarcoma, embryonal rhabdomyosarcoma, synovial sarcoma, alveolar rhabdomyosarcoma, alveolar soft part sarcoma, spindle cell sarcoma, angiosarcoma, epithelialoid sarcoma, inflammatory myofibroblastic tumor, malignant rhadoid tumor
168. The method of any one of embodiments 121-149, wherein said cancer is Ewing's sarcoma, osteosarcoma, ERS, a CNS tumor, or neuroblastoma.
169. The method of any one of embodiments 121-168, wherein said cancer is recurrent.
170. The method of any one of embodiments 121-169, wherein said subject has not received at least one other line of treatment (LOT).
171. The method of any one of embodiments 121-169, wherein said subject has previously received at least one line of treatment (LOT).
172. The method of embodiment 171, wherein said at least one line of treatment is not an immunotherapy treatment
173. The method of embodiment 171 or 172, wherein said cancer is refractory to a previous line of treatment (LOT).
174. The method of any one of embodiments 139-173, wherein the pediatric patient is of about six months to about 18 years of age, about one year to about six years of age, or about six years to about 18 years of age.
175. The method of any one of embodiments 139-174, wherein the administered amount of niraparib is determined by said subject's weight.
176. The method of any one of embodiments 139-175, wherein the administered amount of niraparib is determined by said subject's body surface area (B S A).
177. The method of embodiment 176, wherein the administered amount of niraparib is about 25 mg/m² to about 300 mg/m², about 25 mg/m² to about 275 mg/m², about 25 mg/m² to about 250 mg/m², about 25 mg/m² to about 200 mg/m², about 50 mg/m² to about 300 mg/m², about 50 mg/m² to about 275 mg/m², about 50 mg/m² to about 250 mg/m², about 50 mg/m² to about 200 mg/m², about 75 mg/m² to about 300 mg/m², about 75 mg/m² to about 275 mg/m², about 75 mg/m² to about 250 mg/m², about 75 mg/m² to about 200 mg/m², about 100 mg/m² to about 300 mg/m², about 100 mg/m² to about 275 mg/m², about 100 mg/m² to about 250 mg/m², about 100 mg/m² to about 200 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 105 mg/m², about 110 mg/m², about 115 mg/m², about 120 mg/m², about 125 mg/m², about 130 mg/m², about 135 mg/m², about 140 mg/m², about 145 mg/m², about 150 mg/m², about 155 mg/m², about 160 mg/m², about 165 mg/m², about 170 mg/m², about 175 mg/m², about 180 mg/m², about 185 mg/m², about 190 mg/m², about 195 mg/m², or about 200 mg/m².
178. The method of any one of embodiments 139-175, wherein the administered amount of niraparib is a flat dose.
179. The method of any one of embodiments 139-178, wherein niraparib is orally administered once daily.
180. The method of any one of embodiments 139-178, wherein niraparib is orally administered once every two days, once every three days, once every four days, once every five days, once every six days, or once every seven days.
181. The method of any one of embodiments 139-180, wherein niraparib is orally administered in an amount that is about 25 mg to about 300 mg or about 25 mg to about 500 mg.
182. The method of embodiment 180, wherein said niraparib is orally administered in an amount that is:
   about 25 mg, about 50 mg, about 75 mg, about 100 mg, about 125 mg, about 150 mg, about 175 mg, or about 200 mg;
   about 75 mg, about 100 mg, about 130 mg, or about 160 mg;
   about 150 mg, about 200 mg, about 260 mg, or about 320 mg; or
   about 225 mg, about 300 mg, about 390 mg, or about 480 mg.
183. The method of any one of embodiments 139-182, wherein two different amounts of niraparib are administered to the subject on alternating days on which dosages are administered to said subject.
184. The method of any one of embodiments 139-183, wherein said niraparib is administered as a unit dose form that is a capsule comprising about 50 mg niraparib.
185. The method of any one of embodiments 121-184, wherein the method further comprises administering another therapeutic agent or treatment.
186. The method of embodiment 185, wherein the method further comprises administering one or more of surgery, a radiotherapy, a chemotherapy, an immunotherapy, an anti-angiogenic agent, or an anti-inflammatory.
187. The method of embodiment 185 or 186, wherein the subject has been further administered or will be administered an immune checkpoint inhibitor.
188. The method of embodiment 187, wherein the immune checkpoint inhibitor is selected from an inhibitor of PD-1, LAG-3, CTLA-4, TIM-3, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R.
189. The method of embodiment 188, wherein the immune checkpoint inhibitor is an agent that inhibits PD-1, LAG-3, TIM-3, CTLA-4, TIGIT, IDO, or CSF1R.
190. The method of embodiment 189, wherein the immune checkpoint inhibitor is an agent that inhibits PD-1.
191. The method of embodiment 190, wherein the PD-1 inhibitor is a small molecule, a nucleic acid, a polypeptide, a carbohydrate, a lipid, a metal, a toxin, or a PD-1 binding agent.
192. The method of embodiment 190 or 191, wherein the PD-1 inhibitor is a PD-L1/L2 binding agent.
193. The method of embodiment 192, wherein the PD-L1/L2 binding agent is an antibody, an antibody conjugate, or an antigen-binding fragment thereof
194. The method of embodiment 193, wherein the PD-L1/L2 binding agent durvalumab, atezolizumab, avelumab, BGB-A333, SHR-1316, FAZ-053, CK-301, or, PD-L1 millamolecule, or derivatives thereof.
195. The method of embodiment 190 or 191, wherein the PD-1 inhibitor is a PD-1 binding agent.
196. The method of embodiment 195, wherein the PD-1 binding agent is an antibody, an antibody conjugate, or an antigen-binding fragment thereof.
197. The method of embodiment 196, wherein the PD-1 inhibitor is nivolumab, pembrolizumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, AMP-514/MEDI-0680, AGEN-2034, CS1001, TSR-042, Sym-021, PF-06801591, LZMO09, KN-035, AB122, genolimzumab (CBT-501), AK 104, or GLS-010, or derivatives thereof.
198. The method of embodiment 197, wherein the PD-1 inhibitor is TSR-042.
199. The method of any one of embodiments 190-198, wherein the PD-1 inhibitor is administered to the subject periodically at a dose of about 50 mg to about 2000 mg, about 50 mg to about 1000 mg, or about 100 mg to about 500 mg.
200. The method of embodiment 199, wherein the PD-1 inhibitor is administered to the subject periodically at a dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, or about 1700 mg.
201. The method of embodiment 199 or 200, wherein the PD-1 inhibitor is administered to the subject periodically at an administration interval that is once every week, once every two weeks, once every three weeks, once every four weeks, once every five weeks, once every six weeks, once every seven weeks, once every eight weeks, once every nine weeks, or once every ten weeks.
202. The method of embodiment 199 or 200, wherein the PD-1 inhibitor is administered as a first dose once every 3 weeks for 3, 4, or 5 cycles followed by a second dose administered once every six weeks.
203. The method of embodiment 202, wherein the first dose is about 500 mg of the PD-1 inhibitor.
204. The method of embodiment 202 or 203, wherein the second dose is about 1000 mg of the PD-1 inhibitor.
205. The method of any one of embodiments 139-204, wherein niraparib is administered with food.

1. A method of treating cancer, comprising administering to a pediatric human in need thereof an effective amount of niraparib, or a pharmaceutically acceptable salt thereof.

2. The method of claim 0, wherein the pediatric human is about six months of age to about 21 years of age.

3.-5. (canceled)

6. The method of claim 1, wherein the method further comprises administering another therapeutic agent or treatment.

7. The method of claim 6, wherein the method further comprises administering one or more of surgery, a radiotherapy, a chemotherapy, an immunotherapy, an anti-angiogenic agent, or an anti-inflammatory

8. The method of claim 6, wherein the human has been further administered or will be administered an immune checkpoint inhibitor.

9. The method of claim 8, wherein the immune checkpoint inhibitor is selected from an inhibitor of PD-1, LAG-3, CTLA-4, TIM-3, TIGIT, CEACAM, VISTA, BTLA, LAIR1, CD160, 2B4, CD80, CD86, B7-H3 (CD276), B7-H4 (VTCN1), HVEM, KIR, A2aR, MHC class I, MHC class II, GALS, adenosine, TGFR, B7-H1, B7-H4 (VTCN1), OX-40, CD137, CD40, IDO, or CSF1R.

10. (canceled)

11. The method of claim 9, wherein the immune checkpoint inhibitor is an agent that inhibits PD-1.

12. (canceled)

13. The method of claim 11, wherein the PD-1 inhibitor is a PD-L1/L2 binding agent.

14. (canceled)

15. The method of claim 13, wherein the PD-L1/L2 binding agent durvalumab, atezolizumab, avelumab, BGB-A333, SHR-1316, FAZ-053, CK-301, or, PD-L1 millamolecule, or derivatives thereof.

16. The method of claim 11, wherein the PD-1 inhibitor is a PD-1 binding agent.

17. (canceled)

18. The method of claim 16, wherein the PD-1 inhibitor is nivolumab, pembrolizumab, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, AMP-514/MEDI-0680, AGEN-2034, CS1001, TSR-042, Sym-021, PF-06801591, LZMO09, KN-035, AB122, genolimzumab (CBT-501), AK 104, or GLS-010, or derivatives thereof.

19. The method of claim 18, wherein the PD-1 inhibitor is TSR-042.

20. The method of claim 11, wherein the PD-1 inhibitor is administered to the human subject periodically at a dose of about 0.5 mg/kg to about 10 mg/kg.

21.-24. (canceled)

25. The method of claim 20, wherein the PD-1 inhibitor is administered to the human periodically at an administration interval that is once every week, once every two weeks, once every three weeks, once every four weeks, once every five weeks, once every six weeks, once every seven weeks, once every eight weeks, once every nine weeks, or once every ten weeks.

26.-30. (canceled)

31. The method of claim 1, wherein said cancer is characterized by a mutation in the DNA damage repair (DDR) pathway.

32. The method of claim 1, wherein said cancer is characterized by homologous recombination deficiency (HRD).

33. The method of claim 1, wherein said cancer is characterized by BRCA deficiency.

34.-38. (canceled)

39. The method of claim 1, wherein said cancer is a solid tumor.

40.-54. (canceled)

55. The method of claim 1, wherein said cancer is a sarcoma.

56. The method of claim 55, wherein said cancer is Ewings sarcoma, osteosarcoma, rhabdomyosarcoma, embryonal rhabdomyosarcoma (ERS), synovial sarcoma, alveolar rhabdomyosarcoma, alveolar soft part sarcoma, spindle cell sarcoma, angiosarcoma, epithelialoid sarcoma, inflammatory myofibroblastic tumor, malignant rhadoid tumor.

57. (canceled)

58. The method of claim 1, wherein said cancer is characterized by BRCA deficiency, high tumor mutation burden (TMB), and/or increased PD-L1 expression.

59. (canceled)

60. The method of claim 1, wherein said cancer is recurrent.

61. (canceled)

62. The method of claim 1, wherein said human has previously received at least one line of treatment (LOT).

63. (canceled)

64. The method of claim 62, wherein a previous line of treatment is chemotherapy.

65. The method of claim 62, wherein a previous line of treatment is radiation therapy.

66. The method of claim 62, wherein a previous line of treatment is surgery.

67.-71. (canceled)

72. The method of claim 1, wherein niraparib, or a pharmaceutically acceptable salt thereof, is orally administered once daily.

73.-76. (canceled)

77. The method of claim 1, wherein said niraparib is orally administered in an amount that is about 100 mg or about 200 mg of niraparib free base.

78.-79. (canceled)

80. The method of claim 1, wherein said niraparib, or pharmaceutically acceptable salt thereof, is administered as a unit dose form that is a capsule.

81.-84. (canceled)

85. The method of claim 80, wherein the contents of the capsule are sprinkled onto food or administered via a feeding tube.

86. (canceled)

87. The method of claim 1, wherein said niraparib, or a pharmaceutically acceptable salt thereof, is administered as a unit dose form that is a tablet.

88.-101. (canceled)

102. The method of claim 1, wherein niraparib is administered as niraparib tosylate monohydrate.

103.-105. (canceled)
